SEQUENCE LISTING (43 sequences)

SEQ ID NO: 1 - protein, 346 aa, Homo sapiens
  REGION 1..346: note = MISC_FEATURE - NP_000691.1 annexin A1
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 2 - protein, 357 aa, Homo sapiens
  REGION 1..357: note = MISC_FEATURE - NP_001002858.1 annexin A2 isoform 1
MGRQLAGCGDAGKKASFKMSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 3 - protein, 339 aa, Homo sapiens
  REGION 1..339: note = MISC_FEATURE - NP_001129487.1 annexin A2 isoform 2
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 4 - protein, 323 aa, Homo sapiens
  REGION 1..323: note = MISC_FEATURE - NP_005130.1 annexin A3
MASIWVGHRGTVRDYPDFSPSVDAEAIQKAIRGIGTDEKMLISILTERSNAQRQLIVKEYQAAYGKELKDDLKGDLSGHFEHLMVALVTPPAVFDAKQLKKSMKGAGTNEDALIEILTTRTSRQMKDISQAYYTVYKKSLGDDISSETSGDFRKALLTLADGRRDESLKVDEHLAKQDAQILYKAGENRWGTDEDKFTEILCLRSFPQLKLTFDEYRNISQKDIVDSIKGELSGHFEDLLLAIVNCVRNTPAFLAERLHRALKGIGTDEFTLNRIMVSRSEIDLLDIRTEFKKHYGYSLYSAIKSDTSGDYEITLLKICGGDD

SEQ ID NO: 5 - protein, 321 aa, Homo sapiens
  REGION 1..321: note = MISC_FEATURE - NP_001144.1 annexin A4 isoform a
MAMATKGGTVKAASGFNAMEDAQTLRKAMKGLGTDEDAIISVLAYRNTAQRQEIRTAYKSTIGRDLIDDLKSELSGNFEQVIVGMMTPTVLYDVQELRRAMKGAGTDEGCLIEILASRTPEEIRRISQTYQQQYGRSLEDDIRSDTSFMFQRVLVSLSAGGRDEGNYLDDALVRQDAQDLYEAGEKKWGTDEVKFLTVLCSRNRNHLLHVFDEYKRISQKDIEQSIKSETSGSFEDALLAIVKCMRNKSAYFAEKLYKSMKGLGTDDNTLIRVMVSRAEIDMLDIRAHFKRLYGKSLYSFIKGDTSGDYRKVLLVLCGGDD

SEQ ID NO: 6 - protein, 320 aa, Homo sapiens
  REGION 1..320: note = MISC_FEATURE - NP_001145.1 annexin A5
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 7 - protein, 673 aa, Homo sapiens
  REGION 1..673: note = MISC_FEATURE - NP_001146.2 annexin A6 isoform 1
MAKPAQGAKYRGSIHDFPGFDPNQDAEALYTAMKGFGSDKEAILDIITSRSNRQRQEVCQSYKSLYGKDLIADLKYELTGKFERLIVGLMRPPAYCDAKEIKDAISGIGTDEKCLIEILASRTNEQMHQLVAAYKDAYERDLEADIIGDTSGHFQKMLVVLLQGTREEDDVVSEDLVQQDVQDLYEAGELKWGTDEAQFIYILGNRSKQHLRLVFDEYLKTTGKPIEASIRGELSGDFEKLMLAVVKCIRSTPEYFAERLFKAMKGLGTRDNTLIRIMVSRSELDMLDIREIFRTKYEKSLYSMIKNDTSGEYKKTLLKLSGGDDDAAGQFFPEAAQVAYQMWELSAVARVELKGTVRPANDFNPDADAKALRKAMKGLGTDEDTIIDIITHRSNVQRQQIRQTFKSHFGRDLMTDLKSEISGDLARLILGLMMPPAHYDAKQLKKAMEGAGTDEKALIEILATRTNAEIRAINEAYKEDYHKSLEDALSSDTSGHFRRILISLATGHREEGGENLDQAREDAQVAAEILEIADTPSGDKTSLETRFMTILCTRSYPHLRRVFQEFIKMTNYDVEHTIKKEMSGDVRDAFVAIVQSVKNKPLFFADKLYKSMKGAGTDEKTLTRIMVSRSEIDLLNIRREFIEKYDKSLHQAIEGDTSGDFLKALLALCGGED

SEQ ID NO: 8 - protein, 641 aa, Homo sapiens
  REGION 1..641: note = MISC_FEATURE - NP_001180473.1 annexin A6 isoform 2
MKGFGSDKEAILDIITSRSNRQRQEVCQSYKSLYGKDLIADLKYELTGKFERLIVGLMRPPAYCDAKEIKDAISGIGTDEKCLIEILASRTNEQMHQLVAAYKDAYERDLEADIIGDTSGHFQKMLVVLLQGTREEDDVVSEDLVQQDVQDLYEAGELKWGTDEAQFIYILGNRSKQHLRLVFDEYLKTTGKPIEASIRGELSGDFEKLMLAVVKCIRSTPEYFAERLFKAMKGLGTRDNTLIRIMVSRSELDMLDIREIFRTKYEKSLYSMIKNDTSGEYKKTLLKLSGGDDDAAGQFFPEAAQVAYQMWELSAVARVELKGTVRPANDFNPDADAKALRKAMKGLGTDEDTIIDIITHRSNVQRQQIRQTFKSHFGRDLMTDLKSEISGDLARLILGLMMPPAHYDAKQLKKAMEGAGTDEKALIEILATRTNAEIRAINEAYKEDYHKSLEDALSSDTSGHFRRILISLATGHREEGGENLDQAREDAQVAAEILEIADTPSGDKTSLETRFMTILCTRSYPHLRRVFQEFIKMTNYDVEHTIKKEMSGDVRDAFVAIVQSVKNKPLFFADKLYKSMKGAGTDEKTLTRIMVSRSEIDLLNIRREFIEKYDKSLHQAIEGDTSGDFLKALLALCGGED

SEQ ID NO: 9 - protein, 466 aa, Homo sapiens
  REGION 1..466: note = MISC_FEATURE - NP_001147.1 annexin A7 isoform 1
MSYPGYPPTGYPPFPGYPPAGQESSFPPSGQYPYPSGFPPMGGGAYPQVPSSGYPGAGGYPAPGGYPAPGGYPGAPQPGGAPSYPGVPPGQGFGVPPGGAGFSGYPQPPSQSYGGGPAQVPLPGGFPGGQMPSQYPGGQPTYPSQPATVTQVTQGTIRPAANFDAIRDAEILRKAMKGFGTDEQAIVDVVANRSNDQRQKIKAAFKTSYGKDLIKDLKSELSGNMEELILALFMPPTYYDAWSLRKAMQGAGTQERVLIEILCTRTNQEIREIVRCYQSEFGRDLEKDIRSDTSGHFERLLVSMCQGNRDENQSINHQMAQEDAQRLYQAGEGRLGTDESCFNMILATRSFPQLRATMEAYSRMANRDLLSSVSREFSGYVESGLKTILQCALNRPAFFAERLYYAMKGAGTDDSTLVRIVVTRSEIDLVQIKQMFAQMYQKTLGTMIAGDTSGDYRRLLLAIVGQ

SEQ ID NO: 10 - protein, 488 aa, Homo sapiens
  REGION 1..488: note = MISC_FEATURE - NP_004025.1 annexin A7 isoform 2
MSYPGYPPTGYPPFPGYPPAGQESSFPPSGQYPYPSGFPPMGGGAYPQVPSSGYPGAGGYPAPGGYPAPGGYPGAPQPGGAPSYPGVPPGQGFGVPPGGAGFSGYPQPPSQSYGGGPAQVPLPGGFPGGQMPSQYPGGQPTYPSQINTDSFSSYPVFSPVSLDYSSEPATVTQVTQGTIRPAANFDAIRDAEILRKAMKGFGTDEQAIVDVVANRSNDQRQKIKAAFKTSYGKDLIKDLKSELSGNMEELILALFMPPTYYDAWSLRKAMQGAGTQERVLIEILCTRTNQEIREIVRCYQSEFGRDLEKDIRSDTSGHFERLLVSMCQGNRDENQSINHQMAQEDAQRLYQAGEGRLGTDESCFNMILATRSFPQLRATMEAYSRMANRDLLSSVSREFSGYVESGLKTILQCALNRPAFFAERLYYAMKGAGTDDSTLVRIVVTRSEIDLVQIKQMFAQMYQKTLGTMIAGDTSGDYRRLLLAIVGQ

SEQ ID NO: 11 - protein, 365 aa, Homo sapiens
  REGION 1..365: note = MISC_FEATURE - NP_001258631.1 annexin A8 isoform 1
MAWWKSWIEQEGVTVKSSSHFNPDPDAETLYKAMKGIGVGSQLLSHQAAAFAFPSSALTSVSPWGQQGHLCCNPAGTNEQAIIDVLTKRSNTQRQQIAKSFKAQFGKDLTETLKSELSGKFERLIVALMYPPYRYEAKELHDAMKGLGTKEGVIIEILASRTKNQLREIMKAYEEDYGSSLEEDIQADTSGYLERILVCLLQGSRDDVSSFVDPGLALQDAQDLYAAGEKIRGTDEMKFITILCTRSATHLLRVFEEYEKIANKSIEDSIKSETHGSLEEAMLTVVKCTQNLHSYFAERLYYAMKGAGTRDGTLIRNIVSRSEIDLNLIKCHFKKMYGKTLSSMIMEDTSGDYKNALLSLVGSDP

SEQ ID NO: 12 - protein, 327 aa, Homo sapiens
  REGION 1..327: note = MISC_FEATURE - NP_001035173.1 annexin A8 isoform 2
MAWWKSWIEQEGVTVKSSSHFNPDPDAETLYKAMKGIGTNEQAIIDVLTKRSNTQRQQIAKSFKAQFGKDLTETLKSELSGKFERLIVALMYPPYRYEAKELHDAMKGLGTKEGVIIEILASRTKNQLREIMKAYEEDYGSSLEEDIQADTSGYLERILVCLLQGSRDDVSSFVDPGLALQDAQDLYAAGEKIRGTDEMKFITILCTRSATHLLRVFEEYEKIANKSIEDSIKSETHGSLEEAMLTVVKCTQNLHSYFAERLYYAMKGAGTRDGTLIRNIVSRSEIDLNLIKCHFKKMYGKTLSSMIMEDTSGDYKNALLSLVGSDP

SEQ ID NO: 13 - protein, 345 aa, Homo sapiens
  REGION 1..345: note = MISC_FEATURE - NP_003559.2 annexin A9
MSVTGGKMAPSLTQEILSHLGLASKTAAWGTLGTLRTFLNFSVDKDAQRLLRAITGQGVDRSAIVDVLTNRSREQRQLISRNFQERTQQDLMKSLQAALSGNLERIVMALLQPTAQFDAQELRTALKASDSAVDVAIEILATRTPPQLQECLAVYKHNFQVEAVDDITSETSGILQDLLLALAKGGRDSYSGIIDYNLAEQDVQALQRAEGPSREETWVPVFTQRNPEHLIRVFDQYQRSTGQELEEAVQNRFHGDAQVALLGLASVIKNTPLYFADKLHQALQETEPNYQVLIRILISRCETDLLSIRAEFRKKFGKSLYSSLQDAVKGDCQSALLALCRAEDM

SEQ ID NO: 14 - protein, 324 aa, Homo sapiens
  REGION 1..324: note = MISC_FEATURE - NP_009124.2 annexin A10
MFCGDYVQGTIFPAPNFNPIMDAQMLGGALQGFDCDKDMLINILTQRCNAQRMMIAEAYQSMYGRDLIGDMREQLSDHFKDVMAGLMYPPPLYDAHELWHAMKGVGTDENCLIEILASRTNGEIFQMREAYCLQYSNNLQEDIYSETSGHFRDTLMNLVQGTREEGYTDPAMAAQDAMVLWEACQQKTGEHKTMLQMILCNKSYQQLRLVFQEFQNISGQDMVDAINECYDGYFQELLVAIVLCVRDKPAYFAYRLYSAIHDFGFHNKTVIRILIARSEIDLLTIRKRYKERYGKSLFHDIRNFASGHYKKALLAICAGDAEDY

SEQ ID NO: 15 - protein, 505 aa, Homo sapiens
  REGION 1..505: note = MISC_FEATURE - NP_665875.1 annexin A11 isoform 1
MSYPGYPPPPGGYPPAAPGGGPWGGAAYPPPPSMPPIGLDNVATYAGQFNQDYLSGMAANMSGTFGGANMPNLYPGAPGAGYPPVPPGGFGQPPSAQQPVPPYGMYPPPGGNPPSRMPSYPPYPGAPVPGQPMPPPGQQPPGAYPGQPPVTYPGQPPVPLPGQQQPVPSYPGYPGSGTVTPAVPPTQFGSRGTITDAPGFDPLRDAEVLRKAMKGFGTDEQAIIDCLGSRSNKQRQQILLSFKTAYGKDLIKDLKSELSGNFEKTILALMKTPVLFDIYEIKEAIKGVGTDEACLIEILASRSNEHIRELNRAYKAEFKKTLEEAIRSDTSGHFQRLLISLSQGNRDESTNVDMSLAQRDAQELYAAGENRLGTDESKFNAVLCSRSRAHLVAVFNEYQRMTGRDIEKSICREMSGDLEEGMLAVVKCLKNTPAFFAERLNKAMRGAGTKDRTLIRIMVSRSETDLLDIRSEYKRMYGKSLYHDISGDTSGDYRKILLKICGGND

SEQ ID NO: 16 - protein, 472 aa, Homo sapiens
  REGION 1..472: note = MISC_FEATURE - NP_001265338.1 annexin A11 isoform 2
MPPIGLDNVATYAGQFNQDYLSGMAANMSGTFGGANMPNLYPGAPGAGYPPVPPGGFGQPPSAQQPVPPYGMYPPPGGNPPSRMPSYPPYPGAPVPGQPMPPPGQQPPGAYPGQPPVTYPGQPPVPLPGQQQPVPSYPGYPGSGTVTPAVPPTQFGSRGTITDAPGFDPLRDAEVLRKAMKGFGTDEQAIIDCLGSRSNKQRQQILLSFKTAYGKDLIKDLKSELSGNFEKTILALMKTPVLFDIYEIKEAIKGVGTDEACLIEILASRSNEHIRELNRAYKAEFKKTLEEAIRSDTSGHFQRLLISLSQGNRDESTNVDMSLAQRDAQELYAAGENRLGTDESKFNAVLCSRSRAHLVAVFNEYQRMTGRDIEKSICREMSGDLEEGMLAVVKCLKNTPAFFAERLNKAMRGAGTKDRTLIRIMVSRSETDLLDIRSEYKRMYGKSLYHDISGDTSGDYRKILLKICGGND

SEQ ID NO: 17 - protein, 316 aa, Homo sapiens
  REGION 1..316: note = MISC_FEATURE - NP_004297.2 annexin A13 isoform a
MGNRHAKASSPQGFDVDRDAKKLNKACKGMGTNEAAIIEILSGRTSDERQQIKQKYKATYGKELEEVLKSELSGNFEKTALALLDRPSEYAARQLQKAMKGLGTDESVLIEVLCTRTNKEIIAIKEAYQRLFDRSLESDVKGDTSGNLKKILVSLLQANRNEGDDVDKDLAGQDAKDLYDAGEGRWGTDELAFNEVLAKRSYKQLRATFQAYQILIGKDIEEAIEEETSGDLQKAYLTLVRCAQDCEDYFAERLYKSMKGAGTDEETLIRIVVTRAEVDLQGIKAKFQEKYQKSLSDMVRSDTSGDFRKLLVALLH

SEQ ID NO: 18 - protein, 358 aa, Homo sapiens
  REGION 1..358: note = MISC_FEATURE - NP_001003954.1 annexin A13 isoform b
IMGNRHSQSYTLSEGSQQLPKGDSQPSTVVQPLSHPSRNGEPEAPQPAKASSPQGFDVDRDAKKLNKACKGMGTNEAAIIEILSGRTSDERQQIKQKYKATYGKELEEVLKSELSGNFEKTALALLDRPSEYAARQLQKAMKGLGTDESVLIEVLCTRTNKEIIAIKEAYQRLFDRSLESDVKGDTSGNLKKILVSLLQANRNEGDDVDKDLAGQDAKDLYDAGEGRWGTDELAFNEVLAKRSYKQLRATFQAYQILIGKDIEEAIEEETSGDLQKAYLTLVRCAQDCEDYFAERLYKSMKGAGTDEETLIRIVVTRAEVDLQGIKAKFQEKYQKSLSDMVRSDTSGDFRKLLVALLH

SEQ ID NO: 19 - DNA, 1401 bp, Homo sapiens
  misc_feature 1..1401: note = NM_000700.3 Homo sapiens annexin A1 (ANXA1), mRNA
  source 1..1401: mol_type = other DNA
agtgtgaaatcttcagagaagaatttctctttagttctttgcaagaaggtagagataaagacactttttcaaaaatggcaatggtatcagaattcctcaagcaggcctggtttattgaaaatgaagagcaggaatatgttcaaactgtgaagtcatccaaaggtggtcccggatcagcggtgagcccctatcctaccttcaatccatcctcggatgtcgctgccttgcataaggccataatggttaaaggtgtggatgaagcaaccatcattgacattctaactaagcgaaacaatgcacagcgtcaacagatcaaagcagcatatctccaggaaacaggaaagcccctggatgaaacactgaagaaagcccttacaggtcaccttgaggaggttgttttagctctgctaaaaactccagcgcaatttgatgctgatgaacttcgtgctgccatgaagggccttggaactgatgaagatactctaattgagattttggcatcaagaactaacaaagaaatcagagacattaacagggtctacagagaggaactgaagagagatctggccaaagacataacctcagacacatctggagattttcggaacgctttgctttctcttgctaagggtgaccgatctgaggactttggtgtgaatgaagacttggctgattcagatgccagggccttgtatgaagcaggagaaaggagaaaggggacagacgtaaacgtgttcaataccatccttaccaccagaagctatccacaacttcgcagagtgtttcagaaatacaccaagtacagtaagcatgacatgaacaaagttctggacctggagttgaaaggtgacattgagaaatgcctcacagctatcgtgaagtgcgccacaagcaaaccagctttctttgcagagaagcttcatcaagccatgaaaggtgttggaactcgccataaggcattgatcaggattatggtttcccgttctgaaattgacatgaatgatatcaaagcattctatcagaagatgtatggtatctccctttgccaagccatcctggatgaaaccaaaggagattatgagaaaatcctggtggctctttgtggaggaaactaaacattcccttgatggtctcaagctatgatcagaagactttaattatatattttcatcctataagcttaaataggaaagtttcttcaacaggattacagtgtagctacctacatgctgaaaaatatagcctttaaatcatttttatattataactctgtataatagagataagtccattttttaaaaatgttttccccaaaccataaaaccctatacaagttgttctagtaacaatacatgagaaagatgtctatgtagctgaaaataaaatgacgtcacaagacaa

SEQ ID NO: 20 - DNA, 1632 bp, Homo sapiens
  misc_feature 1..1632: note = NM_001002858.2 Homo sapiens annexin A2 (ANXA2), transcriptvariant 1, mRNA
  source 1..1632: mol_type = other DNA
gctcagcatttggggacgctctcagctctcggcgcacggcccaggtaagcggggcgcgccctgcccgcccgcgatgggccgccagctagcggggtgtggagacgctgggaagaaggcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaacagatgaataaactgaatttgtactttagaaacacgtactttgtggccctgctttcaactgaattgtttgaaaattaaacgtgcttggggttcagctggtgaggctgtccctgtaggaagaaagctctgggactgagctgtacagtatggttgcccctatccaagtgtcgctatttaagttaaatttaaatgaaataaaataaaataaaatcaaaaaaa

SEQ ID NO: 21 - DNA, 1665 bp, Homo sapiens
  misc_feature 1..1665: note = NM_001136015.2 Homo sapiens annexin A2 (ANXA2), transcriptvariant 4, mRNA
  source 1..1665: mol_type = other DNA
gctcagcatttggggacgctctcagctctcggcgcacggcccagggtgaaaatgtttgccattaaactcacatgaagtaggaaatatttatatggatacaaaaggcacctgcatgggataatgtcaaatttcatagatactgctttgtgcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaacagatgaataaactgaatttgtactttagaaacacgtactttgtggccctgctttcaactgaattgtttgaaaattaaacgtgcttggggttcagctggtgaggctgtccctgtaggaagaaagctctgggactgagctgtacagtatggttgcccctatccaagtgtcgctatttaagttaaatttaaatgaaataaaataaaataaaatcaaaaaaa

SEQ ID NO: 22 - DNA, 1432 bp, Homo sapiens
  misc_feature 1..1432: note = NM_005139.3 Homo sapiens annexin A3 (ANXA3), mRNA
  source 1..1432: mol_type = other DNA
agcgcggagcacctgcgcccgcggctgacaccttcgctcgcagtttgttcgcagtttactcgcacaccagtttcccccaccgcgctttggattagtgtgatctcagctcaaggcaaaggtgggatatcatggcatctatctgggttggacaccgaggaacagtaagagattatccagactttagcccatcagtggatgctgaagctattcagaaagcaatcagaggaattggaactgatgagaaaatgctcatcagcattctgactgagaggtcaaatgcacagcggcagctgattgttaaggaatatcaagcagcatatggaaaggagctgaaagatgacttgaagggtgatctctctggccactttgagcatctcatggtggccctagtgactccaccagcagtctttgatgcaaagcagctaaagaaatccatgaagggcgcgggaacaaacgaagatgccttgattgaaatcttaactaccaggacaagcaggcaaatgaaggatatctctcaagcctattatacagtatacaagaagagtcttggagatgacattagttccgaaacatctggtgacttccggaaagctctgttgactttggcagatggcagaagagatgaaagtctgaaagtggatgagcatctggccaaacaagatgcccagattctctataaagctggtgagaacagatggggcacggatgaagacaaattcactgagatcctgtgtttaaggagctttcctcaattaaaactaacatttgatgaatacagaaatatcagccaaaaggacattgtggacagcataaaaggagaattatctgggcattttgaagacttactgttggccatagttaattgtgtgaggaacacgccggcctttttagccgaaagactgcatcgagccttgaagggtattggaactgatgagtttactctgaaccgaataatggtgtccagatcagaaattgaccttttggacattcgaacagagttcaagaagcattatggctattccctatattcagcaattaaatcggatacttctggagactatgaaatcacactcttaaaaatctgtggtggagatgactgaaccaagaagataatctccaaaggtccacgatgggctttcccaacagctccaccttacttcttctcatactatttaagagaacaagcaaatataaacagcaacttgtgttcctaacaggaattttcattgttctataacaacaacaacaaaagcgattattattttagagcatctcatttataatgtagcagctcataaatgaaattgaaaatggtattaaagatctgcaactactatccaacttatatttctgctttcaaagttaagaatctttatagttctactccattaaatataaagcaagataataaaaattgttgcttttgttaaaa

SEQ ID NO: 23 - DNA, 2651 bp, Homo sapiens
  misc_feature 1..2651: note = NM_001153.5 Homo sapiens annexin A4 (ANXA4), transcript variant2, mRNA
  source 1..2651: mol_type = other DNA
gtgacctccgcagccgcagaggaggagcgcagcccggcctcgaagaacttctgcttgggtggctgaactctgatcttgacctagagtcatggccatggcaaccaaaggaggtactgtcaaagctgcttcaggattcaatgccatggaagatgcccagaccctgaggaaggccatgaaagggctcggcaccgatgaagacgccattattagcgtccttgcctaccgcaacaccgcccagcgccaggagatcaggacagcctacaagagcaccatcggcagggacttgatagacgacctgaagtcagaactgagtggcaacttcgagcaggtgattgtggggatgatgacgcccacggtgctgtatgacgtgcaagagctgcgaagggccatgaagggagccggcactgatgagggctgcctaattgagatcctggcctcccggacccctgaggagatccggcgcataagccaaacctaccagcagcaatatggacggagccttgaagatgacattcgctctgacacatcgttcatgttccagcgagtgctggtgtctctgtcagctggtgggagggatgaaggaaattatctggacgatgctctcgtgagacaggatgcccaggacctgtatgaggctggagagaagaaatgggggacagatgaggtgaaatttctaactgttctctgttcccggaaccgaaatcacctgttgcatgtgtttgatgaatacaaaaggatatcacagaaggatattgaacagagtattaaatctgaaacatctggtagctttgaagatgctctgctggctatagtaaagtgcatgaggaacaaatctgcatattttgctgaaaagctctataaatcgatgaagggcttgggcaccgatgataacaccctcatcagagtgatggtttctcgagcagaaattgacatgttggatatccgggcacacttcaagagactctatggaaagtctctgtactcgttcatcaagggtgacacatctggagactacaggaaagtactgcttgttctctgtggaggagatgattaaaataaaaatcccagaaggacaggaggattctcaacactttgaatttttttaacttcatttttctacactgctattatcattatctcagaatgcttatttccaattaaaacgcctacagctgcctcctagaatatagactgtctgtattattattcacctataattagtcattatgatgctttaaagctgtacttgcatttcaaagcttataagatataaatggagattttaaagtagaaataaatatgtattccatgtttttaaaagattactttctactttgtgtttcacagacattgaatatattaaattattccatattttcttttcagtgaaaaattttttaaatggaagactgttctaaaatcacttttttccctaatccaatttttagagtggctagtagtttcttcatttgaaattgtaagcatccggtcagtaagaatgcccatccagttttctatatttcatagtcaaagccttgaaagcatctacaaatctctttttttaggttttgtccatagcatcagttgatccttactaagtttttcatgggagacttccttcatcacatcttatgttgaaatcactttctgtagtcaaagtataccaaaaccaatttatctgaactaaattctaaagtatggttatacaaaccatatacatctggttaccaaacataaatgctgaacattccatattattatagttaatgtcttaatccagcttgcaagtgaatggaaaaaaaaataagcttcaaactaggtattctgggaatgatgtaatgctctgaatttagtatgatataaagaaaacttttttgtgctaaaaatactttttaaaatcaattttgttgattgtagtaatttctatttgcactgtgcctttcaactccagaaacattctgaagatgtacttggatttaattaaaaagttcactttgtaagaacgtggaaaaataattttaatttaaaaatggtgtttttaggccgggggcgggggctcacgccagtaatcccaacactttgggaggccaaggcgggtggatcacctaaggtcaggagttcaagactagcctggccaacatggagaaactgcatctctactaaaaatataaaaattagccgggtgtggtggctggtgcctgtaatcccagccactcggaggctgagtcagggagaactgcttgaacccaggaggcaggaggcaaaggttgcagtgagccgagatcacgccagcctgggcgacagagcgagaatccatctaaaaaaaaaaaaaaaaaaagtgtctttaaagtgaggtatagtctttctctgatccacttttcaccttctgaggtttttcatcttggcccctgaaaggagctatttttgaaggacttgtgttactcagtttctacaggaattacaagataagaaaaaaaaaatcatatttagtcttatgcgtgcctactggctaatgttcacatatgccaaacactactcaataacataaaataatgtatgaacttattctctggaaatgagtgatgccctctgctctaagtagaccatttatattaaatatcataaatgtataaaggacattcatattctta

SEQ ID NO: 24 - DNA, 1638 bp, Homo sapiens
  misc_feature 1..1638: note = NM_001154.4 Homo sapiens annexin A5 (ANXA5), mRNA
  source 1..1638: mol_type = other DNA
agtctaggtgcagctgccggatccttcagcgtctgcatctcggcgtcgccccgcgtaccgtcgcccggctctccgccgctctcccggggtttcggggcacttgggtcccacagtctggtcctgcttcaccttcccctgacctgagtagtcgccatggcacaggttctcagaggcactgtgactgacttccctggatttgatgagcgggctgatgcagaaactcttcggaaggctatgaaaggcttgggcacagatgaggagagcatcctgactctgttgacatcccgaagtaatgctcagcgccaggaaatctctgcagcttttaagactctgtttggcagggatcttctggatgacctgaaatcagaactaactggaaaatttgaaaaattaattgtggctctgatgaaaccctctcggctttatgatgcttatgaactgaaacatgccttgaagggagctggaacaaatgaaaaagtactgacagaaattattgcttcaaggacacctgaagaactgagagccatcaaacaagtttatgaagaagaatatggctcaagcctggaagatgacgtggtgggggacacttcagggtactaccagcggatgttggtggttctccttcaggctaacagagaccctgatgctggaattgatgaagctcaagttgaacaagatgctcaggctttatttcaggctggagaacttaaatgggggacagatgaagaaaagtttatcaccatctttggaacacgaagtgtgtctcatttgagaaaggtgtttgacaagtacatgactatatcaggatttcaaattgaggaaaccattgaccgcgagacttctggcaatttagagcaactactccttgctgttgtgaaatctattcgaagtatacctgcctaccttgcagagaccctctattatgctatgaagggagctgggacagatgatcataccctcatcagagtcatggtttccaggagtgagattgatctgtttaacatcaggaaggagtttaggaagaattttgccacctctctttattccatgattaagggagatacatctggggactataagaaagctcttctgctgctctgtggagaagatgactaacgtgtcacggggaagagctccctgctgtgtgcctgcaccaccccactgccttccttcagcacctttagctgcatttgtatgccagtgcttaacacattgccttattcatactagcatgctcatgaccaacacatacacgtcatagaagaaaatagtggtgcttctttctgatctctagtggagatctctttgactgctgtagtactaaagtgtacttaatgttactaagtttaatgcctggccattttccatttatatatattttttaagaggctagagtgcttttagccttttttaaaaactccatttatattacatttgtaaccatgatactttaatcagaagcttagccttgaaattgtgaactcttggaaatgttattagtgaagttcgcaactaaactaaacctgtaaaattatgatgattgtattcaaaagattaatgaaaaataaacatttctgtccccctgaattatgtgtacatgtgtgtttagatttattattaaatttatttaacaatgtt

SEQ ID NO: 25 - DNA, 2889 bp, Homo sapiens
  misc_feature 1..2889: note = NM_001155.5 Homo sapiens annexin A6 (ANXA6), transcript variant1, mRNA
  source 1..2889: mol_type = other DNA
gcggttgctgctgggctaacgggctccgatccagcgagcgctgcgtcctcgagtccctgcgcccgtgcgtccgtctgcgacccgaggcctccgctgcgcgtggattctgctgcgaaccggagaccatggccaaaccagcacagggtgccaagtaccggggctccatccatgacttcccaggctttgaccccaaccaggatgccgaggctctgtacactgccatgaagggctttggcagtgacaaggaggccatactggacataatcacctcacggagcaacaggcagaggcaggaggtctgccagagctacaagtccctctacggcaaggacctcattgctgatttaaagtatgaattgacgggcaagtttgaacggttgattgtgggcctgatgaggccacctgcctattgtgatgccaaagaaattaaagatgccatctcgggcattggcactgatgagaagtgcctcattgagatcttggcttcccggaccaatgagcagatgcaccagctggtggcagcatacaaagatgcctacgagcgggacctggaggctgacatcatcggcgacacctctggccacttccagaagatgcttgtggtcctgctccagggaaccagggaggaggatgacgtagtgagcgaggacctggtacaacaggatgtccaggacctatacgaggcaggggaactgaaatggggaacagatgaagcccagttcatttacatcttgggaaatcgcagcaagcagcatcttcggttggtgttcgatgagtatctgaagaccacagggaagccgattgaagccagcatccgaggggagctgtctggggactttgagaagctaatgctggccgtagtgaagtgtatccggagcaccccggaatattttgctgaaaggctcttcaaggctatgaagggcctggggactcgggacaacaccctgatccgcatcatggtctcccgtagtgagttggacatgctcgacattcgggagatcttccggaccaagtatgagaagtccctctacagcatgatcaagaatgacacctctggcgagtacaagaagactctgctgaagctgtctgggggagatgatgatgctgctggccagttcttcccggaggcagcgcaggtggcctatcagatgtgggaacttagtgcagtggcccgagtagagctgaagggaactgtgcgcccagccaatgacttcaaccctgacgcagatgccaaagcgctgcggaaagccatgaagggactcgggactgacgaagacacaatcatcgatatcatcacgcaccgcagcaatgtccagcggcagcagatccggcagaccttcaagtctcactttggccgggacttaatgactgacctgaagtctgagatctctggagacctggcaaggctgattctggggctcatgatgccaccggcccattacgatgccaagcagttgaagaaggccatggagggagccggcacagatgaaaaggctcttattgaaatcctggccactcggaccaatgctgaaatccgggccatcaatgaggcctataaggaggactatcacaagtccctggaggatgctctgagctcagacacatctggccacttcaggaggatcctcatttctctggccacggggcatcgtgaggagggaggagaaaacctggaccaggcacgggaagatgcccaggtggctgctgagatcttggaaatagcagacacacctagtggagacaaaacttccttggagacacgtttcatgacgatcctgtgtacccggagctatccgcacctccggagagtcttccaggagttcatcaagatgaccaactatgacgtggagcacaccatcaagaaggagatgtctggggatgtcagggatgcatttgtggccattgttcaaagtgtcaagaacaagcctctcttctttgccgacaaactttacaaatccatgaagggtgctggcacagatgagaagactctgaccaggatcatggtatcccgcagtgagattgacctgctcaacatccggagggaattcattgagaaatatgacaagtctctccaccaagccattgagggtgacacctccggagacttcctgaaggccttgctggctctctgtggtggtgaggactagggccacagctttggcgggcacttctgccaagaaatggttatcagcaccagccgccatggccaagcctgattgttccagctccagagactaaggaaggggcaggggtggggggaggggttgggttgggctcttatcttcagtggagcttaggaaacgctcccactcccacgggccatcgagggcccagcacggctgagcggctgaaaaaccgtagccatagatcctgtccacctccactcccctctgaccctcaggctttcccagcttcctccccttgctacagcctctgccctggtttgggctatgtcagatccaaaaacatcctgaacctctgtctgtaaaatgagtagtgtctgtactttgaatgagggggttggtggcaggggccagttgaatgtgctgggcggggtggtgggaaggatagtaaatgtgctggggcaaactgacaaatcttcccatccatttcaccacccatctccatccaggccgcgctagagtactggaccaggaatttggatgcctgggttcaaatctgcatctgccatgcacttgtttctgaccttaggccagcccctttccctccctgagtctctattttcttatctacaatgagacagttggacaaaaaaatcttggcttcccttctaacattaacttcctaaagtatgcctccgattcattcccttgacactttttatttctaaggaagaaataaaaagagatacacaaacacataaacaca

SEQ ID NO: 26 - DNA, 2879 bp, Homo sapiens
  misc_feature 1..2879: note = NM_001193544.1 Homo sapiens annexin A6 (ANXA6), transcriptvariant 2, mRNA
  source 1..2879: mol_type = other DNA
agagaccagagagcatccagaggcctggccggggtcctgcagtgcagacgttgggaggcacggagacggggagagggggaggcggtccaggactcactctgctccacctctgactccttgaagggtgccaagtaccggggctccatccatgacttcccaggctttgaccccaaccaggatgccgaggctctgtacactgccatgaagggctttggcagtgacaaggaggccatactggacataatcacctcacggagcaacaggcagaggcaggaggtctgccagagctacaagtccctctacggcaaggacctcattgctgatttaaagtatgaattgacgggcaagtttgaacggttgattgtgggcctgatgaggccacctgcctattgtgatgccaaagaaattaaagatgccatctcgggcattggcactgatgagaagtgcctcattgagatcttggcttcccggaccaatgagcagatgcaccagctggtggcagcatacaaagatgcctacgagcgggacctggaggctgacatcatcggcgacacctctggccacttccagaagatgcttgtggtcctgctccagggaaccagggaggaggatgacgtagtgagcgaggacctggtacaacaggatgtccaggacctatacgaggcaggggaactgaaatggggaacagatgaagcccagttcatttacatcttgggaaatcgcagcaagcagcatcttcggttggtgttcgatgagtatctgaagaccacagggaagccgattgaagccagcatccgaggggagctgtctggggactttgagaagctaatgctggccgtagtgaagtgtatccggagcaccccggaatattttgctgaaaggctcttcaaggctatgaagggcctggggactcgggacaacaccctgatccgcatcatggtctcccgtagtgagttggacatgctcgacattcgggagatcttccggaccaagtatgagaagtccctctacagcatgatcaagaatgacacctctggcgagtacaagaagactctgctgaagctgtctgggggagatgatgatgctgctggccagttcttcccggaggcagcgcaggtggcctatcagatgtgggaacttagtgcagtggcccgagtagagctgaagggaactgtgcgcccagccaatgacttcaaccctgacgcagatgccaaagcgctgcggaaagccatgaagggactcgggactgacgaagacacaatcatcgatatcatcacgcaccgcagcaatgtccagcggcagcagatccggcagaccttcaagtctcactttggccgggacttaatgactgacctgaagtctgagatctctggagacctggcaaggctgattctggggctcatgatgccaccggcccattacgatgccaagcagttgaagaaggccatggagggagccggcacagatgaaaaggctcttattgaaatcctggccactcggaccaatgctgaaatccgggccatcaatgaggcctataaggaggactatcacaagtccctggaggatgctctgagctcagacacatctggccacttcaggaggatcctcatttctctggccacggggcatcgtgaggagggaggagaaaacctggaccaggcacgggaagatgcccaggtggctgctgagatcttggaaatagcagacacacctagtggagacaaaacttccttggagacacgtttcatgacgatcctgtgtacccggagctatccgcacctccggagagtcttccaggagttcatcaagatgaccaactatgacgtggagcacaccatcaagaaggagatgtctggggatgtcagggatgcatttgtggccattgttcaaagtgtcaagaacaagcctctcttctttgccgacaaactttacaaatccatgaagggtgctggcacagatgagaagactctgaccaggatcatggtatcccgcagtgagattgacctgctcaacatccggagggaattcattgagaaatatgacaagtctctccaccaagccattgagggtgacacctccggagacttcctgaaggccttgctggctctctgtggtggtgaggactagggccacagctttggcgggcacttctgccaagaaatggttatcagcaccagccgccatggccaagcctgattgttccagctccagagactaaggaaggggcaggggtggggggaggggttgggttgggctcttatcttcagtggagcttaggaaacgctcccactcccacgggccatcgagggcccagcacggctgagcggctgaaaaaccgtagccatagatcctgtccacctccactcccctctgaccctcaggctttcccagcttcctccccttgctacagcctctgccctggtttgggctatgtcagatccaaaaacatcctgaacctctgtctgtaaaatgagtagtgtctgtactttgaatgagggggttggtggcaggggccagttgaatgtgctgggcggggtggtgggaaggatagtaaatgtgctggggcaaactgacaaatcttcccatccatttcaccacccatctccatccaggccgcgctagagtactggaccaggaatttggatgcctgggttcaaatctgcatctgccatgcacttgtttctgaccttaggccagcccctttccctccctgagtctctattttcttatctacaatgagacagttggacaaaaaaatcttggcttcccttctaacattaacttcctaaagtatgcctccgattcattcccttgacactttttatttctaaggaagaaataaaaagagatacacaaacacataaacacaaaaaaaaaaa

SEQ ID NO: 27 - DNA, 2443 bp, Homo sapiens
  misc_feature 1..2443: note = NM_001156.5 Homo sapiens annexin A7 (ANXA7), transcript variant1, mRNA
  source 1..2443: mol_type = other DNA
atcttgcgggagaccgggttgggctgtgacgctgctgctggggtcagaatgtcatacccaggctatcccccaacaggctacccacctttccctggatatcctcctgcaggtcaggagtcatcttttcccccttctggtcagtatccttatcctagtggctttcctccaatgggaggaggtgcctacccacaagtgccaagtagtggctacccaggagctggaggctaccctgcgcctggaggttatccagcccctggaggctatcctggtgccccacagccagggggagctccatcctatcccggagttcctccaggccaaggatttggagtcccaccaggtggagcaggcttttctgggtatccacagccaccttcacagtcttatggaggtggtccagcacaggttccactacctggtggctttcctggaggacagatgccttctcagtatcctggaggacaacctacttaccctagtcagcctgccacagtgactcaggtcactcaaggaactatccgaccagctgccaacttcgatgctataagagatgcagaaattcttcgtaaggcaatgaagggttttgggacagatgagcaggcaattgtggatgtggtggccaaccgttccaatgatcagaggcaaaaaattaaagcagcatttaagacctcctatggcaaggatttaatcaaagatctcaaatcagagttaagtggaaatatggaagaactgatcctggccctcttcatgcctcctacgtattacgatgcctggagcttacggaaagcaatgcagggagcaggaactcaggaacgtgtattgattgagattttgtgcacaagaacaaatcaggaaatccgagaaattgtcagatgttatcagtcagaatttggacgagaccttgaaaaggacattaggtcagatacatcaggacattttgaacgtttacttgtgtccatgtgccagggaaatcgtgatgagaaccagagtataaaccaccaaatggctcaggaagatgctcagcgtctctatcaagctggtgaggggagactagggaccgatgaatcttgctttaacatgatccttgccacaagaagctttcctcagctgagagctaccatggaggcttattctaggatggctaatcgagacttgttaagcagtgtgagccgtgagttttccggatatgtagaaagtggtttgaagaccatcttgcagtgtgccctgaaccgccctgccttctttgctgagaggctctactatgctatgaaaggtgctggcacagatgactccaccctggtccggattgtggtcactcgaagtgagattgaccttgtacaaataaaacagatgttcgctcagatgtatcagaagactctgggcacaatgattgcaggtgacacgagtggagattaccgaagacttcttctggctattgtgggccagtaggagggatttttttttttttaatgaaaaaaaatttctattcatagcttatccttcagagcaatgacctgcatgcagcaatatcaaacatcagctaaccgaaagagctttctgtcaaggaccgtatcagggtaatgtgcttggtttgcacatgttgttattgccttaattctaattttattttgttctctacatacaatcaatgtaaagccatatcacaatgatacagtaatattgcaatgtttgtaaaccttcattcttactagtttcattctaatcaagatgtcaaattgaataaaaatcacagcaatctctgattctgtgtaataatattgaataattttttagaaggttactgaaagctctgccttccggaatccctctaagtctgcttgatagagtggatagtgtgttaaaactgtgtactttaaaaaaaaattcaacctttacatctagaataatttgcatctcattttgcctaaattggttctgtattcataaacactttccacatagaaaatagattagtattacctgtggcaccttttaagaaagggtcaaatgtttatatgcttaagatacatagcctacttttttttcgcagttgttttctttttttaaattgagttatgacaaataaaaaattgcatatatttaaggtgtacaatatggtgttttgatatcagcattccttgtgtaatgattccacaattaaggtcaggctaattacgtatctgtcaccttgacatagttaccattttttcatgtgtggtgaaaacacttaagatctactaccttagcaaattttaagtgttcagtacattattaactatagatactgtgctctacattaaacctctagcatttattcgttttataactgaaagtttataccctttgaccaacatctccccattttccccacctctcacctggacaaccaccactgtgtttaagttcagctattttagattccacgtataaatggtatacaata

SEQ ID NO: 28 - DNA, 2550 bp, Homo sapiens
  misc_feature 1..2550: note = NM_004034.3 Homo sapiens annexin A7 (ANXA7), transcript variant2, mRNA
  source 1..2550: mol_type = other DNA
gcccaccctgggcccgcccccggctccatcttgcgggagaccgggttgggctgtgacgctgctgctggggtcagaatgtcatacccaggctatcccccaacaggctacccacctttccctggatatcctcctgcaggtcaggagtcatcttttcccccttctggtcagtatccttatcctagtggctttcctccaatgggaggaggtgcctacccacaagtgccaagtagtggctacccaggagctggaggctaccctgcgcctggaggttatccagcccctggaggctatcctggtgccccacagccagggggagctccatcctatcccggagttcctccaggccaaggatttggagtcccaccaggtggagcaggcttttctgggtatccacagccaccttcacagtcttatggaggtggtccagcacaggttccactacctggtggctttcctggaggacagatgccttctcagtatcctggaggacaacctacttaccctagtcagatcaatacagattctttttcttcctatcctgttttctctcctgtttctttggattatagcagtgaacctgccacagtgactcaggtcactcaaggaactatccgaccagctgccaacttcgatgctataagagatgcagaaattcttcgtaaggcaatgaagggttttgggacagatgagcaggcaattgtggatgtggtggccaaccgttccaatgatcagaggcaaaaaattaaagcagcatttaagacctcctatggcaaggatttaatcaaagatctcaaatcagagttaagtggaaatatggaagaactgatcctggccctcttcatgcctcctacgtattacgatgcctggagcttacggaaagcaatgcagggagcaggaactcaggaacgtgtattgattgagattttgtgcacaagaacaaatcaggaaatccgagaaattgtcagatgttatcagtcagaatttggacgagaccttgaaaaggacattaggtcagatacatcaggacattttgaacgtttacttgtgtccatgtgccagggaaatcgtgatgagaaccagagtataaaccaccaaatggctcaggaagatgctcagcgtctctatcaagctggtgaggggagactagggaccgatgaatcttgctttaacatgatccttgccacaagaagctttcctcagctgagagctaccatggaggcttattctaggatggctaatcgagacttgttaagcagtgtgagccgtgagttttccggatatgtagaaagtggtttgaagaccatcttgcagtgtgccctgaaccgccctgccttctttgctgagaggctctactatgctatgaaaggtgctggcacagatgactccaccctggtccggattgtggtcactcgaagtgagattgaccttgtacaaataaaacagatgttcgctcagatgtatcagaagactctgggcacaatgattgcaggtgacacgagtggagattaccgaagacttcttctggctattgtgggccagtaggagggatttttttttttttaatgaaaaaaaatttctattcatagcttatccttcagagcaatgacctgcatgcagcaatatcaaacatcagctaaccgaaagagctttctgtcaaggaccgtatcagggtaatgtgcttggtttgcacatgttgttattgccttaattctaattttattttgttctctacatacaatcaatgtaaagccatatcacaatgatacagtaatattgcaatgtttgtaaaccttcattcttactagtttcattctaatcaagatgtcaaattgaataaaaatcacagcaatctctgattctgtgtaataatattgaataattttttagaaggttactgaaagctctgccttccggaatccctctaagtctgcttgatagagtggatagtgtgttaaaactgtgtactttaaaaaaaaattcaacctttacatctagaataatttgcatctcattttgcctaaattggttctgtattcataaacactttccacatagaaaatagattagtattacctgtggcaccttttaagaaagggtcaaatgtttatatgcttaagatacatagcctacttttttttcgcagttgttttctttttttaaattgagttatgacaaataaaaaattgcatatatttaaggtgtacaatatggtgttttgatatcagcattccttgtgtaatgattccacaattaaggtcaggctaattacgtatctgtcaccttgacatagttaccattttttcatgtgtggtgaaaacacttaagatctactaccttagcaaattttaagtgttcagtacattattaactatagatactgtgctctacattaaacctctagcatttattcgttttataactgaaagtttataccctttgaccaacatctccccattttccccacctctcacctggacaaccaccactgtgtttaagttcagctattttagattccacgtataaatggtatacaataaaaaaaaaaaaaaa

SEQ ID NO: 29 - DNA, 2205 bp, Homo sapiens
  misc_feature 1..2205: note = NM_001271702.1 Homo sapiens annexin A8 (ANXA8), transcriptvariant 1, mRNA
  source 1..2205: mol_type = other DNA
ctgggtggggcctgggagccacaggagatgcccaaagccaggcagagcccgggggcgaggggacggcaggcaggtgtggcgctgccctgggcgggcttgcacccccacacccaagtgagcggcctgctcactcctcagctgcaggagccagacgtgtggagtcccagcagaggccaacctgtgtctcttcatctccctgggaaaggtgcccccgaggtgaaagagatggcctggtggaaatcctggattgaacaggagggtgtcacagtgaagagcagctcccacttcaacccagaccctgatgcagagaccctctacaaagccatgaaggggatcggtgtcgggtcccaactgctcagccaccaagcagctgccttcgccttcccctcctccgccctcaccagtgtgtcaccctgggggcagcagggtcacttgtgctgtaaccctgcagggaccaacgagcaggctatcatcgatgtgctcaccaagagaagcaacacgcagcggcagcagatcgccaagtccttcaaggctcagttcggcaaggacctcactgagaccttgaagtctgagctcagtggcaagtttgagaggctcattgtggcccttatgtacccgccatacagatacgaagccaaggagctgcatgacgccatgaagggcttaggaaccaaggagggtgtcatcattgagatcctggcctctcggaccaagaaccagctgcgggagataatgaaggcgtatgaggaagactatgggtccagcctggaggaggacatccaagcagacacaagtggctacctggagaggatcctggtgtgcctcctgcagggcagcagggatgatgtgagcagctttgtggacccaggactggccctccaagacgcacaggatctgtatgcggcaggcgagaagattcgtgggactgatgagatgaaattcatcaccatcctgtgcacgcgcagtgccactcacctgctgagagtgtttgaagagtatgagaaaattgccaacaagagcattgaggacagcatcaagagtgagacccatggctcactggaggaggccatgctcactgtggtgaaatgcacccaaaacctccacagctactttgcagagagactctactatgccatgaagggagcagggacgcgtgatgggaccctgataagaaacatcgtttcaaggagcgagattgacttaaatcttatcaaatgtcacttcaagaagatgtacggcaagaccctcagcagcatgatcatggaagacaccagcggtgactacaagaacgccctgctgagcctggtgggcagcgacccctgaggcacagaagaacaagagcaaagaccatgaagccagagtctccaggactcctcactcaacctcggccatggacgcaggttgggtgtgaggggggtcccagcctttcggtcttctatttccctatttccagtgctttccagccgggtttctgacccagagggtggaaccggcctggactcctcttcccaacttcctccaggtcatttcccagtgtgagcacaatgccaaccttagtgtttctccagccagacagatgcctcagcatgaagggcttggggacttgtggatcattccttcctccctgcaggagcttcccaagctggtcacagagtctcctgggcacaggttatacagaccccagccccattcccatctactgaaacagggtctccacaagaggggccagggaatatgggtttttaacaagcgtcttacaaaacacttctctatcatgcagccggagagctggctgggagcccttttgttttagaacacacatccttcagcagctgagaaacgaacacgaatccatcccaaccgagatgccattaacattcatctaaaaatgttaggctctaaatggacgaaaaattctctcgccatcttaataacaaaataaactacaaattcctgacccaaggacactgtgttataagaggcgtgggctcccctggtggctgaccaggtcagctgccctggccttgcacccctctgcatgcagcacagaagggtgtgaccatgccctcagcaccactcttgtccccactgaacggcaactgagactgggtacctggagattctgaagtgcctttgctgtggttttcaaaataataaagatttgtattcaactcaaaaaaaaaaaaaaaaaaaaaaaaaaaaaaa

SEQ ID NO: 30 - DNA, 2070 bp, Homo sapiens
  misc_feature 1..2070: note = NM_001040084.2 Homo sapiens annexin A8 (ANXA8), transcriptvariant 2, mRNA
  source 1..2070: mol_type = other DNA
ctgggtggggcctgggagccacaggagatgcccaaagccaggcagagcccgggggcgaggggacggcaggcaggtgtggcgctgccctgggcgggcttgcacccccacacccaagtgagcggcctgctcactcctcagctgcaggagccagacgtgtggagtcccagcagaggccaacctgtgtctcttcatctccctgggaaaggtgcccccgaggtgaaagagatggcctggtggaaatcctggattgaacaggagggtgtcacagtgaagagcagctcccacttcaacccagaccctgatgcagagaccctctacaaagccatgaaggggatcgggaccaacgagcaggctatcatcgatgtgctcaccaagagaagcaacacgcagcggcagcagatcgccaagtccttcaaggctcagttcggcaaggacctcactgagaccttgaagtctgagctcagtggcaagtttgagaggctcattgtggcccttatgtacccgccatacagatacgaagccaaggagctgcatgacgccatgaagggcttaggaaccaaggagggtgtcatcattgagatcctggcctctcggaccaagaaccagctgcgggagataatgaaggcgtatgaggaagactatgggtccagcctggaggaggacatccaagcagacacaagtggctacctggagaggatcctggtgtgcctcctgcagggcagcagggatgatgtgagcagctttgtggacccaggactggccctccaagacgcacaggatctgtatgcggcaggcgagaagattcgtgggactgatgagatgaaattcatcaccatcctgtgcacgcgcagtgccactcacctgctgagagtgtttgaagagtatgagaaaattgccaacaagagcattgaggacagcatcaagagtgagacccatggctcactggaggaggccatgctcactgtggtgaaatgcacccaaaacctccacagctactttgcagagagactctactatgccatgaagggagcagggacgcgtgatgggaccctgataagaaacatcgtttcaaggagcgagattgacttaaatcttatcaaatgtcacttcaagaagatgtacggcaagaccctcagcagcatgatcatggaagacaccagcggtgactacaagaacgccctgctgagcctggtgggcagcgacccctgaggcacagaagaacaagagcaaagaccatgaagccagagtctccaggactcctcactcaacctcggccatggacgcaggttgggtgtgaggggggtcccagcctttcggtcttctatttccctatttccagtgctttccagccgggtttctgacccagagggtggaaccggcctggactcctcttcccaacttcctccaggtcatttcccagtgtgagcacaatgccaaccttagtgtttctccagccagacagatgcctcagcatgaagggcttggggacttgtggatcattccttcctccctgcaggagcttcccaagctggtcacagagtctcctgggcacaggttatacagaccccagccccattcccatctactgaaacagggtctccacaagaggggccagggaatatgggtttttaacaagcgtcttacaaaacacttctctatcatgcagccggagagctggctgggagcccttttgttttagaacacacatccttcagcagctgagaaacgaacacgaatccatcccaaccgagatgccattaacattcatctaaaaatgttaggctctaaatggacgaaaaattctctcgccatcttaataacaaaataaactacaaattcctgacccaaggacactgtgttataagaggcgtgggctcccctggtggctgaccaggtcagctgccctggccttgcacccctctgcatgcagcacagaagggtgtgaccatgccctcagcaccactcttgtccccactgaacggcaactgagactgggtacctggagattctgaagtgcctttgctgtggttttcaaaataataaagatttgtattcaactcaaaaaaaaaa

SEQ ID NO: 31 - DNA, 1551 bp, Homo sapiens
  misc_feature 1..1551: note = NM_003568.3 Homo sapiens annexin A9 (ANXA9), mRNA
  source 1..1551: mol_type = other DNA
ctctaccaggccacaccggaggcagtgctcacacaggcaagctaccaggccacaacaacgacacccacctcacctctggcacctctgagcatccacgtacttgcaagaactcttgctcacatcagctaagagattgcacctgctgacctagagattccggcctgtgctcctgtgctgctgagcagggcaaccagtagcaccatgtctgtgactggcgggaagatggcaccgtccctcacccaggagatcctcagccacctgggcctggccagcaagactgcagcgtgggggaccctgggcaccctcaggaccttcttgaacttcagcgtggacaaggatgcgcagaggctactgagggccattactggccaaggcgtggaccgcagtgccattgtggacgtgctgaccaaccggagcagagagcaaaggcagctcatctcacgaaacttccaggagcgcacccaacaggacctgatgaagtctctacaggcagcactttccggcaacctggagaggattgtgatggctctgctgcagcccacagcccagtttgacgcccaggaattgaggacagctctgaaggcctcagattctgctgtggacgtggccattgaaattcttgccactcgaaccccaccccagctgcaggagtgcctggcagtctacaaacacaatttccaggtggaggctgtggatgacatcacatctgagaccagtggcatcttgcaggacctgctgttggccctggccaaggggggccgtgacagctactctggaatcattgactataatctggcagaacaagatgtccaggcactgcagcgggcagaaggacctagcagagaggaaacatgggtcccagtcttcacccagcgaaatcctgaacacctcatccgagtgtttgatcagtaccagcggagcactgggcaagagctggaggaggctgtccagaaccgtttccatggagatgctcaggtggctctgctcggcctagcttcggtgatcaagaacacaccgctgtactttgctgacaaacttcatcaagccctccaggaaactgagcccaattaccaagtcctgattcgcatccttatctctcgatgtgagactgaccttctgagtatcagagctgagttcaggaagaaatttgggaagtccctctactcttctctccaggatgcagtgaaaggggattgccagtcagccctcctggccttgtgcagggctgaagacatgtgagacttccctgccccaccccacatgacatccgaggatctgagatttccgtgtttggctgaacctgggagaccagctgggcctccaagtaggataacccctcactgagcaccacattctctagcttcttgttgaggctggaactgtttctttaaaatcccttaattttcccatctcaaaattatatctgtacctgggtcatccagctccttcttgggtgtggggaaatgagttttctttgatagtttctgcctcactcatccctcctgtaccctggccagaacatctcactgatactcgaattcttttggcaaa

SEQ ID NO: 32 - DNA, 1447 bp, Homo sapiens
  misc_feature 1..1447: note = NM_007193.4 Homo sapiens annexin A10 (ANXA10), mRNA
  source 1..1447: mol_type = other DNA
atccagatttgcttttacattttcttgcctgagtctgaggtgaacagtgaacatatttacatttgatttaacagtgaaccttaattctttctggcttcacagtgaaacaagtttatgcaatcgatcaaatattttcatccctgaggttaacaattaccatcaaaatgttttgtggagactatgtgcaaggaaccatcttcccagctcccaatttcaatcccataatggatgcccaaatgctaggaggagcactccaaggatttgactgtgacaaagacatgctgatcaacattctgactcagcgctgcaatgcacaaaggatgatgattgcagaggcataccagagcatgtatggccgggacctgattggggatatgagggagcagctttcggatcacttcaaagatgtgatggctggcctcatgtacccaccaccactgtatgatgctcatgagctctggcatgccatgaagggagtaggcactgatgagaattgcctcattgaaatactagcttcaagaacaaatggagaaattttccagatgcgagaagcctactgcttgcaatacagcaataacctccaagaggacatttattcagagacctcaggacacttcagagatactctcatgaacttggtccaggggaccagagaggaaggatatacagaccctgcgatggctgctcaggatgcaatggtcctatgggaagcctgtcagcagaagacgggggagcacaaaaccatgctgcaaatgatcctgtgcaacaagagctaccagcagctgcggctggttttccaggaatttcaaaatatttctgggcaagatatggtagatgccattaatgaatgttatgatggatactttcaggagctgctggttgcaattgttctctgtgttcgagacaaaccagcctattttgcttatagattatatagtgcaattcatgactttggtttccataataaaactgtaatcaggattctcattgccagaagtgaaatagacctgctgaccataaggaaacgatacaaagagcgatatggaaaatccctatttcatgatatcagaaattttgcttcagggcattataagaaagcactgcttgccatctgtgctggtgatgctgaggactactaaaatgaagaggacttggagtactgtgcactcctctttctagacacttccaaatagagattttctcacaaatttgtactgttcatggcactattaacaaaactatacaatcatattttctcttctatctttgaaattattctaagccaaagaaaactatgaatgaaagtatatgatactgaatttgcctactatcctgaatttgcctactatctaatcagcaattaaataaattgtgcatgatggaataatagaaaaattgcattggaatagattttatttaaatgtgaaccatcaacaacctacaacaa

SEQ ID NO: 33 - DNA, 6734 bp, Homo sapiens
  misc_feature 1..6734: note = NM_145868.2 Homo sapiens annexin A11 (ANXA11), transcript variantb, mRNA
  source 1..6734: mol_type = other DNA
ggagttttccgcccggcgctgacggctgctgcgcccgcggctccccagtgccccgagtgccccgcgggccccgcgagcgggagtgggacccagcccctaggcagaacccaggcgccgcgcccgggacgcccgcggagagagccactcccgcccacgtcccatttcgcccctcgcgtccggagtccccgtggccaggtgtgtgtctggggaagagacttacagaagtggagttgctgagtcaaagatctaaccatgagctaccctggctatcccccgcccccaggtggctacccaccagctgcaccaggtggtggtccctggggaggtgctgcctaccctcctccgcccagcatgccccccatcgggctggataacgtggccacctatgcggggcagttcaaccaggactatctctcgggaatggcggccaacatgtctgggacatttggaggagccaacatgcccaacctgtaccctggggcccctggggctggctacccaccagtgccccctggcggctttgggcagcccccctctgcccagcagcctgttcctccctatgggatgtatccacccccaggaggaaacccaccctccaggatgccctcatatccgccatacccaggggcccctgtgccgggccagcccatgccaccccccggacagcagcccccaggggcctaccctgggcagccaccagtgacctaccctggtcagcctccagtgccactccctgggcagcagcagccagtgccgagctacccaggatacccggggtctgggactgtcacccccgctgtgcccccaacccagtttggaagccgaggcaccatcactgatgctcccggctttgaccccctgcgagatgccgaggtcctgcggaaggccatgaaaggcttcgggacggatgagcaggccatcattgactgcctggggagtcgctccaacaagcagcggcagcagatcctactttccttcaagacggcttacggcaaggatttgatcaaagatctgaaatctgaactgtcaggaaactttgagaagacaatcttggctctgatgaagaccccagtcctctttgacatttatgagataaaggaagccatcaagggggttggcactgatgaagcctgcctgattgagatcctcgcttcccgcagcaatgagcacatccgagaattaaacagagcctacaaagcagaattcaaaaagaccctggaagaggccattcgaagcgacacatcagggcacttccagcggctcctcatctctctctctcagggaaaccgtgatgaaagcacaaacgtggacatgtcactcgcccagagagatgcccaggagctgtatgcggccggggagaaccgcctgggaacagacgagtccaagttcaatgcggttctgtgctcccggagccgggcccacctggtagcagttttcaatgagtaccagagaatgacaggccgggacattgagaagagcatctgccgggagatgtccggggacctggaggagggcatgctggccgtggtgaaatgtctcaagaataccccagccttctttgcggagaggctcaacaaggccatgaggggggcaggaacaaaggaccggaccctgattcgcatcatggtgtctcgcagcgagaccgacctcctggacatcagatcagagtataagcggatgtacggcaagtcgctgtaccacgacatctcgggagatacttcaggggattaccggaagattctgctgaagatctgtggtggcaatgactgaacagtgactggtggctcacttctgcccacctgccggcaacaccagtgccaggaaaaggccaaaagaatgtctgtttctaacaaatccacaaatagccccgagattcaccgtcctagagcttaggcctgtcttccacccctcctgacccgtatagtgtgccacaggacctgggtcggtctagaactctctcaggatgccttttctaccccatccctcacagcctcttgctgctaaaatagatgtttcatttttctgactcatgcaatcattcccctttgcctgtggctaagacttggcttcatttcgtcatgtaattgtatatttttatttggaggcatattttcttttcttacagtcattgccagacagaggcatacaagtctgtttgctgcatacacatttctggtgagggcgactgggtgggtgaagcaccgtgtcctcgctgaggagagaaagggaggcgtgcctgagaaggtagcctgtgcatctggtgagtgtgtcacgagctttgttactgccaaactcactcctttttagaaaaaacaaaaaaaaagggccagaaagtcattccttccatcttccttgcagaaaccacgagaacaaagccagttccctgtcagtgacagggcttcttgtaatttgtggtatgtgccttaaacctgaatgtctgtagccaaaacttgtttccacattaagagtcagccagctctggaatggtctggaaatgtcttcctggtaccaacttgttttcttctgcttgattctgccctgtggctcagaggtctggccttatcagccagtgaaagttcatgtaaccttacgtagagatttgtgtgcaggaaaccctgagcatacactagtttgcagggactcgtaaggacatgggaagggaggttcccgaaatccaggcaggaggcccagacacctgaaaggcaaagggatcttggttggttgcaggtgcagtgaagtccactgaaggtgtggtgcgaagaatgcagtccttcacccaggtcccaggagggaagaagggtgtgtgctaattcctggtgcccctcggcgggggccagagagaaggatggggacaacccagagagtcacaagaccagtgcctcccctcagggtgcctccaggctgaaaggggctcctggctctggtctctggggaccctgtgcccgttggttggtggtgtgagggaagagaatccataagagagtttctgagaattatggtgtcatgtccagaagctagagcttaccttgcatcaggggtctccacccactccttttccaacctcctgcgttgaggtttagaaaagagagaatcgactaggcactatggctcacgcctgtaatccaaggactttgggaagctgaggtgagaggatcacttgagctcaggagttcaagactagcctagccaacagcgagacccctgtctctactaaaaaatttggccaggcgtggtggctcacggctgtaatcccagcactttgggaggtgaggcgggcagatcacctgaggtcaggagttcgagacccagcctggccaacatggtgaaaccccatctctactaaaaatacaaaaattagccaggcatggtggcacattcctgtaatcccagctacacaggatgctgaggcaggagaatcacttgaacccaggaggcagaggttgtagtgagctgagatcacaccattgcacttcaacctgggtggacagagtgagactctgtctcaaaaaaaaaaaaaaatttacctggcattgtagtgcattccctatagtcggctactctggaggctgaggcaggaagatccttagagcccaagaaattgaggccgtagtaagctgtgattacaccactgcactccagcctggacaacagagcgagaccttgtctcaaatgagaaaaaaacaaaaagaaatgggagaatccagagagactaggctagatcaagcctgctgggtcctggcaggagccccagggagtagctcatctgcagacatttgcttgaggactaccccctaaacataaaggaagaatgacatccgaagggtgtggagcagccatgagctgagaactagcctggtctacctgagattgatggcaggtcctggtcaacacgtcagctctgcgtcagagtccatgcctcaagcccaagctgaagccccatccctgctgctctcccaagaactcctctgctagggcaggccccttgcccttgggtgccaggtgggacctgcctgatgggatggggtgcttggcatatacaacttgccatgaactcaaggtgaccctgggggcctcctgaattgtgatggggcctagaaccaatgtgctctgatgtgaccatattctgtgacattaccttgccctgtttactccaaagttcccagcctggtgcccagcaggcaatattgcacctacagacacatttactttggtttccaaagtgtttttagacatttgaatttgttgccaacatttaaacattgagagatttcatatttttaaaaatctggaattctggcttctcttgaaaactcagaaattctggcactatggggcttgcattcctgcatggctggagctgagttgcagctgcccctttaggcctgtactccttatttgctataggctccgtcttgtattacactaagcccatgtcacccatttggctcctgcaggcctttgggtttgagaccctggtctacacacttggagaccacctgttgtaaagtacatggatgtgctttggtcaaggaatagaccaaggtggatatccaggccagagtgactcagcgagtttaggtcacaggcgtatactccacttgttatataacctgcttgtgtaagttcatacttggctcaaagccactattgtttggaaaaggtataactgccctgctgacgctgtacagatgttcttgggctcggatgggcatggctccacgtggtgtgcactagcacccagagagagtgaagctattgacccctgtaagggagagtgaccatctggcagatagatagaggggagccaggacatggctcagcttgtgcccagagggagagttaagccgctgaccctgtagccagggagtgcacctgcaagcatgggggtggcaggagccacagagctggctgctgagaggagctgcagatctggagaagacagcctaggtaaaggtggacagtgtgagagctgctgatgagatagctgctgaataaaactacattttacctgcctatggcccgccaggttttctttcagctatcgcccatccacccagtcccctcgaacctcagcatgggctggaacctgaccctgggcatgacatttggcatagttgtggacctgacacctgtgtttgtcctagtcctgtttctccctgccttcctgttcctctcgctgccctcatggtcactcccaagagatccaacccatgttaagtatgggctggaggactgcatgaatgcctcatgatcttcccagaggcaaaggcacctactgccttccaaggtcagtgggaggttgggatcaacactgtttattatgcttaggacaaaaaagatagggagaaagatgtgcaaccttacaggtcatctttctgggatagaacacaatgggtcttctcctgcctcctggatatgttagtcaaggccagtccatgctacacatctagtctgacttctaaaatagaagcaccagatgaattcagccctgagagaattttcagcagctgtgggggcgctggaggaaacactattaaatagttttgcacctgagacagatagcctcactcgcctcaccctagtcctggtggcatttgtctcaggtgcaaaatttaagaaagaaaccttggagtgctcaccctgtggctgggtagatggtcctaaagtggtggttttcaagcctgagtgtgtatcaggatcatcaggggagcttgctaaagagcagttcctgcggtcagaccctcatgcattttgagcaggtgtggggactgggaaactgcatctgtaacctgctgtaatctaacgcttatctaaatactactgtgctcacacagagaacaccgcaaaagtagaggtgttcctccagagggcaggtgagcagatggcacagtctgcttggaattcagtcaggtgatgagagatgagatgaggcactcctagctttgggaagagggagctgaaagatgaacctttgcaggtgcccacggtcaaagtggtggtttaatgccatgccatgcccattttctgttggccttggcagggagttacagccctaccttaggacctggctccttatttctgctgtaggctctttcctgccctggccgagatggagtggaatgagacctagaaacatcaagctaaatacatgtcctcagaaagataaaggtttacattttcacccccatcaaatctgaaagctctctgcctgtgtttttctaagggatagggacatcattactcagtccacaacctggactcatgtagggtcccctgtcagtaaaggagtcagtcaagcccaccaggtataccaaggactcttaccctcagcccctactccttggaaagctgccccttggcctaatattggtgtttagcttgagcctgactccttctcaacactaagagctgatgaagtcctgaagcagaaagagctctgacctgagagtcaaacatccttattctgatctcagctcagcccctgatttgttgtgtgaccctggatatgtcacttcctgtctttttgactttttaaaatgaagggtagactagaggagagcttctaaaactttaatgtggtcaacgaaatggaataggaaattccacaagtctgtccttccacaaaagcagcaaataaggtggcaaaaactcaaatttatgggaactctggaaacgaattgaaagtttacagcaatcaggtgaatacctaagaataaaagctggatttagtaaga

SEQ ID NO: 34 - DNA, 2804 bp, Homo sapiens
  misc_feature 1..2804: note = NM_001278409.1 Homo sapiens annexin A11 (ANXA11), transcriptvariant f, mRNA
  source 1..2804: mol_type = other DNA
gcactgcctctggcacctggggcagccgcgcccgcggagttttccgcccggcgctgacggctgctgcgcccgcggctccccagtgccccgagtgccccgcgggccccgcgagcgggagtgggacccagcccctaggcagaacccaggcgccgcgcccgggacgcccgcggagagagccactcccgcccacgtcccatttcgcccctcgcgtccggagtccccgtggccaggtgtgtgtctggggaagagacttacagaagtggagttgctgagtcaaagatctaaccatgagctaccctggctatcccccgcccccaggtggctacccaccagctgcaccaggttggctggcactggcctgggttctctctctatagtagaaatcctgccatccagatcctgccactgccacctttgctagcacagctgagcagcctctgagcagcaagagaggaggaggcaggaaatttagggaaggttcttcctggagggtctggagccctggagatgaagagccgatccgaagctgccatgtagaggaaagcatctaacaggccagaggccccatgatgatgtcgaatgcccatcgggcacccagctgagccctgcaggtggtggtccctggggaggtgctgcctaccctcctccgcccagcatgccccccatcgggctggataacgtggccacctatgcggggcagttcaaccaggactatctctcgggaatggcggccaacatgtctgggacatttggaggagccaacatgcccaacctgtaccctggggcccctggggctggctacccaccagtgccccctggcggctttgggcagcccccctctgcccagcagcctgttcctccctatgggatgtatccacccccaggaggaaacccaccctccaggatgccctcatatccgccatacccaggggcccctgtgccgggccagcccatgccaccccccggacagcagcccccaggggcctaccctgggcagccaccagtgacctaccctggtcagcctccagtgccactccctgggcagcagcagccagtgccgagctacccaggatacccggggtctgggactgtcacccccgctgtgcccccaacccagtttggaagccgaggcaccatcactgatgctcccggctttgaccccctgcgagatgccgaggtcctgcggaaggccatgaaaggcttcgggacggatgagcaggccatcattgactgcctggggagtcgctccaacaagcagcggcagcagatcctactttccttcaagacggcttacggcaaggatttgatcaaagatctgaaatctgaactgtcaggaaactttgagaagacaatcttggctctgatgaagaccccagtcctctttgacatttatgagataaaggaagccatcaagggggttggcactgatgaagcctgcctgattgagatcctcgcttcccgcagcaatgagcacatccgagaattaaacagagcctacaaagcagaattcaaaaagaccctggaagaggccattcgaagcgacacatcagggcacttccagcggctcctcatctctctctctcagggaaaccgtgatgaaagcacaaacgtggacatgtcactcgcccagagagatgcccaggagctgtatgcggccggggagaaccgcctgggaacagacgagtccaagttcaatgcggttctgtgctcccggagccgggcccacctggtagcagttttcaatgagtaccagagaatgacaggccgggacattgagaagagcatctgccgggagatgtccggggacctggaggagggcatgctggccgtggtgaaatgtctcaagaataccccagccttctttgcggagaggctcaacaaggccatgaggggggcaggaacaaaggaccggaccctgattcgcatcatggtgtctcgcagcgagaccgacctcctggacatcagatcagagtataagcggatgtacggcaagtcgctgtaccacgacatctcgggagatacttcaggggattaccggaagattctgctgaagatctgtggtggcaatgactgaacagtgactggtggctcacttctgcccacctgccggcaacaccagtgccaggaaaaggccaaaagaatgtctgtttctaacaaatccacaaatagccccgagattcaccgtcctagagcttaggcctgtcttccacccctcctgacccgtatagtgtgccacaggacctgggtcggtctagaactctctcaggatgccttttctaccccatccctcacagcctcttgctgctaaaatagatgtttcatttttctgactcatgcaatcattcccctttgcctgtggctaagacttggcttcatttcgtcatgtaattgtatatttttatttggaggcatattttcttttcttacagtcattgccagacagaggcatacaagtctgtttgctgcatacacatttctggtgagggcgactgggtgggtgaagcaccgtgtcctcgctgaggagagaaagggaggcgtgcctgagaaggtagcctgtgcatctggtgagtgtgtcacgagctttgttactgccaaactcactcctttttagaaaaaacaaaaaaaaagggccagaaagtcattccttccatcttccttgcagaaaccacgagaacaaagccagttccctgtcagtgacagggcttcttgtaatttgtggtatgtgccttaaacctgaatgtctgtagccaaaacttgtttccacattaagagtcagccagctctggaatggtctggaaatgtca

SEQ ID NO: 35 - DNA, 1456 bp, Homo sapiens
  misc_feature 1..1456: note = NM_004306.4 Homo sapiens annexin A13 (ANXA13), transcript variant1, mRNA
  source 1..1456: mol_type = other DNA
gcctgtaggaggactgatctcttgatgaaatacagaaaaaccatctcagaaaaaggaaaatgggcaatcgtcatgctaaagcgagcagtcctcagggttttgatgtggatcgagatgccaaaaagctgaacaaagcctgcaaaggaatggggaccaatgaagcagccatcattgaaatcttatcgggcaggacatcagatgagaggcaacaaatcaagcaaaagtacaaggcaacgtacggcaaggagctggaggaagtactcaagagtgagctgagtggaaacttcgagaagacagcgttggcccttctggaccgtcccagcgagtacgccgcccggcagctgcagaaggctatgaagggtctgggcacagatgagtccgtcctcattgaggtcctgtgcacgaggaccaataaggaaatcatcgccattaaagaggcctaccaaaggctatttgataggagcctcgaatcagatgtcaaaggtgatacaagtggaaacctaaaaaaaatcctggtgtctctgctgcaggctaatcgcaatgaaggagatgacgtggacaaagatctagctggtcaggatgccaaagatctgtatgatgcaggggaaggccgctggggcactgatgagcttgcgttcaatgaagtcctggccaagaggagctacaagcagttacgagccacctttcaagcctatcaaattctcattggcaaagacatagaagaagccattgaagaagaaacatcaggcgacttgcagaaggcctatttaactctcgtgagatgtgcccaggattgtgaggactattttgctgaacgtctgtacaagtcgatgaagggtgcggggaccgatgaggagacgttgattcgcatagtcgtgaccagggccgaggtggaccttcaggggatcaaagcaaagttccaagagaagtatcagaagtctctctctgacatggttcgctcagatacctccggggacttccggaaactgctagtagccctcttgcactgagccaagccagggcaataggaacacagggtggaaccgcctttgtcaagagcacattccaaatcaaacttgcaaatgagactcccgcacgaaaacccttaagagtcccggattactttcttggcagcttaagtggcgcagccaggccaagctgtgtaagttaagggcagtaacgttaagatgcgtgggcagggcaccttgaactctggcttagcaagcatctaggctgcctcttcactttcttttagcatggtaactggatgttttctaaacactaatgaaatcagcagttgatgaaaaaactatgcatttgtaatggcacatttagaaggatatgcatcacacaagtaaggtacaggaaagacaaaattaaacaatttattaattttccttctgtgtgttcaatttgaaagcctcattgttaattaaagttgtggattatgcctcta

SEQ ID NO: 36 - DNA, 1633 bp, Homo sapiens
  misc_feature 1..1633: note = NM_001003954.2 Homo sapiens annexin A13 (ANXA13), transcriptvariant 2, mRNA
  source 1..1633: mol_type = other DNA
attatgtccggggggaaaactgttgtaaactttgcctgtaggaggactgatctcttaatgaaatacagaaaaaccatctcagaaaaaggaaaatgggcaatcgtcatagccagtcgtacaccctctcagaaggcagtcaacagttgcctaaaggggactcccaaccctcgacagtcgtgcagcctctcagccacccatcacggaatggagagccagaggccccacagcctgctaaagcgagcagtcctcagggttttgatgtggatcgagatgccaaaaagctgaacaaagcctgcaaaggaatggggaccaatgaagcagccatcattgaaatcttatcgggcaggacatcagatgagaggcaacaaatcaagcaaaagtacaaggcaacgtacggcaaggagctggaggaagtactcaagagtgagctgagtggaaacttcgagaagacagcgttggcccttctggaccgtcccagcgagtacgccgcccggcagctgcagaaggctatgaagggtctgggcacagatgagtccgtcctcattgaggtcctgtgcacgaggaccaataaggaaatcatcgccattaaagaggcctaccaaaggctatttgataggagcctcgaatcagatgtcaaaggtgatacaagtggaaacctaaaaaaaatcctggtgtctctgctgcaggctaatcgcaatgaaggagatgacgtggacaaagatctagctggtcaggatgccaaagatctgtatgatgcaggggaaggccgctggggcactgatgagcttgcgttcaatgaagtcctggccaagaggagctacaagcagttacgagccacctttcaagcctatcaaattctcattggcaaagacatagaagaagccattgaagaagaaacatcaggcgacttgcagaaggcctatttaactctcgtgagatgtgcccaggattgtgaggactattttgctgaacgtctgtacaagtcgatgaagggtgcggggaccgatgaggagacgttgattcgcatagtcgtgaccagggccgaggtggaccttcaggggatcaaagcaaagttccaagagaagtatcagaagtctctctctgacatggttcgctcagatacctccggggacttccggaaactgctagtagccctcttgcactgagccaagccagggcaataggaacacagggtggaaccgcctttgtcaagagcacattccaaatcaaacttgcaaatgagactcccgcacgaaaacccttaagagtcccggattactttcttggcagcttaagtggcgcagccaggccaagctgtgtaagttaagggcagtaacgttaagatgcgtgggcagggcaccttgaactctggcttagcaagcatctaggctgcctcttcactttcttttagcatggtaactggatgttttctaaacactaatgaaatcagcagttgatgaaaaaactatgcatttgtaatggcacatttagaaggatatgcatcacacaagtaaggtacaggaaagacaaaattaaacaatttattaattttccttctgtgtgttcaatttgaaagcctcattgttaattaaagttgtggattatgcctctaaaaaaaaaaaaaaaaaaaaaa

SEQ ID NO: 37 - protein, 667 aa, Homo sapiens
  REGION 1..667: note = MISC_FEATURE - NP_001350043.1 annexin A6 isoform 3
MAKPAQGAKYRGSIHDFPGFDPNQDAEALYTAMKGFGSDKEAILDIITSRSNRQRQEVCQSYKSLYGKDLIADLKYELTGKFERLIVGLMRPPAYCDAKEIKDAISGIGTDEKCLIEILASRTNEQMHQLVAAYKDAYERDLEADIIGDTSGHFQKMLVVLLQGTREEDDVVSEDLVQQDVQDLYEAGELKWGTDEAQFIYILGNRSKQHLRLVFDEYLKTTGKPIEASIRGELSGDFEKLMLAVVKCIRSTPEYFAERLFKAMKGLGTRDNTLIRIMVSRSELDMLDIREIFRTKYEKSLYSMIKNDTSGEYKKTLLKLSGGDDDAAGQFFPEAAQVAYQMWELSAVARVELKGTVRPANDFNPDADAKALRKAMKGLGTDEDTIIDIITHRSNVQRQQIRQTFKSHFGRDLMTDLKSEISGDLARLILGLMMPPAHYDAKQLKKAMEGAGTDEKALIEILATRTNAEIRAINEAYKEDYHKSLEDALSSDTSGHFRRILISLATGHREEGGENLDQAREDAQEIADTPSGDKTSLETRFMTILCTRSYPHLRRVFQEFIKMTNYDVEHTIKKEMSGDVRDAFVAIVQSVKNKPLFFADKLYKSMKGAGTDEKTLTRIMVSRSEIDLLNIRREFIEKYDKSLHQAIEGDTSGDFLKALLALCGGED

SEQ ID NO: 38 - DNA, 2871 bp, Homo sapiens
  misc_feature 1..2871: note = NM_001363114.2 Homo sapiens annexin A6 (ANXA6), transcriptvariant 3, mRNA
  source 1..2871: mol_type = other DNA
gcggttgctgctgggctaacgggctccgatccagcgagcgctgcgtcctcgagtccctgcgcccgtgcgtccgtctgcgacccgaggcctccgctgcgcgtggattctgctgcgaaccggagaccatggccaaaccagcacagggtgccaagtaccggggctccatccatgacttcccaggctttgaccccaaccaggatgccgaggctctgtacactgccatgaagggctttggcagtgacaaggaggccatactggacataatcacctcacggagcaacaggcagaggcaggaggtctgccagagctacaagtccctctacggcaaggacctcattgctgatttaaagtatgaattgacgggcaagtttgaacggttgattgtgggcctgatgaggccacctgcctattgtgatgccaaagaaattaaagatgccatctcgggcattggcactgatgagaagtgcctcattgagatcttggcttcccggaccaatgagcagatgcaccagctggtggcagcatacaaagatgcctacgagcgggacctggaggctgacatcatcggcgacacctctggccacttccagaagatgcttgtggtcctgctccagggaaccagggaggaggatgacgtagtgagcgaggacctggtacaacaggatgtccaggacctatacgaggcaggggaactgaaatggggaacagatgaagcccagttcatttacatcttgggaaatcgcagcaagcagcatcttcggttggtgttcgatgagtatctgaagaccacagggaagccgattgaagccagcatccgaggggagctgtctggggactttgagaagctaatgctggccgtagtgaagtgtatccggagcaccccggaatattttgctgaaaggctcttcaaggctatgaagggcctggggactcgggacaacaccctgatccgcatcatggtctcccgtagtgagttggacatgctcgacattcgggagatcttccggaccaagtatgagaagtccctctacagcatgatcaagaatgacacctctggcgagtacaagaagactctgctgaagctgtctgggggagatgatgatgctgctggccagttcttcccggaggcagcgcaggtggcctatcagatgtgggaacttagtgcagtggcccgagtagagctgaagggaactgtgcgcccagccaatgacttcaaccctgacgcagatgccaaagcgctgcggaaagccatgaagggactcgggactgacgaagacacaatcatcgatatcatcacgcaccgcagcaatgtccagcggcagcagatccggcagaccttcaagtctcactttggccgggacttaatgactgacctgaagtctgagatctctggagacctggcaaggctgattctggggctcatgatgccaccggcccattacgatgccaagcagttgaagaaggccatggagggagccggcacagatgaaaaggctcttattgaaatcctggccactcggaccaatgctgaaatccgggccatcaatgaggcctataaggaggactatcacaagtccctggaggatgctctgagctcagacacatctggccacttcaggaggatcctcatttctctggccacggggcatcgtgaggagggaggagaaaacctggaccaggcacgggaagatgcccaggaaatagcagacacacctagtggagacaaaacttccttggagacacgtttcatgacgatcctgtgtacccggagctatccgcacctccggagagtcttccaggagttcatcaagatgaccaactatgacgtggagcacaccatcaagaaggagatgtctggggatgtcagggatgcatttgtggccattgttcaaagtgtcaagaacaagcctctcttctttgccgacaaactttacaaatccatgaagggtgctggcacagatgagaagactctgaccaggatcatggtatcccgcagtgagattgacctgctcaacatccggagggaattcattgagaaatatgacaagtctctccaccaagccattgagggtgacacctccggagacttcctgaaggccttgctggctctctgtggtggtgaggactagggccacagctttggcgggcacttctgccaagaaatggttatcagcaccagccgccatggccaagcctgattgttccagctccagagactaaggaaggggcaggggtggggggaggggttgggttgggctcttatcttcagtggagcttaggaaacgctcccactcccacgggccatcgagggcccagcacggctgagcggctgaaaaaccgtagccatagatcctgtccacctccactcccctctgaccctcaggctttcccagcttcctccccttgctacagcctctgccctggtttgggctatgtcagatccaaaaacatcctgaacctctgtctgtaaaatgagtagtgtctgtactttgaatgagggggttggtggcaggggccagttgaatgtgctgggcggggtggtgggaaggatagtaaatgtgctggggcaaactgacaaatcttcccatccatttcaccacccatctccatccaggccgcgctagagtactggaccaggaatttggatgcctgggttcaaatctgcatctgccatgcacttgtttctgaccttaggccagcccctttccctccctgagtctctattttcttatctacaatgagacagttggacaaaaaaatcttggcttcccttctaacattaacttcctaaagtatgcctccgattcattcccttgacactttttatttctaaggaagaaataaaaagagatacacaaacacataaacaca

SEQ ID NO: 39 - DNA, 20 bp, synthetic construct
  misc_feature 1..20: note = Synthetic
  source 1..20: mol_type = other DNA
gcttgctctgtgtggcggag

SEQ ID NO: 40 - DNA, 20 bp, synthetic construct
  misc_feature 1..20: note = Synthetic
  source 1..20: mol_type = other DNA
agagggggccctctgaggtc

SEQ ID NO: 41 - DNA, 18 bp, synthetic construct
  misc_feature 1..18: note = Synthetic
  source 1..18: mol_type = other DNA
ctaggccgatggctgcta

SEQ ID NO: 42 - DNA, 20 bp, synthetic construct
  misc_feature 1..20: note = Synthetic
  source 1..20: mol_type = other DNA
caatggcttggtcaggtcac

SEQ ID NO: 43 - DNA, 23 bp, synthetic construct
  misc_feature 1..23: note = Synthetic
  source 1..23: mol_type = other DNA
acttctcgatgcgggtgttggtg

(19) United States
(12) Patent Application Publication
(10) Pub. No.: US 2025/0195609 A1
(43) Pub. Date: Jun. 19, 2025

(54) USE OF ANNEXINS IN PREVENTING AND TREATING CARDIAC NEURONAL CELL MEMBRANE INJURY AND DISEASE

(71) Applicant: NORTHWESTERN UNIVERSITY, Evanston, IL (US)

(72) Inventors: Alexis R. Demonbreun, Mokena, IL (US); Dominic E. Fullenkamp, Evanston, IL (US); Katherine Rose Sadleir, Evanston, IL (US); Robert J. Vassar, Evanston, IL (US); Elizabeth M. McNally, Oak Park, IL (US)

(21) Appl. No.: 18/838,195

(22) PCT Filed: Feb. 13, 2023
(86) PCT No.: PCT/US23/62489

§ 371 Date: Aug. 13, 2024

Related U.S. Application Data
(60) Provisional application No. 63/377,274, filed on Sep. 27, 2022.
(60) Provisional application No. 63/309,925, filed on Feb. 14, 2022.

(51) Int. Cl. A61K 38/17; A61K 45/06; A61P 9/10; A61P 25/28

(52) U.S. Cl. CPC A61K 38/1709; A61K 45/06; A61P 9/10; A61P 25/28

(57) ABSTRACT

The present disclosure generally provides compositions and methods for increasing the activity of an annexin protein to treat a cellular membrane injury in a patient in need thereof. In some aspects, the disclosure provides methods of treating a patient suffering from a nerve injury comprising administering a therapeutically effective amount of a composition comprising an agent that increases the activity of an annexin protein. In further aspects, the disclosure provides methods of reducing serum or plasma level of lactate dehydrogenase (LDH), cardiac troponin T, cardiac troponin I, creatine kinase (CK), or a combination thereof, in a patient in need thereof, comprising administering a therapeutically effective amount of an agent that increases the activity of an annexin protein to the patient.

### CROSS-REFERENCE TO RELATED APPLICATIONS

This application claims the priority benefit under 35 U.S.C. § 119(e) of U.S. Provisional Patent Application No. 63/309,925, filed Feb. 14, 2022 and U.S. Provisional Application No. 63/377,274, filed Sep. 27, 2022, which are each incorporated herein by reference in their entirety.

### STATEMENT OF GOVERNMENT INTEREST

This invention was made with government support under grant numbers U54 AR052646, 5F32HL154712-02, 5R01AG030142-10, SUB00002815//2P50AR052646-16A1, and RO1 NS047726 awarded by the National Institutes of Health. The government has certain rights in the invention.

### INCORPORATION BY REFERENCE OF MATERIALS SUBMITTED ELECTRONICALLY

This application contains, as a separate part of the disclosure, a Sequence Listing in computer readable form (Filename: 2022-008RSeqListing.xml; Size: 110,592 bytes; Created: Feb. 13, 2023), which is incorporated by reference in its entirety.

### BACKGROUND

The plasma membrane is frequently exposed to mechanical disruption resulting in membrane lesions, which may vary in shape and size depending on cell type and function. Mutations in genes that function to either stabilize or repair the plasma membrane are associated with multiple distinct conditions including muscular dystrophy, cardiomyopathy, and neuropathy (Dias C, and Nylandsted J. Plasma membrane integrity in health and disease: significance and therapeutic potential. Cell Discov. 2021; 7(1):4; Ammendolia D A, Bement W M, and Brumell J H. Plasma membrane integrity: implications for health and disease. BMC Biol. 2021; 19(1):71).

Annexins are Ca²⁺-binding proteins that regulate lipid binding, cytoskeletal reorganization, and bleb formation, steps necessary for membrane repair (Bizzarro et al., 2012; Boye et al., 2018; Boye et al., 2017; Grewal et al., 2017; Jimenez and Perez, 2017; Lauritzen et al., 2015). Annexins have a high affinity for phosphatidylserine, phosphatidylinositol, and cholesterol, which are highly enriched in the sarcolemma (Fiehn et al., 1971; Gerke et al., 2005).

### SUMMARY

Defects in membrane stability contribute to and exacerbate chronic disorders like muscular dystrophy, cardiomyopathy, and neuropathy where the target cell type is highly dependent on repair. Additionally, membrane injury can result from acute events such as overuse, trauma, burn, chemical exposure and chronic disease. Currently, there is a lack of agents that prevent or treat membrane damage. Annexins are calcium-binding proteins that have a high affinity for membrane lipids. Annexins are sequentially recruited to the lesion forming a repair complex nucleated by annexin A6.

Applications of the technology described herein include, but are not limited to:
Treatment or prevention of cardiomyocyte membrane injury
Treatment or prevention of cardiac injury
Treatment or prevention of cardiac disease
Enhancement of cardiomyocyte membrane repair
Treatment or prevention of neuronal membrane injury
Treatment or prevention of neuronal injury
Treatment or prevention of neuronal disease
Enhancement of neuronal membrane repair
Treatment or prevention of brain injury
Treatment or prevention of brain disease
Enhancement of blood brain barrier repair
Treatment or prevention of disorders disclosed herein

Advantages of the technology described herein include, but are not limited to:
Regents that treat or prevent membrane injury are virtually non-existant
Recombinant annexin A6 can be dosed per body weight
Recombinant annexin A6 can be administered acutely or chronically
Recombinant annexin A6 can be administered independent of age
Recombinant annexin A6 can be administered for acute or chronic forms of injury
Systemic or local injection may be used

In some aspects the disclosure provides a method of treating a patient suffering from a disorder comprising administering to the patient a therapeutically effective amount of a composition comprising an agent that increases the activity of an annexin protein, wherein the disorder is stroke, dementia, Alzheimer Dementia, Frontotemporal Dementia, Parkinsons Disease, spinal cord injury, small vessel disease, transient ischemic attack, cerebrovascular accident, dementia due to small vessel disease, Guillain Barré, Acute Inflammatory Demyelinating, Polyradiculopathy, Peripheral nerve disease, neuropathy, diabetic neuropathy, acute myocardial infarction, hypertrophic cardiomyopathy, dilated cardiomyopathy, myocarditis, arrhythmogenic cardiomyopathy, restrictive cardiomyopathy, ischemic cardiomyopathy, myocardiac injury acute, or myocardial injury. In some aspects, the disclosure provides a method of delaying onset, enhancing recovery from cellular membrane injury, or preventing a disorder comprising administering to a patient in need thereof a therapeutically effective amount of a composition comprising an agent that increases the activity of an annexin protein, wherein the disorder is stroke, dementia, Alzheimer Dementia, Frontotemporal Dementia, Parkinsons Disease, spinal cord injury, small vessel disease, transient ischemic attack, cerebrovascular accident, dementia due to small vessel disease, Guillain Barré, Acute Inflammatory Demyelinating, Polyradiculopathy, Peripheral nerve disease, neuropathy, diabetic neuropathy, acute myocardial infarction, hypertrophic cardiomyopathy, dilated cardiomyopathy, myocarditis, arrhythmogenic cardiomyopathy, restrictive cardiomyopathy, ischemic cardiomyopathy, myocardiac injury acute, or myocardial injury. In some embodiments, the agent is a recombinant protein, a steroid, a polynucleotide capable of expressing an annexin protein, or a combination thereof. In further embodiments, the steroid is a corticosteroid or a glucocorticoid. In still further embodiments, the recombinant protein is an annexin protein or a modified form thereof. In some embodiments, the annexin protein is annexin A6 (SEQ ID NO: 7, SEQ ID NO: 8, SEQ ID NO: 37, or a combination thereof) or a modified form thereof. In some embodiments, a method of the disclosure further comprises administering an effective amount of a second agent, wherein the second agent is mitsugumin 53 (MG53), a modulator of latent TGF-β binding protein 4 (LTBP4), a modulator of transforming growth factor β (TGF-β activity, a modulator of androgen response, a modulator of an inflammatory response, a promoter of muscle growth, a chemotherapeutic agent, a modulator of fibrosis, or a combination thereof.

In some embodiments, the polynucleotide is associated with a nanoparticle. In further embodiments, the polynucleotide is contained in a vector. In some embodiments, the vector is within a chloroplast. In further embodiments, the vector is a viral vector. In still further embodiments, the viral vector is a herpes virus vector, an adeno-associated virus (AAV) vector, an adeno virus vector, or a lentiviral vector. In some embodiments, the AAV vector is recombinant AAV5, AAV6, AAV8, AAV9, or AAV74. In some embodiments, the AAV74 vector is AAVrh74. In various embodiments, the composition increases the activity of annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 37, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), annexin A13 (SEQ ID NO: 17 or SEQ ID NO: 18), or a combination thereof. In some embodiments, the composition increases the activity of annexin A1 (SEQ ID NO: 1), annexin A2 (SEQ ID NO: 2 or SEQ ID NO: 3), and annexin A6 (SEQ ID NO: 7, SEQ ID NO: 8, SEQ ID NO: 37, or a combination thereof). In some embodiments, the composition increases the activity of annexin A2 (SEQ ID NO: 2 or SEQ ID NO: 3) and annexin A6 (SEQ ID NO: 7, SEQ ID NO: 8, SEQ ID NO: 37, or a combination thereof). In some embodiments, the composition increases the activity of annexin A1 (SEQ ID NO: 1) and annexin A6 (SEQ ID NO: 7, SEQ ID NO: 8, SEQ ID NO: 37, or a combination thereof). In some embodiments, the composition increases the activity of annexin A6 (SEQ ID NO: 7, SEQ ID NO: 8, SEQ ID NO: 37, or a combination thereof). In any of the aspects or embodiments of the disclosure a method of the disclosure uses a composition as described herein.

In some aspects, the disclosure provides a pharmaceutical composition comprising an annexin protein, or a modified form thereof, and a pharmaceutically acceptable carrier, buffer, and/or diluent. In some embodiments, the annexin protein is annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 37, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), annexin A13 (SEQ ID NO: 17 or SEQ ID NO: 18), or a combination thereof. In some embodiments, the annexin protein is annexin A6 (SEQ ID NO: 7, SEQ ID NO: 8, SEQ ID NO: 37, or a combination thereof). In some embodiments, the pharmaceutical composition comprises annexin A1 (SEQ ID NO: 1), annexin A2 (SEQ ID NO: 2 or SEQ ID NO: 3), and annexin A6 (SEQ ID NO: 7, SEQ ID NO: 8, SEQ ID NO: 37, or a combination thereof). In some embodiments, the pharmaceutical composition comprises annexin A2 (SEQ ID NO: 2 or SEQ ID NO: 3) and annexin A6 (SEQ ID NO: 7, SEQ ID NO: 8, SEQ ID NO: 37, or a combination thereof). In some embodiments, the pharmaceutical composition comprises annexin A1 (SEQ ID NO: 1) and annexin A6 (SEQ ID NO: 7, SEQ ID NO: 8, SEQ ID NO: 37, or a combination thereof). In some embodiments, the composition further comprises a steroid. In further embodiments, the steroid is a corticosteroid or a glucocorticoid. In some embodiments, the composition further comprises an effective amount of a second agent, wherein the second agent is mitsugumin 53 (MG53), a modulator of latent TGF-β binding protein 4 (LTBP4), a modulator of transforming growth factor β (TGF-β activity, a modulator of androgen response, a modulator of an inflammatory response, a promoter of muscle growth, a chemotherapeutic agent, a modulator of fibrosis, or a combination thereof. In some embodiments, purity of the annexin protein in the composition is about 90% or higher as measured by standard release assay. In further embodiments, the composition has an endotoxin level that is less than about 0.50000 endotoxin units per milligram (EU/mg).

In some aspects, the disclosure provides a pharmaceutical composition comprising annexin A6 (SEQ ID NO: 7, SEQ ID NO: 8, SEQ ID NO: 37, or a combination thereof), or a modified form thereof, and a pharmaceutically acceptable carrier, buffer, and/or diluent. In some embodiments, the composition further comprises annexin A1 (SEQ ID NO: 1), or a modified form thereof, and annexin A2 (SEQ ID NO: 2 or SEQ ID NO: 3), or a modified form thereof. In some embodiments, the composition further comprises annexin A2 (SEQ ID NO: 2 or SEQ ID NO: 3), or a modified form thereof. In some embodiments, the composition further comprises annexin A1 (SEQ ID NO: 1), or a modified form thereof. In some embodiments, the composition further comprises a steroid. In further embodiments, the steroid is a corticosteroid or a glucocorticoid. In some embodiments, the composition further comprises an effective amount of a second agent, wherein the second agent is mitsugumin 53 (MG53), a modulator of latent TGF-β binding protein 4 (LTBP4), a modulator of transforming growth factor β (TGF-β activity, a modulator of androgen response, a modulator of an inflammatory response, a promoter of muscle growth, a chemotherapeutic agent, a modulator of fibrosis, or a combination thereof. In some embodiments, purity of the annexin protein in the composition is about 90% or higher as measured by standard release assay. In some embodiments, the composition has an endotoxin level that is less than about 0.50000 endotoxin units per milligram (EU/mg). In further embodiments, the annexin A6 (SEQ ID NO: 7, SEQ ID NO: 8, SEQ ID NO: 37, or a combination thereof), or a modified form thereof, is produced in a prokaryotic cell.

In some aspects, the disclosure provides a method of treating a patient suffering from a nerve injury comprising administering to the patient a therapeutically effective amount of a composition comprising an agent that increases the activity of an annexin protein. In further aspects, the disclosure provides a method of delaying onset, enhancing recovery from a nerve injury, or preventing a nerve injury, comprising administering to a patient in need thereof a therapeutically effective amount of a composition comprising an agent that increases the activity of an annexin protein. In some embodiments, the nerve injury is an acute nerve injury or a chronic nerve injury. In some embodiments, the nerve injury is a partially transected nerve, a wholly transected nerve, a nerve injury due to ischemia, a nerve injury due to infection, a nerve injury due to trauma, or a combination thereof. In further embodiments, the patient has a crush injury, a concussion, traumatic brain injury (TBI), or peripheral nerve disease.

In some aspects, the disclosure provides a method comprising administering a therapeutically effective amount of a composition comprising an agent that increases the activity of an annexin protein to a patient, wherein the patient has an elevated serum or plasma level of lactate dehydrogenase (LDH), cardiac troponin T, cardiac troponin I, creatine kinase (CK), or a combination thereof, relative to a control level. In some aspects, the disclosure provides a method of reducing serum or plasma level of lactate dehydrogenase (LDH), cardiac troponin T, cardiac troponin I, creatine kinase (CK), or a combination thereof, in a patient in need thereof, comprising administering a therapeutically effective amount of a composition comprising an agent that increases the activity of an annexin protein to the patient, thereby reducing the serum or plasma level of lactate dehydrogenase (LDH), cardiac troponin T, cardiac troponin I, creatine kinase (CK), or a combination thereof in the patient. In some embodiments, the serum or plasma level of LDH in the patient prior to administration of the agent is elevated about 1.25-fold or more over a normal control range. In some embodiments, the serum or plasma level of cardiac troponin T and/or cardiac troponin I in the patient prior to administration of the agent is elevated about 1.25-fold or more over a normal control range. In some embodiments, the serum or plasma level of CK in the patient prior to administration of the agent is elevated about 1.25-fold or more over a normal control range. In further embodiments, the serum or plasma level of LDH in the patient is reduced by about 25% or more 24-48 hours after administration of the agent. In some embodiments, the serum or plasma level of cardiac troponin T and/or cardiac troponin I in the patient is reduced by about 25% or more 24-48 hours after administration of the agent. In further embodiments, the serum or plasma level of CK in the patient is reduced by about 25% or more 24-48 hours after administration of the agent. In some embodiments, the agent is a recombinant protein, a steroid, a polynucleotide capable of expressing an annexin protein, or a combination thereof. In further embodiments, the steroid is a corticosteroid or a glucocorticoid. In some embodiments, the recombinant protein is an annexin protein. In some embodiments, the annexin protein is annexin A6 (SEQ ID NO: 7, SEQ ID NO: 8, SEQ ID NO: 37, or a combination thereof) or a modified form thereof. In some embodiments, the patient suffers from an acute injury. In further embodiments, the acute injury results from surgery, a burn, a toxin, a chemical, radiation-induced injury, acute myocardial injury, acute muscle injury, acute lung injury, acute epithelial injury, acute epidermal injury, acute kidney injury, acute liver injury, vascular injury, an excessive mechanical force, trauma, acute brain injury from stroke or trauma, myositis, or acute cardiac injury. In some embodiments, the patient suffers from a chronic disorder. In further embodiments, the chronic disorder is Becker Muscular Dystrophy (BMD), Duchenne Muscular Dystrophy (DMD), Limb Girdle Muscular Dystrophy, Friedreich's Ataxia, congenital Muscular Dystrophy, Emery-Dreifuss Muscular Dystrophy (EDMD), Myotonic Dystrophy, Fascioscapulohumeral Dystrophy (FSHD), Oculopharyngeal Muscular Dystrophy, Distal Muscular Dystrophy, cystic fibrosis, pulmonary fibrosis, muscle atrophy, cerebral palsy, an epithelial disorder, an epidermal disorder, a kidney disorder, a liver disorder, sarcopenia, chronic cardiac injury, or cardiomyopathy (hypertrophic, dilated, congenital, arrhythmogenic, restrictive, ischemic, heart failure). In still further embodiments, the cardiomyopathy is hypertrophic, dilated, congenital, arrhythmogenic, restrictive, ischemic, Friedreich Ataxia, or heart failure. In some embodiments, a method of the disclosure further comprises administering an effective amount of a second agent, wherein the second agent is mitsugumin 53 (MG53), a modulator of latent TGF-β binding protein 4 (LTBP4), a modulator of transforming growth factor β (TGF-β activity, a modulator of androgen response, a modulator of an inflammatory response, a promoter of muscle growth, a chemotherapeutic agent, a modulator of fibrosis, or a combination thereof. In some embodiments, the polynucleotide is associated with a nanoparticle. In some embodiments, the polynucleotide is contained in a vector. In further embodiments, the vector is within a chloroplast. In still further embodiments, the vector is a viral vector. In some embodiments, the viral vector is a herpes virus vector, an adeno-associated virus (AAV) vector, an adeno virus vector, or a lentiviral vector. In further embodiments, the AAV vector is recombinant AAV5, AAV6, AAV8, AAV9, or AAV74. In still further embodiments, the AAV74 vector is AAVrh74. In some embodiments, the composition increases the activity of annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 37, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), annexin A13 (SEQ ID NO: 17 or SEQ ID NO: 18), or a combination thereof. In some embodiments, the composition increases the activity of annexin A1 (SEQ ID NO: 1), annexin A2 (SEQ ID NO: 2 or SEQ ID NO: 3), and annexin A6 (SEQ ID NO: 7, SEQ ID NO: 8, SEQ ID NO: 37, or a combination thereof). In some embodiments, the composition increases the activity of annexin A2 (SEQ ID NO: 2 or SEQ ID NO: 3) and annexin A6 (SEQ ID NO: 7, SEQ ID NO: 8, SEQ ID NO: 37, or a combination thereof). In some embodiments, the composition increases the activity of annexin A1 (SEQ ID NO: 1) and annexin A6 (SEQ ID NO: 7, SEQ ID NO: 8, SEQ ID NO: 37, or a combination thereof). In some embodiments, the composition increases the activity of annexin A6 (SEQ ID NO: 7, SEQ ID NO: 8, SEQ ID NO: 37, or a combination thereof). In any of the aspects or embodiments of the disclosure, the composition is a pharmaceutical composition as described herein. In some aspects, the disclosure provides a pharmaceutical composition comprising an annexin protein, or a modified form thereof, and a pharmaceutically acceptable carrier, buffer, and/or diluent. In some embodiments, the annexin protein is annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 37, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), annexin A13 (SEQ ID NO: 17 or SEQ ID NO: 18), or a combination thereof. In some embodiments, the annexin protein is annexin A6 (SEQ ID NO: 7, SEQ ID NO: 8, SEQ ID NO: 37, or a combination thereof). In some embodiments, the pharmaceutical composition comprises annexin A1 (SEQ ID NO: 1), annexin A2 (SEQ ID NO: 2 or SEQ ID NO: 3), and annexin A6 (SEQ ID NO: 7, SEQ ID NO: 8, SEQ ID NO: 37, or a combination thereof). In some embodiments, the pharmaceutical composition comprises annexin A2 (SEQ ID NO: 2 or SEQ ID NO: 3) and annexin A6 (SEQ ID NO: 7, SEQ ID NO: 8, SEQ ID NO: 37, or a combination thereof). In some embodiments, the pharmaceutical composition comprises annexin A1 (SEQ ID NO: 1) and annexin A6 (SEQ ID NO: 7, SEQ ID NO: 8, SEQ ID NO: 37, or a combination thereof). In some embodiments, the composition further comprises a steroid. In further embodiments, the steroid is a corticosteroid or a glucocorticoid. In some embodiments, the composition further comprises an effective amount of a second agent, wherein the second agent is mitsugumin 53 (MG53), a modulator of latent TGF-β binding protein 4 (LTBP4), a modulator of transforming growth factor β (TGF-β activity, a modulator of androgen response, a modulator of an inflammatory response, a promoter of muscle growth, a chemotherapeutic agent, a modulator of fibrosis, or a combination thereof. In some embodiments, purity of the annexin protein in the composition is about 90% or higher as measured by standard release assay. In some embodiments, the composition has an endotoxin level that is less than about 0.50000 endotoxin units per milligram (EU/mg). In further aspects, the disclosure provides a pharmaceutical composition comprising annexin A6 (SEQ ID NO: 7, SEQ ID NO: 8, SEQ ID NO: 37, or a combination thereof), or a modified form thereof, and a pharmaceutically acceptable carrier, buffer, and/or diluent. In some embodiments, the composition further comprises annexin A1 (SEQ ID NO: 1), or a modified form thereof, and annexin A2 (SEQ ID NO: 2 or SEQ ID NO: 3), or a modified form thereof. In some embodiments, the composition further comprises annexin A2 (SEQ ID NO: 2 or SEQ ID NO: 3), or a modified form thereof. In some embodiments, the composition further comprises annexin A1 (SEQ ID NO: 1), or a modified form thereof. In some embodiments, the composition further comprises a steroid. In further embodiments, the steroid is a corticosteroid or a glucocorticoid. In some embodiments, the composition further comprises an effective amount of a second agent, wherein the second agent is mitsugumin 53 (MG53), a modulator of latent TGF-β binding protein 4 (LTBP4), a modulator of transforming growth factor β (TGF-β activity, a modulator of androgen response, a modulator of an inflammatory response, a promoter of muscle growth, a chemotherapeutic agent, a modulator of fibrosis, or a combination thereof. In some embodiments, purity of the annexin protein in the composition is about 90% or higher as measured by standard release assay. In some embodiments, the composition has an endotoxin level that is less than about 0.50000 endotoxin units per milligram (EU/mg). In some embodiments, the annexin A6 (SEQ ID NO: 7, SEQ ID NO: 8, SEQ ID NO: 37, or a combination thereof), or a modified form thereof is produced in a prokaryotic cell.

In some aspects, the disclosure provides a method of treating preclinical Alzheimer's disease or mild-to-moderate congnitive impairment comprising administering a therapeutically effective amount of a composition comprising an agent that increases the activity of an annexin protein to a patient in need thereof. In some embodiments, the patient has a plasma level of Aβ42 that is greater than zero and less than about 1000 picograms per milliliter (pg/ml). In some embodiments, the patient has a cerebrospinal fluid (CSF) ratio of Aβ42/Aβ40 ratio that is less than about 0.07. In some embodiments, the patient has a serum or plasma level of phosphorylated tau protein that is about 24 picograms per milliliter (pg/ml) or greater. In additional embodiments, the patient has an amount of phosphorylated tau protein in their cerebrospinal fluid (CSF) that is about 52 picograms per milliliter (pg/ml) or greater. In some embodiments, the patient has an amount of amyloid plaques in their brain that is about 10 to about 60 centiloids. In further embodiments, the patient was previously diagnosed with preclinical Alzheimer's disease or mild-to-moderate cognitive impairment via cognitive testing. In some embodiments, the method includes diagnosing the patient with preclinical Alzheimer's disease or mild-to-moderate cognitive impairment via cognitive testing. In some embodiments, the patient is amyloid positive, tau negative, and neurodegeneration negative. In some embodiments, the patient is amyloid positive, tau positive, and neurodegeneration negative. In further embodiments, the patient is amyloid positive, tau negative, and neurodegeneration positive. In still further embodiments, the patient is amyloid positive, tau positive, and neurodegeneration positive. In some aspects, the disclosure provides a method comprising administering a therapeutically effective amount of a composition comprising an agent that increases the activity of an annexin protein to a patient having preclinical Alzheimer's disease or mild-to-moderate cognitive impairment. In some embodiments, the patient has: (i) a cerebrospinal fluid (CSF) ratio of Aβ42/Aβ40 ratio that is less than about 0.07; (ii) a plasma level of Aβ42 that is greater than zero and less than about 1000 pg/ml; (iii) a serum or plasma level of phosphorylated tau protein that is about 24 pg/ml or greater; (iv) an amount of phosphorylated tau protein in their cerebrospinal fluid (CSF) that is about 52 pg/ml or greater; and/or (v) an amount of amyloid plaques in their brain that is about 10 to about 60 centiloids. In some embodiments, the patient was previously diagnosed with preclinical Alzheimer's disease or mild-to-moderate cognitive impairment via cognitive testing. In some embodiments, the method includes diagnosing the patient with preclinical Alzheimer's disease or mild-to-moderate cognitive impairment via cognitive testing. In various embodiments, the phosphorylated tau protein is p-tau181, p-tau231, p-tau217, or a combination thereof. In some embodiments, the phosphorylated tau protein is p-tau181. In various embodiments, the agent is a recombinant protein, a steroid, a polynucleotide capable of expressing an annexin protein, or a combination thereof. In further embodiments, the steroid is a corticosteroid or a glucocorticoid. In some embodiments, the recombinant protein is an annexin protein or a modified form thereof. In further embodiments, the annexin protein is annexin A6 (SEQ ID NO: 7, SEQ ID NO: 8, SEQ ID NO: 37, or a combination thereof) or a modified form thereof. In various embodiments, a method of the disclosure further comprises administering an effective amount of a second agent, wherein the second agent is an acetylcholinesterase inhibitor, a mild to moderate NMDA-receptor antagonist, an anti-amyloid antibody, a beta-secretase enzyme inhibitor, an anti-tau antibody, a modulator of microglial activity, mitsugumin 53 (MG53), a modulator of latent TGF-β binding protein 4 (LTBP4), a modulator of transforming growth factor β (TGF-β activity, a modulator of androgen response, a modulator of an inflammatory response, a chemotherapeutic agent, or a combination thereof. In some embodiments, the polynucleotide is associated with a nanoparticle. In further embodiments, the polynucleotide is contained in a vector. In still further embodiments, the vector is within a chloroplast. In yet additional embodiments, the vector is a viral vector. In further embodiments, the viral vector is a herpes virus vector, an adeno-associated virus (AAV) vector, an adeno virus vector, or a lentiviral vector. In still further embodiments, the AAV vector is recombinant AAV1, AAV2, AAV4, AAV5, AAV7, AAV8, AAV9, AAVrh10, AAV PHP.B, or AAV PDP.eB. In various embodiments, the composition increases the activity of annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 37, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), annexin A13 (SEQ ID NO: 17 or SEQ ID NO: 18), or a combination thereof. In further embodiments, the composition increases the activity of annexin A1 (SEQ ID NO: 1), annexin A2 (SEQ ID NO: 2 or SEQ ID NO: 3), and annexin A6 (SEQ ID NO: 7, SEQ ID NO: 8, SEQ ID NO: 37, or a combination thereof). In some embodiments, the composition increases the activity of annexin A2 (SEQ ID NO: 2 or SEQ ID NO: 3) and annexin A6 (SEQ ID NO: 7, SEQ ID NO: 8, SEQ ID NO: 37, or a combination thereof). In some embodiments, the composition increases the activity of annexin A1 (SEQ ID NO: 1) and annexin A6 (SEQ ID NO: 7, SEQ ID NO: 8, SEQ ID NO: 37, or a combination thereof). In some embodiments, the composition increases the activity of annexin A6 (SEQ ID NO: 7, SEQ ID NO: 8, SEQ ID NO: 37, or a combination thereof). In any of the aspects or embodiments of the disclosure, the composition is a pharmaceutical composition as described herein.

Other features and advantages of the disclosure will be better understood by reference to the following detailed description, including the figures and the examples.

### BRIEF DESCRIPTION OF THE FIGURES

FIG. 1 shows the generation strategy of iPSC-CMs and quality assessment. A) Overview of generation, enrichment, and expansion strategy with quality assessment by cardiac troponin T flow cytometry. After differentiation, iPSC-CM are first enriched using the Miltenyi MACs system followed by expansion. B) Representative cardiac troponin T staining as assessed by flow cytometry before and after enrichment with increase in cardiac troponin T positivity from 58.7% to 95.7%. C) Validation of enrichment strategy, showing change in cardiac troponin T positivity pre- and post-enrichment for DMD-G01 line.

FIG. 2 shows that DMD iPSC-CMs show a differential response to equibiaxial strain. A) Schematic of application of mechanical stress using the Flexcell system that deforms iPSC-CMs adhered to flexible silicone elastomer membranes using a rigid post, imparting equibiaxial strain. B) Overview of injury protocol timeline. iPSC-CMs are subjected to mechanical stress for 2 h followed by a 2 h recovery period. Media is then harvested to determine total LDH release. (C) Control iPSC-CMs do not show a significant increase in the release of LDH compared to unflexed conditions at 5% and 10% strain. At 15% strain, LDH fold release increased by 2.3 (95% Cl: 0.2 to 4.4, *p=0.032). n≥9 from multiple differentiations. D) DMD iPSC-CMs show an increase susceptibility to mechanical stress-induced injury compared to healthy control iPSC-CMs. At 5%, 10%, and 15%, LDH fold release increased relative to unflexed conditions by 1.2 (95% Cl: 0.02 to 2.5, *p=0.045), 2.51 (95% Cl: 1.3 to 3.7, ****p<0.0001), and 3.4 (95% Cl: 2.2 to 4.6, ****p<0.0001), respectively. n≥6 from multiple differentiations.

FIG. 3 shows that recombinant annexin A6 enhances repair in healthy control iPSC-CMs. A) Schematic of injury protocol comparing 2 h and 24 h at 10% strain since healthy control iPSC-CMs require greater duration of mechanical stress to induce injury. B) LDH release fold change increased by 5.1 (95% Cl: 2.9 to 7.2, ****p<0.0001) at 24 h compared to 2 h of 10% strain in control iPSC-CMs. n≥12 from multiple differentiations. C) Fold change of relative fluorescence intensity increased by 3.7 (95% Cl: 2.5 to 5.0, ****p<0.0001) in control iPSC-CMs treated with fluorescently labelled recombinant annexin A6, which was added for the last 1 h of a 10% strain protocol lasting 24 h. n 6 from multiple differentiations. D) Overview of assessment of efficacy of membrane repair for recombinant annexin A6 with a 24 h injury protocol. E) LDH release fold change increased by 1.7 (95% Cl: 0.4 to 3.0, **p=0.01) relative to unflexed control iPSC-CMs after a 24 h 10% strain protocol. Recombinant annexin A6 reduced LDH fold release by 2.1 (95% Cl: 0.8 to 3.3, **p=0.003) under a 10% strain protocol relative to untreated strained iPSC-CMs. No significant difference was observed between unflexed and treated 10% strained iPSC-CMs (95% Cl: −1.6 to 1.0, p=0.79). n≥5 from multiple differentiations. F) Troponin release fold change increased by 5.1 (95% Cl: 3.5 to 6.7, ****p<0.0001) after 10% strain 24 h protocol, while treatment with recombinant annexin A6 under the same protocol reduced troponin release fold change by 4.0 (95% Cl: 2.4 to 5.5, ****p<0.0001) with no significant difference compared to the unflexed condition (95% Cl: −0.4 to 1.1, p=0.19). n≥6 from multiple differentiations.

FIG. 4 shows that recombinant annexin A6 enhances repair in DMD iPSC-CMs. A) Fold change of mean fluorescence intensity increased by 3.4 (95% Cl: 2.2 to 4.5, ****p<0.0001) in DMD iPSC-CMs treated with fluorescently labelled recombinant annexin A6, which was added for the last 1 h of a 10% strain protocol lasting 24 h. n 6 from multiple differentiations. B) Overview of assessment of efficacy of membrane repair for recombinant annexin A6 with a 24 h injury protocol. C) LDH release fold change increased by 4.1 (95% Cl: 1.2 to 7.0, **p=0.005) relative to unflexed DMD iPSC-CMs after a 24 h 10% strain protocol. Recombinant annexin A6 reduced LDH release change by 4.0 (95% Cl: 1.2 to 6.9, **p=0.005) under a 10% strain protocol relative to untreated strained iPSC-CMs. No significant difference was observed between unflexed and treated 10% strained iPSC-CMs (95% Cl: −2.8 to 2.9, p=0.9989). n≥8 from multiple differentiations. D) Troponin release fold change increased by 3.9 (95% Cl: 3.1 to 4.7, ****p<0.0001) after 10% strain 24 h protocol, while treatment with recombinant annexin A6 under the same protocol reduced fold change troponin release by 3.5 (95% Cl: 2.7 to 4.3, ****p<0.0001) with no significant difference compared to the unflexed condition (95% Cl: −0.4 to 1.1, p=0.52). n≥8 from multiple differentiations.

FIG. 5 is a schematic showing that recombinant annexin A6 promotes membrane repair in iPSC-CMs. Equibiaxial strain was employed in order to promote membrane damage in control and DMD iPSC-CMs. Recombinant annexin A6 promotes membrane repair as evidenced by decreased biomarker release in control and DMD iPSC-CMs.

FIG. 6 depicts the generation of genomically-encoded annexin A6GFP using CRISPR/Cas9 genome editing. A) Targeting strategy for generating genomically encoded annexin A6GFP at the endogenous annexin A6 locus. Bold lettering indicates PAM sequence. Lower case lettering indicates synonymous mutations in targeted allele. B) Anxa6gfp mouse generation strategy. C) Genotyping schematic and PCR screening of Anxa6gfp of 6 heterozygous N1 offspring lines. D) Representative sequence chromatograms of in-frame GFP insertion into the annexin A6 locus in Anxa6gfp CRISPR/Cas9 edited mouse line 46.

FIG. 7 shows that genomic A6GFP protein localizes to the site of muscle membrane injury. A) Quantitative PCR demonstrates reduced Anxa6 levels in quadriceps from heterozygous and homozygous Anxa6gfpmice compared to WT controls. B) Anti-annexin A6 immunoblots demonstrate reduced ANXA6 protein levels in quadriceps muscles from heterozygous and homozygous Anxa6gfp mice. Anti-GFP immunoblots confirmed increasing expression of annexin A6GFP protein in quadriceps from heterozygous and homozygous Anxa6gfp) mice. The loading control is a 42Kda band detected by MemCode reversible protein stain. C) Upon laser-induced membrane injury, annexin A6GFP localized to the repair cap (white arrow) with a visible clearance zone (orange arrow) beneath the membrane lesion in Anxa6gfp myofibers. D) Genomically-encoded annexin A6GFP membranous blebs (white arrow) erupt from the site of membrane injury. Z-stack images from an injured myofiber. Scale bar 5 μm. n≥3. *P<0.05.

FIG. 8 shows A) Grossly normal muscle histopathology in heterozygous and homozygous Anxa6gfp tissue compared to WT control muscle. B) Similar FM 4-64 uptake after laser-induced membrane injury of WT and homozygous Anxa6gfp myofibers. Scale 5 μm. n=3 mice per genotype.

FIG. 9 shows that genomically-encoded annexin A6GFP colocalizes with repair complex members at the site of injury. Myofibers were isolated from heterozygous Anxa6gfp mice and electroporated with td-Tomato (red) tagged annexin A1, A2, or A6. Genomically-encoded annexin A6GFP (green) colocalizes with annexin A1, A2 and A6 at the site of membrane damage (merge, yellow). Z-stack images from an injured myofiber. Scale 5 μm.

FIG. 10 shows that genomically encoded annexin A6GFP localizes at the site of cardiomyocyte membrane injury. A) Quantitative PCR demonstrates reduced Anxa6 levels in heart lysates from heterozygous and homozygous Anxa6gfp mice compared to WT controls. B) Anti-annexin A6 immunoblots demonstrate reduced ANXA6 protein levels in hearts from heterozygous and homozygous Anxa6gfp mice. Anti-GFP immunoblots confirmed increasing expression of annexin A6GFP protein in hearts from heterozygous and homozygous Anxa6gfp mice. The loading control is a 42KDa band detected by MemCode reversible protein stain. C) Adult ventricular cardiomyocytes were isolated from Anxa6gfp mice and subsequently laser-damaged. A6GFP (green) quickly localizes to the cardiomyocyte repair cap (white arrow). Scale bar 10 μm. Higher magnification image of the cardiomyocyte A6GFP repair cap located within the white dotted box is located on the right. Scale bar 5 μm. D) Cardiomyocyte A6GFP repair cap develops at the site of injury over 50 seconds of imaging (white arrow). Scale bar 5 μm. n≥3 mice. *P<0.05.

FIG. 11 shows that annexin A6GFP localizes at the site of neuron membrane injury. A) Anti-GFP (green, arrow) antibody detects genomically-encoded A6GFP protein in Anxa6gfp adult cortex and midbrain, but not in wildtype mice. Dapi (blue) marks nuclei. Anti-NeuN (red) marks mature neurons. B) Anti-GFP (green, arrow) antibody, which detects genomically-encoded A6GFP protein, localized to the peripheral membrane of NeuN+ cortical neurons as visualized with high magnification confocal imaging. C) Embryonic neurons were isolated from Anxa6gfpmice. Genomically-encoded A6GFP expression increases with maturation. After 10 days, neurons expressed 2-fold more A6GFP than at 4 days. D) Isolated neurons were injured with a confocal laser. Genomically-encoded A6GFP (green) quickly localized into a repair cap (white arrow) visible 4 seconds post injury. Multiple cells from n≥3 mice. *P<0.05.

FIG. 12 shows that recombinant annexin A6 binds phosphatidylserine. A) Recombinant annexin A6 preferentially bound phosphatidylserine and Ptlns(5)P on membrane lipid arrays. B) SPR sensograms showing that recombinant annexin A6 binds phosphatidylserine (PS)-containing liposomes at approximately 100 nM over a range of Ca²⁺ concentrations ranging from 0-2500 μM (0, 25, 52, 104, 208, 417, 833, and 2500 μM). C) SPR sensograms showing that recombinant annexin A6 bound PS-containing lipid bilayer. The dose-dependent kinetics are shown as colored lines representing different annexin A6 concentrations. Black lines represent fittings with 1:1 kinetics interaction model. Kinetics parameters association rate (kₐ), dissociation rate (k_{d}), and binding affinity (K_{D}) derived from the fits are shown in the table (below). The closeness of fit χ² is also provided. D) Wortmannin treatment depleted PIP2 in myofibers as visualized by reduced PLC Δ PH-EGFP signal (top panel). Additionally, wortmannin treatment reduced genomically encoded annexin A6GFP cap area after laser-induced injury. (n=10 from 5 isolations; P<0.002).

FIG. 13 shows that recombinant annexin A6 binds injured muscle membrane in a concentration dependent manner. A) Rat L6 myoblasts were injured with LLO and then incubated with recombinant annexin A6 conjugated to 488 (A6-488). The percentage of annexin A6-488 positive cells increased with increasing concentrations of annexin protein. B) Total fluorescence intensity of annexin A6-488 positive cells increased with increasing concentrations of annexin A6-488; normalized to 1.0 μg/ml. C) Increasing annexin A6-488 incubation time increased fluorescence signal of injured cells, but not non-injured control cells. D) Incubation of myofibers in recombinant annexin A6 for either 5 or 60 min both reduced FM 4-64 dye uptake after injury compared to BSA-treated control myofibers. Scale bar 5 μm. n≥2 mice. *P<0.05.

FIG. 14 shows that recombinant annexin A6 cap size increases in a dose-dependent fashion correlating with improved repair capacity. A) Myofibers were isolated from Anxa6gfpmice and laser damaged in the presence of a6-tdTomato. Recombinant annexin A6 tdTomato (rA6-tomato)(red) colocalized with genomically-encoded annexin A6GFP (green) at the site of muscle membrane injury (white arrow). B) rA6-tomato cap size increased with increasing concentrations of rA6-tdTomato, 1.3-130 μg/ml. Genomically-encoded A6GFP cap size did not change with increasing concentrations of rA6-tdTomato. C) rA6-tomato formed membranous blebs at the site of membrane injury. D) Dose-dependent reduction of FM 4-64 dye (red) uptake, a marker of membrane injury, with increasing concentrations of recombinant annexin A6. E) Anxa6gfp mice were crossed with mdx mice to generate mdx mice expressing genomically-encoded A6GFP. F & G) In Anxa6gfp mdx myofibers, rA6-tomato cap size increased with increasing concentrations of rA6-tdTomato, 1.3-130 μg/ml. Genomically-encoded A6GFP cap size did not change significantly with varying concentrations of rA6-tdTomato in Anxa6gfp mdx myofibers. H) Increasing concentrations of recombinant annexin A6, resulted in a dose-dependent reduction of FM 4-64 dye (red) uptake in dystrophic myofibers. Scale bar 5 μm. multiple myofibers from n≥2 mice. *P<0.05.

FIG. 15 shows that recombinant annexin A6 binds neuronal membrane lesions. A) Embryonic neurons were isolated from Anxa6gfpmice, matured, and laser damaged in the presence of rA6-tdTomato. rA6-tdTomato (red) colocalizes with genomically-encoded annexin A6GFP (green) at the site of muscle membrane injury(white arrow). Neuron outlined in white dotted line. B) After Anxa6gfpneuronal process transection, rA6-tdTomato (red) localizes at the stumps of the severed process (white arrows). WGA-350 (blue) outlines neuron. C) rA6-tdTomato fluorescent signal increases at the process stumps with time (white arrows). Multiple neurons from n≥3 mice.

FIG. 16 depicts a model of annexin A6 mediated membrane repair in skeletal muscle, cardiomyocytes, and neurons. Upon plasma membrane breach, extracellular Ca²⁺ enters the damaged cell. Annexin A6 (A6) binds Ca²⁺, translocates to the site of membrane injury targeting exposed phospholipids such as PS, and forms a repair cap at the lesion. Extracellular recombinant annexin A6 (rA6) localizes to the repair cap at the site of injury enhancing repair capacity. Annexin A6 positive blebs emanate from the repair cap during the repair process.

FIG. 17 shows that dystrophic neurites are sites of elevated Ca²⁺, membrane damage and disrupted tubulin. A) Live 2P-imaging of 5XFAD brain slice with ratiometric Ca²⁺ sensing dye Indo-1 shows elevated ratio of Ca²⁺ bound (400 nm, blue) to Ca²⁺ unbound (475 nm, green) Indo-1 indicating elevated free Ca²⁺. Red=Thiazine red, *=plaques, dotted lines outline DNs with elevated Ca²⁺. B) Average Indo-1 400 nm/475 nm ratio in DNs compared to nearby neuropil; student's t-test, ***=p<0.001, line=mean. C) EM image showing two vesicle-filled, myelinated dystrophic axons near plaque in 5XFAD mouse; the one on left shows loss of myelin and membrane rupture (arrows). D) Axon of primary murine neuron showing disorganized tubulin in swellings that accumulated BACE1 after exposure to Aβ42, analogous to DN formation in vivo. E) Live imaging time-course of GFP-tubulin-expressing primary neurons exposed to Aβ42 shows increasing neuritic beading with time. Varicosities appeared at 60 min and progressed (black arrows) with more appearing at 90 min (red arrows). (F and G) Schematic of neuron near growing plaque and enlargement of axon with normal membrane, microtubules (green) and vesicle trafficking (F) and the axon after plaque growth and Aβ contact causing membrane damage, Ca²⁺ influx (red dots), disrupted microtubules and vesicle accumulation.

FIG. 18 shows membrane repair protein annexin A6 was expressed in murine primary neurons. A) Ca²⁺ and phospholipid binding face of annexin A6. B) Schematic of annexin A6 repair cap complex after acute membrane injury. PS=phosphatidylserine, PIP2=phosphatidylinositol biphosphate. C) Immunoblot of Anxa6em1 (GFP) primary cortical neuron lysates showing increasing A6-GFP during maturation. Days in vitro are indicated. WT=wild-type neurons. D) A6-GFP immunoblot signals in C were normalized to GAPDH and graphed as fold change relative to day 4 in vitro. Error bars=SEM. *, p<0.05 by ANOVA.

FIG. 19 shows genomically expressed annexin A6-GFP and exogenous recombinant A6-tdTomato localized to sites of membrane damage in primary neurons. A) Time course showing rapid accumulation of A6-GFP (green) at the site of laser injury on the soma membrane of an Anxa6em1 (GFP) primary neuron, consistent with repair cap formation. B) Exogenous recombinant A6-tdTomato (red) colocalizes with genomic A6-GFP (green) at the site of laser injury in an Anxa6em1 (GFP) primary neuron. White dots outline the neuron cell body. C) Time course showing rapid accumulation of exogenous recombinant A6-TdTomato at the laser injury site in an Anxa6em1(GFP) neurite, suggesting repair cap formation. Fluorophore-conjugated wheat-germ agglutinin (WGA, blue) marks membrane. D) Anxa6em1 (GFP) primary neuron treated with Aβ42 oligomers shows localization of A6-GFP (green) at sites of contact with Aβ42 (red). Arrows indicate sites of laser injury (A-C) or examples of A6-GFP colocalization with Aβ42 (D).

FIG. 20 shows that A6-GFP localized to neuronal and DN membranes in Anxa6em1(GFP) and Anxa6em1(GFP); 5XFAD mouse brains. A) Anxa6em1(GFP) and wild-type (WT) cortical sections immunostained for neuronal marker NeuN (red), GFP (green), and DAPI (blue). Note A6-GFP in large NeuN+ Anxa6em1 (GFP) neurons and no GFP staining in WT cortex. B) Anxa6em1 (GFP); 5XFAD cortical sections immunostained for GFP (green), BACE1 to label DNs (red), DAPI (blue), and Aβ (antibody 3D6; white). Arrows indicate examples of A6-GFP-labeled DNs around plaques. Bars=25 μm, *=plaque cores.

FIG. 21 shows that endogenous annexin A6 localized to neuronal and DN membranes in 5XFAD mouse and human AD brains. A) 5XFAD cortex section immunostained for annexin A6 (red, top and bottom), BACE1 (DN marker, green, bottom), and NeuN (white, bottom) shows A6 localization to neuronal (arrows) and DN (arrowheads) membranes. *=plaque cores. B) Immunoblot of lysates of primary neurons treated for 72 hrs with Aβ42 oligomers or vehicle shows increased annexin A6, LAMP1, BACE1 and LC3B-II, all found in DNs. C) Quantification of immunoblot in B normalized to GAPDH, except LC3B-II normalized to LC3B-I. D) Human AD hippocampal section immunostained for annexin A6 (red, top and bottom) and NeuN (green, bottom) shows A6 localized to the membrane of neurons (arrows). E) Human AD hippocampal section immunostained for annexin A6 (red, top and bottom), BACE1 (green, bottom), and MeXO4 (blue, top and bottom) shows A6 localized to the membranes of BACE1+ DNs (arrows), similar to 5XFAD brain. Note that DNs in AD plaques are more dispersed in the amyloid deposit than in 5XFAD plaques, in which DNs tend to form a halo around the Aβ core8. Bars=25 μm. In C, line=mean; error bars=SEM.

FIG. 22 shows that A6-GFP overexpressed via AAV localized correctly to neuronal and DN membranes in 5XFAD brain. A) syn-A6-GFP AAV-injected 5XFAD brain section immunostained for A6-GFP (green), BACE1 (red), Aβ42 (white), and DAPI (blue) shows A6-GFP localized to neuronal and BACE1+DN membranes (arrows). *=plaques. Bar=1 μm. B) Immunoblot of brain homogenates from 5XFAD and non-transgenic (non-Tg) mice labeled with antibodies against annexin A6 and tubulin. syn-GFP AAV-injected 5XFAD (green), syn-A6-GFP AAV-injected 5XFAD (red), syn-GFP AAV-injected non-Tg (black), syn-A6-GFP AAVinjected non-Tg (blue). C) Quantification of immunoblot in B showing fold expression of A6-GFP over endogenous A6; A6-GFP is expressed at 6-12 fold over endogenous A6. Blue dots=syn-A6-GFP AAV-injected non-Tg, red dots=syn-A6-GFP AAV-injected 5XFAD. In C, line=mean; error bars=SEM.

FIG. 23 shows that A6-GFP overexpression reduced DNs without affecting Aβ deposition, microglia, or astrocytes in 5XFAD mice. A) Representative images of coronal brain sections of syn-GFP AAV and syn-A6-GFP AAV-injected 5XFAD mice immunostained for Aβ42 (red), LAMP1 (green), and DAPI (blue). Left panels: low magnification of sections showing cortex and hippocampus; Bars=500 μm. Right panels: high magnification of similar sized Aβ42 plaque cores and surrounding LAMP1+DN halos; note the significant reduction of the DN halo for the plaque in the syn-A6-GFP AAV-injected brain section; Bars=10 μm. B) Quantification of the average ratios per mouse of LAMP1/Aβ42 immunosignals binned into the indicated plaque core area ranges in μm². Smaller plaques in the 0-50 and 50-200 μm² area ranges in the cortex and the 0-50 μm² range in the hippocampus had significantly reduced LAMP1/Aβ42 ratios in syn-A6-GFP AAV compared to syn-GFP AAV injected brains. A trend toward reduced LAMP1/Aβ42 ratio was observed for the 50-200 μm² range in the syn-A6-GFP AAV injected hippocampus (p=0.15). ***, p<0.001. 2-way ANOVA: p<0.0001, cortex; p=0.0006, hippocampus. C) Total percent Aβ42+ and LAMP1+ areas are unchanged and significantly reduced, respectively, in sections from syn-A6-GFP AAV compared to syn-GFP AAV injected brains. *, p<0.05, Student's t-test. D) The average area of GFP+ DNs per mouse is significantly reduced in syn-A6-GFP AAV compared to syn-GFP AAV injected brains. E) The ratios of Iba1 and GFAP to amyloid (MeXO4) within 15 μm of plaque cores are unchanged by A6-GFP overexpression compared to GFP, indicating no change in recruitment of microglia or astrocytes to plaques. In B-E, line=mean; error bars=SEM.

FIG. 24 shows that p-tau181, p-tau231 and tau kinases accumulated in DNs of 5XFAD mouse and human AD brains, and A6-GFP overexpression decreases p-tau181 in 5XFAD mice. A) and B) 5XFAD (left) and 5XFAD; Tau^{-/-} (right) cortex sections immunostained for p-tau231 (A, red, top and bottom), p-tau181 (B, red, top and bottom), NeuN (A, blue, top and bottom), DAPI (B, blue, top and bottom), and LAMP1 (A and B, green, bottom). Both p-tau181 and p-tau231 accumulate in LAMP1+ DNs in 5XFAD brain (arrows denote examples). Absence of p-tau181 and p-tau231 signals in 5XFAD; Tau^{-/-} sections validates both ptau antibodies for immunostaining. C) Average ratio of ptau181/Thiazine red (amyloid) signal per mouse in cortex sections of 5XFAD mice injected with syn-GFP AAV or syn-A6-GFP AAV. Line=mean; error bars=SEM. D-F) 5XFAD cortex sections immunostained for p-tau181 (red; D, E), DAPI (blue, D, F), BACE1 (green, D; red, F), p-JNK (green, E), NeuN (blue, E), p-CamKII (green, F) show accumulation of p-tau181, p-JNK, and p-CamKII in DNs. G-I) Human AD hippocampal sections immunostained for p-tau181 (red, G-I), Tau5 total tau (green, G), MeXO4 stained amyloid (blue, G-I), BACE1 (green, H), and APP (green, I). Total tau and p-tau181 colocalize, confirming specificity of p-tau181 antibody. J) Human AD hippocampal section immunostained for p-tau231 (green), p-JNK (red), APP (white), MeXO4 and DAPI (blue) shows p-tau231, p-JNK and APP colocalization in DNs (arrows). *=plaque cores. All bars=10 μm.

FIG. 25 depicts cortical sections from 5XFAD mouse that received a single ICV injection of A6-HIS (3.3 mg/kg) and brain harvest 3 hrs later. Sections were immunostained for α-HIS (red, left and right panels), NeuN (blue, left and right panels), MeXO4 (blue, left and right panels), BACE1 (green, right panels). A6-HIS localized in patches to the membranes of BACE1+ DNs (arrows). Whole DNs were occasionally enveloped in A6-HIS (top panels, far left arrows). A6-HIS also labeled plaques, likely due to PS association with Aβ from membrane damage. Bars=1 μm. *=plaque cores.

FIG. 26 shows that single tail vein injection of syn-GFP PHP.eB AAV in 5XFAD mice resulted in widespread neuronal GFP expression and GFP accumulation in DNs. A) Parasagittal brain section of 5XFAD mouse tail-vein injected with a single dose of syn-GFP PHP.eB AAV (1×10¹² VG). GFP, green; MeXO4 & DAPI, blue. Bar=500 μm. B) High magnification of the cortex showing GFP expressing neurons. Bar=50 μm. C) High magnification showing GFP+ DNs (green, arrows) surrounding MeXO4 labeled plaques (blue). Bar=25 μm. *=plaque cores in B,C.

### DETAILED DESCRIPTION

As used in this specification and the enumerated paragraphs herein, the singular forms “a,” “an,” and “the” include plural reference unless the context clearly dictates otherwise.

As used herein, an “effective amount” refers to an amount of a substance, such as an agent and/or additional agent as described herein, sufficient to elicit the desired biological response, e.g., treating the condition. As will be appreciated by those of ordinary skill in this art, the effective amount may vary depending on such factors as the desired biological endpoint, the pharmacokinetics of the compound, the condition being treated, the mode of administration, and the age and health of the subject. An effective amount encompasses therapeutic and prophylactic treatment.

As used herein, an agent that “increases the activity of an annexin protein” is one that increases a property of an annexin protein as a calcium-binding membrane associated repair protein that enhances restoration of membrane integrity. The enhancement to restoring membrane integrity may be through facilitating the formation of a macromolecular repair complex at the membrane lesion including proteins such as, without limitation, annexin A1 (SEQ ID NO: 1), annexin A2 (SEQ ID NO: 2 or SEQ ID NO: 3), EHD2, dysferlin, and MG53. Thus, administration of the agent results in an overall increase in the activity (i.e., the increase in activity derived from administration of the agent plus any endogenous activity) of one or more annexin proteins as disclosed herein.

As used herein, the term “treating” or “treatment” refers to an intervention performed with the intention of preventing the further development of or altering the pathology of a disease or infection. Accordingly, “treatment” refers to both therapeutic treatment and prophylactic or preventative measures. Of course, when “treatment” is used in conjunction with a form of the separate term “prophylaxis,” it is understood that “treatment” refers to the narrower meaning of altering the pathology of a disease or condition. “Preventing” refers to a preventative measure taken with a subject not having a condition or disease. A therapeutic agent may directly decrease the pathology of a disease, or render the disease more susceptible to treatment by another therapeutic agent(s) or, for example, the host's own cellular membrane repair system. Treatment of patients suffering from clinical, biochemical, or subjective symptoms of a disease may include alleviating one or more of such symptoms or reducing the predisposition to the disease. Improvement after treatment may be manifested as a decrease or elimination of one or more of such symptoms.

### Annexin Proteins

With membrane breach, the influx of extracellular Ca²⁺ can initiate plasma membrane repair. In the case of small membrane lesions, several models of plasma membrane repair have been proposed (Andrews N W, and Corrotte M. Plasma membrane repair. Curr Biol. 2018; 28(8):R392-R7; Cooper S T, and McNeil P L. Membrane Repair: Mechanisms and Pathophysiology. Physiol Rev. 2015; 95(4):1205-40; Koerdt S N, Ashraf APK, and Gerke V. Annexins and plasma membrane repair. Curr Top Membr. 2019; 84:43-65). In one model, intracellular vesicles are recruited to site of injury in a Ca²⁺-dependent manner where they fuse with each other and to the membrane lesion (McDade J R, and Michele D E. Membrane damage-induced vesicle-vesicle fusion of dysferlin-containing vesicles in muscle cells requires microtubules and kinesin. Human molecular genetics. 2014; 23(7):1677-86; Lek A, Evesson F J, Lemckert F A, Redpath G M, Lueders A K, Turnbull L, et al. Calpains, cleaved mini-dysferlinC72, and L-type channels underpin calcium-dependent muscle membrane repair. J Neurosci. 2013; 33(12):5085-94; Davenport N R, Sonnemann K J, Eliceiri K W, and Bement W M. Membrane dynamics during cellular wound repair. Mol Biol Cell. 2016; 27(14):2272-85). A second model implicates constriction of the injured membrane followed by budding and shedding of the injured membrane into the extracellular space (Jimenez A J, Maiuri P, Lafaurie-Janvore J, Divoux S, Piel M, and Perez F. ESCRT machinery is required for plasma membrane repair. Science. 2014; 343(6174):1247136; Bement W M, Mandato C A, and Kirsch M N. Wound-induced assembly and closure of an actomyosin purse string in Xenopus oocytes. Curr Biol. 1999; 9(11):579-87; Babiychuk E B, Monastyrskaya K, Potez S, and Draeger A. Intracellular Ca(2+) operates a switch between repair and lysis of streptolysin O-perforated cells. Cell Death Differ. 2009; 16(8):1126-34; Babiychuk E B, Monastyrskaya K, Potez S, and Draeger A. Blebbing confers resistance against cell lysis. Cell Death Differ. 2011; 18(1):80-9). Others have described endocytosis of the injured membrane area in addition to lateral diffusion of membrane to the site of injury (Demonbreun A R, and McNally E M. Plasma Membrane Repair in Health and Disease. Curr Top Membr. 2016; 77:67-96; Idone V, Tam C, Goss J W, Toomre D, Pypaert M, and Andrews N W. Repair of injured plasma membrane by rapid Ca²⁺-dependent endocytosis. The Journal of cell biology. 2008; 180(5):905-14; McDade J R, Archambeau A, and Michele D E. Rapid actin-cytoskeleton-dependent recruitment of plasma membrane-derived dysferlin at wounds is critical for muscle membrane repair. FASEB J. 2014; 28(8):3660-70; Corrotte M, Almeida P E, Tam C, Castro-Gomes T, Fernandes M C, Millis B A, et al. Caveolae internalization repairs wounded cells and muscle fibers. Elife. 2013; 2:e00926). The machinery that mediates membrane repair participates in other cellular transport processes, scaffolding receptor and signaling complexes, and modulating actin dynamics, which are not exclusively dedicated to membrane repair.

The annexin protein family is characterized by the ability to bind phospholipids and actin in a Ca²⁺-dependent manner. Annexins preferentially bind phosphatidylserine, phosphatidylinositols, and cholesterol (Gerke et al., 2005). In humans, dominant or recessive mutations in annexin genes have not been associated with muscle disease. However, annexin A5 genetic variants are associated with pregnancy loss (de Laat et al., 2006). The annexin family is known to comprise over 160 distinct proteins that are present in more than 65 unique species (Gerke and Moss, 2002). Humans have 12 different annexin genes, characterized by distinct tissue expression and localization. Annexins are involved in a variety of cellular processes including membrane permeability, mobility, vesicle fusion, and membrane bending. These properties are Ca²⁺-dependent. Although annexins do not contain EF hand domains, calcium ions bind to the individual annexin repeat domains. Differential Ca²⁺ affinity allows each annexin protein to respond to changes in intracellular calcium levels under unique spatiotemporal conditions (Blackwood and Ernst, 1990).

Structurally, the annexin family of proteins contains a conserved carboxy-terminal core domain composed of multiple annexin repeats and a variable amino-terminal head. The amino-terminus differs in length and amino acid sequence amongst the annexin family members. Additionally, post-translational modifications alter protein function and protein localization (Goulet et al., 1992; Kaetzel et al., 2001). Annexin proteins have the potential to self-oligomerize and interact with membrane surfaces and actin in the presence of Ca²⁺ (Zaks and Creutz, 1991, Hayes et al., 2006), Jaiswal et al., 2014)). The amino-terminal region is thought to bind actin or one lipid membrane in a Ca²⁺-dependent manner, while the annexin core region binds an additional lipid membrane.

Annexins do not contain a predicted hydrophobic signal sequence targeting the annexins for classical secretion through the endoplasmic reticulum, yet annexins are found both on the interior and exterior of the cell (Christmas et al., 1991; Deora et al., 2004; Wallner et al., 1986). The process by which the annexins are externalized remains unknown. It is hypothesized that annexins may be released through exocytosis or cell lysis, although the method of externalization may vary by cell type. Functionally, localization both inside and outside the cell adds to the complexity of the roles annexins play within tissues and cell types. Annexin A5 is used commonly as a marker for apoptosis due to its high affinity to phosphatidylserine (PS). During cell death and injury, PS reverses membrane orientation from the inner to outer membrane, providing access for annexin binding from the cell exterior. Annexins have been shown to have anti-inflammatory, pro-fibrinolytic, and anti-thrombotic effects.

Efficient sarcolemma repair is also critical for cardiomyocyte survival as these cells are terminally differentiated and have limited capacity for self-regeneration. Annexin A5 and annexin A6 are the most abundantly expressed annexin proteins in the heart (Doubell A F, Lazure C, Charbonneau C, and Thibault G. Identification and immunolocalisation of annexins V and VI, the major cardiac annexins, in rat heart. Cardiovasc Res. 1993; 27(7):1359-67).

Like skeletal and cardiac muscle, mechanical stress results in plasma membrane disruption of neurons (Kilinc D, Gallo G, and Barbee K A. Mechanically-induced membrane poration causes axonal beading and localized cytoskeletal damage. Exp Neurol. 2008; 212(2):422-30; Prado G R, and LaPlaca M C. Neuronal Plasma Membrane Integrity is Transiently Disturbed by Traumatic Loading. Neurosci Insights. 2020; 15:2633105520946090). Disruption of axonal membranes is an early event following traumatic brain injury in humans and experimental animal models of traumatic brain injury (LaPlaca M C, Prado G R, Cullen D K, and Irons H R. High rate shear insult delivered to cortical neurons produces heterogeneous membrane permeability alterations. Conf Proc IEEE Eng Med Biol Soc. 2006; 2006:2384-7; LaPlaca M C, Prado G R, Cullen D, and Simon C M. Plasma membrane damage as a marker of neuronal injury. Annu Int Conf IEEE Eng Med Biol Soc. 2009; 2009:1113-6). Similar to skeletal muscle, repair of neuronal membrane after mechanical disruption is dependent on calcium and actin cytoskeletal dynamics (Prado G R, and LaPlaca M C. Neuronal Plasma Membrane Integrity is Transiently Disturbed by Traumatic Loading. Neurosci Insights. 2020; 15:2633105520946090).

Annexin A6 in cardiac injury and disease. Cardiomyocytes are a terminally differentiated cell type with limited ability to regenerate. Therefore, cell survival is crucial for preserving cardiac function. Membrane repair is vital for cardiac membrane stability and impairments in membrane repair may lead to heart disease and failure (reviewed in Kitmitto A, Baudoin F, and Cartwright E J. Cardiomyocyte damage control in heart failure and the role of the sarcolemma. J Muscle Res Cell Motil. 2019; 40(3-4):319-33). Annexin A6 is expressed in the healthy heart at levels higher than annexin A2 and A5 (Benevolensky D, Belikova Y, Mohammadzadeh R, TrouvéP, Marotte F, Russo-Marie F, et al. Expression and Localization of the Annexins II, V, and VI in Myocardium from Patients with End-Stage Heart Failure. Laboratory Investigation. 2000; 80(2):123-33; Song G, Campos B, Wagoner L E, Dedman J R, and Walsh R A. Altered cardiac annexin mRNA and protein levels in the left ventricle of patients with end-stage heart failure. J Mol Cell Cardiol. 1998; 30(3):443-51). Data provided herein demonstrate that annexin A6 localizes to the site of cardiomyocyte injury forming a repair cap at the membrane lesion. In some aspects, the disclosure contemplates methods comprising administering one or more agents (e.g., recombinant annexin A6) to treat, prevent, delay onset of, or enhance recovery from heart disease associated with increased cellular breakdown.

Annexins A6 in neuronal injury. Similar to muscle, neuronal membrane damage can occur by physical trauma, degenerative processes or as a secondary consequence of a primary disease. Unrepaired damage in neurons leads to cell degeneration and death, with devasting physical consequences. As in skeletal muscle, an increase in intracellular Ca²⁺ occurs when the membrane is breached. A persistent rise in intracellular Ca²⁺ in neurons may lead to dysregulated ion gradients, protease activation, mitochondrial dysfunction and apoptosis (Hendricks B K, and Shi R. Mechanisms of neuronal membrane sealing following mechanical trauma. Neurosci Bull. 2014; 30(4):627-44). Therefore, timely repair of lesions in neuronal membranes is crucial. The present disclosure demonstrates that genomically-encoded annexin A6 is expressed in maturing primary neurons at levels sufficient to form a repair cap at the site of injury. The immediate localization of annexin A6 into a repair cap in neurons is similar to that of muscle, and shows that annexin A6 orchestrates repair of neuronal membranes. This is further supported by the observation described herein (e.g., Example 2) that externally delivered recombinant annexin A6 bound to the damaged area on the neuronal plasma membrane both after generation of a small lesion and after transection.

### Agents

In some aspects, the disclosure provides methods of the disclosure contemplate treating a cellular membrane injury comprising administering to a patient in need thereof a therapeutically effective amount of a composition comprising an agent that increases the activity of an annexin protein. In further aspects, methods of delaying onset, preventing a cellular membrane injury, or enhancing recovery from cellular membrane injury are provided, comprising administering to a patient in need thereof a therapeutically effective amount of a composition comprising an agent that increases the activity of an annexin protein. In some aspects, the disclosure provides methods of treating a patient suffering from a nerve injury comprising administering to the patient a therapeutically effective amount of a composition comprising an agent that increases the activity of an annexin protein. The disclosure also provides, in various aspects, methods of delaying onset, enhancing recovery from a nerve injury, or preventing a nerve injury, comprising administering to a patient in need thereof a therapeutically effective amount of a composition comprising an agent that increases the activity of an annexin protein. In still further aspects, the disclosure provides methods comprising administering a therapeutically effective amount of an agent that increases the activity of an annexin protein to a patient, wherein the patient has an elevated serum or plasma level of lactate dehydrogenase (LDH), cardiac troponin T, cardiac troponin I, creatine kinase (CK), or a combination thereof, relative to a control level. The disclosure also provides, in various aspects, methods of reducing serum or plasma level of lactate dehydrogenase (LDH), cardiac troponin T, cardiac troponin I, creatine kinase (CK), or a combination thereof, in a patient in need thereof, comprising administering a therapeutically effective amount of an agent that increases the activity of an annexin protein to the patient, thereby reducing the serum or plasma level of lactate dehydrogenase (LDH), cardiac troponin T, creatine kinase (CK), or a combination thereof in the patient. “Increase the activity of an annexin protein” means that administration of the agent results in an overall increase in the activity (i.e., the increase in activity derived from administration of the agent plus any endogenous activity) of one or more annexin proteins as disclosed herein.

The term “agent” as used herein refers to a recombinant protein (e.g., a recombinant annexin protein), a steroid, an annexin peptide, a polynucleotide capable of expressing an annexin protein, or a combination thereof. In any of the aspects or embodiments of the disclosure, the agent is a recombinant annexin protein. In some embodiments, the recombinant annexin protein is recombinant annexin A6. In some embodiments, the agent is a polynucleotide capable of expressing an annexin protein that is secreted extracellularly. Thus, in some embodiments the agent is a polynucleotide capable of expressing an extracellular annexin protein (e.g., extracellular annexin A6). As described herein, combinations of agents are also contemplated for use in the methods described herein.

### Proteins/Recombinant Proteins

Methods of the disclosure include those in which a recombinant protein (e.g., one or more annexin proteins) is administered to a patient in need thereof in a therapeutically effective amount. Thus, in any of the aspects or embodiments of the disclosure, the agent that increases the activity of an annexin protein is a recombinant protein (e.g., an annexin protein). As used herein a “protein” refers to a polymer comprised of amino acid residues. “Annexin protein” as used herein includes without limitation a wild type annexin protein, a modified annexin protein, an annexin-like protein, or a fragment, analog, variant, fusion or mimetic, each as described herein. An “annexin peptide” is a shorter version (e.g., about 50 amino acids or less) of a wild type annexin protein, an annexin-like protein, or a fragment, analog, variant, fusion or mimetic that is sufficient to increase the overall activity of the annexin protein to which the annexin peptide is related.

Proteins of the present disclosure may be either naturally occurring or non-naturally occurring. Naturally occurring proteins include without limitation biologically active proteins that exist in nature or can be produced in a form that is found in nature by, for example, chemical synthesis or recombinant expression techniques. Naturally occurring proteins also include post-translationally modified proteins, such as, for example and without limitation, glycosylated proteins. Non-naturally occurring proteins contemplated by the present disclosure include but are not limited to synthetic proteins, as well as fragments, analogs and variants of naturally occurring or non-naturally occurring proteins as defined herein. Non-naturally occurring proteins also include proteins or protein substances that have D-amino acids, modified, derivatized, or non-naturally occurring amino acids in the D- or L-configuration and/or peptidomimetic units as part of their structure. The term “protein” typically refers to large polypeptides. The term “peptide” generally refers to short (e.g., about 50 amino acids or less) polypeptides.

Non-naturally occurring proteins are prepared, for example, using an automated protein synthesizer or, alternatively, using recombinant expression techniques using a modified oligonucleotide which encodes the desired protein.

As used herein a “fragment” of a protein is meant to refer to any portion of a protein smaller than the full-length protein expression product.

As used herein an “analog” refers to any of two or more proteins substantially similar in structure and having the same biological activity, but can have varying degrees of activity, to either the entire molecule, or to a fragment thereof. Analogs differ in the composition of their amino acid sequences based on one or more mutations involving substitution, deletion, insertion and/or addition of one or more amino acids for other amino acids. Substitutions can be conservative or non-conservative based on the physico-chemical or functional relatedness of the amino acid that is being replaced and the amino acid replacing it.

As used herein a “variant” refers to a protein or analog thereof that is modified to comprise additional chemical moieties not normally a part of the molecule. Such moieties may modulate, for example and without limitation, the molecule's solubility, absorption, and/or biological half-life. Moieties capable of mediating such effects are disclosed in Remington's Pharmaceutical Sciences (1980). Procedures for coupling such moieties to a molecule are well known in the art. In various aspects, polypeptides are modified by biotinylation, glycosylation, PEGylation, and/or polysialylation.

Fusion proteins, including fusion proteins wherein one fusion component is a fragment or a mimetic, are also contemplated. A “mimetic” as used herein means a peptide or protein having a biological activity that is comparable to the protein of which it is a mimetic.

In any of the aspects or embodiments of the disclosure, the recombinant protein is an annexin protein (e.g., a recombinant wild type annexin protein, a modified annexin protein, an annexin-like protein, or a fragment of a wild type annexin protein or annexin-like protein that exhibits one or more biological activities of an annexin protein). By “annexin-like protein” is meant a protein having sufficient amino acid sequence identity to a reference wild type annexin protein to exhibit the activity of an annexin protein, for example and without limitation, activity as a calcium-binding membrane associated repair protein that enhances restoration of membrane integrity through facilitating the formation of a macromolecular repair complex at the membrane lesion including proteins such as annexin A1 (SEQ ID NO: 1), annexin A2 (SEQ ID NO: 2 or SEQ ID NO: 3), EHD2, dysferlin, and MG53. In some embodiments, the annexin-like protein is a protein comprising an amino acid sequence having about or at least about 75% amino acid sequence identity with a reference wild type human annexin protein (e.g., annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 37, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), or annexin A13 (SEQ ID NO: 17 or SEQ ID NO: 18)). In further embodiments, the annexin-like protein is a protein comprising an amino acid sequence having about or at least about 80%, about or at least about 85%, about or at least about 90%, about or at least about 95%, about 99%, or about 100% amino acid sequence identity with a reference wild type human annexin protein (e.g., annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 37, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), or annexin A13 (SEQ ID NO: 17 or SEQ ID NO: 18)).

In some embodiments, an agent of the disclosure is an annexin protein that comprises a post-translational modification. In various embodiments, the post-translational modification increases production of an annexin or annexin-like protein, increases solubility of an annexin or annexin-like protein, decreases aggregation of an annexin or annexin-like protein, increases the half-life of an annexin or annexin-like protein, increases the stability of an annexin or annexin-like protein, enhances target membrane engagement of an annexin or annexin-like protein, or is a codon-optimized version of an annexin or annexin-like protein.

In some embodiments, the agent is a polynucleotide capable of expressing an annexin protein (e.g., annexin A6) that is secreted extracellularly. For example and without limitation, in some embodiments the polynucleotide is capable of expressing an annexin protein that comprises a secretory tag. In some embodiments, a composition comprises one or more polynucleotides capable of expressing one or more annexin proteins (e.g., annexin A6, annexin A1, annexin A2, or a combination thereof) and one or more or all of the annexin proteins are secreted extracellularly.

### Polynucleotides

In some embodiments, an agent of the disclosure is a polynucleotide capable of expressing an annexin protein as described herein. The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polynucleotides and polyribonucleotides can also be prepared enzymatically via, e.g., polymerase chain reaction (PCR). Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

### Steroids

In some embodiments, the agent that increases the activity of an annexin protein is a steroid. In further embodiments, the steroid is a corticosteroid, a glucocorticoid, or a mineralocorticoid. In still further embodiments, the corticosteroid is Betamethasone, Budesonide, Cortisone, Dexamethasone, Hydrocortisone, Methylprednisolone, Prednisolone, or Prednisone. In some embodiments, the corticosteroid is salmeterol, fluticasone, or budesonide.

In some embodiments, the steroid is an anabolic steroid. In further embodiments anabolic steroids, include, but are not limited to, testosterone or related steroid compounds with muscle growth inducing properties, such as cyclostanazol or methadrostenol, prohomones or derivatives thereof, modulators of estrogen, and selective androgen receptor modulators (SARMS).

### Vectors

An appropriate expression vector may be used to deliver exogenous nucleic acid to a recipient muscle cell in the methods of the disclosure. In order to achieve effective gene therapy, the expression vector must be designed for efficient cell uptake and gene product expression. In some embodiments, the vector is within a chloroplast. In some embodiments, the vector is a viral vector. In some embodiments, the viral vector is selected from the group consisting of a herpes virus vector, an adeno-associated virus (AAV) vector, an adeno virus vector, and a lentiviral vector.

Use of adenovirus or adeno-associated virus (AAV) based vectors for gene delivery have been described [Berkner, Current Topics in Microbiol. and Imunol. 158: 39-66 (1992); Stratford-Perricaudet et al., Hum. Gene Ther. 1: 241-256 (1990); Rosenfeld et al., Cell 8: 143-144 (1992); Stratford-Perricaudet et al., J. Clin. Invest. 90: 626-630 (1992)]. In various embodiments, the adeno-associated virus vector is AAV1, AAV2, AAV4, AAV5, AAV6, AAV7, AAV8, AAV9, AAVrh10, AAV PHP.B, AAV PDP.eB, or AAV74. In some embodiments, the adeno-associated virus vector is AAV9. In further embodiments, the adeno-associated virus vector is AAVrh74. The disclosure contemplates use of any adeno-associated virus (AAV) based vector that effectively crosses the blood brain barrier and transduces neurons in a patient.

Specific methods for gene therapy useful in the context of the present disclosure depend largely upon the expression system employed; however, most methods involve insertion of coding sequence at an appropriate position within the expression vector, and subsequent delivery of the expression vector to the target muscle tissue for expression.

Additional delivery systems useful in the practice of the methods of the disclosure are discussed in U.S. Patent Publication Numbers 2012/0046345 and 2012/0039806, each of which is incorporated herein by reference in its entirety.

### Disorders/Injuries

In various aspects, the disclosure provides compositions for treating, delaying onset, enhancing recovery from, or preventing a cellular membrane injury, comprising administering an agent and optionally an additional agent to a patient in need thereof. In some aspects, methods comprising administering a therapeutically effective amount of an agent that increases the activity of an annexin protein to a patient are provided, wherein the patient has an elevated serum or plasma level of lactate dehydrogenase (LDH), cardiac troponin T, cardiac troponin I, creatine kinase (CK), or a combination thereof, relative to a control level. In further aspects, the disclosure provides methods of reducing serum or plasma level of lactate dehydrogenase (LDH), cardiac troponin T, cardiac troponin I, creatine kinase (CK), or a combination thereof, in a patient in need thereof, comprising administering a therapeutically effective amount of an agent that increases the activity of an annexin protein to the patient, thereby reducing the serum or plasma level of lactate dehydrogenase (LDH), cardiac troponin T, cardiac troponin I, creatine kinase (CK), or a combination thereof in the patient. In various embodiments, such a patient is one that is suffering from an acute injury or a chronic injury.

In further embodiments, the patient is suffering from, for example, Duchenne Muscular Dystrophy, Limb Girdle Muscular Dystrophy, Becker Muscular Dystrophy, Emery-Dreifuss Muscular Dystrophy (EDMD), Myotonic Dystrophy, Fascioscapulohumeral Dystrophy (FSHD), Oculopharyngeal Muscular Dystrophy, Distal Muscular Dystrophy, cystic fibrosis, pulmonary fibrosis, muscle atrophy, cerebral palsy, an epithelial disorder, an epidermal disorder, a kidney disorder, a liver disorder, sarcopenia, cardiomyopathy, myopathy, cystic fibrosis, pulmonary fibrosis, cardiomyopathy (including hypertrophic, dilated, congenital, arrhythmogenic, restrictive, ischemic, or heart failure), acute lung injury, acute muscle injury, acute myocardial injury, radiation-induced injury, colon cancer, idiopathic pulmonary fibrosis, idiopathic interstitial pneumonia, autoimmune lung diseases, benign prostate hypertrophy, cerebral infarction, musculoskeletal fibrosis, post-surgical adhesions, liver cirrhosis, renal fibrotic disease, fibrotic vascular disease, neurofibromatosis, Alzheimer's disease, diabetic retinopathy, skin lesions, lymph node fibrosis associated with HIV, chronic obstructive pulmonary disease (COPD), inflammatory pulmonary fibrosis, rheumatoid arthritis; rheumatoid spondylitis; osteoarthritis; gout, other arthritic conditions; sepsis; septic shock; endotoxic shock; gram-negative sepsis; toxic shock syndrome; myofacial pain syndrome (MPS); Shigellosis; asthma; adult respiratory distress syndrome; inflammatory bowel disease; Crohn's disease; psoriasis; eczema; ulcerative colitis; glomerular nephritis; scleroderma; chronic thyroiditis; Grave's disease; Ormond's disease; autoimmune gastritis; myasthenia gravis; autoimmune hemolytic anemia; autoimmune neutropenia; thrombocytopenia; pancreatic fibrosis; chronic active hepatitis including hepatic fibrosis; renal fibrosis, irritable bowel syndrome; pyresis; restenosis; cerebral malaria; stroke and ischemic injury; neural trauma; Huntington's disease; Parkinson's disease; allergies, including allergic rhinitis and allergic conjunctivitis; cachexia; Reiter's syndrome; acute synoviitis; muscle degeneration, bursitis; tendonitis; tenosynoviitis; osteopetrosis; thrombosis; silicosis; pulmonary sarcosis; bone resorption diseases, such as osteoporosis or multiple myeloma-related bone disorders; cancer, including but not limited to metastatic breast carcinoma, colorectal carcinoma, malignant melanoma, gastric cancer, and non-small cell lung cancer; graft-versus-host reaction; and auto-immune diseases, such as multiple sclerosis, lupus and fibromyalgia; viral diseases such as Herpes Zoster, Herpes Simplex I or II, influenza virus, Severe Acute Respiratory Syndrome (SARS) and cytomegalovirus. In some embodiments, the acute injury results from surgery, a burn, a toxin, a chemical, radiation-induced injury, acute myocardial injury, acute muscle injury, acute lung injury, acute epithelial injury, acute epidermal injury, acute kidney injury, acute liver injury, vascular injury, an excessive mechanical force, trauma, acute brain injury from stroke or trauma, myositis, or acute cardiac injury. In some embodiments, the chronic disorder is Becker Muscular Dystrophy (BMD), Duchenne Muscular Dystrophy (DMD), Limb Girdle Muscular Dystrophy, Friedreich's Ataxia, congenital Muscular Dystrophy, Emery-Dreifuss Muscular Dystrophy (EDMD), Myotonic Dystrophy, Fascioscapulohumeral Dystrophy (FSHD), Oculopharyngeal Muscular Dystrophy, Distal Muscular Dystrophy, cystic fibrosis, pulmonary fibrosis, muscle atrophy, cerebral palsy, an epithelial disorder, an epidermal disorder, a kidney disorder, a liver disorder, sarcopenia, chronic cardiac injury, or cardiomyopathy (hypertrophic, dilated, congenital, arrhythmogenic, restrictive, ischemic, heart failure).

As used herein, “cardiomyopathy” refers to any disease or dysfunction of the myocardium (heart muscle) in which the heart is abnormally enlarged, thickened and/or stiffened. As a result, the heart muscle's ability to pump blood is usually weakened, often leading to congestive heart failure. The disease or disorder can be, for example, inflammatory, metabolic, toxic, infiltrative, fibrotic, hematological, genetic, or unknown in origin. Such cardiomyopathies may result from a lack of oxygen. Other diseases include those that result from myocardial injury which involves damage to the muscle or the myocardium in the wall of the heart as a result of disease or trauma. Myocardial injury can be attributed to many things such as, but not limited to, cardiomyopathy, myocardial infarction, or congenital heart disease. The cardiac disorder may be pediatric in origin. Cardiomyopathy includes, but is not limited to, cardiomyopathy (dilated, hypertrophic, restrictive, arrhythmogenic, genetic, idiopathic and unclassified cardiomyopathy), sporadic dilated cardiomyopathy, X-linked Dilated Cardiomyopathy (XLDC), acute and chronic heart failure, right heart failure, left heart failure, biventricular heart failure, congenital heart defects, myocardiac fibrosis, mitral valve stenosis, mitral valve insufficiency, aortic valve stenosis, aortic valve insufficiency, tricuspidal valve stenosis, tricuspidal valve insufficiency, pulmonal valve stenosis, pulmonal valve insufficiency, combined valve defects, myocarditis, acute myocarditis, chronic myocarditis, viral myocarditis, diastolic heart failure, systolic heart failure, diabetic heart failure and accumulation diseases. In further embodiments, the cardiomyopathy is hypertrophic, dilated, congenital, arrhythmogenic, restrictive, ischemic, Friedreich Ataxia, or heart failure.

In some aspects, the disclosure provides methods of treating a patient suffering from a nerve injury comprising administering to the patient a therapeutically effective amount of a composition comprising an agent that increases the activity of an annexin protein. In further aspects, methods of delaying onset, enhancing recovery from a nerve injury, or preventing a nerve injury are provided, comprising administering to a patient in need thereof a therapeutically effective amount of a composition comprising an agent that increases the activity of an annexin protein. In various embodiments, the nerve injury is an acute nerve injury or a chronic nerve injury. In some embodiments, the nerve injury is a partially transected nerve, a wholly transected nerve, a nerve injury due to ischemia, a nerve injury due to infection, a nerve injury due to trauma, or a combination thereof. In further embodiments, the patient has a crush injury, a concussion, traumatic brain injury (TBI), or peripheral nerve disease.

In further aspects of the disclosure, methods of treating a patient suffering from a disorder are provided comprising administering to the patient a therapeutically effective amount of a composition comprising an agent that increases the activity of an annexin protein, wherein the disorder is stroke, dementia, Alzheimer Dementia, Frontotemporal Dementia, Parkinsons Disease, spinal cord injury, small vessel disease, transient ischemic attack, cerebrovascular accident, dementia due to small vessel disease, Guillain Barré, Acute Inflammatory Demyelinating, Polyradiculopathy, Peripheral nerve disease, neuropathy, diabetic neuropathy, acute myocardial infarction, hypertrophic cardiomyopathy, dilated cardiomyopathy, myocarditis, arrhythmogenic cardiomyopathy, restrictive cardiomyopathy, ischemic cardiomyopathy, myocardiac injury acute, or myocardial injury. In some aspects, the disclosure provides methods of delaying onset, enhancing recovery from cellular membrane injury, or preventing a disorder comprising administering to a patient in need thereof a therapeutically effective amount of a composition comprising an agent that increases the activity of an annexin protein, wherein the disorder is stroke, dementia, Alzheimer Dementia, Frontotemporal Dementia, Parkinsons Disease, spinal cord injury, small vessel disease, transient ischemic attack, cerebrovascular accident, dementia due to small vessel disease, Guillain Barré, Acute Inflammatory Demyelinating, Polyradiculopathy, Peripheral nerve disease, neuropathy, diabetic neuropathy, acute myocardial infarction, hypertrophic cardiomyopathy, dilated cardiomyopathy, myocarditis, arrhythmogenic cardiomyopathy, restrictive cardiomyopathy, ischemic cardiomyopathy, myocardiac injury acute, or myocardial injury.

### Preclinical Alzheimer's Disease/Dementia and Mild-to-Moderate Congnitive Impairment

Alzheimer's Disease (AD) is a devastating neurodegenerative disorder that represents a serious national health problem, and therapies that treat the underlying cause of AD are desperately needed. The AD brain is characterized by amyloid plaques containing the β-amyloid peptide, and neurofibrillary tangles containing hyperphosphorylated, aggregated tau. Amyloid plaques form first and likely give rise to tangles, but the mechanistic link between them is unclear. The peri-plaque environment is toxic to neurons, characterized by synaptic loss, activated microglia, and vesicle-filled dystrophic neurites, which accumulate aggregation-prone phosphorylated forms of tau. Although Aβ immunotherapies and tau-targeted therapies are being developed, it is unclear whether any will be successful. Therefore, continued intense efforts devoted to the discovery of safe and effective therapies that target the biological basis of AD are imperative.

Accordingly, in some aspects the present disclosure provides methods of treating preclinical Alzheimer's disease or mild-to-moderate cognitive impairment comprising administering to the patient a therapeutically effective amount of a composition comprising an agent that increases the activity of an annexin protein to a patient in need thereof. In some aspects, the disclosure also provides methods comprising administering a therapeutically effective amount of a composition comprising an agent that increases the activity of an annexin protein to a patient having preclinical Alzheimer's disease or mild-to-moderate cognitive impairment. In various embodiments, the patient additionally has (i) a cerebrospinal fluid (CSF) ratio of Aβ42/Aβ40 ratio that is less than about 0.07; (ii) a plasma level of Aβ42 that is greater than zero and less than about 1000 pg/ml; (iii) a serum or plasma level of phosphorylated tau protein that is about 24 pg/ml or greater; (iv) an amount of phosphorylated tau protein in their cerebrospinal fluid (CSF) that is about 52 pg/ml or greater; and/or (v) an amount of amyloid plaques in their brain that is about 10 to about 60 centiloids. In any of the aspects or embodiments of the disclosure, the composition is a pharmaceutical composition as described herein. As used herein, a “preclinical Alzheimer's disease patient” is a patient that does not exhibit clinical symptoms of Alzheimer's disease but is diagnosed as being a preclinical Alzheimer's disease patient based on a level of one or more Alzheimer's disease biomarkers such as those described herein that is indicative of preclinical Alzheimer's disease. As used herein, a patient with “mild-to-moderate cognitive impairment” is a patient that (i) exhibits mild to moderate clinical symptoms of Alzheimer's disease based on cognitive testing, and optionally (ii) has a level of one or more Alzheimer's disease biomarkers such as those described herein that is indicative of mild-to-moderate cognitive impairment. In some embodiments, the patient was previously diagnosed with preclinical Alzheimer's disease or mild-to-moderate cognitive impairment via cognitive testing. In further embodiments, a method of the disclosure includes diagnosing the patient with preclinical Alzheimer's disease or mild-to-moderate cognitive impairment via cognitive testing.

### Alzheimer's Disease Biomarkers

In some aspects of the disclosure, a preclinical Alzheimer's disease patient or a patient with mild-to-moderate cognitive impairment is identified via testing of one or more Alzheimer's disease biomarkers. In general, Alzheimer's disease biomarkers fall into three categories: amyloid level, tau level, and neurodegeneration level. In some embodiments, the preclinical Alzheimer's disease patient or the patient with mild-to-moderate cognitive impairment is amyloid positive, tau negative, and neurodegeneration negative. In some embodiments, the preclinical Alzheimer's disease patient or the patient with mild-to-moderate cognitive impairment is amyloid positive, tau positive, and neurodegeneration negative. In further embodiments, the preclinical Alzheimer's disease patient or the patient with mild-to-moderate cognitive impairment is amyloid positive, tau negative, and neurodegeneration positive. In still further embodiments, the preclinical Alzheimer's disease patient or the patient with mild-to-moderate cognitive impairment is amyloid positive, tau positive, and neurodegeneration positive. See also Jack Jr. et al., Alzheimers Dement 2018 April; 14(4):535-562 and Erickson et al., Alzheimer's Dement. 2021; 13:e12150, each of which is incorporated by reference herein in its entirety. Suitable methods for measuring a level of a biomarker of the disclosure are known in the art and include, but are not limited to, capture-specific assays, in particular antibody-based assays (e.g., ELISA, single molecule array (SIMOA™) technology, etc.), and high resolution mass spectrometry.

Amyloid level may be determined by any one or more diagnostic tests known in the art, including but not limited to PET amyloid imaging, cerebrospinal fluid (CSF) or plasma biomarkers, or other biological fluid biomarkers. In some embodiments, a preclinical Alzheimer's disease patient or a patient with mild-to-moderate cognitive impairment has a plasma level of Aβ₄₂ that is greater than zero and less than about 1000 picograms per milliliter (pg/ml). In further embodiments, a preclinical Alzheimer's disease patient or a patient with mild-to-moderate cognitive impairment has a plasma level of Aβ₄₂ that is about 10 pg/ml to about 1000 pg/ml, about 10 pg/ml to about 950 pg/ml, about 10 pg/ml to about 900 pg/ml, about 10 pg/ml to about 850 pg/ml, about 10 pg/ml to about 800 pg/ml, about 10 pg/ml to about 700 pg/ml, about 10 pg/ml to about 600 pg/ml, about 10 pg/ml to about 500 pg/ml, about 10 pg/ml to about 400 pg/ml, about 10 pg/ml to about 300 pg/ml, about 10 pg/ml to about 200 pg/ml, or about about 10 pg/ml to about 100 pg/ml. In further embodiments, a preclinical Alzheimer's disease patient or a patient with mild-to-moderate cognitive impairment has a plasma level of Aβ₄₂ that is less than about 1000 pg/ml, less than about 980 pg/ml, less than about 950 pg/ml, less than about 900 pg/ml, less than about 850 pg/ml, less than about 800 pg/ml, less than about 750 pg/ml, less than about 700 pg/ml, less than about 650 pg/ml, less than about 600 pg/ml, less than about 550 pg/ml, less than about 500 pg/ml, less than about 400 pg/ml, less than about 300 pg/ml, less than about 200 pg/ml, or less than about 100 pg/ml. In some embodiments, a preclinical Alzheimer's disease patient or a patient with mild-to-moderate cognitive impairment has a cerebrospinal fluid (CSF) ratio of Aβ₄₂/Aβ₄₀ that is, is about, or is less than about 0.07. In further embodiments, a preclinical Alzheimer's disease patient or a patient with mild-to-moderate cognitive impairment has a cerebrospinal fluid (CSF) ratio of Aβ₄₂/Aβ₄₀ ratio that is, is about, or is less than about 0.06, 0.05, 0.04, 0.03, 0.02, or 0.01. In further embodiments, a preclinical Alzheimer's disease patient or a patient with mild-to-moderate cognitive impairment has an amount of amyloid plaques in their brain that is about 10 to about 60 centiloids, or about 10 to about 50, or about 10 to about 40, or about 10 to about 30, or about 10 to about 20, or about 12 to about 30 centiloids, or about 12 to about 60 centiloids. In various embodiments, a preclinical Alzheimer's disease patient or a patient with mild-to-moderate cognitive impairment has an amount of amyloid plaques in their brain that is or is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 centiloids. The amount of amyloid plaques may be measured, for example and without limitation, by positron emission tomography (PET)-Pittsburgh Compound-B (PIB) (PET-PIB) imaging. Thus, in any of the aspects or embodiments of the disclosure, a preclinical Alzheimer's disease patient or a patient with mild-to-moderate cognitive impairment is characterized as “amyloid positive” when the patient has measurable amyloid deposition according to any one or more of the foregoing amyloid levels as measured by imaging, CSF/plasma/serum analysis, or a combination thereof.

Tau protein level may be determined by any one or more diagnostic tests known in the art, including but not limited to Tau PET imaging (e.g., fluortaucipir) or CSF or plasma or other fluid based biomarkers for tau phosphorylation or aggregation pathology. Tau protein levels may be measured as an amount of phosphorylated tau protein in serum/plasma, cerebrospinal fluid (CSF), or a combination thereof. In various embodiments, the phosphorylated tau protein is p-tau181, p-tau231, p-tau217, or a combination thereof. In some embodiments, the phosphorylated tau protein is p-tau181. In some embodiments, a preclinical Alzheimer's disease patient or a patient with mild-to-moderate cognitive impairment has a serum or plasma level of phosphorylated tau protein (e.g., p-tau181) that is about 20 picograms per milliliter (pg/ml) or greater. In further embodiments, a preclinical Alzheimer's disease patient or a patient with mild-to-moderate cognitive impairment has a serum or plasma level of phosphorylated tau protein (e.g., p-tau181) that is, is about, or is at least about 20 picograms per milliliter (pg/ml), 21 pg/ml, 22 pg/ml, 23 pg/ml, 24 pg/ml, 25 pg/ml, 26 pg/ml, 27 pg/ml, 28 pg/ml, 29 pg/ml, or 30 pg/ml. In some embodiments, a preclinical Alzheimer's disease patient or a patient with mild-to-moderate cognitive impairment has an amount of phosphorylated tau protein in their cerebrospinal fluid (CSF) that is about 52 picograms per milliliter (pg/ml) or greater. In further embodiments, a preclinical Alzheimer's disease patient or a patient with mild-to-moderate cognitive impairment has an amount of phosphorylated tau protein (e.g., p-tau181) in their cerebrospinal fluid (CSF) that is, is about, or is at least about 50 picograms per milliliter (pg/ml), 51 pg/ml, 52 pg/ml, 53 pg/ml, 54 pg/ml, 55 pg/ml, 56 pg/ml, 57 pg/ml, 58 pg/ml, 59 pg/ml, or 60 pg/ml. In some embodiments, a preclinical Alzheimer's disease patient or a patient with mild-to-moderate cognitive impairment has an amount of p-tau 231 in their serum or plasma that is, is about, or is at least about 3.0, 3.1, 3.2, 3.3, 3.4, or 3.5 picograms per milliliter (pg/ml). In some embodiments, a preclinical Alzheimer's disease patient or a patient with mild-to-moderate cognitive impairment has an amount of p-tau 231 in their cerebrospinal fluid (CSF) that is, is about, or is at least about 11.0, 11.1, 11.2, 11.3, 11.4 picograms per milliliter (pg/ml). In further embodiments, a preclinical Alzheimer's disease patient or a patient with mild-to-moderate cognitive impairment has an amount of p-tau 231 in their serum or plasma that is, is about, or is at least about 120, 121, 122, 123, 124, 124.6, or 125 femtograms per milliliter (fg/ml). Thus, in any of the aspects or embodiments of the disclosure, a preclinical Alzheimer's disease patient or a patient with mild-to-moderate cognitive impairment is characterized as “tau positive” when the patient has measurable tau protein level according to any one or more of the foregoing tau protein levels as measured by, for example and without limitation, capture-specific assays, in particular antibody-based assays (e.g., ELISA, single molecule array (SIMOA™) technology, etc.), and/or high resolution mass spectrometry. By contrast, a preclinical Alzheimer's disease patient or a patient with mild-to-moderate cognitive impairment is characterized as “tau negative” when they do not have abnormal tau protein levels according to the foregoing tau protein levels.

Neurodegeneration positivity may be determined by any one or more diagnostic tests known in the art, including but not limited to tests that reveal signs of neurodegeneration or neuronal injury by (18)F-fluorodeoxyglucose (FDG) positron emission tomography (PET) (FDG PET), anatomic magnetic resonance imaging (MRI), fluid based biomarkers such as total tau, or neurofilament light chain. In various embodiments, the following are indicative of neurodegeneration positivity: (a) a plasma neurofilament light chain concentration greater than about 35.02 pg/mL, (90%, or greater of the cognitively unimpaired Aβ-negative patients; (b) a plasma neurofilament light chain concentration greater than about 25.7 pg/mL; (c) a plasma total tau concentration of about 465 pg/ml or greater (INNOTEST® hTau Ag Fujirebio ELISA based Assay); (d) a CSF t-tau concentration greater than about 300 pg/ml. The foregoing may be measured via tests such as, without limitation, ELISA, Simoa, Elecsys, and/or mass spectrometry.

Biomarker levels may also be quantified using a commercial test. Exemplary commercial tests include but are not limited to PrecivityAD™ (C₂N Diagnostics, St. Louis, MO) where the patient has, for example, a test Amyloid Probability Score (APS) of “intermediate” or “high” likelihood of brain amyloid plaque pathology (above 35 on a scale of 100); Elecsys® CSF test (Roche Diagnostics) where the patient has, for example, one or more of the following: Aβ42<1031 pg/ml, pTau181>27 pg/ml, total tau>300 pg/ml, ptau181/Aβ42>0.023, total Tau/Aβ42>0.28; LUMIPULSE® G1200 CSF test (Fujirebio) where the patient has, for example, one or more of the following: >409 ng/L for total tau, >50.2 ng/L for pTau 181, <526 ng/L for β-amyloid 1-42, and <0.072 for the Aβ 1-42/Aβ1-40 ratio; Quest AD-Detect™ plasma test (Quest Diagnostics) where the patient has, for example, Aβ42/Aβ40<0.16.

### Cognitive Testing

In some aspects of the disclosure, a preclinical Alzheimer's disease patient or a patient with mild-to-moderate cognitive impairment is identified via cognitive testing. Thus, in some embodiments a preclinical Alzheimer's disease patient or a patient with mild-to-moderate cognitive impairment is a patient that scores between 27 and 30 (no impairment), 19 and 26 (early AD, mild cognitive impairment) or 10-20 (Mod AD, moderate impairment) on the Mini-Mental State Exam (MMSE). In further embodiments, a preclinical Alzheimer's disease patient or a patient with mild-to-moderate cognitive impairment is a patient that scores between 0-15.5 (normal to to moderate impairment on Clinical Dementia Rating Sum of Boxes (CDR SOB) rating. In further embodiments, a preclinical Alzheimer's disease patient or a patient with mild-to-moderate cognitive impairment is a patient that scores between 27-30 (no impairment), between 26 and 18 (mild cognitive impairment) or 10-17 (moderate cognitive impairment) on the Montreal Cognitive Assessment (MoCA). In still further embodiments, a preclinical Alzheimer's disease patient or a patient with mild-to-moderate cognitive impairment is a patient that scores between 5-1 (normal to positive for dementia) on mini-cog test. In some embodiments, results from more than one of the foregoing tests are used to identify a preclinical Alzheimer's disease patient or a patient with mild-to-moderate cognitive impairment.

### Additional (Second) Agents

In any of the aspects or embodiments of the disclosure a second agent may be administered with the agent that increases the activity of an annexin protein. Nonlimiting examples of the second agent are an acetylcholinesterase inhibitor (e.g., ARICEPT® (donepezil hydrochloride)), an anti-amyloid antibody (e.g., ADUHELM® (aducanumab-avwa)), a beta-secretase enzyme inhibitor, an anti-tau antibody, a modulator of microglial activity, an NMDA (N-methyl-D-aspartate) receptor antagonist (e.g., Namenda® (memantine HCl)), mitsugumin 53 (MG53), micro-dystrophin, a modulator of latent TGF-β binding protein 4 (LTBP4), a modulator of transforming growth factor β (TGF-β activity, a modulator of androgen response, a modulator of an inflammatory response, a promoter of muscle growth, a chemotherapeutic agent, a modulator of fibrosis, and a combination thereof. Further, the methods disclosed herein can, in various embodiments, encompass one or more of such agents, or one or more of such agents in composition with any other active agent(s).

### Modulators of LTBP4

LTBP4 is located on human chromosome 19q13.1-q13.2, and is an extracellular matrix protein that binds and sequesters TGFβ. LTBP4 modifies murine muscular dystrophy through a polymorphism in the Ltbp4 gene. See U.S. Pat. No. 9,873,739, which is incorporated by reference herein in its entirety. There are two common variants of the Ltbp4 gene in mice. Most strains of mice, including the mdx mouse, have the Ltbp4 insertion allele (Ltbp4^{I/I}). Insertion of 36 base pairs (12 amino acids) into the proline-rich region of LTBP4 encoded by Ltbp4^{I/I} leads to milder disease. Deletion of 36 bp/12aa in the proline-rich region is associated with more severe disease (Ltbp4^{D/D}). It was found that the Ltbp4 genotype correlated strongly with two different aspects of muscular dystrophy pathology, i.e., membrane leakage and fibrosis, and these features define DMD pathology.

Modulators of LTBP4 are described in U.S. Pat. No. 9,873,739, which is incorporated by reference herein in its entirety.

### Modulators of TGF-β Activity

Transforming Growth Factor-β (TGF-β superfamily is a family of secreted proteins that is comprised of over 30 members including activins, nodals, bone morphogenic proteins (BMPs) and growth and differentiation factors (GDFs). Superfamily members are generally ubiquitously expressed and regulate numerous cellular processes including growth, development, and regeneration. Mutations in TGF-β superfamily members result in a multitude of diseases including autoimmune disease, cardiac disease, fibrosis and cancer.

TGF-β ligand family includes TGF-β1, TGF-β2, and TGF-β3. TGF-β is secreted into the extracellular matrix in an inactive form bound to latency associated peptide (LAP). Latent TGF-β proteins (LTBPs) binding the TGF-β/LAP complex provide yet another level of regulation. Extracellular proteases cleave LTBP/LAP/TGF-β releasing TGF-β. As a result, TGF-β is free to bind its receptors TGFBRI or TGFBRII. TGF-β/receptor binding, activates downstream canonical and non-canonical SMAD pathways, including activation of SMAD factors, leading to gene transcription. TGF-β signaling has emerged as a prominent mediator of the fibrotic response and disease progression in muscle disease and its expression is upregulated in dystrophy in both mouse and human. Blockade of TGF-β signaling in mice through expression of a dominant negative receptor (TGFBRII) expression, improved the dystrophic pathology, enhanced regeneration, and reduced muscle injury of 5-sarcoglycan-null mice, a mouse model of muscular dystrophy (Accornero, McNally et al Hum Mol Genet 2014). Additionally, antibody-mediated blockade of TGF-β signaling with a pan anti-TGF-β antibody, 1d11 monocloncal antibody, improved respiratory outcome measures in a mouse model of Duchenne muscular dystrophy (Nelson, Wentworth et al Am J Pathol 2011). Thus, therapeutic approaches against TGF-β signaling are contemplated herein to improve repair and delay disease progression.

Therapeutics contemplated as effective against TGF-β signaling include galunisertib (LY2157299 monohydrate), TEW-7917, monoclonal antibodies against TGF-β ligands (TGF-β 1, 2, 3 alone or pan 1,2,3), Fresolimemub (GC-1008), TGF-β peptide P144, LY2382770, small molecule, SB-525334, and GW788388.

### Modulators of an Androgen Response

Selective androgen receptor modulators (SARMs) are a class of androgen receptor ligands that activate androgenic signaling and exist in nonsteroidal and steroidal forms. Studies have shown that SARMs have the potential to increase both muscle and bone mass. Testosterone is one of the most well-known SARMs, which promotes skeletal muscle growth in healthy and diseased tissue. Testosterone and dihydrotestosterone (DHT) promote myocyte differentiation and upregulate follistatin, while also downregulates TGF-β signaling, resulting in muscle growth (Singh et al 2003, Singh et al 2009, Gupta et al 2008). It is conceivable that SARM-mediated inhibition of TGF-β protects against muscle injury and improves repair. SARMS may include, testosterone, estrogen, dihydrotestosterone, estradiol, include dihydronandrolone, nandrolone, nandrolone decanoate, Ostarine, Ligandrol, LGD-3303, andarine, cardarine, 7-alpha methyl, 19-nortestosterone aryl-propionamide, bicyclic hydantoin, quinolinones, tetrahydroquinoline analog, benizimidazole, imidazolopyrazole, indole, and pyrazoline derivatives, azasteroidal derivatives, and aniline, diaryl aniline, and bezoxazepinones derivatives.

### Modulators of an Inflammatory Response

A modulator of an inflammatory response includes the following agents. In one embodiment of the disclosure, the modulator of an inflammatory response is a beta2-adrenergic receptor agonist (e.g., albuterol). The term beta2-adrenergic receptor agonist is used herein to define a class of drugs which act on the 32-adrenergic receptor, thereby causing smooth muscle relaxation resulting in dilation of bronchial passages, vasodilation in muscle and liver, relaxation of uterine muscle and release of insulin. In one embodiment, the beta2-adrenergic receptor agonist for use according to the disclosure is albuterol, an immunosuppressant drug that is widely used in inhalant form for asthmatics. Albuterol is thought to slow disease progression by suppressing the infiltration of macrophages and other immune cells that contribute to inflammatory tissue loss. Albuterol also appears to have some anabolic effects and promotes the growth of muscle tissue. Albuterol may also suppress protein degradation (possibly via calpain inhibition).

In DMD, the loss of dystrophin leads to breaks in muscle cell membrane, and destabilizes neuronal nitric oxide synthase (nNOS), a protein that normally generates nitric oxide (NO). It is thought that at least part of the muscle degeneration observed in DMD patients may result from the reduced production of muscle membrane-associated neuronal nitric oxide synthase. This reduction may lead to impaired regulation of the vasoconstrictor response and eventual muscle damage.

In one embodiment, modulators of an inflammatory response suitable for use in compositions of the disclosure are Nuclear Factor Kappa-B (NF-κB) inhibitors. NF-κB is a major transcription factor modulating cellular immune, inflammatory and proliferative responses. NF-κB functions in activated macrophages to promote inflammation and muscle necrosis and in skeletal muscle fibers to limit regeneration through the inhibition of muscle progenitor cells. The activation of this factor in DMD contributes to diseases pathology. Thus, NF-κB plays an important role in the progression of muscular dystrophy and the IKK/NF-κB signaling pathway is a potential therapeutic target for the treatment of a TGFβ-related disease. Inhibitors of NF-κB (for example and without limitation, IRFI 042, a vitamin E analog) enhance muscle function, decrease serum creatine kinase (CK) level and muscle necrosis and enhance muscle regeneration. Edasalonexent is a small molecule inhibitor NF-κB. Edasalonexent administered orally as 100 mg/kg delayed muscle disease progression in Duchenne muscular dystrophy boys. Furthermore, specific inhibition of NF-κB-mediated signaling by IKK has similar benefits.

In a further embodiment, the modulator of an inflammatory response is a tumor necrosis factor alpha antagonist. TNF-α is one of the key cytokines that triggers and sustains the inflammation response. In one specific embodiment of the disclosure, the modulator of an inflammatory response is the TNF-α antagonist infliximab.

TNF-α antagonists for use according to the disclosure include, in addition to infliximab (Remicade™), a chimeric monoclonal antibody comprising murine VK and VH domains and human constant Fc domains. The drug blocks the action of TNF-α by binding to it and preventing it from signaling the receptors for TNF-α on the surface of cells. Another TNF-α antagonist for use according to the disclosure is adalimumab (Humira™) Adalimumab is a fully human monoclonal antibody. Another TNF-α antagonist for use according to the disclosure is etanercept (Enbrel™). Etanercept is a dimeric fusion protein comprising soluble human TNF receptor linked to an Fc portion of an IgG1. It is a large molecule that binds to TNF-α and thereby blocks its action. Etanercept mimics the inhibitory effects of naturally occurring soluble TNF receptors, but as a fusion protein it has a greatly extended half-life in the bloodstream and therefore a more profound and long-lasting inhibitory effect.

Another TNF-α antagonist for use according to the disclosure is pentoxifylline (Trental™), chemical name 1-(5-oxohexyl)-3,7-dimethylxanthine. The usual dosage in controlled-release tablet form is one tablet (400 mg) three times a day with meals.

Dosing: Remicade is administered by intravenous infusion, typically at 2-month intervals. The recommended dose is 3 mg/kg given as an intravenous infusion followed with additional similar doses at 2 and 6 weeks after the first infusion, then every 8 weeks thereafter. For patients who have an incomplete response, consideration may be given to adjusting the dose up to 10 mg/kg or treating as often as every 4 weeks. Humira is marketed in both preloaded 0.8 ml (40 mg) syringes and also in preloaded pen devices, both injected subcutaneously, typically by the patient at home. Etanercept can be administered at a dose of 25 mg (twice weekly) or 50 mg (once weekly).

In another embodiment of the disclosure, the modulator of an inflammatory response is cyclosporin. Cyclosporin A, the main form of the drug, is a cyclic nonribosomal peptide of 11 amino acids produced by the fungus Tolypocladium inflatum. Cyclosporin is thought to bind to the cytosolic protein cyclophilin (immunophilin) of immunocompetent lymphocytes (especially T-lymphocytes). This complex of cyclosporin and cyclophylin inhibits calcineurin, which under normal circumstances is responsible for activating the transcription of interleukin-2. It also inhibits lymphokine production and interleukin release and therefore leads to a reduced function of effector T-cells. It does not affect cytostatic activity. It has also an effect on mitochondria, preventing the mitochondrial PT pore from opening, thus inhibiting cytochrome c release (a potent apoptotic stimulation factor). Cyclosporin may be administered at a dose of 1-10 mg/kg/day.

### Promoters of Muscle Growth

In some embodiments of the disclosure, a therapeutically effective amount of a promoter of muscle growth is administered to a patient. Promoters of muscle growth contemplated by the disclosure include, but are not limited to, insulin-like growth factor-1 (IGF-1), Akt/protein kinase B, clenbuterol, creatine, decorin (see U.S. Patent Publication Number 20120058955), a steroid (for example and without limitation, a corticosteroid or a glucocorticoid steroid), testosterone and a myostatin antagonist.

### Myostatin Antagonists

Another class of promoters of muscle growth suitable for use in the combinations of the disclosure is myostatin antagonists. Myostatin, also known as growth/differentiation factor 8 (GDF-8) is a transforming growth factor-β (TGFβ) superfamily member involved in the regulation of skeletal muscle mass. Most members of the TGF-β-GDF family are widely expressed and are pleiotropic; however, myostatin is primarily expressed in skeletal muscle tissue where it negatively controls skeletal muscle growth. Myostatin is synthesized as an inactive preproprotein which is activated by proteolyic cleavage. The precursor protein is cleaved to produce an approximately 109-amino-acid COOH-terminal protein which, in the form of a homodimer of about 25 kDa, is the mature, active form. The mature dimer appears to circulate in the blood as an inactive latent complex bound to the propeptide. As used herein the term “myostatin antagonist” defines a class of agents that inhibits or blocks at least one activity of myostatin, or alternatively, blocks or reduces the expression of myostatin or its receptor (for example, by interference with the binding of myostatin to its receptor and/or blocking signal transduction resulting from the binding of myostatin to its receptor). Such agents therefore include agents which bind to myostatin itself or to its receptor.

Myostatin antagonists for use according to the disclosure include antibodies to GDF-8; antibodies to GDF-8 receptors; soluble GDF-8 receptors and fragments thereof (e.g., the ActRIIB fusion polypeptides as described in U.S. Patent Publication Number 2004/0223966, which is incorporated herein by reference in its entirety, including soluble ActRIIB receptors in which ActRIIB is joined to the Fc portion of an immunoglobulin); GDF-8 propeptide and modified forms thereof (e.g., as described in WO 2002/068650 or U.S. Pat. No. 7,202,210, including forms in which GDF-8 propeptide is joined to the Fc portion of an immunoglobulin and/or form in which GDF-8 is mutated at an aspartate (asp) residue, e.g., asp-99 in murine GDF-8 propeptide and asp-100 in human GDF-8 propeptide); a small molecule inhibitor of GDF-8; follistatin (e.g., as described in U.S. Pat. No. 6,004,937, incorporated herein by reference) or follistatin-domain-containing proteins (e.g., GASP-1 or other proteins as described in U.S. Pat. Nos. 7,192,717 and 7,572,763, each incorporated herein by reference); and modulators of metalloprotease activity that affect GDF-8 activation, as described in U.S. Patent Publication Number 2004/0138118, incorporated herein by reference.

Additional myostatin antagonists include myostatin antibodies which bind to and inhibit or neutralize myostatin (including the myostatin proprotein and/or mature protein, in monomeric or dimeric form). Myostatin antibodies are mammalian or non-mammalian derived antibodies, for example an IgNAR antibody derived from sharks, or humanized antibodies, or comprise a functional fragment derived from antibodies. Such antibodies are described, for example, in WO 2005/094446 and WO 2006/116269, the content of which is incorporated herein by reference. Myostatin antibodies also include those antibodies that bind to the myostatin proprotein and prevent cleavage into the mature active form. Additional antibody antagonists include the antibodies described in U.S. Pat. Nos. 6,096,506 and 6,468,535 (each of which is incorporated herein by reference). In some embodiments, the GDF-8 inhibitor is a monoclonal antibody or a fragment thereof that blocks GDF-8 binding to its receptor. Further embodiments include murine monoclonal antibody JA-16 (as described in U.S. Pat. No. 7,320,789 (ATCC Deposit No. PTA-4236); humanized derivatives thereof and fully human monoclonal anti-GDF-8 antibodies (e.g., Myo29, Myo28 and Myo22, ATCC Deposit Nos. PTA-4741, PTA-4740, and PTA-4739, respectively, or derivatives thereof) as described in U.S. Pat. No. 7,261,893 and incorporated herein by reference.

In still further embodiments, myostatin antagonists include soluble receptors which bind to myostatin and inhibit at least one activity thereof. The term “soluble receptor” herein includes truncated versions or fragments of the myostatin receptor that specifically bind myostatin thereby blocking or inhibiting myostatin signal transduction. Truncated versions of the myostatin receptor, for example, include the naturally occurring soluble domains, as well as variations produced by proteolysis of the N- or C-termini. The soluble domain includes all or part of the extracellular domain of the receptor, either alone or attached to additional peptides or other moieties. Because myostatin binds activin receptors (including the activin type IEB receptor (ActRHB) and activin type HA receptor (ActRHA)), activin receptors can form the basis of soluble receptor antagonists. Soluble receptor fusion proteins can also be used, including soluble receptor Fc (see U.S. Patent Publication Number 2004/0223966 and WO 2006/012627, both of which are incorporated herein by reference in their entireties).

Other myostatin antagonists based on the myostatin receptors are ALK-5 and/or ALK-7 inhibitors (see for example WO 2006/025988 and WO 2005/084699, each incorporated herein by reference). As a TGF-β cytokine, myostatin signals through a family of single transmembrane serine/threonine kinase receptors. These receptors can be divided in two classes, the type I or activin-like kinase (ALK) receptors and type II receptors. The ALK receptors are distinguished from the Type II receptors in that the ALK receptors (a) lack the serine/threonine-rich intracellular tail, (b) possess serine/threonine kinase domains that are highly homologous among Type I receptors, and (c) share a common sequence motif called the GS domain, consisting of a region rich in glycine and serine residues. The GS domain is at the amino terminal end of the intracellular kinase domain and is believed to be critical for activation by the Type∥ receptor. Several studies have shown that TGF-β signaling requires both the ALK (Type 1) and Type∥ receptors. Specifically, the Type∥ receptor phosphorylates the GS domain of the Type 1 receptor for TGFβ ALK5, in the presence of TGFβ. The ALK5, in turn, phosphorylates the cytoplasmic proteins smad2 and smad3 at two carboxy terminal serines. Generally, it is believed that in many species, the Type∥ receptors regulate cell proliferation and the Type I receptors regulate matrix production. Various ALK5 receptor inhibitors have been described (see, for example, U.S. Pat. Nos. 6,465,493, 6,906,089, U.S. Patent Publication Numbers 2003/0166633, 2004/0063745 and 2004/0039198, the disclosures of which are incorporated herein by reference). Thus, the myostatin antagonists for use according to the disclosure may comprise the myostatin binding domain of an ALK5 and/or ALK7 receptor.

Other myostatin antagonists include soluble ligand antagonists that compete with myostatin for binding to myostatin receptors. The term “soluble ligand antagonist” herein refers to soluble peptides, polypeptides or peptidomimetics capable of non-productively binding the myostatin receptor(s) (e.g., the activin type HB receptor (ActRHA)) and thereby competitively blocking myostatin-receptor signal transduction. Soluble ligand antagonists include variants of myostatin, also referred to as “myostatin analogs” that have homology to, but not the activity of, myostatin. Such analogs include truncates (such as N- or C-terminal truncations, substitutions, deletions, and other alterations in the amino acid sequence, such as variants having non-amino acid substitutions).

Additional myostatin antagonists contemplated by the disclosure include inhibitory nucleic acids as described herein. These antagonists include antisense or sense polynucleotides comprising a single-stranded polynucleotide sequence (either RNA or DNA) capable of binding to target mRNA (sense) or DNA (antisense) sequences. Thus, RNA interference (RNAi) produced by the introduction of specific small interfering RNA (siRNA), may also be used to inhibit or eliminate the activity of myostatin.

In specific embodiments, myostatin antagonists include, but are not limited to, follistatin, the myostatin prodomain, growth and differentiation factor 11 (GDF-11) prodomain, prodomain fusion proteins, antagonistic antibodies or antibody fragments that bind to myostatin, antagonistic antibodies or antibody fragments that bind to the activin type IEB receptor, soluble activin type IHB receptor, soluble activin type IEB receptor fusion proteins, soluble myostatin analogs (soluble ligands), polynucleotides, small molecules, peptidomimetics, and myostatin binding agents. Other antagonists include the peptide immunogens described in U.S. Pat. No. 6,369,201 and WO 2001/05820 (each of which is incorporated herein by reference) and myostatin multimers and immunoconjugates capable of eliciting an immune response and thereby blocking myostatin activity. Other antagonists include the protein inhibitors of myostatin described in WO 2002/085306 (incorporated herein by reference), which include the truncated Activin type II receptor, the myostatin pro-domain, and follistatin. Other myostatin inhibitors include those released into culture from cells overexpressing myostatin (see WO 2000/43781), dominant negative myostatin proteins (see WO 2001/53350) including the protein encoded by the Piedmontese allele, and mature myostatin peptides having a C-terminal truncation at a position either at or between amino acid positions 335 to 375. The small peptides described in U.S. Patent Publication Number 2004/0181033 (incorporated herein by reference) that comprise the amino acid sequence WMCPP, are also suitable for use in the compositions of the disclosure.

### Chemotherapeutic Agents

Chemotherapeutic agents contemplated for use include, without limitation, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

### Modulators of Fibrosis

A “modulator of fibrosis” as used herein is synonymous with antifibrotic agent. The term “antifibrotic agent” refers to a chemical compound that has antifibrotic activity (i.e., prevents or reduces fibrosis) in mammals. This takes into account the abnormal formation of fibrous connective tissue, which is typically comprised of collagen. These compounds may have different mechanisms of action, some reducing the formation of collagen or another protein, others enhancing the catabolism or removal of collagen in the affected area of the body. All such compounds having activity in the reduction of the presence of fibrotic tissue are included herein, without regard to the particular mechanism of action by which each such drug functions. Antifibrotic agents useful in the methods and compositions of the disclosure include those described in U.S. Pat. No. 5,720,950, incorporated herein by reference. Additional antifibrotic agents contemplated by the disclosure include, but are not limited to, Type II interferon receptor agonists (e.g., interferon-gamma); pirfenidone and pirfenidone analogs; anti-angiogenic agents, such as VEGF antagonists, VEGF receptor antagonists, bFGF antagonists, bFGF receptor antagonists, TGFβ antagonists, TGFβ receptor antagonists; anti-inflammatory agents, IL-1 antagonists, such as IL-1 Ra, angiotensin-converting-enzyme (ACE) inhibitors, angiotensin receptor blockers and aldosterone antagonists.

### Gene Correction Approaches

Gene correction approaches are contemplated by the disclosure to be used in conjunction with the methods and compositions as described herein. As used herein, “gene correction” approaches include, without limitation, technologies related to gene editing (i.e., CRISPR technology), exon skipping, and other technologies known in the art for modifying mRNA). Thus, in some embodiments, methods are provided in which an agent of the disclosure is used to increase the activity of an annexin protein in an individual suffering from Becker muscular dystrophy (BMD), Duchenne muscular dystrophy (DMD), all Limb Girdle muscular dystrophy (LGMD) type 1 subtypes, all LGMD type 2 subtypes, congenital muscular dystrophy, Emery-Dreifuss muscular dystrophy (EDMD), myotonic dystrophy, Fascioscapulohumeral dystrophy (FSHD), Oculopharyngeal muscular dystrophy, and Distal muscular dystrophy, wherein the patient will be, is concurrently being, or has previously been, administered a composition that results in correction of a gene involved in any one of the foregoing disorders. In further embodiments, methods are provided in which an agent of the disclosure is used to increase the activity of an annexin protein in an individual suffering from Becker muscular dystrophy (BMD), Duchenne muscular dystrophy (DMD), all Limb Girdle muscular dystrophy (LGMD) type 1 subtypes, all LGMD type 2 subtypes, congenital muscular dystrophy, Emery-Dreifuss muscular dystrophy (EDMD), myotonic dystrophy, Fascioscapulohumeral dystrophy (FSHD), Oculopharyngeal muscular dystrophy, and Distal muscular dystrophy, wherein the patient will be, is concurrently being, or has previously been, administered a viral-based or non-viral-based composition that results in correction of a gene involved in any one of the foregoing disorders.

Gene correction approaches are known in the art (see, e.g., U.S. Patent Application Publication No. 2016/0130608 and U.S. Pat. No. 9,499,817, respectively, each incorporated by reference herein in their entirety). Further discussion of such methods can be found in Echigoya et al., J Pers Med 8, 2018; Li et al., Trends Pharmacol Sci 39: 982-994, 2018; Min et al., Annu Rev Med, 2018; and Zhang et al., Physiol Rev 98: 1205-1240, 2018.

### Compositions

Any of the agents and/or additional agents described herein (or nucleic acids encoding any of the agents and/or additional agents described herein) also is provided in a composition. In this regard, the agent(s) and/or additional agent(s) is formulated with a physiologically-acceptable (i.e., pharmacologically acceptable) carrier, buffer, or diluent, as described further herein. Optionally, the protein/recombinant protein is in the form of a physiologically acceptable salt, which is encompassed by the disclosure. “Physiologically acceptable salts” means any salts that are pharmaceutically acceptable. Some examples of appropriate salts include acetate, trifluoroacetate, hydrochloride, hydrobromide, sulfate, citrate, tartrate, glycolate, and oxalate. Accordingly, in some aspects the disclosure provides pharmaceutical compositions comprising one or more annexin proteins (e.g., recombinant annexin proteins) and a pharmaceutically acceptable carrier, buffer, and/or diluent. In any of the aspects or embodiments of the disclosure, one or more (or all) annexin proteins in a composition is a modified annexin protein. In any of the aspects or embodiments of the disclosure, one or more (or all) annexin proteins in a composition is a naturally-occurring mammalian annexin protein. In some embodiments, the modified annexin protein is expressed in a prokaryotic cell (for example and without limitation, an E. coli cell). In general, a modified protein is a protein that is altered relative to the version of the protein that normally exists in nature. In some embodiments, a modified protein is one in which at least one amino acid of the modified protein has an altered posttranslational modification relative to the naturally-occurring mammalian protein. By way of example, a naturally-occurring mammalian protein may comprise an amino acid that is phosphorylated while the same amino acid in the modified protein has either a different posttranslational modification or has no posttranslational modification. In some embodiments, the annexin protein is annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 37, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), annexin A13 (SEQ ID NO: 17 or SEQ ID NO: 18), or a combination thereof. In some embodiments, the annexin protein is annexin A6 (SEQ ID NO: 7, SEQ ID NO: 8, SEQ ID NO: 37, or a combination thereof). In some embodiments, and as described herein, the pharmaceutical composition comprises a combination of annexin proteins wherein one or more of the annexin proteins is a modified annexin protein. In some embodiments, the pharmaceutical composition comprises a combination of annexin proteins and each annexin protein is a naturally-occurring mammalian annexin protein. Pharmaceutical compositions of the disclosure comprising one or more annexin proteins are formulated such that the one or more annexin proteins are present in the composition at a high level of purity. By “purity” it is meant that a protein (e.g., an annexin protein) used in a pharmaceutical composition is largely composed of the full-length protein (e.g., annexin protein) that was expressed and is largely free of truncated or degraded protein products. In various embodiments, the one or more annexin proteins that is/are present in a pharmaceutical composition is/are at least 90%, at least 95%, or at least 99% pure as measured by standard release assay including but not limited to one or more of SDS-PAGE, SEC-HPLC, and immunoblot analysis. A pharmaceutical composition of the disclosure is also relatively free of endotoxin. In various embodiments, a pharmaceutical composition of the disclosure has an endotoxin level that is or is less than about 10, is or is less than about 5, is or is less than about 1, is or is less than about 0.50000, is or is less than about 0.40000, is or is less than about 0.30000 endotoxin units per milligram (EU/mg) A280 annexin protein as determined by standard methods.

As disclosed herein, the disclosure provides compositions comprising one or more agents and/or additional agents that increase the activity of an annexin protein. In various embodiments, the annexin protein is annexin A1 (SEQ ID NO: 1), annexin A2 (SEQ ID NO: 2 and/or SEQ ID NO: 3), annexin A3 (SEQ ID NO: 4), annexin A4 (SEQ ID NO: 5), annexin A5 (SEQ ID NO: 6), annexin A6 (SEQ ID NO: 7 and/or SEQ ID NO: 8), annexin A7 (SEQ ID NO: 9 and/or SEQ ID NO: 10), annexin A8 (SEQ ID NO: 11 and/or SEQ ID NO: 12), annexin A9 (SEQ ID NO: 13), annexin A10 (SEQ ID NO: 14), annexin A11 (SEQ ID NO: 15 and/or SEQ ID NO: 16), annexin A13 (SEQ ID NO: 17 and/or SEQ ID NO: 18), or a combination thereof. In some embodiments, the composition increases the activity of annexin A1 (SEQ ID NO: 1), annexin A2 (SEQ ID NO: 2 and/or SEQ ID NO: 3), and annexin A6 (SEQ ID NO: 7 and/or SEQ ID NO: 8). In further embodiments, the composition increases the activity of annexin A2 (SEQ ID NO: 2 and/or SEQ ID NO: 3) and annexin A6 (SEQ ID NO: 7 and/or SEQ ID NO: 8). In still further embodiments, the composition increases the activity of annexin A1 (SEQ ID NO: 1) and annexin A6 (SEQ ID NO: 7 and/or SEQ ID NO: 8).

The disclosure also contemplates, in various embodiments, compositions that increase the activity of annexin A1 (SEQ ID NO: 1), annexin A2 (SEQ ID NO: 2 and/or SEQ ID NO: 3), annexin A3 (SEQ ID NO: 4), annexin A4 (SEQ ID NO: 5), annexin A5 (SEQ ID NO: 6), annexin A6 (SEQ ID NO: 7 and/or SEQ ID NO: 8), annexin A7 (SEQ ID NO: 9 and/or SEQ ID NO: 10), annexin A8 (SEQ ID NO: 11 and/or SEQ ID NO: 12), annexin A9 (SEQ ID NO: 13), annexin A10 (SEQ ID NO: 14), annexin A11 (SEQ ID NO: 15 and/or SEQ ID NO: 16), and annexin A13 (SEQ ID NO: 17 and/or SEQ ID NO: 18) in any combination. Note that when more than one sequence identifier is used to identify an annexin protein herein (e.g., annexin A2 is identified herein by SEQ ID NO: 2 and/or SEQ ID NO: 3) it will be understood that the different sequence identifiers serve to identify isoforms of the particular annexin protein, and that the isoforms may be used interchangeably or in combination in methods and compositions of the disclosure.

Additional sequences (e.g., annexin protein/nucleotide sequences) contemplated by the disclosure are described in International Application Publication No. WO 2020/132647, which is incorporated herein by reference in its entirety.

Refseq Accession Number NP_000691.1 annexin A1 [Homo sapiens](SEQ ID
NO: 1):
MAMVSEFLKQAWFIENEEQEYVQTVKSSKGGPGSAVSPYPTFNPSSDVAALHKAIMVKGV
DEATIIDILTKRNNAQRQQIKAAYLQETGKPLDETLKKALTGHLEEVVLALLKTPAQFDADELR
AAMKGLGTDEDTLIEILASRTNKEIRDINRVYREELKRDLAKDITSDTSGDFRNALLSLAKGD
RSEDFGVNEDLADSDARALYEAGERRKGTDVNVFNTILTTRSYPQLRRVFQKYTKYSKHD
MNKVLDLELKGDIEKCLTAIVKCATSKPAFFAEKLHQAMKGVGTRHKALIRIMVSRSEIDMND
IKAFYQKMYGISLCQAILDETKGDYEKILVALCGGN
Refseq Accession Number NP_001002858.1 annexin A2 isoform 1 [Homo
sapiens](SEQ ID NO: 2):
MGRQLAGCGDAGKKASFKMSTVHEILCKLSLEGDHSTPPSAYGSVKAYTNFDAERDALNIE
TAIKTKGVDEVTIVNILTNRSNAQRQDIAFAYQRRTKKELASALKSALSGHLETVILGLLKTPA
QYDASELKASMKGLGTDEDSLIEIICSRTNQELQEINRVYKEMYKTDLEKDIISDTSGDFRKL
MVALAKGRRAEDGSVIDYELIDQDARDLYDAGVKRKGTDVPKWISIMTERSVPHLQKVFDR
YKSYSPYDMLESIRKEVKGDLENAFLNLVQCIQNKPLYFADRLYDSMKGKGTRDKVLIRIMV
SRSEVDMLKIRSEFKRKYGKSLYYYIQQDTKGDYQKALLYLCGGDD
Refseq Accession Number NP_001129487.1 annexin A2 isoform 2 [Homo
sapiens](SEQ ID NO: 3):
MSTVHEILCKLSLEGDHSTPPSAYGSVKAYTNFDAERDALNIETAIKTKGVDEVTIVNILTNRS
NAQRQDIAFAYQRRTKKELASALKSALSGHLETVILGLLKTPAQYDASELKASMKGLGTDED
SLIEIICSRTNQELQEINRVYKEMYKTDLEKDIISDTSGDFRKLMVALAKGRRAEDGSVIDYELI
DQDARDLYDAGVKRKGTDVPKWISIMTERSVPHLQKVFDRYKSYSPYDMLESIRKEVKGDL
ENAFLNLVQCIQNKPLYFADRLYDSMKGKGTRDKVLIRIMVSRSEVDMLKIRSEFKRKYGKS
LYYYIQQDTKGDYQKALLYLCGGDD
Refseq Accession Number NP_005130.1 annexin A3 [Homo sapiens](SEQ ID
NO: 4):
MASIWVGHRGTVRDYPDFSPSVDAEAIQKAIRGIGTDEKMLISILTERSNAQRQLIVKEYQAA
YGKELKDDLKGDLSGHFEHLMVALVTPPAVFDAKQLKKSMKGAGTNEDALIEILTTRTSRQ
MKDISQAYYTVYKKSLGDDISSETSGDFRKALLTLADGRRDESLKVDEHLAKQDAQILYKAG
ENRWGTDEDKFTEILCLRSFPQLKLTFDEYRNISQKDIVDSIKGELSGHFEDLLLAIVNCVRN
TPAFLAERLHRALKGIGTDEFTLNRIMVSRSEIDLLDIRTEFKKHYGYSLYSAIKSDTSGDYEI
TLLKICGGDD
Refseq Accession Number NP_001144.1 annexin A4 isoform a [Homo sapiens]
(SEQ ID NO: 5):
MAMATKGGTVKAASGFNAMEDAQTLRKAMKGLGTDEDAIISVLAYRNTAQRQEIRTAYKST
IGRDLIDDLKSELSGNFEQVIVGMMTPTVLYDVQELRRAMKGAGTDEGCLIEILASRTPEEIR
RISQTYQQQYGRSLEDDIRSDTSFMFQRVLVSLSAGGRDEGNYLDDALVRQDAQDLYEAG
EKKWGTDEVKFLTVLCSRNRNHLLHVFDEYKRISQKDIEQSIKSETSGSFEDALLAIVKCMR
NKSAYFAEKLYKSMKGLGTDDNTLIRVMVSRAEIDMLDIRAHFKRLYGKSLYSFIKGDTSGD
YRKVLLVLCGGDD
Refseq Accession Number NP_001145.1 annexin A5 [Homo sapiens](SEQ ID
NO: 6):
MAQVLRGTVTDFPGFDERADAETLRKAMKGLGTDEESILTLLTSRSNAQRQEISAAFKTLFG
RDLLDDLKSELTGKFEKLIVALMKPSRLYDAYELKHALKGAGTNEKVLTEIIASRTPEELRAIK
QVYEEEYGSSLEDDVVGDTSGYYQRMLVVLLQANRDPDAGIDEAQVEQDAQALFQAGELK
WGTDEEKFITIFGTRSVSHLRKVFDKYMTISGFQIEETIDRETSGNLEQLLLAVVKSIRSIPAYL
AETLYYAMKGAGTDDHTLIRVMVSRSEIDLFNIRKEFRKNFATSLYSMIKGDTSGDYKKALLL
LCGEDD
Refseq Accession Number NP_001146.2 annexin A6 isoform 1 [Homo sapiens]
(SEQ ID NO: 7):
MAKPAQGAKYRGSIHDFPGFDPNQDAEALYTAMKGFGSDKEAILDIITSRSNRQRQEVCQS
YKSLYGKDLIADLKYELTGKFERLIVGLMRPPAYCDAKEIKDAISGIGTDEKCLIEILASRTNEQ
MHQLVAAYKDAYERDLEADIIGDTSGHFQKMLVVLLQGTREEDDVVSEDLVQQDVQDLYEA
GELKWGTDEAQFIYILGNRSKQHLRLVFDEYLKTTGKPIEASIRGELSGDFEKLMLAVVKCIR
STPEYFAERLFKAMKGLGTRDNTLIRIMVSRSELDMLDIREIFRTKYEKSLYSMIKNDTSGEY
KKTLLKLSGGDDDAAGQFFPEAAQVAYQMWELSAVARVELKGTVRPANDFNPDADAKALR
KAMKGLGTDEDTIIDIITHRSNVQRQQIRQTFKSHFGRDLMTDLKSEISGDLARLILGLMMPP
AHYDAKQLKKAMEGAGTDEKALIEILATRTNAEIRAINEAYKEDYHKSLEDALSSDTSGHFRR
ILISLATGHREEGGENLDQAREDAQVAAEILEIADTPSGDKTSLETRFMTILCTRSYPHLRRV
FQEFIKMTNYDVEHTIKKEMSGDVRDAFVAIVQSVKNKPLFFADKLYKSMKGAGTDEKTLTR
IMVSRSEIDLLNIRREFIEKYDKSLHQAIEGDTSGDFLKALLALCGGED
Refseq Accession Number NP_001180473.1 annexin A6 isoform 2 [Homo
sapiens](SEQ ID NO: 8):
MKGFGSDKEAILDIITSRSNRQRQEVCQSYKSLYGKDLIADLKYELTGKFERLIVGLMRPPAY
CDAKEIKDAISGIGTDEKCLIEILASRTNEQMHQLVAAYKDAYERDLEADIIGDTSGHFQKML
VVLLQGTREEDDVVSEDLVQQDVQDLYEAGELKWGTDEAQFIYILGNRSKQHLRLVFDEYL
KTTGKPIEASIRGELSGDFEKLMLAVVKCIRSTPEYFAERLFKAMKGLGTRDNTLIRIMVSRS
ELDMLDIREIFRTKYEKSLYSMIKNDTSGEYKKTLLKLSGGDDDAAGQFFPEAAQVAYQMW
ELSAVARVELKGTVRPANDFNPDADAKALRKAMKGLGTDEDTIIDIITHRSNVQRQQIRQTF
KSHFGRDLMTDLKSEISGDLARLILGLMMPPAHYDAKQLKKAMEGAGTDEKALIEILATRTN
AEIRAINEAYKEDYHKSLEDALSSDTSGHFRRILISLATGHREEGGENLDQAREDAQVAAEIL
EIADTPSGDKTSLETRFMTILCTRSYPHLRRVFQEFIKMTNYDVEHTIKKEMSGDVRDAFVAI
VQSVKNKPLFFADKLYKSMKGAGTDEKTLTRIMVSRSEIDLLNIRREFIEKYDKSLHQAIEGD
TSGDFLKALLALCGGED
Refseq Accession Number NP_001147.1 annexin A7 isoform 1 [Homo sapiens]
(SEQ ID NO: 9):
MSYPGYPPTGYPPFPGYPPAGQESSFPPSGQYPYPSGFPPMGGGAYPQVPSSGYPGAG
GYPAPGGYPAPGGYPGAPQPGGAPSYPGVPPGQGFGVPPGGAGFSGYPQPPSQSYGG
GPAQVPLPGGFPGGQMPSQYPGGQPTYPSQPATVTQVTQGTIRPAANFDAIRDAEILRKA
MKGFGTDEQAIVDVVANRSNDQRQKIKAAFKTSYGKDLIKDLKSELSGNMEELILALFMPPT
YYDAWSLRKAMQGAGTQERVLIEILCTRTNQEIREIVRCYQSEFGRDLEKDIRSDTSGHFER
LLVSMCQGNRDENQSINHQMAQEDAQRLYQAGEGRLGTDESCFNMILATRSFPQLRATME
AYSRMANRDLLSSVSREFSGYVESGLKTILQCALNRPAFFAERLYYAMKGAGTDDSTLVRIV
VTRSEIDLVQIKQMFAQMYQKTLGTMIAGDTSGDYRRLLLAIVGQ
Refseq Accession Number NP_004025.1 annexin A7 isoform 2 [Homo sapiens]
(SEQ ID NO: 10):
MSYPGYPPTGYPPFPGYPPAGQESSFPPSGQYPYPSGFPPMGGGAYPQVPSSGYPGAG
GYPAPGGYPAPGGYPGAPQPGGAPSYPGVPPGQGFGVPPGGAGFSGYPQPPSQSYGG
GPAQVPLPGGFPGGQMPSQYPGGQPTYPSQINTDSFSSYPVFSPVSLDYSSEPATVTQVT
QGTIRPAANFDAIRDAEILRKAMKGFGTDEQAIVDVVANRSNDQRQKIKAAFKTSYGKDLIKD
LKSELSGNMEELILALFMPPTYYDAWSLRKAMQGAGTQERVLIEILCTRTNQEIREIVRCYQS
EFGRDLEKDIRSDTSGHFERLLVSMCQGNRDENQSINHQMAQEDAQRLYQAGEGRLGTD
ESCFNMILATRSFPQLRATMEAYSRMANRDLLSSVSREFSGYVESGLKTILQCALNRPAFFA
ERLYYAMKGAGTDDSTLVRIVVTRSEIDLVQIKQMFAQMYQKTLGTMIAGDTSGDYRRLLLA
IVGQ
Refseq Accession Number NP_001258631.1 annexin A8 isoform 1 [Homo
sapiens](SEQ ID NO: 11):
MAWWKSWIEQEGVTVKSSSHFNPDPDAETLYKAMKGIGVGSQLLSHQAAAFAFPSSALTS
VSPWGQQGHLCCNPAGTNEQAIIDVLTKRSNTQRQQIAKSFKAQFGKDLTETLKSELSGKF
ERLIVALMYPPYRYEAKELHDAMKGLGTKEGVIIEILASRTKNQLREIMKAYEEDYGSSLEEDI
QADTSGYLERILVCLLQGSRDDVSSFVDPGLALQDAQDLYAAGEKIRGTDEMKFITILCTRS
ATHLLRVFEEYEKIANKSIEDSIKSETHGSLEEAMLTVVKCTQNLHSYFAERLYYAMKGAGT
RDGTLIRNIVSRSEIDLNLIKCHFKKMYGKTLSSMIMEDTSGDYKNALLSLVGSDP
Refseq Accession Number NP_001035173.1 annexin A8 isoform 2 [Homo
sapiens](SEQ ID NO: 12):
MAWWKSWIEQEGVTVKSSSHFNPDPDAETLYKAMKGIGTNEQAIIDVLTKRSNTQRQQIAK
SFKAQFGKDLTETLKSELSGKFERLIVALMYPPYRYEAKELHDAMKGLGTKEGVIIEILASRT
KNQLREIMKAYEEDYGSSLEEDIQADTSGYLERILVCLLQGSRDDVSSFVDPGLALQDAQDL
YAAGEKIRGTDEMKFITILCTRSATHLLRVFEEYEKIANKSIEDSIKSETHGSLEEAMLTVVKC
TQNLHSYFAERLYYAMKGAGTRDGTLIRNIVSRSEIDLNLIKCHFKKMYGKTLSSMIMEDTS
GDYKNALLSLVGSDP
Refseq Accession Number NP_003559.2 annexin A9 [Homo sapiens](SEQ ID
NO: 13):
MSVTGGKMAPSLTQEILSHLGLASKTAAWGTLGTLRTFLNFSVDKDAQRLLRAITGQGVDR
SAIVDVLTNRSREQRQLISRNFQERTQQDLMKSLQAALSGNLERIVMALLQPTAQFDAQEL
RTALKASDSAVDVAIEILATRTPPQLQECLAVYKHNFQVEAVDDITSETSGILQDLLLALAKG
GRDSYSGIIDYNLAEQDVQALQRAEGPSREETWVPVFTQRNPEHLIRVFDQYQRSTGQELE
EAVQNRFHGDAQVALLGLASVIKNTPLYFADKLHQALQETEPNYQVLIRILISRCETDLLSIRA
EFRKKFGKSLYSSLQDAVKGDCQSALLALCRAEDM
Refseq Accession Number NP_009124.2 annexin A10 [Homo sapiens](SEQ ID
NO: 14):
MFCGDYVQGTIFPAPNFNPIMDAQMLGGALQGFDCDKDMLINILTQRCNAQRMMIAEAYQS
MYGRDLIGDMREQLSDHFKDVMAGLMYPPPLYDAHELWHAMKGVGTDENCLIEILASRTN
GEIFQMREAYCLQYSNNLQEDIYSETSGHFRDTLMNLVQGTREEGYTDPAMAAQDAMVLW
EACQQKTGEHKTMLQMILCNKSYQQLRLVFQEFQNISGQDMVDAINECYDGYFQELLVAIV
LCVRDKPAYFAYRLYSAIHDFGFHNKTVIRILIARSEIDLLTIRKRYKERYGKSLFHDIRNFASG
HYKKALLAICAGDAEDY
Refseq Accession Number NP_665875.1 annexin A11 isoform 1 [Homo sapiens]
(SEQ ID NO: 15):
MSYPGYPPPPGGYPPAAPGGGPWGGAAYPPPPSMPPIGLDNVATYAGQFNQDYLSGMA
ANMSGTFGGANMPNLYPGAPGAGYPPVPPGGFGQPPSAQQPVPPYGMYPPPGGNPPSR
MPSYPPYPGAPVPGQPMPPPGQQPPGAYPGQPPVTYPGQPPVPLPGQQQPVPSYPGYP
GSGTVTPAVPPTQFGSRGTITDAPGFDPLRDAEVLRKAMKGFGTDEQAIIDCLGSRSNKQR
QQILLSFKTAYGKDLIKDLKSELSGNFEKTILALMKTPVLFDIYEIKEAIKGVGTDEACLIEILAS
RSNEHIRELNRAYKAEFKKTLEEAIRSDTSGHFQRLLISLSQGNRDESTNVDMSLAQRDAQE
LYAAGENRLGTDESKFNAVLCSRSRAHLVAVFNEYQRMTGRDIEKSICREMSGDLEEGMLA
VVKCLKNTPAFFAERLNKAMRGAGTKDRTLIRIMVSRSETDLLDIRSEYKRMYGKSLYHDIS
GDTSGDYRKILLKICGGND
Refseq Accession Number NP_001265338.1 annexin A11 isoform 2 [Homo
sapiens](SEQ ID NO: 16):
MPPIGLDNVATYAGQFNQDYLSGMAANMSGTFGGANMPNLYPGAPGAGYPPVPPGGFG
QPPSAQQPVPPYGMYPPPGGNPPSRMPSYPPYPGAPVPGQPMPPPGQQPPGAYPGQPP
VTYPGQPPVPLPGQQQPVPSYPGYPGSGTVTPAVPPTQFGSRGTITDAPGFDPLRDAEVL
RKAMKGFGTDEQAIIDCLGSRSNKQRQQILLSFKTAYGKDLIKDLKSELSGNFEKTILALMKT
PVLFDIYEIKEAIKGVGTDEACLIEILASRSNEHIRELNRAYKAEFKKTLEEAIRSDTSGHFQRL
LISLSQGNRDESTNVDMSLAQRDAQELYAAGENRLGTDESKFNAVLCSRSRAHLVAVFNEY
QRMTGRDIEKSICREMSGDLEEGMLAVVKCLKNTPAFFAERLNKAMRGAGTKDRTLIRIMV
SRSETDLLDIRSEYKRMYGKSLYHDISGDTSGDYRKILLKICGGND
Refseq Accession Number NP_004297.2 annexin A13 isoform a [Homo sapiens]
(SEQ ID NO: 17):
MGNRHAKASSPQGFDVDRDAKKLNKACKGMGTNEAAIIEILSGRTSDERQQIKQKYKATYG
KELEEVLKSELSGNFEKTALALLDRPSEYAARQLQKAMKGLGTDESVLIEVLCTRTNKEIIAIK
EAYQRLFDRSLESDVKGDTSGNLKKILVSLLQANRNEGDDVDKDLAGQDAKDLYDAGEGR
WGTDELAFNEVLAKRSYKQLRATFQAYQILIGKDIEEAIEEETSGDLQKAYLTLVRCAQDCE
DYFAERLYKSMKGAGTDEETLIRIVVTRAEVDLQGIKAKFQEKYQKSLSDMVRSDTSGDFR
KLLVALLH
Refseq Accession Number NP_001003954.1 annexin A13 isoform b [Homo
sapiens](SEQ ID NO: 18):
MGNRHSQSYTLSEGSQQLPKGDSQPSTVVQPLSHPSRNGEPEAPQPAKASSPQGFDVDR
DAKKLNKACKGMGTNEAAIIEILSGRTSDERQQIKQKYKATYGKELEEVLKSELSGNFEKTAL
ALLDRPSEYAARQLQKAMKGLGTDESVLIEVLCTRTNKEIIAIKEAYQRLFDRSLESDVKGDT
SGNLKKILVSLLQANRNEGDDVDKDLAGQDAKDLYDAGEGRWGTDELAFNEVLAKRSYKQ
LRATFQAYQILIGKDIEEAIEEETSGDLQKAYLTLVRCAQDCEDYFAERLYKSMKGAGTDEET
LIRIVVTRAEVDLQGIKAKFQEKYQKSLSDMVRSDTSGDFRKLLVALLH
Refseq Accession Number NP_001350043.1 annexin A6 isoform 3 [Homo
sapiens](SEQ ID NO: 37):
MAKPAQGAKYRGSIHDFPGFDPNQDAEALYTAMKGFGSDKEAILDIITSRSNRQRQEVCQS
YKSLYGKDLIADLKYELTGKFERLIVGLMRPPAYCDAKEIKDAISGIGTDEKCLIEILASRTNEQ
MHQLVAAYKDAYERDLEADIIGDTSGHFQKMLVVLLQGTREEDDVVSEDLVQQDVQDLYEA
GELKWGTDEAQFIYILGNRSKQHLRLVFDEYLKTTGKPIEASIRGELSGDFEKLMLAVVKCIR
STPEYFAERLFKAMKGLGTRDNTLIRIMVSRSELDMLDIREIFRTKYEKSLYSMIKNDTSGEY
KKTLLKLSGGDDDAAGQFFPEAAQVAYQMWELSAVARVELKGTVRPANDFNPDADAKALR
KAMKGLGTDEDTIIDIITHRSNVQRQQIRQTFKSHFGRDLMTDLKSEISGDLARLILGLMMPP
AHYDAKQLKKAMEGAGTDEKALIEILATRTNAEIRAINEAYKEDYHKSLEDALSSDTSGHFRR
ILISLATGHREEGGENLDQAREDAQEIADTPSGDKTSLETRFMTILCTRSYPHLRRVFQEFIK
MTNYDVEHTIKKEMSGDVRDAFVAIVQSVKNKPLFFADKLYKSMKGAGTDEKTLTRIMVSR
SEIDLLNIRREFIEKYDKSLHQAIEGDTSGDFLKALLALCGGED

The disclosure also contemplates corresponding polynucleotides that encode each of the foregoing annexin proteins. The following polynucleotides are contemplated for use according to the disclosure. Specifically, the following polynucleotides are messenger RNA (mRNA) sequences contemplated for use with a vector of the disclosure to increase activity of an annexin protein. As discussed above, when more than one sequence identifier is used to identify an mRNA sequence in relation to the same annexin species herein (e.g., mRNA sequences relating to annexin A2 are identified herein by SEQ ID NO: 20 and SEQ ID NO: 21) it will be understood that the different sequence identifiers serve to identify transcript variants that may be utilized with a vector of the disclosure to be translated into the particular annexin protein, and that the transcript variants may be used interchangeably or in combination in the methods and compositions of the disclosure.

NM_000700.3 Homo sapiens annexin A1 (ANXA1), mRNA
(SEQ ID NO: 19)
AGTGTGAAATCTTCAGAGAAGAATTTCTCTTTAGTTCTTTGCAAGAAG
GTAGAGATAAAGACACTTTTTCAAAAATGGCAATGGTATCAGAATTCC
TCAAGCAGGCCTGGTTTATTGAAAATGAAGAGCAGGAATATGTTCAAA
CTGTGAAGTCATCCAAAGGTGGTCCCGGATCAGCGGTGAGCCCCTATC
CTACCTTCAATCCATCCTCGGATGTCGCTGCCTTGCATAAGGCCATAA
TGGTTAAAGGTGTGGATGAAGCAACCATCATTGACATTCTAACTAAGC
GAAACAATGCACAGCGTCAACAGATCAAAGCAGCATATCTCCAGGAAA
CAGGAAAGCCCCTGGATGAAACACTGAAGAAAGCCCTTACAGGTCACC
TTGAGGAGGTTGTTTTAGCTCTGCTAAAAACTCCAGCGCAATTTGATG
CTGATGAACTTCGTGCTGCCATGAAGGGCCTTGGAACTGATGAAGATA
CTCTAATTGAGATTTTGGCATCAAGAACTAACAAAGAAATCAGAGACA
TTAACAGGGTCTACAGAGAGGAACTGAAGAGAGATCTGGCCAAAGACA
TAACCTCAGACACATCTGGAGATTTTCGGAACGCTTTGCTTTCTCTTG
CTAAGGGTGACCGATCTGAGGACTTTGGTGTGAATGAAGACTTGGCTG
ATTCAGATGCCAGGGCCTTGTATGAAGCAGGAGAAAGGAGAAAGGGGA
CAGACGTAAACGTGTTCAATACCATCCTTACCACCAGAAGCTATCCAC
AACTTCGCAGAGTGTTTCAGAAATACACCAAGTACAGTAAGCATGACA
TGAACAAAGTTCTGGACCTGGAGTTGAAAGGTGACATTGAGAAATGCC
TCACAGCTATCGTGAAGTGCGCCACAAGCAAACCAGCTTTCTTTGCAG
AGAAGCTTCATCAAGCCATGAAAGGTGTTGGAACTCGCCATAAGGCAT
TGATCAGGATTATGGTTTCCCGTTCTGAAATTGACATGAATGATATCA
AAGCATTCTATCAGAAGATGTATGGTATCTCCCTTTGCCAAGCCATCC
TGGATGAAACCAAAGGAGATTATGAGAAAATCCTGGTGGCTCTTTGTG
GAGGAAACTAAACATTCCCTTGATGGTCTCAAGCTATGATCAGAAGAC
TTTAATTATATATTTTCATCCTATAAGCTTAAATAGGAAAGTTTCTTC
AACAGGATTACAGTGTAGCTACCTACATGCTGAAAAATATAGCCTTTA
AATCATTTTTATATTATAACTCTGTATAATAGAGATAAGTCCATTTTT
TAAAAATGTTTTCCCCAAACCATAAAACCCTATACAAGTTGTTCTAGT
AACAATACATGAGAAAGATGTCTATGTAGCTGAAAATAAAATGACGTC
ACAAGACAA
NM_001002858.2 Homo sapiens annexin A2 (ANXA2),
transcript variant 1, mRNA
(SEQ ID NO: 20)
GCTCAGCATTTGGGGACGCTCTCAGCTCTCGGCGCACGGCCCAGGTAA
GCGGGGCGCGCCCTGCCCGCCCGCGATGGGCCGCCAGCTAGCGGGGT
GTGGAGACGCTGGGAAGAAGGCTTCCTTCAAAATGTCTACTGTTCACG
AAATCCTGTGCAAGCTCAGCTTGGAGGGTGATCACTCTACACCCCCAA
GTGCATATGGGTCTGTCAAAGCCTATACTAACTTTGATGCTGAGCGGG
ATGCTTTGAACATTGAAACAGCCATCAAGACCAAAGGTGTGGATGAGG
TCACCATTGTCAACATTTTGACCAACCGCAGCAATGCACAGAGACAGG
ATATTGCCTTCGCCTACCAGAGAAGGACCAAAAAGGAACTTGCATCAG
CACTGAAGTCAGCCTTATCTGGCCACCTGGAGACGGTGATTTTGGGCC
TATTGAAGACACCTGCTCAGTATGACGCTTCTGAGCTAAAAGCTTCCA
TGAAGGGGCTGGGAACCGACGAGGACTCTCTCATTGAGATCATCTGCT
CCAGAACCAACCAGGAGCTGCAGGAAATTAACAGAGTCTACAAGGAAA
TGTACAAGACTGATCTGGAGAAGGACATTATTTCGGACACATCTGGTG
ACTTCCGCAAGCTGATGGTTGCCCTGGCAAAGGGTAGAAGAGCAGAGG
ATGGCTCTGTCATTGATTATGAACTGATTGACCAAGATGCTCGGGATC
TCTATGACGCTGGAGTGAAGAGGAAAGGAACTGATGTTCCCAAGTGGA
TCAGCATCATGACCGAGCGGAGCGTGCCCCACCTCCAGAAAGTATTTG
ATAGGTACAAGAGTTACAGCCCTTATGACATGTTGGAAAGCATCAGGA
AAGAGGTTAAAGGAGACCTGGAAAATGCTTTCCTGAACCTGGTTCAGT
GCATTCAGAACAAGCCCCTGTATTTTGCTGATCGGCTGTATGACTCCA
TGAAGGGCAAGGGGACGCGAGATAAGGTCCTGATCAGAATCATGGTCT
CCCGCAGTGAAGTGGACATGTTGAAAATTAGGTCTGAATTCAAGAGAA
AGTACGGCAAGTCCCTGTACTATTATATCCAGCAAGACACTAAGGGCG
ACTACCAGAAAGCGCTGCTGTACCTGTGTGGTGGAGATGACTGAAGCC
CGACACGGCCTGAGCGTCCAGAAATGGTGCTCACCATGCTTCCAGCTA
ACAGGTCTAGAAAACCAGCTTGCGAATAACAGTCCCCGTGGCCATCCC
TGTGAGGGTGACGTTAGCATTACCCCCAACCTCATTTTAGTTGCCTAA
GCATTGCCTGGCCTTCCTGTCTAGTCTCTCCTGTAAGCCAAAGAAATG
AACATTCCAAGGAGTTGGAAGTGAAGTCTATGATGTGAAACACTTTGC
CTCCTGTGTACTGTGTCATAAACAGATGAATAAACTGAATTTGTACTT
TAGAAACACGTACTTTGTGGCCCTGCTTTCAACTGAATTGTTTGAAAA
TTAAACGTGCTTGGGGTTCAGCTGGTGAGGCTGTCCCTGTAGGAAGAA
AGCTCTGGGACTGAGCTGTACAGTATGGTTGCCCCTATCCAAGTGTCG
CTATTTAAGTTAAATTTAAATGAAATAAAATAAAATAAAATCAAAAAA
A
NM_001136015.2 Homo sapiens annexin A2 (ANXA2),
transcript variant 4, mRNA
(SEQ ID NO: 21)
GCTCAGCATTTGGGGACGCTCTCAGCTCTCGGCGCACGGCCCAGGGTG
AAAATGTTTGCCATTAAACTCACATGAAGTAGGAAATATTTATATGG
ATACAAAAGGCACCTGCATGGGATAATGTCAAATTTCATAGATACTGC
TTTGTGCTTCCTTCAAAATGTCTACTGTTCACGAAATCCTGTGCAAGC
TCAGCTTGGAGGGTGATCACTCTACACCCCCAAGTGCATATGGGTCTG
TCAAAGCCTATACTAACTTTGATGCTGAGCGGGATGCTTTGAACATTG
AAACAGCCATCAAGACCAAAGGTGTGGATGAGGTCACCATTGTCAACA
TTTTGACCAACCGCAGCAATGCACAGAGACAGGATATTGCCTTCGCCT
ACCAGAGAAGGACCAAAAAGGAACTTGCATCAGCACTGAAGTCAGCCT
TATCTGGCCACCTGGAGACGGTGATTTTGGGCCTATTGAAGACACCTG
CTCAGTATGACGCTTCTGAGCTAAAAGCTTCCATGAAGGGGCTGGGAA
CCGACGAGGACTCTCTCATTGAGATCATCTGCTCCAGAACCAACCAGG
AGCTGCAGGAAATTAACAGAGTCTACAAGGAAATGTACAAGACTGATC
TGGAGAAGGACATTATTTCGGACACATCTGGTGACTTCCGCAAGCTGA
TGGTTGCCCTGGCAAAGGGTAGAAGAGCAGAGGATGGCTCTGTCATTG
ATTATGAACTGATTGACCAAGATGCTCGGGATCTCTATGACGCTGGAG
TGAAGAGGAAAGGAACTGATGTTCCCAAGTGGATCAGCATCATGACCG
AGCGGAGCGTGCCCCACCTCCAGAAAGTATTTGATAGGTACAAGAGTT
ACAGCCCTTATGACATGTTGGAAAGCATCAGGAAAGAGGTTAAAGGAG
ACCTGGAAAATGCTTTCCTGAACCTGGTTCAGTGCATTCAGAACAAGC
CCCTGTATTTTGCTGATCGGCTGTATGACTCCATGAAGGGCAAGGGGA
CGCGAGATAAGGTCCTGATCAGAATCATGGTCTCCCGCAGTGAAGTGG
ACATGTTGAAAATTAGGTCTGAATTCAAGAGAAAGTACGGCAAGTCCC
TGTACTATTATATCCAGCAAGACACTAAGGGCGACTACCAGAAAGCGC
TGCTGTACCTGTGTGGTGGAGATGACTGAAGCCCGACACGGCCTGAGC
GTCCAGAAATGGTGCTCACCATGCTTCCAGCTAACAGGTCTAGAAAAC
CAGCTTGCGAATAACAGTCCCCGTGGCCATCCCTGTGAGGGTGACGTT
AGCATTACCCCCAACCTCATTTTAGTTGCCTAAGCATTGCCTGGCCTT
CCTGTCTAGTCTCTCCTGTAAGCCAAAGAAATGAACATTCCAAGGAGT
TGGAAGTGAAGTCTATGATGTGAAACACTTTGCCTCCTGTGTACTGTG
TCATAAACAGATGAATAAACTGAATTTGTACTTTAGAAACACGTACTT
TGTGGCCCTGCTTTCAACTGAATTGTTTGAAAATTAAACGTGCTTGGG
GTTCAGCTGGTGAGGCTGTCCCTGTAGGAAGAAAGCTCTGGGACTGAG
CTGTACAGTATGGTTGCCCCTATCCAAGTGTCGCTATTTAAGTTAAAT
TTAAATGAAATAAAATAAAATAAAATCAAAAAAA
NM_005139.3 Homo sapiens annexin A3 (ANXA3), mRNA
(SEQ ID NO: 22)
AGCGCGGAGCACCTGCGCCCGCGGCTGACACCTTCGCTCGCAGTTTGT
TCGCAGTTTACTCGCACACCAGTTTCCCCCACCGCGCTTTGGATTAGT
GTGATCTCAGCTCAAGGCAAAGGTGGGATATCATGGCATCTATCTGGG
TTGGACACCGAGGAACAGTAAGAGATTATCCAGACTTTAGCCCATCAG
TGGATGCTGAAGCTATTCAGAAAGCAATCAGAGGAATTGGAACTGATG
AGAAAATGCTCATCAGCATTCTGACTGAGAGGTCAAATGCACAGCGGC
AGCTGATTGTTAAGGAATATCAAGCAGCATATGGAAAGGAGCTGAAAG
ATGACTTGAAGGGTGATCTCTCTGGCCACTTTGAGCATCTCATGGTGG
CCCTAGTGACTCCACCAGCAGTCTTTGATGCAAAGCAGCTAAAGAAAT
CCATGAAGGGCGCGGGAACAAACGAAGATGCCTTGATTGAAATCTTAA
CTACCAGGACAAGCAGGCAAATGAAGGATATCTCTCAAGCCTATTATA
CAGTATACAAGAAGAGTCTTGGAGATGACATTAGTTCCGAAACATCTG
GTGACTTCCGGAAAGCTCTGTTGACTTTGGCAGATGGCAGAAGAGATG
AAAGTCTGAAAGTGGATGAGCATCTGGCCAAACAAGATGCCCAGATTC
TCTATAAAGCTGGTGAGAACAGATGGGGCACGGATGAAGACAAATTCA
CTGAGATCCTGTGTTTAAGGAGCTTTCCTCAATTAAAACTAACATTTG
ATGAATACAGAAATATCAGCCAAAAGGACATTGTGGACAGCATAAAAG
GAGAATTATCTGGGCATTTTGAAGACTTACTGTTGGCCATAGTTAATT
GTGTGAGGAACACGCCGGCCTTTTTAGCCGAAAGACTGCATCGAGCCT
TGAAGGGTATTGGAACTGATGAGTTTACTCTGAACCGAATAATGGTGT
CCAGATCAGAAATTGACCTTTTGGACATTCGAACAGAGTTCAAGAAGC
ATTATGGCTATTCCCTATATTCAGCAATTAAATCGGATACTTCTGGAG
ACTATGAAATCACACTCTTAAAAATCTGTGGTGGAGATGACTGAACCA
AGAAGATAATCTCCAAAGGTCCACGATGGGCTTTCCCAACAGCTCCAC
CTTACTTCTTCTCATACTATTTAAGAGAACAAGCAAATATAAACAGCA
ACTTGTGTTCCTAACAGGAATTTTCATTGTTCTATAACAACAACAACA
AAAGCGATTATTATTTTAGAGCATCTCATTTATAATGTAGCAGCTCAT
AAATGAAATTGAAAATGGTATTAAAGATCTGCAACTACTATCCAACTT
ATATTTCTGCTTTCAAAGTTAAGAATCTTTATAGTTCTACTCCATTAA
ATATAAAGCAAGATAATAAAAATTGTTGCTTTTGTTAAAA
NM_001153.5 Homo sapiens annexin A4 (ANXA4),
transcript variant 2, mRNA
(SEQ ID NO: 23)
GTGACCTCCGCAGCCGCAGAGGAGGAGCGCAGCCCGGCCTCGAAGAAC
TTCTGCTTGGGTGGCTGAACTCTGATCTTGACCTAGAGTCATGGCCA
TGGCAACCAAAGGAGGTACTGTCAAAGCTGCTTCAGGATTCAATGCCA
TGGAAGATGCCCAGACCCTGAGGAAGGCCATGAAAGGGCTCGGCACCG
ATGAAGACGCCATTATTAGCGTCCTTGCCTACCGCAACACCGCCCAGC
GCCAGGAGATCAGGACAGCCTACAAGAGCACCATCGGCAGGGACTTGA
TAGACGACCTGAAGTCAGAACTGAGTGGCAACTTCGAGCAGGTGATTG
TGGGGATGATGACGCCCACGGTGCTGTATGACGTGCAAGAGCTGCGAA
GGGCCATGAAGGGAGCCGGCACTGATGAGGGCTGCCTAATTGAGATCC
TGGCCTCCCGGACCCCTGAGGAGATCCGGCGCATAAGCCAAACCTACC
AGCAGCAATATGGACGGAGCCTTGAAGATGACATTCGCTCTGACACAT
CGTTCATGTTCCAGCGAGTGCTGGTGTCTCTGTCAGCTGGTGGGAGGG
ATGAAGGAAATTATCTGGACGATGCTCTCGTGAGACAGGATGCCCAGG
ACCTGTATGAGGCTGGAGAGAAGAAATGGGGGACAGATGAGGTGAAAT
TTCTAACTGTTCTCTGTTCCCGGAACCGAAATCACCTGTTGCATGTGT
TTGATGAATACAAAAGGATATCACAGAAGGATATTGAACAGAGTATTA
AATCTGAAACATCTGGTAGCTTTGAAGATGCTCTGCTGGCTATAGTAA
AGTGCATGAGGAACAAATCTGCATATTTTGCTGAAAAGCTCTATAAAT
CGATGAAGGGCTTGGGCACCGATGATAACACCCTCATCAGAGTGATGG
TTTCTCGAGCAGAAATTGACATGTTGGATATCCGGGCACACTTCAAGA
GACTCTATGGAAAGTCTCTGTACTCGTTCATCAAGGGTGACACATCTG
GAGACTACAGGAAAGTACTGCTTGTTCTCTGTGGAGGAGATGATTAAA
ATAAAAATCCCAGAAGGACAGGAGGATTCTCAACACTTTGAATTTTTT
TAACTTCATTTTTCTACACTGCTATTATCATTATCTCAGAATGCTTAT
TTCCAATTAAAACGCCTACAGCTGCCTCCTAGAATATAGACTGTCTGT
ATTATTATTCACCTATAATTAGTCATTATGATGCTTTAAAGCTGTACT
TGCATTTCAAAGCTTATAAGATATAAATGGAGATTTTAAAGTAGAAAT
AAATATGTATTCCATGTTTTTAAAAGATTACTTTCTACTTTGTGTTTC
ACAGACATTGAATATATTAAATTATTCCATATTTTCTTTTCAGTGAAA
AATTTTTTAAATGGAAGACTGTTCTAAAATCACTTTTTTCCCTAATCC
AATTTTTAGAGTGGCTAGTAGTTTCTTCATTTGAAATTGTAAGCATCC
GGTCAGTAAGAATGCCCATCCAGTTTTCTATATTTCATAGTCAAAGCC
TTGAAAGCATCTACAAATCTCTTTTTTTAGGTTTTGTCCATAGCATCA
GTTGATCCTTACTAAGTTTTTCATGGGAGACTTCCTTCATCACATCTT
ATGTTGAAATCACTTTCTGTAGTCAAAGTATACCAAAACCAATTTATC
TGAACTAAATTCTAAAGTATGGTTATACAAACCATATACATCTGGTTA
CCAAACATAAATGCTGAACATTCCATATTATTATAGTTAATGTCTTAA
TCCAGCTTGCAAGTGAATGGAAAAAAAAATAAGCTTCAAACTAGGTAT
TCTGGGAATGATGTAATGCTCTGAATTTAGTATGATATAAAGAAAACT
TTTTTGTGCTAAAAATACTTTTTAAAATCAATTTTGTTGATTGTAGTA
ATTTCTATTTGCACTGTGCCTTTCAACTCCAGAAACATTCTGAAGATG
TACTTGGATTTAATTAAAAAGTTCACTTTGTAAGAACGTGGAAAAATA
ATTTTAATTTAAAAATGGTGTTTTTAGGCCGGGGGCGGGGGCTCACGC
CAGTAATCCCAACACTTTGGGAGGCCAAGGCGGGTGGATCACCTAAGG
TCAGGAGTTCAAGACTAGCCTGGCCAACATGGAGAAACTGCATCTCTA
CTAAAAATATAAAAATTAGCCGGGTGTGGTGGCTGGTGCCTGTAATCC
CAGCCACTCGGAGGCTGAGTCAGGGAGAACTGCTTGAACCCAGGAGGC
AGGAGGCAAAGGTTGCAGTGAGCCGAGATCACGCCAGCCTGGGCGACA
GAGCGAGAATCCATCTAAAAAAAAAAAAAAAAAAAGTGTCTTTAAAGT
GAGGTATAGTCTTTCTCTGATCCACTTTTCACCTTCTGAGGTTTTTCA
TCTTGGCCCCTGAAAGGAGCTATTTTTGAAGGACTTGTGTTACTCAGT
TTCTACAGGAATTACAAGATAAGAAAAAAAAAATCATATTTAGTCTTA
TGCGTGCCTACTGGCTAATGTTCACATATGCCAAACACTACTCAATAA
CATAAAATAATGTATGAACTTATTCTCTGGAAATGAGTGATGCCCTCT
GCTCTAAGTAGACCATTTATATTAAATATCATAAATGTATAAAGGACA
TTCATATTCTTA
NM_001154.4 Homo sapiens annexin A5 (ANXA5), mRNA
(SEQ ID NO: 24)
AGTCTAGGTGCAGCTGCCGGATCCTTCAGCGTCTGCATCTCGGCGTCG
CCCCGCGTACCGTCGCCCGGCTCTCCGCCGCTCTCCCGGGGTTTCGG
GGCACTTGGGTCCCACAGTCTGGTCCTGCTTCACCTTCCCCTGACCTG
AGTAGTCGCCATGGCACAGGTTCTCAGAGGCACTGTGACTGACTTCCC
TGGATTTGATGAGCGGGCTGATGCAGAAACTCTTCGGAAGGCTATGAA
AGGCTTGGGCACAGATGAGGAGAGCATCCTGACTCTGTTGACATCCCG
AAGTAATGCTCAGCGCCAGGAAATCTCTGCAGCTTTTAAGACTCTGTT
TGGCAGGGATCTTCTGGATGACCTGAAATCAGAACTAACTGGAAAATT
TGAAAAATTAATTGTGGCTCTGATGAAACCCTCTCGGCTTTATGATGC
TTATGAACTGAAACATGCCTTGAAGGGAGCTGGAACAAATGAAAAAGT
ACTGACAGAAATTATTGCTTCAAGGACACCTGAAGAACTGAGAGCCAT
CAAACAAGTTTATGAAGAAGAATATGGCTCAAGCCTGGAAGATGACGT
GGTGGGGGACACTTCAGGGTACTACCAGCGGATGTTGGTGGTTCTCCT
TCAGGCTAACAGAGACCCTGATGCTGGAATTGATGAAGCTCAAGTTGA
ACAAGATGCTCAGGCTTTATTTCAGGCTGGAGAACTTAAATGGGGGAC
AGATGAAGAAAAGTTTATCACCATCTTTGGAACACGAAGTGTGTCTCA
TTTGAGAAAGGTGTTTGACAAGTACATGACTATATCAGGATTTCAAAT
TGAGGAAACCATTGACCGCGAGACTTCTGGCAATTTAGAGCAACTACT
CCTTGCTGTTGTGAAATCTATTCGAAGTATACCTGCCTACCTTGCAGA
GACCCTCTATTATGCTATGAAGGGAGCTGGGACAGATGATCATACCCT
CATCAGAGTCATGGTTTCCAGGAGTGAGATTGATCTGTTTAACATCAG
GAAGGAGTTTAGGAAGAATTTTGCCACCTCTCTTTATTCCATGATTAA
GGGAGATACATCTGGGGACTATAAGAAAGCTCTTCTGCTGCTCTGTGG
AGAAGATGACTAACGTGTCACGGGGAAGAGCTCCCTGCTGTGTGCCTG
CACCACCCCACTGCCTTCCTTCAGCACCTTTAGCTGCATTTGTATGCC
AGTGCTTAACACATTGCCTTATTCATACTAGCATGCTCATGACCAACA
CATACACGTCATAGAAGAAAATAGTGGTGCTTCTTTCTGATCTCTAGT
GGAGATCTCTTTGACTGCTGTAGTACTAAAGTGTACTTAATGTTACTA
AGTTTAATGCCTGGCCATTTTCCATTTATATATATTTTTTAAGAGGCT
AGAGTGCTTTTAGCCTTTTTTAAAAACTCCATTTATATTACATTTGTA
ACCATGATACTTTAATCAGAAGCTTAGCCTTGAAATTGTGAACTCTTG
GAAATGTTATTAGTGAAGTTCGCAACTAAACTAAACCTGTAAAATTAT
GATGATTGTATTCAAAAGATTAATGAAAAATAAACATTTCTGTCCCCC
TGAATTATGTGTACATGTGTGTTTAGATTTATTATTAAATTTATTTAA
CAATGTT
NM_001155.5 Homo sapiens annexin A6 (ANXA6),
transcript variant 1, mRNA
(SEQ ID NO: 25)
GCGGTTGCTGCTGGGCTAACGGGCTCCGATCCAGCGAGCGCTGCGTCC
TCGAGTCCCTGCGCCCGTGCGTCCGTCTGCGACCCGAGGCCTCCGCT
GCGCGTGGATTCTGCTGCGAACCGGAGACCATGGCCAAACCAGCACAG
GGTGCCAAGTACCGGGGCTCCATCCATGACTTCCCAGGCTTTGACCCC
AACCAGGATGCCGAGGCTCTGTACACTGCCATGAAGGGCTTTGGCAGT
GACAAGGAGGCCATACTGGACATAATCACCTCACGGAGCAACAGGCAG
AGGCAGGAGGTCTGCCAGAGCTACAAGTCCCTCTACGGCAAGGACCTC
ATTGCTGATTTAAAGTATGAATTGACGGGCAAGTTTGAACGGTTGATT
GTGGGCCTGATGAGGCCACCTGCCTATTGTGATGCCAAAGAAATTAAA
GATGCCATCTCGGGCATTGGCACTGATGAGAAGTGCCTCATTGAGATC
TTGGCTTCCCGGACCAATGAGCAGATGCACCAGCTGGTGGCAGCATAC
AAAGATGCCTACGAGCGGGACCTGGAGGCTGACATCATCGGCGACACC
TCTGGCCACTTCCAGAAGATGCTTGTGGTCCTGCTCCAGGGAACCAGG
GAGGAGGATGACGTAGTGAGCGAGGACCTGGTACAACAGGATGTCCAG
GACCTATACGAGGCAGGGGAACTGAAATGGGGAACAGATGAAGCCCAG
TTCATTTACATCTTGGGAAATCGCAGCAAGCAGCATCTTCGGTTGGTG
TTCGATGAGTATCTGAAGACCACAGGGAAGCCGATTGAAGCCAGCATC
CGAGGGGAGCTGTCTGGGGACTTTGAGAAGCTAATGCTGGCCGTAGTG
AAGTGTATCCGGAGCACCCCGGAATATTTTGCTGAAAGGCTCTTCAAG
GCTATGAAGGGCCTGGGGACTCGGGACAACACCCTGATCCGCATCATG
GTCTCCCGTAGTGAGTTGGACATGCTCGACATTCGGGAGATCTTCCGG
ACCAAGTATGAGAAGTCCCTCTACAGCATGATCAAGAATGACACCTCT
GGCGAGTACAAGAAGACTCTGCTGAAGCTGTCTGGGGGAGATGATGAT
GCTGCTGGCCAGTTCTTCCCGGAGGCAGCGCAGGTGGCCTATCAGATG
TGGGAACTTAGTGCAGTGGCCCGAGTAGAGCTGAAGGGAACTGTGCGC
CCAGCCAATGACTTCAACCCTGACGCAGATGCCAAAGCGCTGCGGAAA
GCCATGAAGGGACTCGGGACTGACGAAGACACAATCATCGATATCATC
ACGCACCGCAGCAATGTCCAGCGGCAGCAGATCCGGCAGACCTTCAAG
TCTCACTTTGGCCGGGACTTAATGACTGACCTGAAGTCTGAGATCTCT
GGAGACCTGGCAAGGCTGATTCTGGGGCTCATGATGCCACCGGCCCAT
TACGATGCCAAGCAGTTGAAGAAGGCCATGGAGGGAGCCGGCACAGAT
GAAAAGGCTCTTATTGAAATCCTGGCCACTCGGACCAATGCTGAAATC
CGGGCCATCAATGAGGCCTATAAGGAGGACTATCACAAGTCCCTGGAG
GATGCTCTGAGCTCAGACACATCTGGCCACTTCAGGAGGATCCTCATT
TCTCTGGCCACGGGGCATCGTGAGGAGGGAGGAGAAAACCTGGACCAG
GCACGGGAAGATGCCCAGGTGGCTGCTGAGATCTTGGAAATAGCAGAC
ACACCTAGTGGAGACAAAACTTCCTTGGAGACACGTTTCATGACGATC
CTGTGTACCCGGAGCTATCCGCACCTCCGGAGAGTCTTCCAGGAGTTC
ATCAAGATGACCAACTATGACGTGGAGCACACCATCAAGAAGGAGATG
TCTGGGGATGTCAGGGATGCATTTGTGGCCATTGTTCAAAGTGTCAAG
AACAAGCCTCTCTTCTTTGCCGACAAACTTTACAAATCCATGAAGGGT
GCTGGCACAGATGAGAAGACTCTGACCAGGATCATGGTATCCCGCAGT
GAGATTGACCTGCTCAACATCCGGAGGGAATTCATTGAGAAATATGAC
AAGTCTCTCCACCAAGCCATTGAGGGTGACACCTCCGGAGACTTCCTG
AAGGCCTTGCTGGCTCTCTGTGGTGGTGAGGACTAGGGCCACAGCTTT
GGCGGGCACTTCTGCCAAGAAATGGTTATCAGCACCAGCCGCCATGGC
CAAGCCTGATTGTTCCAGCTCCAGAGACTAAGGAAGGGGCAGGGGTGG
GGGGAGGGGTTGGGTTGGGCTCTTATCTTCAGTGGAGCTTAGGAAACG
CTCCCACTCCCACGGGCCATCGAGGGCCCAGCACGGCTGAGCGGCTGA
AAAACCGTAGCCATAGATCCTGTCCACCTCCACTCCCCTCTGACCCTC
AGGCTTTCCCAGCTTCCTCCCCTTGCTACAGCCTCTGCCCTGGTTTGG
GCTATGTCAGATCCAAAAACATCCTGAACCTCTGTCTGTAAAATGAGT
AGTGTCTGTACTTTGAATGAGGGGGTTGGTGGCAGGGGCCAGTTGAAT
GTGCTGGGGGGGGTGGTGGGAAGGATAGTAAATGTGCTGGGGCAAACT
GACAAATCTTCCCATCCATTTCACCACCCATCTCCATCCAGGCCGCGC
TAGAGTACTGGACCAGGAATTTGGATGCCTGGGTTCAAATCTGCATCT
GCCATGCACTTGTTTCTGACCTTAGGCCAGCCCCTTTCCCTCCCTGAG
TCTCTATTTTCTTATCTACAATGAGACAGTTGGACAAAAAAATCTTGG
CTTCCCTTCTAACATTAACTTCCTAAAGTATGCCTCCGATTCATTCCC
TTGACACTTTTTATTTCTAAGGAAGAAATAAAAAGAGATACACAAACA
CATAAACACA
NM_001193544.1 Homo sapiens annexin A6 (ANXA6),
transcript variant 2, mRNA
(SEQ ID NO: 26)
AGAGACCAGAGAGCATCCAGAGGCCTGGCCGGGGTCCTGCAGTGCAGA
CGTTGGGAGGCACGGAGACGGGGAGAGGGGGAGGCGGTCCAGGACTC
ACTCTGCTCCACCTCTGACTCCTTGAAGGGTGCCAAGTACCGGGGCTC
CATCCATGACTTCCCAGGCTTTGACCCCAACCAGGATGCCGAGGCTCT
GTACACTGCCATGAAGGGCTTTGGCAGTGACAAGGAGGCCATACTGGA
CATAATCACCTCACGGAGCAACAGGCAGAGGCAGGAGGTCTGCCAGAG
CTACAAGTCCCTCTACGGCAAGGACCTCATTGCTGATTTAAAGTATGA
ATTGACGGGCAAGTTTGAACGGTTGATTGTGGGCCTGATGAGGCCACC
TGCCTATTGTGATGCCAAAGAAATTAAAGATGCCATCTCGGGCATTGG
CACTGATGAGAAGTGCCTCATTGAGATCTTGGCTTCCCGGACCAATGA
GCAGATGCACCAGCTGGTGGCAGCATACAAAGATGCCTACGAGCGGGA
CCTGGAGGCTGACATCATCGGCGACACCTCTGGCCACTTCCAGAAGAT
GCTTGTGGTCCTGCTCCAGGGAACCAGGGAGGAGGATGACGTAGTGAG
CGAGGACCTGGTACAACAGGATGTCCAGGACCTATACGAGGCAGGGGA
ACTGAAATGGGGAACAGATGAAGCCCAGTTCATTTACATCTTGGGAAA
TCGCAGCAAGCAGCATCTTCGGTTGGTGTTCGATGAGTATCTGAAGAC
CACAGGGAAGCCGATTGAAGCCAGCATCCGAGGGGAGCTGTCTGGGGA
CTTTGAGAAGCTAATGCTGGCCGTAGTGAAGTGTATCCGGAGCACCCC
GGAATATTTTGCTGAAAGGCTCTTCAAGGCTATGAAGGGCCTGGGGAC
TCGGGACAACACCCTGATCCGCATCATGGTCTCCCGTAGTGAGTTGGA
CATGCTCGACATTCGGGAGATCTTCCGGACCAAGTATGAGAAGTCCCT
CTACAGCATGATCAAGAATGACACCTCTGGCGAGTACAAGAAGACTCT
GCTGAAGCTGTCTGGGGGAGATGATGATGCTGCTGGCCAGTTCTTCCC
GGAGGCAGCGCAGGTGGCCTATCAGATGTGGGAACTTAGTGCAGTGGC
CCGAGTAGAGCTGAAGGGAACTGTGCGCCCAGCCAATGACTTCAACCC
TGACGCAGATGCCAAAGCGCTGCGGAAAGCCATGAAGGGACTCGGGAC
TGACGAAGACACAATCATCGATATCATCACGCACCGCAGCAATGTCCA
GCGGCAGCAGATCCGGCAGACCTTCAAGTCTCACTTTGGCCGGGACTT
AATGACTGACCTGAAGTCTGAGATCTCTGGAGACCTGGCAAGGCTGAT
TCTGGGGCTCATGATGCCACCGGCCCATTACGATGCCAAGCAGTTGAA
GAAGGCCATGGAGGGAGCCGGCACAGATGAAAAGGCTCTTATTGAAAT
CCTGGCCACTCGGACCAATGCTGAAATCCGGGCCATCAATGAGGCCTA
TAAGGAGGACTATCACAAGTCCCTGGAGGATGCTCTGAGCTCAGACAC
ATCTGGCCACTTCAGGAGGATCCTCATTTCTCTGGCCACGGGGCATCG
TGAGGAGGGAGGAGAAAACCTGGACCAGGCACGGGAAGATGCCCAGGT
GGCTGCTGAGATCTTGGAAATAGCAGACACACCTAGTGGAGACAAAAC
TTCCTTGGAGACACGTTTCATGACGATCCTGTGTACCCGGAGCTATCC
GCACCTCCGGAGAGTCTTCCAGGAGTTCATCAAGATGACCAACTATGA
CGTGGAGCACACCATCAAGAAGGAGATGTCTGGGGATGTCAGGGATGC
ATTTGTGGCCATTGTTCAAAGTGTCAAGAACAAGCCTCTCTTCTTTGC
CGACAAACTTTACAAATCCATGAAGGGTGCTGGCACAGATGAGAAGAC
TCTGACCAGGATCATGGTATCCCGCAGTGAGATTGACCTGCTCAACAT
CCGGAGGGAATTCATTGAGAAATATGACAAGTCTCTCCACCAAGCCAT
TGAGGGTGACACCTCCGGAGACTTCCTGAAGGCCTTGCTGGCTCTCTG
TGGTGGTGAGGACTAGGGCCACAGCTTTGGCGGGCACTTCTGCCAAGA
AATGGTTATCAGCACCAGCCGCCATGGCCAAGCCTGATTGTTCCAGCT
CCAGAGACTAAGGAAGGGGCAGGGGTGGGGGGAGGGGTTGGGTTGGGC
TCTTATCTTCAGTGGAGCTTAGGAAACGCTCCCACTCCCACGGGCCAT
CGAGGGCCCAGCACGGCTGAGCGGCTGAAAAACCGTAGCCATAGATCC
TGTCCACCTCCACTCCCCTCTGACCCTCAGGCTTTCCCAGCTTCCTCC
CCTTGCTACAGCCTCTGCCCTGGTTTGGGCTATGTCAGATCCAAAAAC
ATCCTGAACCTCTGTCTGTAAAATGAGTAGTGTCTGTACTTTGAATGA
GGGGGTTGGTGGCAGGGGCCAGTTGAATGTGCTGGGGGGGGTGGTGGG
AAGGATAGTAAATGTGCTGGGGCAAACTGACAAATCTTCCCATCCATT
TCACCACCCATCTCCATCCAGGCCGCGCTAGAGTACTGGACCAGGAAT
TTGGATGCCTGGGTTCAAATCTGCATCTGCCATGCACTTGTTTCTGAC
CTTAGGCCAGCCCCTTTCCCTCCCTGAGTCTCTATTTTCTTATCTACA
ATGAGACAGTTGGACAAAAAAATCTTGGCTTCCCTTCTAACATTAACT
TCCTAAAGTATGCCTCCGATTCATTCCCTTGACACTTTTTATTTCTAA
GGAAGAAATAAAAAGAGATACACAAACACATAAACACAAAAAAAAAAA
NM_001156.5 Homo sapiens annexin A7 (ANXA7),
transcript variant 1, mRNA
(SEQ ID NO: 27)
ATCTTGCGGGAGACCGGGTTGGGCTGTGACGCTGCTGCTGGGGTCAGA
ATGTCATACCCAGGCTATCCCCCAACAGGCTACCCACCTTTCCCTGG
ATATCCTCCTGCAGGTCAGGAGTCATCTTTTCCCCCTTCTGGTCAGTA
TCCTTATCCTAGTGGCTTTCCTCCAATGGGAGGAGGTGCCTACCCACA
AGTGCCAAGTAGTGGCTACCCAGGAGCTGGAGGCTACCCTGCGCCTGG
AGGTTATCCAGCCCCTGGAGGCTATCCTGGTGCCCCACAGCCAGGGGG
AGCTCCATCCTATCCCGGAGTTCCTCCAGGCCAAGGATTTGGAGTCCC
ACCAGGTGGAGCAGGCTTTTCTGGGTATCCACAGCCACCTTCACAGTC
TTATGGAGGTGGTCCAGCACAGGTTCCACTACCTGGTGGCTTTCCTGG
AGGACAGATGCCTTCTCAGTATCCTGGAGGACAACCTACTTACCCTAG
TCAGCCTGCCACAGTGACTCAGGTCACTCAAGGAACTATCCGACCAGC
TGCCAACTTCGATGCTATAAGAGATGCAGAAATTCTTCGTAAGGCAAT
GAAGGGTTTTGGGACAGATGAGCAGGCAATTGTGGATGTGGTGGCCAA
CCGTTCCAATGATCAGAGGCAAAAAATTAAAGCAGCATTTAAGACCTC
CTATGGCAAGGATTTAATCAAAGATCTCAAATCAGAGTTAAGTGGAAA
TATGGAAGAACTGATCCTGGCCCTCTTCATGCCTCCTACGTATTACGA
TGCCTGGAGCTTACGGAAAGCAATGCAGGGAGCAGGAACTCAGGAACG
TGTATTGATTGAGATTTTGTGCACAAGAACAAATCAGGAAATCCGAGA
AATTGTCAGATGTTATCAGTCAGAATTTGGACGAGACCTTGAAAAGGA
CATTAGGTCAGATACATCAGGACATTTTGAACGTTTACTTGTGTCCAT
GTGCCAGGGAAATCGTGATGAGAACCAGAGTATAAACCACCAAATGGC
TCAGGAAGATGCTCAGCGTCTCTATCAAGCTGGTGAGGGGAGACTAGG
GACCGATGAATCTTGCTTTAACATGATCCTTGCCACAAGAAGCTTTCC
TCAGCTGAGAGCTACCATGGAGGCTTATTCTAGGATGGCTAATCGAGA
CTTGTTAAGCAGTGTGAGCCGTGAGTTTTCCGGATATGTAGAAAGTGG
TTTGAAGACCATCTTGCAGTGTGCCCTGAACCGCCCTGCCTTCTTTGC
TGAGAGGCTCTACTATGCTATGAAAGGTGCTGGCACAGATGACTCCAC
CCTGGTCCGGATTGTGGTCACTCGAAGTGAGATTGACCTTGTACAAAT
AAAACAGATGTTCGCTCAGATGTATCAGAAGACTCTGGGCACAATGAT
TGCAGGTGACACGAGTGGAGATTACCGAAGACTTCTTCTGGCTATTGT
GGGCCAGTAGGAGGGATTTTTTTTTTTTTAATGAAAAAAAATTTCTAT
TCATAGCTTATCCTTCAGAGCAATGACCTGCATGCAGCAATATCAAAC
ATCAGCTAACCGAAAGAGCTTTCTGTCAAGGACCGTATCAGGGTAATG
TGCTTGGTTTGCACATGTTGTTATTGCCTTAATTCTAATTTTATTTTG
TTCTCTACATACAATCAATGTAAAGCCATATCACAATGATACAGTAAT
ATTGCAATGTTTGTAAACCTTCATTCTTACTAGTTTCATTCTAATCAA
GATGTCAAATTGAATAAAAATCACAGCAATCTCTGATTCTGTGTAATA
ATATTGAATAATTTTTTAGAAGGTTACTGAAAGCTCTGCCTTCCGGAA
TCCCTCTAAGTCTGCTTGATAGAGTGGATAGTGTGTTAAAACTGTGTA
CTTTAAAAAAAAATTCAACCTTTACATCTAGAATAATTTGCATCTCAT
TTTGCCTAAATTGGTTCTGTATTCATAAACACTTTCCACATAGAAAAT
AGATTAGTATTACCTGTGGCACCTTTTAAGAAAGGGTCAAATGTTTAT
ATGCTTAAGATACATAGCCTACTTTTTTTTCGCAGTTGTTTTCTTTTT
TTAAATTGAGTTATGACAAATAAAAAATTGCATATATTTAAGGTGTAC
AATATGGTGTTTTGATATCAGCATTCCTTGTGTAATGATTCCACAATT
AAGGTCAGGCTAATTACGTATCTGTCACCTTGACATAGTTACCATTTT
TTCATGTGTGGTGAAAACACTTAAGATCTACTACCTTAGCAAATTTTA
AGTGTTCAGTACATTATTAACTATAGATACTGTGCTCTACATTAAACC
TCTAGCATTTATTCGTTTTATAACTGAAAGTTTATACCCTTTGACCAA
CATCTCCCCATTTTCCCCACCTCTCACCTGGACAACCACCACTGTGTT
TAAGTTCAGCTATTTTAGATTCCACGTATAAATGGTATACAATA
NM_004034.3 Homo sapiens annexin A7 (ANXA7),
transcript variant 2, mRNA
(SEQ ID NO: 28)
GCCCACCCTGGGCCCGCCCCCGGCTCCATCTTGCGGGAGACCGGGTTG
GGCTGTGACGCTGCTGCTGGGGTCAGAATGTCATACCCAGGCTATCC
CCCAACAGGCTACCCACCTTTCCCTGGATATCCTCCTGCAGGTCAGGA
GTCATCTTTTCCCCCTTCTGGTCAGTATCCTTATCCTAGTGGCTTTCC
TCCAATGGGAGGAGGTGCCTACCCACAAGTGCCAAGTAGTGGCTACCC
AGGAGCTGGAGGCTACCCTGCGCCTGGAGGTTATCCAGCCCCTGGAGG
CTATCCTGGTGCCCCACAGCCAGGGGGAGCTCCATCCTATCCCGGAGT
TCCTCCAGGCCAAGGATTTGGAGTCCCACCAGGTGGAGCAGGCTTTTC
TGGGTATCCACAGCCACCTTCACAGTCTTATGGAGGTGGTCCAGCACA
GGTTCCACTACCTGGTGGCTTTCCTGGAGGACAGATGCCTTCTCAGTA
TCCTGGAGGACAACCTACTTACCCTAGTCAGATCAATACAGATTCTTT
TTCTTCCTATCCTGTTTTCTCTCCTGTTTCTTTGGATTATAGCAGTGA
ACCTGCCACAGTGACTCAGGTCACTCAAGGAACTATCCGACCAGCTGC
CAACTTCGATGCTATAAGAGATGCAGAAATTCTTCGTAAGGCAATGAA
GGGTTTTGGGACAGATGAGCAGGCAATTGTGGATGTGGTGGCCAACCG
TTCCAATGATCAGAGGCAAAAAATTAAAGCAGCATTTAAGACCTCCTA
TGGCAAGGATTTAATCAAAGATCTCAAATCAGAGTTAAGTGGAAATAT
GGAAGAACTGATCCTGGCCCTCTTCATGCCTCCTACGTATTACGATGC
CTGGAGCTTACGGAAAGCAATGCAGGGAGCAGGAACTCAGGAACGTGT
ATTGATTGAGATTTTGTGCACAAGAACAAATCAGGAAATCCGAGAAAT
TGTCAGATGTTATCAGTCAGAATTTGGACGAGACCTTGAAAAGGACAT
TAGGTCAGATACATCAGGACATTTTGAACGTTTACTTGTGTCCATGTG
CCAGGGAAATCGTGATGAGAACCAGAGTATAAACCACCAAATGGCTCA
GGAAGATGCTCAGCGTCTCTATCAAGCTGGTGAGGGGAGACTAGGGAC
CGATGAATCTTGCTTTAACATGATCCTTGCCACAAGAAGCTTTCCTCA
GCTGAGAGCTACCATGGAGGCTTATTCTAGGATGGCTAATCGAGACTT
GTTAAGCAGTGTGAGCCGTGAGTTTTCCGGATATGTAGAAAGTGGTTT
GAAGACCATCTTGCAGTGTGCCCTGAACCGCCCTGCCTTCTTTGCTGA
GAGGCTCTACTATGCTATGAAAGGTGCTGGCACAGATGACTCCACCCT
GGTCCGGATTGTGGTCACTCGAAGTGAGATTGACCTTGTACAAATAAA
ACAGATGTTCGCTCAGATGTATCAGAAGACTCTGGGCACAATGATTGC
AGGTGACACGAGTGGAGATTACCGAAGACTTCTTCTGGCTATTGTGGG
CCAGTAGGAGGGATTTTTTTTTTTTTAATGAAAAAAAATTTCTATTCA
TAGCTTATCCTTCAGAGCAATGACCTGCATGCAGCAATATCAAACATC
AGCTAACCGAAAGAGCTTTCTGTCAAGGACCGTATCAGGGTAATGTGC
TTGGTTTGCACATGTTGTTATTGCCTTAATTCTAATTTTATTTTGTTC
TCTACATACAATCAATGTAAAGCCATATCACAATGATACAGTAATATT
GCAATGTTTGTAAACCTTCATTCTTACTAGTTTCATTCTAATCAAGAT
GTCAAATTGAATAAAAATCACAGCAATCTCTGATTCTGTGTAATAATA
TTGAATAATTTTTTAGAAGGTTACTGAAAGCTCTGCCTTCCGGAATCC
CTCTAAGTCTGCTTGATAGAGTGGATAGTGTGTTAAAACTGTGTACTT
TAAAAAAAAATTCAACCTTTACATCTAGAATAATTTGCATCTCATTTT
GCCTAAATTGGTTCTGTATTCATAAACACTTTCCACATAGAAAATAGA
TTAGTATTACCTGTGGCACCTTTTAAGAAAGGGTCAAATGTTTATATG
CTTAAGATACATAGCCTACTTTTTTTTCGCAGTTGTTTTCTTTTTTTA
AATTGAGTTATGACAAATAAAAAATTGCATATATTTAAGGTGTACAAT
ATGGTGTTTTGATATCAGCATTCCTTGTGTAATGATTCCACAATTAAG
GTCAGGCTAATTACGTATCTGTCACCTTGACATAGTTACCATTTTTTC
ATGTGTGGTGAAAACACTTAAGATCTACTACCTTAGCAAATTTTAAGT
GTTCAGTACATTATTAACTATAGATACTGTGCTCTACATTAAACCTCT
AGCATTTATTCGTTTTATAACTGAAAGTTTATACCCTTTGACCAACAT
CTCCCCATTTTCCCCACCTCTCACCTGGACAACCACCACTGTGTTTAA
GTTCAGCTATTTTAGATTCCACGTATAAATGGTATACAATAAAAAAAA
AAAAAAA
NM_001271702.1 Homo sapiens annexin A8 (ANXA8),
transcript variant 1, mRNA
(SEQ ID NO: 29)
CTGGGTGGGGCCTGGGAGCCACAGGAGATGCCCAAAGCCAGGCAGAGC
CCGGGGGCGAGGGGACGGCAGGCAGGTGTGGCGCTGCCCTGGGGGGC
TTGCACCCCCACACCCAAGTGAGCGGCCTGCTCACTCCTCAGCTGCAG
GAGCCAGACGTGTGGAGTCCCAGCAGAGGCCAACCTGTGTCTCTTCAT
CTCCCTGGGAAAGGTGCCCCCGAGGTGAAAGAGATGGCCTGGTGGAAA
TCCTGGATTGAACAGGAGGGTGTCACAGTGAAGAGCAGCTCCCACTTC
AACCCAGACCCTGATGCAGAGACCCTCTACAAAGCCATGAAGGGGATC
GGTGTCGGGTCCCAACTGCTCAGCCACCAAGCAGCTGCCTTCGCCTTC
CCCTCCTCCGCCCTCACCAGTGTGTCACCCTGGGGGCAGCAGGGTCAC
TTGTGCTGTAACCCTGCAGGGACCAACGAGCAGGCTATCATCGATGTG
CTCACCAAGAGAAGCAACACGCAGCGGCAGCAGATCGCCAAGTCCTTC
AAGGCTCAGTTCGGCAAGGACCTCACTGAGACCTTGAAGTCTGAGCTC
AGTGGCAAGTTTGAGAGGCTCATTGTGGCCCTTATGTACCCGCCATAC
AGATACGAAGCCAAGGAGCTGCATGACGCCATGAAGGGCTTAGGAACC
AAGGAGGGTGTCATCATTGAGATCCTGGCCTCTCGGACCAAGAACCAG
CTGCGGGAGATAATGAAGGCGTATGAGGAAGACTATGGGTCCAGCCTG
GAGGAGGACATCCAAGCAGACACAAGTGGCTACCTGGAGAGGATCCTG
GTGTGCCTCCTGCAGGGCAGCAGGGATGATGTGAGCAGCTTTGTGGAC
CCAGGACTGGCCCTCCAAGACGCACAGGATCTGTATGCGGCAGGCGAG
AAGATTCGTGGGACTGATGAGATGAAATTCATCACCATCCTGTGCACG
CGCAGTGCCACTCACCTGCTGAGAGTGTTTGAAGAGTATGAGAAAATT
GCCAACAAGAGCATTGAGGACAGCATCAAGAGTGAGACCCATGGCTCA
CTGGAGGAGGCCATGCTCACTGTGGTGAAATGCACCCAAAACCTCCAC
AGCTACTTTGCAGAGAGACTCTACTATGCCATGAAGGGAGCAGGGACG
CGTGATGGGACCCTGATAAGAAACATCGTTTCAAGGAGCGAGATTGAC
TTAAATCTTATCAAATGTCACTTCAAGAAGATGTACGGCAAGACCCTC
AGCAGCATGATCATGGAAGACACCAGCGGTGACTACAAGAACGCCCTG
CTGAGCCTGGTGGGCAGCGACCCCTGAGGCACAGAAGAACAAGAGCAA
AGACCATGAAGCCAGAGTCTCCAGGACTCCTCACTCAACCTCGGCCAT
GGACGCAGGTTGGGTGTGAGGGGGGTCCCAGCCTTTCGGTCTTCTATT
TCCCTATTTCCAGTGCTTTCCAGCCGGGTTTCTGACCCAGAGGGTGGA
ACCGGCCTGGACTCCTCTTCCCAACTTCCTCCAGGTCATTTCCCAGTG
TGAGCACAATGCCAACCTTAGTGTTTCTCCAGCCAGACAGATGCCTCA
GCATGAAGGGCTTGGGGACTTGTGGATCATTCCTTCCTCCCTGCAGGA
GCTTCCCAAGCTGGTCACAGAGTCTCCTGGGCACAGGTTATACAGACC
CCAGCCCCATTCCCATCTACTGAAACAGGGTCTCCACAAGAGGGGCCA
GGGAATATGGGTTTTTAACAAGCGTCTTACAAAACACTTCTCTATCAT
GCAGCCGGAGAGCTGGCTGGGAGCCCTTTTGTTTTAGAACACACATCC
TTCAGCAGCTGAGAAACGAACACGAATCCATCCCAACCGAGATGCCAT
TAACATTCATCTAAAAATGTTAGGCTCTAAATGGACGAAAAATTCTCT
CGCCATCTTAATAACAAAATAAACTACAAATTCCTGACCCAAGGACAC
TGTGTTATAAGAGGCGTGGGCTCCCCTGGTGGCTGACCAGGTCAGCTG
CCCTGGCCTTGCACCCCTCTGCATGCAGCACAGAAGGGTGTGACCATG
CCCTCAGCACCACTCTTGTCCCCACTGAACGGCAACTGAGACTGGGTA
CCTGGAGATTCTGAAGTGCCTTTGCTGTGGTTTTCAAAATAATAAAGA
TTTGTATTCAACTCAAAAAAAAAAAAAAAAAAAAAAAAAAAAAAA
NM_001040084.2 Homo sapiens annexin A8 (ANXA8),
transcript variant 2, mRNA
(SEQ ID NO: 30)
CTGGGTGGGGCCTGGGAGCCACAGGAGATGCCCAAAGCCAGGCAGAGC
CCGGGGGCGAGGGGACGGCAGGCAGGTGTGGCGCTGCCCTGGGGGGG
CTTGCACCCCCACACCCAAGTGAGCGGCCTGCTCACTCCTCAGCTGCA
GGAGCCAGACGTGTGGAGTCCCAGCAGAGGCCAACCTGTGTCTCTTCA
TCTCCCTGGGAAAGGTGCCCCCGAGGTGAAAGAGATGGCCTGGTGGAA
ATCCTGGATTGAACAGGAGGGTGTCACAGTGAAGAGCAGCTCCCACTT
CAACCCAGACCCTGATGCAGAGACCCTCTACAAAGCCATGAAGGGGAT
CGGGACCAACGAGCAGGCTATCATCGATGTGCTCACCAAGAGAAGCAA
CACGCAGCGGCAGCAGATCGCCAAGTCCTTCAAGGCTCAGTTCGGCAA
GGACCTCACTGAGACCTTGAAGTCTGAGCTCAGTGGCAAGTTTGAGAG
GCTCATTGTGGCCCTTATGTACCCGCCATACAGATACGAAGCCAAGGA
GCTGCATGACGCCATGAAGGGCTTAGGAACCAAGGAGGGTGTCATCAT
TGAGATCCTGGCCTCTCGGACCAAGAACCAGCTGCGGGAGATAATGAA
GGCGTATGAGGAAGACTATGGGTCCAGCCTGGAGGAGGACATCCAAGC
AGACACAAGTGGCTACCTGGAGAGGATCCTGGTGTGCCTCCTGCAGGG
CAGCAGGGATGATGTGAGCAGCTTTGTGGACCCAGGACTGGCCCTCCA
AGACGCACAGGATCTGTATGCGGCAGGCGAGAAGATTCGTGGGACTGA
TGAGATGAAATTCATCACCATCCTGTGCACGCGCAGTGCCACTCACCT
GCTGAGAGTGTTTGAAGAGTATGAGAAAATTGCCAACAAGAGCATTGA
GGACAGCATCAAGAGTGAGACCCATGGCTCACTGGAGGAGGCCATGCT
CACTGTGGTGAAATGCACCCAAAACCTCCACAGCTACTTTGCAGAGAG
ACTCTACTATGCCATGAAGGGAGCAGGGACGCGTGATGGGACCCTGAT
AAGAAACATCGTTTCAAGGAGCGAGATTGACTTAAATCTTATCAAATG
TCACTTCAAGAAGATGTACGGCAAGACCCTCAGCAGCATGATCATGGA
AGACACCAGCGGTGACTACAAGAACGCCCTGCTGAGCCTGGTGGGCAG
CGACCCCTGAGGCACAGAAGAACAAGAGCAAAGACCATGAAGCCAGAG
TCTCCAGGACTCCTCACTCAACCTCGGCCATGGACGCAGGTTGGGTGT
GAGGGGGGTCCCAGCCTTTCGGTCTTCTATTTCCCTATTTCCAGTGCT
TTCCAGCCGGGTTTCTGACCCAGAGGGTGGAACCGGCCTGGACTCCTC
TTCCCAACTTCCTCCAGGTCATTTCCCAGTGTGAGCACAATGCCAACC
TTAGTGTTTCTCCAGCCAGACAGATGCCTCAGCATGAAGGGCTTGGGG
ACTTGTGGATCATTCCTTCCTCCCTGCAGGAGCTTCCCAAGCTGGTCA
CAGAGTCTCCTGGGCACAGGTTATACAGACCCCAGCCCCATTCCCATC
TACTGAAACAGGGTCTCCACAAGAGGGGCCAGGGAATATGGGTTTTTA
ACAAGCGTCTTACAAAACACTTCTCTATCATGCAGCCGGAGAGCTGGC
TGGGAGCCCTTTTGTTTTAGAACACACATCCTTCAGCAGCTGAGAAAC
GAACACGAATCCATCCCAACCGAGATGCCATTAACATTCATCTAAAAA
TGTTAGGCTCTAAATGGACGAAAAATTCTCTCGCCATCTTAATAACAA
AATAAACTACAAATTCCTGACCCAAGGACACTGTGTTATAAGAGGCGT
GGGCTCCCCTGGTGGCTGACCAGGTCAGCTGCCCTGGCCTTGCACCCC
TCTGCATGCAGCACAGAAGGGTGTGACCATGCCCTCAGCACCACTCTT
GTCCCCACTGAACGGCAACTGAGACTGGGTACCTGGAGATTCTGAAGT
GCCTTTGCTGTGGTTTTCAAAATAATAAAGATTTGTATTCAACTCAAA
AAAAAAA
NM_003568.3 Homo sapiens annexin A9 (ANXA9), mRNA
(SEQ ID NO: 31)
CTCTACCAGGCCACACCGGAGGCAGTGCTCACACAGGCAAGCTACCA
GGCCACAACAACGACACCCACCTCACCTCTGGCACCTCTGAGCATCCA
CGTACTTGCAAGAACTCTTGCTCACATCAGCTAAGAGATTGCACCTGC
TGACCTAGAGATTCCGGCCTGTGCTCCTGTGCTGCTGAGCAGGGCAAC
CAGTAGCACCATGTCTGTGACTGGCGGGAAGATGGCACCGTCCCTCAC
CCAGGAGATCCTCAGCCACCTGGGCCTGGCCAGCAAGACTGCAGCGTG
GGGGACCCTGGGCACCCTCAGGACCTTCTTGAACTTCAGCGTGGACAA
GGATGCGCAGAGGCTACTGAGGGCCATTACTGGCCAAGGCGTGGACCG
CAGTGCCATTGTGGACGTGCTGACCAACCGGAGCAGAGAGCAAAGGCA
GCTCATCTCACGAAACTTCCAGGAGCGCACCCAACAGGACCTGATGAA
GTCTCTACAGGCAGCACTTTCCGGCAACCTGGAGAGGATTGTGATGGC
TCTGCTGCAGCCCACAGCCCAGTTTGACGCCCAGGAATTGAGGACAGC
TCTGAAGGCCTCAGATTCTGCTGTGGACGTGGCCATTGAAATTCTTGC
CACTCGAACCCCACCCCAGCTGCAGGAGTGCCTGGCAGTCTACAAACA
CAATTTCCAGGTGGAGGCTGTGGATGACATCACATCTGAGACCAGTGG
CATCTTGCAGGACCTGCTGTTGGCCCTGGCCAAGGGGGGCCGTGACAG
CTACTCTGGAATCATTGACTATAATCTGGCAGAACAAGATGTCCAGGC
ACTGCAGCGGGCAGAAGGACCTAGCAGAGAGGAAACATGGGTCCCAGT
CTTCACCCAGCGAAATCCTGAACACCTCATCCGAGTGTTTGATCAGTA
CCAGCGGAGCACTGGGCAAGAGCTGGAGGAGGCTGTCCAGAACCGTTT
CCATGGAGATGCTCAGGTGGCTCTGCTCGGCCTAGCTTCGGTGATCAA
GAACACACCGCTGTACTTTGCTGACAAACTTCATCAAGCCCTCCAGGA
AACTGAGCCCAATTACCAAGTCCTGATTCGCATCCTTATCTCTCGATG
TGAGACTGACCTTCTGAGTATCAGAGCTGAGTTCAGGAAGAAATTTGG
GAAGTCCCTCTACTCTTCTCTCCAGGATGCAGTGAAAGGGGATTGCCA
GTCAGCCCTCCTGGCCTTGTGCAGGGCTGAAGACATGTGAGACTTCCC
TGCCCCACCCCACATGACATCCGAGGATCTGAGATTTCCGTGTTTGGC
TGAACCTGGGAGACCAGCTGGGCCTCCAAGTAGGATAACCCCTCACTG
AGCACCACATTCTCTAGCTTCTTGTTGAGGCTGGAACTGTTTCTTTAA
AATCCCTTAATTTTCCCATCTCAAAATTATATCTGTACCTGGGTCATC
CAGCTCCTTCTTGGGTGTGGGGAAATGAGTTTTCTTTGATAGTTTCTG
CCTCACTCATCCCTCCTGTACCCTGGCCAGAACATCTCACTGATACTC
GAATTCTTTTGGCAAA
NM_007193.4 Homo sapiens annexin A10 (ANXA10),
mRNA
(SEQ ID NO: 32)
ATCCAGATTTGCTTTTACATTTTCTTGCCTGAGTCTGAGGTGAACAGT
GAACATATTTACATTTGATTTAACAGTGAACCTTAATTCTTTCTGGC
TTCACAGTGAAACAAGTTTATGCAATCGATCAAATATTTTCATCCCTG
AGGTTAACAATTACCATCAAAATGTTTTGTGGAGACTATGTGCAAGGA
ACCATCTTCCCAGCTCCCAATTTCAATCCCATAATGGATGCCCAAATG
CTAGGAGGAGCACTCCAAGGATTTGACTGTGACAAAGACATGCTGATC
AACATTCTGACTCAGCGCTGCAATGCACAAAGGATGATGATTGCAGAG
GCATACCAGAGCATGTATGGCCGGGACCTGATTGGGGATATGAGGGAG
CAGCTTTCGGATCACTTCAAAGATGTGATGGCTGGCCTCATGTACCCA
CCACCACTGTATGATGCTCATGAGCTCTGGCATGCCATGAAGGGAGTA
GGCACTGATGAGAATTGCCTCATTGAAATACTAGCTTCAAGAACAAAT
GGAGAAATTTTCCAGATGCGAGAAGCCTACTGCTTGCAATACAGCAAT
AACCTCCAAGAGGACATTTATTCAGAGACCTCAGGACACTTCAGAGAT
ACTCTCATGAACTTGGTCCAGGGGACCAGAGAGGAAGGATATACAGAC
CCTGCGATGGCTGCTCAGGATGCAATGGTCCTATGGGAAGCCTGTCAG
CAGAAGACGGGGGAGCACAAAACCATGCTGCAAATGATCCTGTGCAAC
AAGAGCTACCAGCAGCTGCGGCTGGTTTTCCAGGAATTTCAAAATATT
TCTGGGCAAGATATGGTAGATGCCATTAATGAATGTTATGATGGATAC
TTTCAGGAGCTGCTGGTTGCAATTGTTCTCTGTGTTCGAGACAAACCA
GCCTATTTTGCTTATAGATTATATAGTGCAATTCATGACTTTGGTTTC
CATAATAAAACTGTAATCAGGATTCTCATTGCCAGAAGTGAAATAGAC
CTGCTGACCATAAGGAAACGATACAAAGAGCGATATGGAAAATCCCTA
TTTCATGATATCAGAAATTTTGCTTCAGGGCATTATAAGAAAGCACTG
CTTGCCATCTGTGCTGGTGATGCTGAGGACTACTAAAATGAAGAGGAC
TTGGAGTACTGTGCACTCCTCTTTCTAGACACTTCCAAATAGAGATTT
TCTCACAAATTTGTACTGTTCATGGCACTATTAACAAAACTATACAAT
CATATTTTCTCTTCTATCTTTGAAATTATTCTAAGCCAAAGAAAACTA
TGAATGAAAGTATATGATACTGAATTTGCCTACTATCCTGAATTTGCC
TACTATCTAATCAGCAATTAAATAAATTGTGCATGATGGAATAATAGA
AAAATTGCATTGGAATAGATTTTATTTAAATGTGAACCATCAACAACC
TACAACAA
NM_145868.2 Homo sapiens annexin A11 (ANXA11),
transcript variant b, mRNA
(SEQ ID NO: 33)
GGAGTTTTCCGCCCGGCGCTGACGGCTGCTGCGCCCGCGGCTCCCCAG
TGCCCCGAGTGCCCCGCGGGCCCCGCGAGCGGGAGTGGGACCCAGCC
CCTAGGCAGAACCCAGGCGCCGCGCCCGGGACGCCCGCGGAGAGAGCC
ACTCCCGCCCACGTCCCATTTCGCCCCTCGCGTCCGGAGTCCCCGTGG
CCAGGTGTGTGTCTGGGGAAGAGACTTACAGAAGTGGAGTTGCTGAGT
CAAAGATCTAACCATGAGCTACCCTGGCTATCCCCCGCCCCCAGGTGG
CTACCCACCAGCTGCACCAGGTGGTGGTCCCTGGGGAGGTGCTGCCTA
CCCTCCTCCGCCCAGCATGCCCCCCATCGGGCTGGATAACGTGGCCAC
CTATGCGGGGCAGTTCAACCAGGACTATCTCTCGGGAATGGCGGCCAA
CATGTCTGGGACATTTGGAGGAGCCAACATGCCCAACCTGTACCCTGG
GGCCCCTGGGGCTGGCTACCCACCAGTGCCCCCTGGCGGCTTTGGGCA
GCCCCCCTCTGCCCAGCAGCCTGTTCCTCCCTATGGGATGTATCCACC
CCCAGGAGGAAACCCACCCTCCAGGATGCCCTCATATCCGCCATACCC
AGGGGCCCCTGTGCCGGGCCAGCCCATGCCACCCCCCGGACAGCAGCC
CCCAGGGGCCTACCCTGGGCAGCCACCAGTGACCTACCCTGGTCAGCC
TCCAGTGCCACTCCCTGGGCAGCAGCAGCCAGTGCCGAGCTACCCAGG
ATACCCGGGGTCTGGGACTGTCACCCCCGCTGTGCCCCCAACCCAGTT
TGGAAGCCGAGGCACCATCACTGATGCTCCCGGCTTTGACCCCCTGCG
AGATGCCGAGGTCCTGCGGAAGGCCATGAAAGGCTTCGGGACGGATGA
GCAGGCCATCATTGACTGCCTGGGGAGTCGCTCCAACAAGCAGCGGCA
GCAGATCCTACTTTCCTTCAAGACGGCTTACGGCAAGGATTTGATCAA
AGATCTGAAATCTGAACTGTCAGGAAACTTTGAGAAGACAATCTTGGC
TCTGATGAAGACCCCAGTCCTCTTTGACATTTATGAGATAAAGGAAGC
CATCAAGGGGGTTGGCACTGATGAAGCCTGCCTGATTGAGATCCTCGC
TTCCCGCAGCAATGAGCACATCCGAGAATTAAACAGAGCCTACAAAGC
AGAATTCAAAAAGACCCTGGAAGAGGCCATTCGAAGCGACACATCAGG
GCACTTCCAGCGGCTCCTCATCTCTCTCTCTCAGGGAAACCGTGATGA
AAGCACAAACGTGGACATGTCACTCGCCCAGAGAGATGCCCAGGAGCT
GTATGCGGCCGGGGAGAACCGCCTGGGAACAGACGAGTCCAAGTTCAA
TGCGGTTCTGTGCTCCCGGAGCCGGGCCCACCTGGTAGCAGTTTTCAA
TGAGTACCAGAGAATGACAGGCCGGGACATTGAGAAGAGCATCTGCCG
GGAGATGTCCGGGGACCTGGAGGAGGGCATGCTGGCCGTGGTGAAATG
TCTCAAGAATACCCCAGCCTTCTTTGCGGAGAGGCTCAACAAGGCCAT
GAGGGGGGCAGGAACAAAGGACCGGACCCTGATTCGCATCATGGTGTC
TCGCAGCGAGACCGACCTCCTGGACATCAGATCAGAGTATAAGCGGAT
GTACGGCAAGTCGCTGTACCACGACATCTCGGGAGATACTTCAGGGGA
TTACCGGAAGATTCTGCTGAAGATCTGTGGTGGCAATGACTGAACAGT
GACTGGTGGCTCACTTCTGCCCACCTGCCGGCAACACCAGTGCCAGGA
AAAGGCCAAAAGAATGTCTGTTTCTAACAAATCCACAAATAGCCCCGA
GATTCACCGTCCTAGAGCTTAGGCCTGTCTTCCACCCCTCCTGACCCG
TATAGTGTGCCACAGGACCTGGGTCGGTCTAGAACTCTCTCAGGATGC
CTTTTCTACCCCATCCCTCACAGCCTCTTGCTGCTAAAATAGATGTTT
CATTTTTCTGACTCATGCAATCATTCCCCTTTGCCTGTGGCTAAGACT
TGGCTTCATTTCGTCATGTAATTGTATATTTTTATTTGGAGGCATATT
TTCTTTTCTTACAGTCATTGCCAGACAGAGGCATACAAGTCTGTTTGC
TGCATACACATTTCTGGTGAGGGCGACTGGGTGGGTGAAGCACCGTGT
CCTCGCTGAGGAGAGAAAGGGAGGCGTGCCTGAGAAGGTAGCCTGTGC
ATCTGGTGAGTGTGTCACGAGCTTTGTTACTGCCAAACTCACTCCTTT
TTAGAAAAAACAAAAAAAAAGGGCCAGAAAGTCATTCCTTCCATCTTC
CTTGCAGAAACCACGAGAACAAAGCCAGTTCCCTGTCAGTGACAGGGC
TTCTTGTAATTTGTGGTATGTGCCTTAAACCTGAATGTCTGTAGCCAA
AACTTGTTTCCACATTAAGAGTCAGCCAGCTCTGGAATGGTCTGGAAA
TGTCTTCCTGGTACCAACTTGTTTTCTTCTGCTTGATTCTGCCCTGTG
GCTCAGAGGTCTGGCCTTATCAGCCAGTGAAAGTTCATGTAACCTTAC
GTAGAGATTTGTGTGCAGGAAACCCTGAGCATACACTAGTTTGCAGGG
ACTCGTAAGGACATGGGAAGGGAGGTTCCCGAAATCCAGGCAGGAGGC
CCAGACACCTGAAAGGCAAAGGGATCTTGGTTGGTTGCAGGTGCAGTG
AAGTCCACTGAAGGTGTGGTGCGAAGAATGCAGTCCTTCACCCAGGTC
CCAGGAGGGAAGAAGGGTGTGTGCTAATTCCTGGTGCCCCTCGGCGGG
GGCCAGAGAGAAGGATGGGGACAACCCAGAGAGTCACAAGACCAGTGC
CTCCCCTCAGGGTGCCTCCAGGCTGAAAGGGGCTCCTGGCTCTGGTCT
CTGGGGACCCTGTGCCCGTTGGTTGGTGGTGTGAGGGAAGAGAATCCA
TAAGAGAGTTTCTGAGAATTATGGTGTCATGTCCAGAAGCTAGAGCTT
ACCTTGCATCAGGGGTCTCCACCCACTCCTTTTCCAACCTCCTGCGTT
GAGGTTTAGAAAAGAGAGAATCGACTAGGCACTATGGCTCACGCCTGT
AATCCAAGGACTTTGGGAAGCTGAGGTGAGAGGATCACTTGAGCTCAG
GAGTTCAAGACTAGCCTAGCCAACAGCGAGACCCCTGTCTCTACTAAA
AAATTTGGCCAGGCGTGGTGGCTCACGGCTGTAATCCCAGCACTTTGG
GAGGTGAGGGGGGCAGATCACCTGAGGTCAGGAGTTCGAGACCCAGCC
TGGCCAACATGGTGAAACCCCATCTCTACTAAAAATACAAAAATTAGC
CAGGCATGGTGGCACATTCCTGTAATCCCAGCTACACAGGATGCTGAG
GCAGGAGAATCACTTGAACCCAGGAGGCAGAGGTTGTAGTGAGCTGAG
ATCACACCATTGCACTTCAACCTGGGTGGACAGAGTGAGACTCTGTCT
CAAAAAAAAAAAAAAATTTACCTGGCATTGTAGTGCATTCCCTATAGT
CGGCTACTCTGGAGGCTGAGGCAGGAAGATCCTTAGAGCCCAAGAAAT
TGAGGCCGTAGTAAGCTGTGATTACACCACTGCACTCCAGCCTGGACA
ACAGAGCGAGACCTTGTCTCAAATGAGAAAAAAACAAAAAGAAATGGG
AGAATCCAGAGAGACTAGGCTAGATCAAGCCTGCTGGGTCCTGGCAGG
AGCCCCAGGGAGTAGCTCATCTGCAGACATTTGCTTGAGGACTACCCC
CTAAACATAAAGGAAGAATGACATCCGAAGGGTGTGGAGCAGCCATGA
GCTGAGAACTAGCCTGGTCTACCTGAGATTGATGGCAGGTCCTGGTCA
ACACGTCAGCTCTGCGTCAGAGTCCATGCCTCAAGCCCAAGCTGAAGC
CCCATCCCTGCTGCTCTCCCAAGAACTCCTCTGCTAGGGCAGGCCCCT
TGCCCTTGGGTGCCAGGTGGGACCTGCCTGATGGGATGGGGTGCTTGG
CATATACAACTTGCCATGAACTCAAGGTGACCCTGGGGGCCTCCTGAA
TTGTGATGGGGCCTAGAACCAATGTGCTCTGATGTGACCATATTCTGT
GACATTACCTTGCCCTGTTTACTCCAAAGTTCCCAGCCTGGTGCCCAG
CAGGCAATATTGCACCTACAGACACATTTACTTTGGTTTCCAAAGTGT
TTTTAGACATTTGAATTTGTTGCCAACATTTAAACATTGAGAGATTTC
ATATTTTTAAAAATCTGGAATTCTGGCTTCTCTTGAAAACTCAGAAAT
TCTGGCACTATGGGGCTTGCATTCCTGCATGGCTGGAGCTGAGTTGCA
GCTGCCCCTTTAGGCCTGTACTCCTTATTTGCTATAGGCTCCGTCTTG
TATTACACTAAGCCCATGTCACCCATTTGGCTCCTGCAGGCCTTTGGG
TTTGAGACCCTGGTCTACACACTTGGAGACCACCTGTTGTAAAGTACA
TGGATGTGCTTTGGTCAAGGAATAGACCAAGGTGGATATCCAGGCCAG
AGTGACTCAGCGAGTTTAGGTCACAGGCGTATACTCCACTTGTTATAT
AACCTGCTTGTGTAAGTTCATACTTGGCTCAAAGCCACTATTGTTTGG
AAAAGGTATAACTGCCCTGCTGACGCTGTACAGATGTTCTTGGGCTCG
GATGGGCATGGCTCCACGTGGTGTGCACTAGCACCCAGAGAGAGTGAA
GCTATTGACCCCTGTAAGGGAGAGTGACCATCTGGCAGATAGATAGAG
GGGAGCCAGGACATGGCTCAGCTTGTGCCCAGAGGGAGAGTTAAGCCG
CTGACCCTGTAGCCAGGGAGTGCACCTGCAAGCATGGGGGTGGCAGGA
GCCACAGAGCTGGCTGCTGAGAGGAGCTGCAGATCTGGAGAAGACAGC
CTAGGTAAAGGTGGACAGTGTGAGAGCTGCTGATGAGATAGCTGCTGA
ATAAAACTACATTTTACCTGCCTATGGCCCGCCAGGTTTTCTTTCAGC
TATCGCCCATCCACCCAGTCCCCTCGAACCTCAGCATGGGCTGGAACC
TGACCCTGGGCATGACATTTGGCATAGTTGTGGACCTGACACCTGTGT
TTGTCCTAGTCCTGTTTCTCCCTGCCTTCCTGTTCCTCTCGCTGCCCT
CATGGTCACTCCCAAGAGATCCAACCCATGTTAAGTATGGGCTGGAGG
ACTGCATGAATGCCTCATGATCTTCCCAGAGGCAAAGGCACCTACTGC
CTTCCAAGGTCAGTGGGAGGTTGGGATCAACACTGTTTATTATGCTTA
GGACAAAAAAGATAGGGAGAAAGATGTGCAACCTTACAGGTCATCTTT
CTGGGATAGAACACAATGGGTCTTCTCCTGCCTCCTGGATATGTTAGT
CAAGGCCAGTCCATGCTACACATCTAGTCTGACTTCTAAAATAGAAGC
ACCAGATGAATTCAGCCCTGAGAGAATTTTCAGCAGCTGTGGGGGCGC
TGGAGGAAACACTATTAAATAGTTTTGCACCTGAGACAGATAGCCTCA
CTCGCCTCACCCTAGTCCTGGTGGCATTTGTCTCAGGTGCAAAATTTA
AGAAAGAAACCTTGGAGTGCTCACCCTGTGGCTGGGTAGATGGTCCTA
AAGTGGTGGTTTTCAAGCCTGAGTGTGTATCAGGATCATCAGGGGAGC
TTGCTAAAGAGCAGTTCCTGCGGTCAGACCCTCATGCATTTTGAGCAG
GTGTGGGGACTGGGAAACTGCATCTGTAACCTGCTGTAATCTAACGCT
TATCTAAATACTACTGTGCTCACACAGAGAACACCGCAAAAGTAGAGG
TGTTCCTCCAGAGGGCAGGTGAGCAGATGGCACAGTCTGCTTGGAATT
CAGTCAGGTGATGAGAGATGAGATGAGGCACTCCTAGCTTTGGGAAGA
GGGAGCTGAAAGATGAACCTTTGCAGGTGCCCACGGTCAAAGTGGTGG
TTTAATGCCATGCCATGCCCATTTTCTGTTGGCCTTGGCAGGGAGTTA
CAGCCCTACCTTAGGACCTGGCTCCTTATTTCTGCTGTAGGCTCTTTC
CTGCCCTGGCCGAGATGGAGTGGAATGAGACCTAGAAACATCAAGCTA
AATACATGTCCTCAGAAAGATAAAGGTTTACATTTTCACCCCCATCAA
ATCTGAAAGCTCTCTGCCTGTGTTTTTCTAAGGGATAGGGACATCATT
ACTCAGTCCACAACCTGGACTCATGTAGGGTCCCCTGTCAGTAAAGGA
GTCAGTCAAGCCCACCAGGTATACCAAGGACTCTTACCCTCAGCCCCT
ACTCCTTGGAAAGCTGCCCCTTGGCCTAATATTGGTGTTTAGCTTGAG
CCTGACTCCTTCTCAACACTAAGAGCTGATGAAGTCCTGAAGCAGAAA
GAGCTCTGACCTGAGAGTCAAACATCCTTATTCTGATCTCAGCTCAGC
CCCTGATTTGTTGTGTGACCCTGGATATGTCACTTCCTGTCTTTTTGA
CTTTTTAAAATGAAGGGTAGACTAGAGGAGAGCTTCTAAAACTTTAAT
GTGGTCAACGAAATGGAATAGGAAATTCCACAAGTCTGTCCTTCCACA
AAAGCAGCAAATAAGGTGGCAAAAACTCAAATTTATGGGAACTCTGGA
AACGAATTGAAAGTTTACAGCAATCAGGTGAATACCTAAGAATAAAAG
CTGGATTTAGTAAGA
NM_001278409.1 Homo sapiens annexin A11 (ANXA11),
transcript variant f, mRNA
(SEQ ID NO: 34)
GCACTGCCTCTGGCACCTGGGGCAGCCGCGCCCGCGGAGTTTTCCGCC
CGGCGCTGACGGCTGCTGCGCCCGCGGCTCCCCAGTGCCCCGAGTGC
CCCGCGGGCCCCGCGAGCGGGAGTGGGACCCAGCCCCTAGGCAGAACC
CAGGCGCCGCGCCCGGGACGCCCGCGGAGAGAGCCACTCCCGCCCACG
TCCCATTTCGCCCCTCGCGTCCGGAGTCCCCGTGGCCAGGTGTGTGTC
TGGGGAAGAGACTTACAGAAGTGGAGTTGCTGAGTCAAAGATCTAACC
ATGAGCTACCCTGGCTATCCCCCGCCCCCAGGTGGCTACCCACCAGCT
GCACCAGGTTGGCTGGCACTGGCCTGGGTTCTCTCTCTATAGTAGAAA
TCCTGCCATCCAGATCCTGCCACTGCCACCTTTGCTAGCACAGCTGAG
CAGCCTCTGAGCAGCAAGAGAGGAGGAGGCAGGAAATTTAGGGAAGGT
TCTTCCTGGAGGGTCTGGAGCCCTGGAGATGAAGAGCCGATCCGAAGC
TGCCATGTAGAGGAAAGCATCTAACAGGCCAGAGGCCCCATGATGATG
TCGAATGCCCATCGGGCACCCAGCTGAGCCCTGCAGGTGGTGGTCCCT
GGGGAGGTGCTGCCTACCCTCCTCCGCCCAGCATGCCCCCCATCGGGC
TGGATAACGTGGCCACCTATGCGGGGCAGTTCAACCAGGACTATCTCT
CGGGAATGGCGGCCAACATGTCTGGGACATTTGGAGGAGCCAACATGC
CCAACCTGTACCCTGGGGCCCCTGGGGCTGGCTACCCACCAGTGCCCC
CTGGCGGCTTTGGGCAGCCCCCCTCTGCCCAGCAGCCTGTTCCTCCCT
ATGGGATGTATCCACCCCCAGGAGGAAACCCACCCTCCAGGATGCCCT
CATATCCGCCATACCCAGGGGCCCCTGTGCCGGGCCAGCCCATGCCAC
CCCCCGGACAGCAGCCCCCAGGGGCCTACCCTGGGCAGCCACCAGTGA
CCTACCCTGGTCAGCCTCCAGTGCCACTCCCTGGGCAGCAGCAGCCAG
TGCCGAGCTACCCAGGATACCCGGGGTCTGGGACTGTCACCCCCGCTG
TGCCCCCAACCCAGTTTGGAAGCCGAGGCACCATCACTGATGCTCCCG
GCTTTGACCCCCTGCGAGATGCCGAGGTCCTGCGGAAGGCCATGAAAG
GCTTCGGGACGGATGAGCAGGCCATCATTGACTGCCTGGGGAGTCGCT
CCAACAAGCAGCGGCAGCAGATCCTACTTTCCTTCAAGACGGCTTACG
GCAAGGATTTGATCAAAGATCTGAAATCTGAACTGTCAGGAAACTTTG
AGAAGACAATCTTGGCTCTGATGAAGACCCCAGTCCTCTTTGACATTT
ATGAGATAAAGGAAGCCATCAAGGGGGTTGGCACTGATGAAGCCTGCC
TGATTGAGATCCTCGCTTCCCGCAGCAATGAGCACATCCGAGAATTAA
ACAGAGCCTACAAAGCAGAATTCAAAAAGACCCTGGAAGAGGCCATTC
GAAGCGACACATCAGGGCACTTCCAGCGGCTCCTCATCTCTCTCTCTC
AGGGAAACCGTGATGAAAGCACAAACGTGGACATGTCACTCGCCCAGA
GAGATGCCCAGGAGCTGTATGCGGCCGGGGAGAACCGCCTGGGAACAG
ACGAGTCCAAGTTCAATGCGGTTCTGTGCTCCCGGAGCCGGGCCCACC
TGGTAGCAGTTTTCAATGAGTACCAGAGAATGACAGGCCGGGACATTG
AGAAGAGCATCTGCCGGGAGATGTCCGGGGACCTGGAGGAGGGCATGC
TGGCCGTGGTGAAATGTCTCAAGAATACCCCAGCCTTCTTTGCGGAGA
GGCTCAACAAGGCCATGAGGGGGGCAGGAACAAAGGACCGGACCCTGA
TTCGCATCATGGTGTCTCGCAGCGAGACCGACCTCCTGGACATCAGAT
CAGAGTATAAGCGGATGTACGGCAAGTCGCTGTACCACGACATCTCGG
GAGATACTTCAGGGGATTACCGGAAGATTCTGCTGAAGATCTGTGGTG
GCAATGACTGAACAGTGACTGGTGGCTCACTTCTGCCCACCTGCCGGC
AACACCAGTGCCAGGAAAAGGCCAAAAGAATGTCTGTTTCTAACAAAT
CCACAAATAGCCCCGAGATTCACCGTCCTAGAGCTTAGGCCTGTCTTC
CACCCCTCCTGACCCGTATAGTGTGCCACAGGACCTGGGTCGGTCTAG
AACTCTCTCAGGATGCCTTTTCTACCCCATCCCTCACAGCCTCTTGCT
GCTAAAATAGATGTTTCATTTTTCTGACTCATGCAATCATTCCCCTTT
GCCTGTGGCTAAGACTTGGCTTCATTTCGTCATGTAATTGTATATTTT
TATTTGGAGGCATATTTTCTTTTCTTACAGTCATTGCCAGACAGAGGC
ATACAAGTCTGTTTGCTGCATACACATTTCTGGTGAGGGCGACTGGGT
GGGTGAAGCACCGTGTCCTCGCTGAGGAGAGAAAGGGAGGCGTGCCTG
AGAAGGTAGCCTGTGCATCTGGTGAGTGTGTCACGAGCTTTGTTACTG
CCAAACTCACTCCTTTTTAGAAAAAACAAAAAAAAAGGGCCAGAAAGT
CATTCCTTCCATCTTCCTTGCAGAAACCACGAGAACAAAGCCAGTTCC
CTGTCAGTGACAGGGCTTCTTGTAATTTGTGGTATGTGCCTTAAACCT
GAATGTCTGTAGCCAAAACTTGTTTCCACATTAAGAGTCAGCCAGCTC
TGGAATGGTCTGGAAATGTCA
NM_004306.4 Homo sapiens annexin A13 (ANXA13),
transcript variant 1, mRNA
(SEQ ID NO: 35)
GCCTGTAGGAGGACTGATCTCTTGATGAAATACAGAAAAACCATCTCA
GAAAAAGGAAAATGGGCAATCGTCATGCTAAAGCGAGCAGTCCTCAG
GGTTTTGATGTGGATCGAGATGCCAAAAAGCTGAACAAAGCCTGCAAA
GGAATGGGGACCAATGAAGCAGCCATCATTGAAATCTTATCGGGCAGG
ACATCAGATGAGAGGCAACAAATCAAGCAAAAGTACAAGGCAACGTAC
GGCAAGGAGCTGGAGGAAGTACTCAAGAGTGAGCTGAGTGGAAACTTC
GAGAAGACAGCGTTGGCCCTTCTGGACCGTCCCAGCGAGTACGCCGCC
CGGCAGCTGCAGAAGGCTATGAAGGGTCTGGGCACAGATGAGTCCGTC
CTCATTGAGGTCCTGTGCACGAGGACCAATAAGGAAATCATCGCCATT
AAAGAGGCCTACCAAAGGCTATTTGATAGGAGCCTCGAATCAGATGTC
AAAGGTGATACAAGTGGAAACCTAAAAAAAATCCTGGTGTCTCTGCTG
CAGGCTAATCGCAATGAAGGAGATGACGTGGACAAAGATCTAGCTGGT
CAGGATGCCAAAGATCTGTATGATGCAGGGGAAGGCCGCTGGGGCACT
GATGAGCTTGCGTTCAATGAAGTCCTGGCCAAGAGGAGCTACAAGCAG
TTACGAGCCACCTTTCAAGCCTATCAAATTCTCATTGGCAAAGACATA
GAAGAAGCCATTGAAGAAGAAACATCAGGCGACTTGCAGAAGGCCTAT
TTAACTCTCGTGAGATGTGCCCAGGATTGTGAGGACTATTTTGCTGAA
CGTCTGTACAAGTCGATGAAGGGTGCGGGGACCGATGAGGAGACGTTG
ATTCGCATAGTCGTGACCAGGGCCGAGGTGGACCTTCAGGGGATCAAA
GCAAAGTTCCAAGAGAAGTATCAGAAGTCTCTCTCTGACATGGTTCGC
TCAGATACCTCCGGGGACTTCCGGAAACTGCTAGTAGCCCTCTTGCAC
TGAGCCAAGCCAGGGCAATAGGAACACAGGGTGGAACCGCCTTTGTCA
AGAGCACATTCCAAATCAAACTTGCAAATGAGACTCCCGCACGAAAAC
CCTTAAGAGTCCCGGATTACTTTCTTGGCAGCTTAAGTGGCGCAGCCA
GGCCAAGCTGTGTAAGTTAAGGGCAGTAACGTTAAGATGCGTGGGCAG
GGCACCTTGAACTCTGGCTTAGCAAGCATCTAGGCTGCCTCTTCACTT
TCTTTTAGCATGGTAACTGGATGTTTTCTAAACACTAATGAAATCAGC
AGTTGATGAAAAAACTATGCATTTGTAATGGCACATTTAGAAGGATAT
GCATCACACAAGTAAGGTACAGGAAAGACAAAATTAAACAATTTATTA
ATTTTCCTTCTGTGTGTTCAATTTGAAAGCCTCATTGTTAATTAAAGT
TGTGGATTATGCCTCTA
NM_001003954.2 Homo sapiens annexin A13 (ANXA13),
transcript variant 2,
mRNA
(SEQ ID NO: 36)
ATTATGTCCGGGGGGAAAACTGTTGTAAACTTTGCCTGTAGGAGGACT
GATCTCTTAATGAAATACAGAAAAACCATCTCAGAAAAAGGAAAATG
GGCAATCGTCATAGCCAGTCGTACACCCTCTCAGAAGGCAGTCAACAG
TTGCCTAAAGGGGACTCCCAACCCTCGACAGTCGTGCAGCCTCTCAGC
CACCCATCACGGAATGGAGAGCCAGAGGCCCCACAGCCTGCTAAAGCG
AGCAGTCCTCAGGGTTTTGATGTGGATCGAGATGCCAAAAAGCTGAAC
AAAGCCTGCAAAGGAATGGGGACCAATGAAGCAGCCATCATTGAAATC
TTATCGGGCAGGACATCAGATGAGAGGCAACAAATCAAGCAAAAGTAC
AAGGCAACGTACGGCAAGGAGCTGGAGGAAGTACTCAAGAGTGAGCTG
AGTGGAAACTTCGAGAAGACAGCGTTGGCCCTTCTGGACCGTCCCAGC
GAGTACGCCGCCCGGCAGCTGCAGAAGGCTATGAAGGGTCTGGGCACA
GATGAGTCCGTCCTCATTGAGGTCCTGTGCACGAGGACCAATAAGGAA
ATCATCGCCATTAAAGAGGCCTACCAAAGGCTATTTGATAGGAGCCTC
GAATCAGATGTCAAAGGTGATACAAGTGGAAACCTAAAAAAAATCCTG
GTGTCTCTGCTGCAGGCTAATCGCAATGAAGGAGATGACGTGGACAAA
GATCTAGCTGGTCAGGATGCCAAAGATCTGTATGATGCAGGGGAAGGC
CGCTGGGGCACTGATGAGCTTGCGTTCAATGAAGTCCTGGCCAAGAGG
AGCTACAAGCAGTTACGAGCCACCTTTCAAGCCTATCAAATTCTCATT
GGCAAAGACATAGAAGAAGCCATTGAAGAAGAAACATCAGGCGACTTG
CAGAAGGCCTATTTAACTCTCGTGAGATGTGCCCAGGATTGTGAGGAC
TATTTTGCTGAACGTCTGTACAAGTCGATGAAGGGTGCGGGGACCGAT
GAGGAGACGTTGATTCGCATAGTCGTGACCAGGGCCGAGGTGGACCTT
CAGGGGATCAAAGCAAAGTTCCAAGAGAAGTATCAGAAGTCTCTCTCT
GACATGGTTCGCTCAGATACCTCCGGGGACTTCCGGAAACTGCTAGTA
GCCCTCTTGCACTGAGCCAAGCCAGGGCAATAGGAACACAGGGTGGAA
CCGCCTTTGTCAAGAGCACATTCCAAATCAAACTTGCAAATGAGACTC
CCGCACGAAAACCCTTAAGAGTCCCGGATTACTTTCTTGGCAGCTTAA
GTGGCGCAGCCAGGCCAAGCTGTGTAAGTTAAGGGCAGTAACGTTAAG
ATGCGTGGGCAGGGCACCTTGAACTCTGGCTTAGCAAGCATCTAGGCT
GCCTCTTCACTTTCTTTTAGCATGGTAACTGGATGTTTTCTAAACACT
AATGAAATCAGCAGTTGATGAAAAAACTATGCATTTGTAATGGCACAT
TTAGAAGGATATGCATCACACAAGTAAGGTACAGGAAAGACAAAATTA
AACAATTTATTAATTTTCCTTCTGTGTGTTCAATTTGAAAGCCTCATT
GTTAATTAAAGTTGTGGATTATGCCTCTAAAAAAAAAAAAAAAAAAAA
AA
NM_001363114.2 Homo sapiens annexin A6 (ANXA6),
transcript variant 3, mRNA
(SEQ ID NO: 38):
GCGGTTGCTGCTGGGCTAACGGGCTCCGATCCAGCGAGCGCTGCGTCC
TCGAGTCCCTGCGCCCGTGCGTCCGTCTGCGACCCGAGGCCTCCGCTG
CGCGTGGATTCTGCTGCGAACCGGAGACCATGGCCAAACCAGCACAGG
GTGCCAAGTACCGGGGCTCCATCCATGACTTCCCAGGCTTTGACCCCA
ACCAGGATGCCGAGGCTCTGTACACTGCCATGAAGGGCTTTGGCAGTG
ACAAGGAGGCCATACTGGACATAATCACCTCACGGAGCAACAGGCAGA
GGCAGGAGGTCTGCCAGAGCTACAAGTCCCTCTACGGCAAGGACCTCA
TTGCTGATTTAAAGTATGAATTGACGGGCAAGTTTGAACGGTTGATTG
TGGGCCTGATGAGGCCACCTGCCTATTGTGATGCCAAAGAAATTAAAG
ATGCCATCTCGGGCATTGGCACTGATGAGAAGTGCCTCATTGAGATCT
TGGCTTCCCGGACCAATGAGCAGATGCACCAGCTGGTGGCAGCATACA
AAGATGCCTACGAGCGGGACCTGGAGGCTGACATCATCGGCGACACCT
CTGGCCACTTCCAGAAGATGCTTGTGGTCCTGCTCCAGGGAACCAGGG
AGGAGGATGACGTAGTGAGCGAGGACCTGGTACAACAGGATGTCCAGG
ACCTATACGAGGCAGGGGAACTGAAATGGGGAACAGATGAAGCCCAGT
TCATTTACATCTTGGGAAATCGCAGCAAGCAGCATCTTCGGTTGGTGT
TCGATGAGTATCTGAAGACCACAGGGAAGCCGATTGAAGCCAGCATCC
GAGGGGAGCTGTCTGGGGACTTTGAGAAGCTAATGCTGGCCGTAGTGA
AGTGTATCCGGAGCACCCCGGAATATTTTGCTGAAAGGCTCTTCAAGG
CTATGAAGGGCCTGGGGACTCGGGACAACACCCTGATCCGCATCATGG
TCTCCCGTAGTGAGTTGGACATGCTCGACATTCGGGAGATCTTCCGGA
CCAAGTATGAGAAGTCCCTCTACAGCATGATCAAGAATGACACCTCTG
GCGAGTACAAGAAGACTCTGCTGAAGCTGTCTGGGGGAGATGATGATG
CTGCTGGCCAGTTCTTCCCGGAGGCAGCGCAGGTGGCCTATCAGATGT
GGGAACTTAGTGCAGTGGCCCGAGTAGAGCTGAAGGGAACTGTGCGCC
CAGCCAATGACTTCAACCCTGACGCAGATGCCAAAGCGCTGCGGAAAG
CCATGAAGGGACTCGGGACTGACGAAGACACAATCATCGATATCATCA
CGCACCGCAGCAATGTCCAGCGGCAGCAGATCCGGCAGACCTTCAAGT
CTCACTTTGGCCGGGACTTAATGACTGACCTGAAGTCTGAGATCTCTG
GAGACCTGGCAAGGCTGATTCTGGGGCTCATGATGCCACCGGCCCATT
ACGATGCCAAGCAGTTGAAGAAGGCCATGGAGGGAGCCGGCACAGATG
AAAAGGCTCTTATTGAAATCCTGGCCACTCGGACCAATGCTGAAATCC
GGGCCATCAATGAGGCCTATAAGGAGGACTATCACAAGTCCCTGGAGG
ATGCTCTGAGCTCAGACACATCTGGCCACTTCAGGAGGATCCTCATTT
CTCTGGCCACGGGGCATCGTGAGGAGGGAGGAGAAAACCTGGACCAGG
CACGGGAAGATGCCCAGGAAATAGCAGACACACCTAGTGGAGACAAAA
CTTCCTTGGAGACACGTTTCATGACGATCCTGTGTACCCGGAGCTATC
CGCACCTCCGGAGAGTCTTCCAGGAGTTCATCAAGATGACCAACTATG
ACGTGGAGCACACCATCAAGAAGGAGATGTCTGGGGATGTCAGGGATG
CATTTGTGGCCATTGTTCAAAGTGTCAAGAACAAGCCTCTCTTCTTTG
CCGACAAACTTTACAAATCCATGAAGGGTGCTGGCACAGATGAGAAGA
CTCTGACCAGGATCATGGTATCCCGCAGTGAGATTGACCTGCTCAACA
TCCGGAGGGAATTCATTGAGAAATATGACAAGTCTCTCCACCAAGCCA
TTGAGGGTGACACCTCCGGAGACTTCCTGAAGGCCTTGCTGGCTCTCT
GTGGTGGTGAGGACTAGGGCCACAGCTTTGGCGGGCACTTCTGCCAAG
AAATGGTTATCAGCACCAGCCGCCATGGCCAAGCCTGATTGTTCCAGC
TCCAGAGACTAAGGAAGGGGCAGGGGTGGGGGGAGGGGTTGGGTTGGG
CTCTTATCTTCAGTGGAGCTTAGGAAACGCTCCCACTCCCACGGGCCA
TCGAGGGCCCAGCACGGCTGAGCGGCTGAAAAACCGTAGCCATAGATC
CTGTCCACCTCCACTCCCCTCTGACCCTCAGGCTTTCCCAGCTTCCTC
CCCTTGCTACAGCCTCTGCCCTGGTTTGGGCTATGTCAGATCCAAAAA
CATCCTGAACCTCTGTCTGTAAAATGAGTAGTGTCTGTACTTTGAATG
AGGGGGTTGGTGGCAGGGGCCAGTTGAATGTGCTGGGGGGGGTGGTGG
GAAGGATAGTAAATGTGCTGGGGCAAACTGACAAATCTTCCCATCCAT
TTCACCACCCATCTCCATCCAGGCCGCGCTAGAGTACTGGACCAGGAA
TTTGGATGCCTGGGTTCAAATCTGCATCTGCCATGCACTTGTTTCTGA
CCTTAGGCCAGCCCCTTTCCCTCCCTGAGTCTCTATTTTCTTATCTAC
AATGAGACAGTTGGACAAAAAAATCTTGGCTTCCCTTCTAACATTAAC
TTCCTAAAGTATGCCTCCGATTCATTCCCTTGACACTTTTTATTTCTA
AGGAAGAAATAAAAAGAGATACACAAACACATAAACACA

### Therapeutic Endpoints

In various aspects of the disclosure, use of the agent(s) and optional additional agent(s) as described herein provide one or more benefits related to specific therapeutic endpoints relative to a patient not receiving the agent(s) and/or additional agent(s).

Creatine kinase (CK) is a clinically validated serum biomarker of skeletal muscle, cardiac, kidney, and brain injury. Lactate dehydrogenase (LDH) is a clinically validated serum biomarker of skeletal muscle, cardiac, kidney, liver, lung, and brain injury. Creatine kinase and lactate dehydrogenase levels in serum are elevated with both acute and chronic tissue injury. In theoretical or verified conditions of comparable muscle mass levels, a reduction in creatine kinase and/or lactate dehydrogenase and/or cardiac troponin T and/or cardiac troponin I may be indicative of enhanced repair or protection against injury. Aspartate aminotransferase (AST) is yet another clinically validated serum biomarker of skeletal muscle, cardiac, kidney, liver, and brain injury. Additionally, increased serum troponin (e.g., cardiac troponin T and/or cardiac troponin I) is indicative of cardiac injury, while elevated alanine transaminase (ALT) is a biomarker of liver injury. Reduction in AST, ALT, or troponin in the acute period following injury may indicate enhanced repair or protection against injury. Evan's blue due is a vital dye that binds serum albumin and is normally excluded from healthy, intact muscle. Membrane disruption due to acute or chronic injury promotes the influx of dye into the damaged cell. Evan's blue dye is commonly used to quantify cellular damage in experimental settings, measuring inherent dye fluorescence and/or through measuring radiolabeled-dye uptake. Reduction in dye uptake after acute injury or in models of chronic damage would indicate protection against injury and/or enhanced repair. Indocyanine green (ICG) is a near-infared dye that binds plasma proteins and is used clinically to evaluate blood flow and tissue damage (ischemia; necrosis) in organs including heart, liver, kidney, skin, vasculature, lung, muscle and eye. Improved blood flow and reduction in ischemic areas indicate protection from injury and/or improved repair.

In some aspects, the disclosure provides methods of identifying patients who would benefit from administration of agent(s) and optional additional agent(s) of the disclosure. In any of the aspects or embodiments of the disclosure, such patients are identified by a serum or plasma level (e.g., an elevated level relative to a normal control range) of one or more biomarkers as described herein. Thus, in some embodiments such patients have not yet been diagnosed with a disease/disorder described herein. Accordingly, in some aspects, the disclosure provides methods comprising administering a therapeutically effective amount of an agent that increases the activity of an annexin protein to a patient, wherein the patient has an elevated serum or plasma level of lactate dehydrogenase (LDH), cardiac troponin T, cardiac troponin I, creatine kinase (CK), or a combination thereof, relative to a control level. In further aspects, the disclosure provides methods of reducing serum or plasma level of lactate dehydrogenase (LDH), cardiac troponin T, cardiac troponin I, creatine kinase (CK), or a combination thereof, in a patient in need thereof, comprising administering a therapeutically effective amount of an agent that increases the activity of an annexin protein to the patient, thereby reducing the serum or plasma level of lactate dehydrogenase (LDH), cardiac troponin T, cardiac troponin I, creatine kinase (CK), or a combination thereof in the patient relative to a previously measured serum or plasma level.

Serum or plasma levels of biomarkers described herein (e.g., lactate dehydrogenase (LDH), cardiac troponin T, cardiac troponin I, creatine kinase (CK), or a combination thereof) are measured via tests known in the art and described herein. These tests include, but are not limited to, standard clinical assays for molecule quantitation in blood, serum or plasma samples, such as enzymatic dosing (colorimetry), enzyme-linked immunosorbent assay (ELISA), radioimmunoassay (RIA), blood monitoring devices (glucometer). A “normal control range” of a biomarker level refers to the range that is present in a healthy population and is expressed in units that are particular to the assay used to measure the level of the biomarker (e.g., lactate dehydrogenase (LDH), cardiac troponin T, cardiac troponin I, creatine kinase (CK), or a combination thereof). It will be appreciated that different assays for measuring a level of a biomarker may yield results in different units. Thus, a “normal control range” expressed in units obtained using a particular assay may be numerically different than a “normal control range” expressed in units obtained using a different assay, but regardless of the assay that is used, the range present in a healthy population is known in the art. Strictly by way of example, a normal range of the serum or plasma level of LDH is about 140-280 U/L, a normal range of cardiac troponin T and/or cardiac troponin I is 10 ng/L for women, 15 ng/L for men, and a normal range of CK is about 22-198 U/L (and can vary with age, sex, race).

In some aspects, the disclosure provides methods of reducing serum or plasma level of lactate dehydrogenase (LDH), cardiac troponin T, cardiac troponin I, creatine kinase (CK), or a combination thereof, in a patient in need thereof, comprising administering a therapeutically effective amount of an agent that increases the activity of an annexin protein to the patient, thereby reducing the serum or plasma level of lactate dehydrogenase (LDH), cardiac troponin T, cardiac troponin I, creatine kinase (CK), or a combination thereof in the patient. In various embodiments, the serum or plasma level of LDH, cardiac troponin T, cardiac troponin I, CK, or a combination thereof in the patient prior to administration of the agent is elevated about 1.25-fold or more over a normal control range. In further embodiments, the serum or plasma level of LDH, cardiac troponin T, cardiac troponin I, CK, or a combination thereof in the patient prior to administration of the agent is elevated about, at least about, or less than about 1.25-fold, 1.5-fold, 2-fold, 2.5-fold, 3-fold, 4-fold, 5-fold, or more over a normal control range.

In various aspects, administration of one or more agents that increases the activity of an annexin protein results in a reduction in the serum or plasma level of one or more biomarkers as described herein (e.g., lactate dehydrogenase (LDH), cardiac troponin T, cardiac troponin I, creatine kinase (CK), or a combination thereof) in the patient. The reduction is, for example, relative to a previous serum or plasma level of the biomarker(s) in the patient. In some embodiments, following administration of one or more agents that increase the activity of an annexin protein there is about a 25% reduction in the serum or plasma level of a biomarker of the disclosure (e.g., lactate dehydrogenase (LDH), cardiac troponin T, cardiac troponin I, creatine kinase (CK), or a combination thereof) relative to a previously measured level of the biomarker. In further embodiments, following administration of one or more agents that increase the activity of an annexin protein there is about, at least about, or less than about a 5%, 10%, 15%, 20%, 25%, 30%, 35%, 40%, 50%, 60%, or 70% reduction in the serum or plasma level of a biomarker of the disclosure relative to a previously measured level of the biomarker. In some embodiments, the serum or plasma level of a biomarker is measured about or at least about 24 hours after administration of the agent. In some embodiments, the serum or plasma level of a biomarker is measured about 24-48, 24-72, or 48-72 hours after administration of the agent. In further embodiments, the serum or plasma level of a biomarker is measured about, less than about, or at least about, 24, 30, 36, 48, 60, or 72 hours after administration of the agent.

It is also contemplated that increasing membrane integrity and repair results in enhanced function measured through multiple modalities including plethysmography, echocardiography, muscle force, 6-min walk test. Additionally, histological benefits may be noted, including decreased necrosis, decreased inflammation, reduced fibrosis, reduced fatty infiltrate and reduced edema. These beneficial effects may also be visible through MR and PET imaging.

### Dosing/Administration/Kits

A particular administration regimen for a particular subject will depend, in part, upon the agent and optional additional agent used, the amount of the agent and optional additional agent administered, the route of administration, the particular ailment being treated, and the cause and extent of any side effects. The amount of agent and optional additional agent administered to a subject (e.g., a mammal, such as a human) is sufficient to effect the desired response. Dosage typically depends upon a variety of factors, including the particular agent and/or additional agent employed, the age and body weight of the subject, as well as the existence and severity of any disease or disorder in the subject. The size of the dose also will be determined by the route, timing, and frequency of administration. Accordingly, the clinician may titer the dosage and modify the route of administration to obtain optimal therapeutic effect, and conventional range-finding techniques are known to those of ordinary skill in the art. Purely by way of illustration, in some embodiments, the method comprises administering an agent (e.g., a protein), e.g., from about 0.1 pg/kg up to about 100 mg/kg or more, depending on the factors mentioned above. In other embodiments, the dosage may range from 1 μg/kg up to about 75 mg/kg; or 5 μg/kg up to about 50 mg/kg; or 10 μg/kg up to about 20 mg/kg. In certain embodiments, the dose comprises about 0.5 mg/kg to about 20 mg/kg (e.g., about 1 mg/kg, 1.5 mg/kg, 2 mg/kg, 2.3 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 8 mg/kg, 9 mg/kg, or 10 mg/kg) of agent and optional additional agent. In embodiments in which an agent and additional agent are administered, the above dosages are contemplated to represent the amount of each agent administered, or in further embodiments the dosage represents the total dosage administered. In some embodiments wherein a chronic condition is treated, it is envisioned that a subject will receive the agent and/or additional agent over a treatment course lasting weeks, months, or years, and may require one or more doses daily or weekly. In some embodiments, a subject will receive the agnet and/or additional agent via continuous infusion. In any of the aspects or embodiments of the disclosure, the amount of an annexin protein in a pharmaceutical composition is from about 0.1 pg/kg up to about 100 mg/kg or more, depending on the factors mentioned above. In other embodiments, the dosage may range from 1 μg/kg up to about 75 mg/kg; or 5 μg/kg up to about 50 mg/kg; or 10 μg/kg up to about 20 mg/kg. In some embodiments, the dose comprises about 0.5 mg/kg to about 20 mg/kg (e.g., about 1 mg/kg, 1.5 mg/kg, 2 mg/kg, 2.3 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 8 mg/kg, 9 mg/kg, or 10 mg/kg) of annexin protein. Dosages are also contemplated for once daily, twice daily (BID) or three times daily (TID) dosing. A unit dose may be formulated in either capsule or tablet form. In other embodiments, the agent and optional additional agent is administered to treat an acute condition (e.g., acute muscle injury or acute myocardial injury) for a relatively short treatment period, e.g., one to 14 days.

Suitable methods of administering a physiologically-acceptable composition, such as a pharmaceutical composition comprising an agent (e.g., a recombinant protein) and optional additional agent described herein, are well known in the art. Although more than one route can be used to administer an agent and/or additional agent, a particular route can provide a more immediate and more effective avenue than another route. Depending on the circumstances, a pharmaceutical composition is applied or instilled into body cavities, absorbed through the skin or mucous membranes, ingested, inhaled, and/or introduced into circulation. In some embodiments, a composition comprising an agent and/or additional agent is administered intravenously, intraarterially, or intraperitoneally to introduce an agent and optional additional agent into circulation. Non-intravenous administration also is appropriate, particularly with respect to low molecular weight therapeutics. In certain circumstances, it is desirable to deliver a pharmaceutical composition comprising the agent and/or additional agent orally, topically, sublingually, vaginally, rectally; through injection by intracerebral (intra-parenchymal), intracerebroventricular, intramuscular, intra-ocular, intraportal, intralesional, intramedullary, intrathecal, intraventricular, transdermal, subcutaneous, intranasal, urethral, or enteral means; by sustained release systems; or by implantation devices. If desired, the agent and/or additional agent is administered regionally via intraarterial or intravenous administration to a region of interest, e.g., via the femoral artery for delivery to the leg. In one embodiment, the composition is administered via implantation of a membrane, sponge, or another appropriate material within or upon which the desired agent and optional additional agent has been absorbed or encapsulated. Where an implantation device is used, the device in one aspect is implanted into any suitable tissue, and delivery of the desired agent and/or additional agent is, in various embodiments, effected via diffusion, time-release bolus, or continuous administration. In other embodiments, the agent and optional additional agent is administered directly to exposed tissue during surgical procedures or treatment of injury, or is administered via transfusion of blood products. Therapeutic delivery approaches are well known to the skilled artisan, some of which are further described, for example, in U.S. Pat. No. 5,399,363.

In some embodiments facilitating administration, the agent and optional additional agent in one embodiment is formulated into a physiologically acceptable composition comprising a carrier (i.e., vehicle, adjuvant, buffer, or diluent). The particular carrier employed is limited only by chemico-physical considerations, such as solubility and lack of reactivity with the agent and/or additional agent, by the route of administration, and by the requirement of compatibility with the recipient organism. Physiologically acceptable carriers are well known in the art. Illustrative pharmaceutical forms suitable for injectable use include, without limitation, sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers. eds., pages 238-250 (1982), and ASHP Handbook on Injectable Drugs, Toissel, 4th ed., pages 622-630 (1986), incorporated herein by reference).

A pharmaceutical composition comprising an agent (e.g., a recombinant protein) and optional additional agent as provided herein is optionally placed within containers/kits, along with packaging material that provides instructions regarding the use of such pharmaceutical compositions. Generally, such instructions include a tangible expression describing the reagent concentration, as well as, in certain embodiments, relative amounts of excipient ingredients or diluents that may be necessary to reconstitute the pharmaceutical composition.

The disclosure thus includes, in various embodiments, administering to a subject one or more agent(s), in combination with one or more additional agent(s), each being administered according to a regimen suitable for that medicament. In some embodiments, the agent is a recombinant protein such as an annexin protein (e.g., annexin A6). Administration strategies include concurrent administration (i.e., substantially simultaneous administration) and non-concurrent administration (i.e., administration at different times, in any order, whether overlapping or not) of the agent and one or more additional agents(s). It will be appreciated that different components are optionally administered in the same or in separate compositions, and by the same or different routes of administration.

All publications, patents and patent applications cited in this specification are herein incorporated by reference as if each individual publication or patent application were specifically and individually indicated to be incorporated by reference. In addition, the entire document is intended to be related as a unified disclosure, and it should be understood that all combinations of features described herein are contemplated, even if the combination of features are not found together in the same sentence, or paragraph, or section of this document. For example, where protein therapy is described, embodiments involving polynucleotide therapy (using polynucleotides/vectors that encode the protein) are specifically contemplated, and the reverse also is true. With respect to elements described as one or more members of a set, it should be understood that all combinations within the set are contemplated.

### EXAMPLES

### Example 1

This Example describes experiments designed to assess the impact physiologic mechanical stress on healthy control and patient-derived Duchenne-muscular dystrophy (DMD) induced pluripotent stem cell-derived cardiomyocytes (iPSC-CMs) and to use this platform to test recombinant annexin A6 as a membrane resealing agent.

Heart failure is a major source of mortality in Duchenne muscular dystrophy (DMD). DMD arises from mutations that ablate expression of the protein dystrophin, disrupting the dystrophin-associated protein complex and rendering the plasma membrane unusually fragile and prone to disruption. In DMD patients, cardiomyopathy develops from repeated mechanical stress leading to membrane damage and cardiomyocyte loss. Induced pluripotent stem cell-derived cardiomyocytes (iPSC-CMs) offer the opportunity to study specific mutations in the context of a human cell, but these models can be improved by modulating conditions including the use of physiologic stressors. In this example, the primary defect underlying DMD was modeled by subjecting DMD iPSC-CMs to equibiaxial mechanical strain. DMD iPSC-CMs demonstrated an increased susceptibility to equibiaxial strain after 2 hours at 10% strain relative to control, measured by increased lactate dehydrogenase (LDH) release. After 24 hours, both DMD and healthy control iPSC-CMs showed evidence of injury with release of both LDH and cardiac troponin T. The iPSC-CMs were exposed to recombinant annexin A6, a protein resealing agent, and reduced LDH and troponin release was found in DMD and control iPSC-CMs that had been subjected to 24 hour strain at 10%. By developing an injury assay that specifically targets the mechanism of injury seen in DMD-related cardiomyopathy, the potential therapeutic efficacy of protein membrane resealer, recombinant annexin A6, for the treatment of DMD-related cardiomyopathy and general cardiac injury, was demonstrated.

### Introduction

Duchenne muscular dystrophy (DMD) is an X-linked disease that results from mutations in DMD, which codes for the protein dystrophin (1). Clinically, DMD presents in the first decade with weakness and markedly elevated serum biomarkers like creatine kinase (2). Cardiac involvement is uniformly present by the second decade and contributes to morbidity and mortality in DMD (3). Dystrophin localizes to the plasma membrane and is concentrated in the membrane above the Z-disc, colocalizing with other proteins of the dystrophin complex, including the sarcoglycans and dystroglycans (4-6). This complex forms a critical transmembrane structural and signaling connection between the sarcomere and the extracellular matrix (4,7-10). Disruptions along this axis produce membrane fragility and account for multiple forms of muscular dystrophy with cardiac involvement (11-13). Daily glucocorticoid administration is the mainstay of current therapy, which delays a loss of mobility by 2-4 years (14). While early initiation of ACE inhibitors has been shown to delay the progression of cardiomyopathy (15-17), other aspects of cardiomyopathy prevention and treatment of heart failure rely on standard heart failure strategies (3,18). Novel therapeutics for the treatment of DMD are currently under investigation, including gene replacement therapy with micro-dystrophins, gene editing approaches, and membrane re-sealants (19-22). Several antisense-mediated exon skipping agents are now approved for use in DMD, but these agents have relatively poor penetration into the myocardium and are useful for less than 25% of DMD mutations (23,24). For clinical agents that treat skeletal muscle in DMD, most studies have relied on endpoints like time to loss of mobility or measures of muscle strength or performance. Clinical trials for DMD cardiomyopathy are complicated by patients having reduced or no ambulatory capabilities (24).

iPSC-derived cardiomyocytes (iPSC-CMs) are now commonly used to evaluate patient-specific therapies in a human cell context (25). However, iPSC-CMs are immature in nature and are generally cultured under conditions that fail to mimic the load and strain seen by the human heart (26,27). Engineered heart tissues promote greater cellular maturity compared to two-dimensional iPSC-CM cultures, and the most common methods rely on allowing cells to organize around microfabricated anchor points (28,29). Despite progress with engineered heart tissues, approaches to evaluate dynamic physiologic mechanical stress are still under development. The effects of mechanical stress have previously been studied to understand early signaling responses that lead to cardiac hypertrophy (30) and pathological signaling responses in nuclear membrane defects (31), and these prior studies utilized rat neonatal cardiomyocytes or mouse embryonic fibroblasts. The differential response of mechanical stress on DMD and healthy control iPSC-CMs and the response to a resealing protein, recombinant annexin A6, which was previously identified as a genetic modifier of muscular dystrophy and a potential therapeutic target (32,33), was investigated.

### Methods

Briefly, DMD and healthy control iPSC-CMs were cultured and subjected to 5, 10, and 15% equibiaxial strain for 2 and 24 h time periods. Release of the clinically relevant biomarkers lactate dehydrogenase (LDH) and cardiac troponin T were measured, and the effects of recombinant annexin A6 on biomarker release were determined.

iPSC generation, iPSC culture, cardiac differentiation, enrichment, and expansion. Urine-derived epithelial cells were obtained from a DMD patient and reprogrammed using published methods to generate the cell line DMD-G01 (34). The control line iPSC line (GM033488) has been previously published (35). iPSC culture and differentiation were performed per previously published methods (35,36). At day 8-10 post initiation of differentiation with CHIR99021, iPSC-CMs were harvested by collagenase digestion for 2 h per Breckwoldt et al (28) with the following modified digestion solution: 1 mg/mL collagenase II mg/mL (Worthington, LS0041762), 10 mM HEPES, 2 μM thiazovivin (Stemcell Technologies, 72254), and 30 pM N-benzyl-p-toluenesulfonamide (TCI, B3082-5G) in Hank's balanced salt solution (Gibco, 14175095). iPSC-CMs were separated from non-cardiomyocytes by magnetic labeling of non-iPSC-CMs, using a commercially available kit (Miltenyi Biotec, 130-110-188). Manufacturer instructions were followed, with the following modifications: (1) MACS buffer was defined as 0.5% KOSR (Gibco, 10828028), 2 mM EDTA in calcium and magnesium free DPBS (Gibco, 14190144) and (2) only the first negative selection step was performed, and the second positive selection step was omitted. Enriched iPSC-CMs were expanded in a protocol adapted from Buikema et al (37). iPSC-CMs were replated at 2 million cells per 10 cm plate in B27 (Gibco, 17-504-044) in RPMI 1640 (Gibco, 11875101), 2 pM thiazovivin, and 10% KOSR in RPMI 1640. 10 cm plates were coated with 1:400 Matrigel® (Corning, 354277) in DMEM/F12 (Corning, MT10090CV) for at least 1 h prior to replating. After 24 h media was exchanged with 2 pM CHIR99021 (Tocris 4423) in B27 in RPMI 1640 and exchanged every 48 hours. After 7-10 days of expansion, cells were confluent and harvested for downstream applications by collagenase digestion protocol as above.

Preparation of flexible membranes and application of equibiaxial strain. Silanization of flexible membrane 6 well plates (Bioflex® culture plates, FlexCell International) was performed by adding 1 mL 5% (v/v) 3-aminopropyltriethoxysilane (Acros, AC430941000) in 95% ethanol for 10 min. The solution was aspirated, and 1 mL 100% ethanol was added and immediately aspirated. Plates were incubated at 65 C for 20 minutes, washed once with 1 mL 95% ethanol, twice with 2 mL DPBS, and once with DI water. Plates were then coated with 3 mL 1:400 Matrigel® as per iPSC-CM expansion protocol. Expanded iPSC-CMs were harvested by collagenase digestion as above and plated at a density of 1.5 million cells/well in B27 in RPMI 1640 with 10% FBS (Gibco, 26140079) and 1% penicillin/streptomycin (Gibco, 15070063). Media was exchanged with B27 in RPMI 1640 and 1% penicillin/streptomycin, every-other day. On day 7 post-replating, media was exchanged with fresh B27 in RPMI 1640 and cyclic sinusoidal equibiaxial strain at 1 Hz was applied using a FX-6000T™ Tension System (FlexCell International).

iPSC cardiomyocyte troponin T staining and flow cytometry analysis. iPSC-CMs were collected before or after enrichment in initial experiments and at the time of replating onto flexible membranes for all differentiations. All centrifugation steps in this protocol were performed at 600 g for 5 min. 500,000-1,000,000 cells were resuspended in 2 mL DPBS in FACS tubes (Falcon, 352057), centrifuged and decanted. Cells were resuspended in 1 mL DPBS and 1 mL 8% paraformaldehyde (EMS, 15710) in DPBS was added. Cells were incubated in a 37 C shaker for 10 min and then centrifuged and decanted. Cells were resuspended in 200 μL of ice cold 90% methanol, 10% DPBS and stored at −20 C until staining for flow cytometry. Fresh incubation buffer was prepared: 0.5% w/v bovine serum albumin (Sigma Aldrich, A7906) in DPBS. 1:200 Alexa Fluor®647 mouse anti-cardiac troponin T (BD Biosciences 565744) and 1:200 Alexa Fluor®647 mouse IgG1 κ isotype control (BD Biosciences 557732) was prepared in incubation buffer. Cells were split evenly into two FACS tubes and 2 mL incubation buffer was added to each tube and centrifuged and decanted. Cells were incubated with 100 μL primary antibody or isotype control solution and incubated at room temperature in the dark for 1 h. 4 mL incubation buffer was then added to each tube, they were then centrifuged and decanted. Cells were then resuspended in 100 μL DPBS and analyzed with a BD Accuri C6 Plus flow cytometer. If iPSC-CM purity was <85%, cells were rejected for downstream applications.

Recombinant annexin A6. iPSC-CMs were treated with recombinant annexin A6 (33) at a concentration of 10 μg/mL. In the case of binding studies, iPSC-CMs were strained at 10% for 23 h, followed by addition of recombinant annexin A6-488, which was strained for 1 additional h, incubated for an additional 2 hours, followed by 2×2 mL wash with Hanks balanced salt solution (Gibco, 14175095), collagenase digestion as described above, harvested after 2 h by quenching with an equal volume of media, centrifuged for 10 m at 100 g, resuspended in 100 μL DPBS and analyzed by flow cytometry as described above.

Biomarker measurement. LDH and cardiac troponin T release was quantified per manufacturer instructions using Promega LDH-Glo™ Cytotoxicity Assay (Promega J2380) and human cardiac troponin T ELISA kit (Abcam, ab223860).

Statistical methods. Data was analyzed using Prism 9.3.0. Where comparisons of two conditions were made an unpaired t test was used. Where comparisons of more than two conditions were made an ordinary one-way ANOVA was used with Tukey correction for multiple comparisons. In all cases, p<0.05 was defined as statistically significant. Statistical data is reported as mean±SEM. Confidence intervals are reported as 95% (95% Cl).

### Results

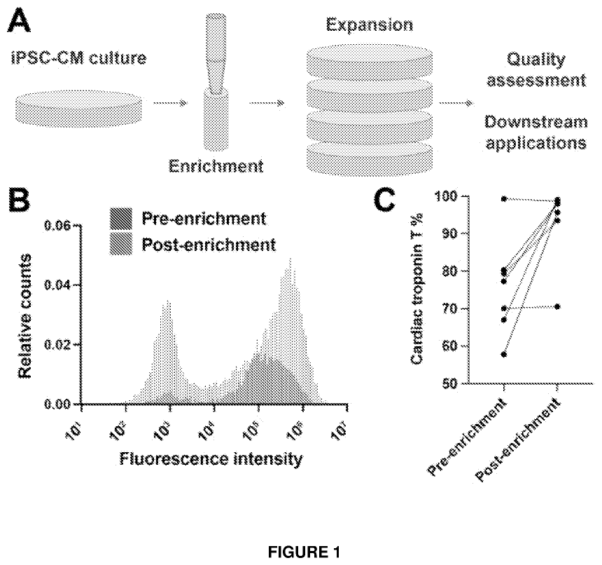

Generation, differentiation and expansion of high-quality iPSC-CMs. IPSCs were generated from a DMD patient who with an out of frame, large deletion spanning DMD exons (46-47). The patient had a typical DMD course with loss of ambulation before the age of 11 and developed an associated severe cardiomyopathy with LVEF ˜10% despite guideline directed therapy and biventricular chronic resynchronization therapy. To improve variability in iPSC-CM differentiation, a two-step iPSC-CM enrichment was employed (FIG. 1A). iPSCs were initially differentiated into iPSC-CMs by conventional methods (35,36), followed by a second step in which iPSC-CMs were enriched using a magnetic separation system. Assessment, pre- and post-enrichment by magnetic separation confirmed improved cardiac troponin T positivity (FIGS. 1B and 1C). This enriched iPSC-CM cell population was then expanded by adapting a recently published method (37). Combining iPSC-CM enrichment with expansion generated sufficient numbers of high-quality iPSC-CMs for downstream applications.

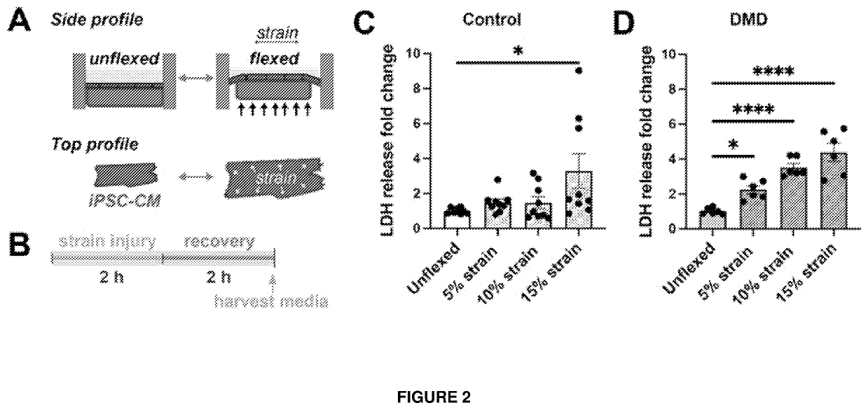

Increased susceptibility of dystrophic iPSC-CMs to mechanical strain. Dystrophin deficient cardiomyocytes from animal models have increased susceptibility to mechanical stress relative to controls (19,38). Similarly, serum biomarkers reflective of membrane leak are elevated in DMD patients (39,40). Therefore, it was initially sought to define a physiologic degree of mechanical stress to impart on iPSC-CMs that differentiated DMD iPSC-CMs from healthy control iPSC-CMs. iPSC-CMs were plated onto flexible membranes in a 6 well plate format and radial deformation was applied to impart a homogenous equibiaxial strain on plated cells in vitro (FIG. 2A). Healthy control iPSC-CMs and DMD iPSC-CMs were subjected to 2 h of 0% (unflexed), 5%, 10% or 15% strain and the cell culture media was collected for biomarker determination (FIG. 2B). Lactate dehydrogenase (LDH) is a clinically relevant serum biomarker of tissue injury, including cardiac injury (41). Control iPSC-CM media LDH levels after 5% and 10% strain remained similar to that of unflexed cells (FIG. 2C). At 15% strain there was an increase LDH release, however there was also an increase in the variability of the data, likely from the severity of the injury. Media collected from DMD iPSC-CMs showed a dose-dependent increase in LDH levels following strain injury (FIG. 2D), demonstrating that dystrophic iPSC-CMs are more susceptible to strain-induced injury compared to control iPSC-CMs. Similar to control iPSC-CMs, the variability of LDH release for DMD iPSC-CMs increased at 15% strain, likely related to the severe injury at this high level of strain. Based on initial considerations to define a physiologic degree of mechanical stress, it was observed that 10% strain did not result in significant LDH release in control iPSC-CMs, while it resulted in a significant increase LDH release in DMD iPSC-CMs. Thus, subsequent experiments were performed at 10% strain.

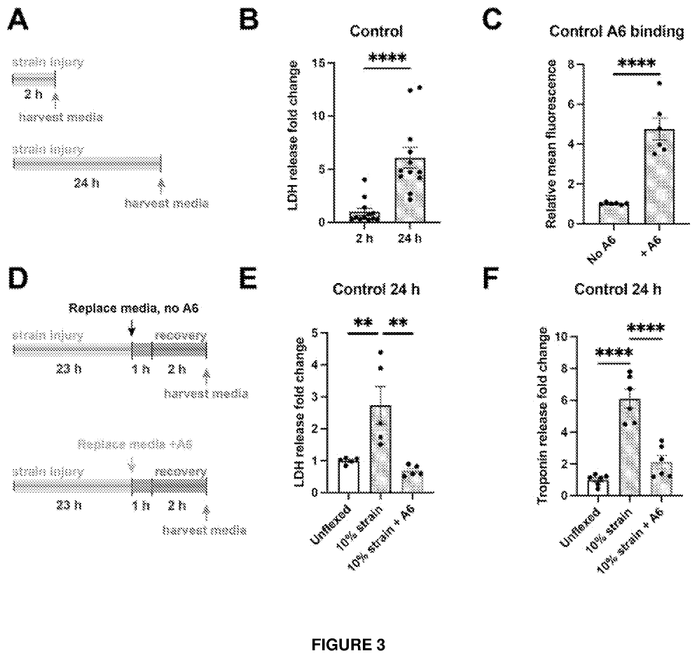

Recombinant annexin A6 promotes membrane repair in control iPSC-CMs. Having defined a strain exposure that differentiated between DMD and healthy control iPSC-CMs, whether applying longer exposure to strain could induce injury in healthy control iPSC-CMs (FIG. 3A) was tested. As shown in FIG. 3B, LDH release fold change increased by 5.1 (95% Cl: 2.9 to 7.2, ****p<0.0001) after 24 h of flexing compared to the non-injury-inducing 2 h time period. This demonstrated that 24 h of 10% strain surpassed the threshold required to induce mechanical membrane injury sufficient for LDH leak in control iPSC-CMs. Recombinant annexin A6 was previously shown to promote resealing in mouse skeletal myofibers injured with a laser (32,33), so the efficacy of recombinant annexin A6 to enhance repair in cardiomyocytes injury using this mechanical injury model was assessed. It was first assessed whether fluorescently labelled recombinant annexin A6 binds control iPSC-CMs that had been strained. As show in FIG. 3C, relative mean fluorescent intensity increased by 3.7 (95% Cl: 2.5 to 5.0, ****p<0.0001) in treated compared to untreated control iPSC-CMs as assessed by flow cytometry, consistent with recombinant annexin A6-iPSC-CM binding. FIG. 3D depicts the experimental strategy for assessing membrane repair and response to recombinant annexin A6 in which membrane damage is followed by exposure to recombinant annexin A6 or vehicle, and then strain was continued for 1 h, followed by a 2 h recovery period. In the absence of annexin A6, strain resulted in an increase in LDH release fold change of 1.7 (95% Cl: 0.4 to 3.0, **p=0.01) compared to unflexed controls (FIG. 3E). When annexin A6 is present during the post-injury recovery period, LDH levels were similar to LDH levels of unflexed cells (95% Cl: −1.6 to 1.0, p=0.79). To corroborate these findings, troponin release was also quantified (FIG. 3F), and was found to be similarly increase with application of mechanical stress (95% Cl: 3.5 to 6.7, ****p<0.0001) and reduce to near baseline levels with recombinant annexin A6 treatment (95% Cl: −0.4 to 1.1, p=0.19). Together these data demonstrated that recombinant annexin A6 promoted repair of injured control iPSC-CMs.

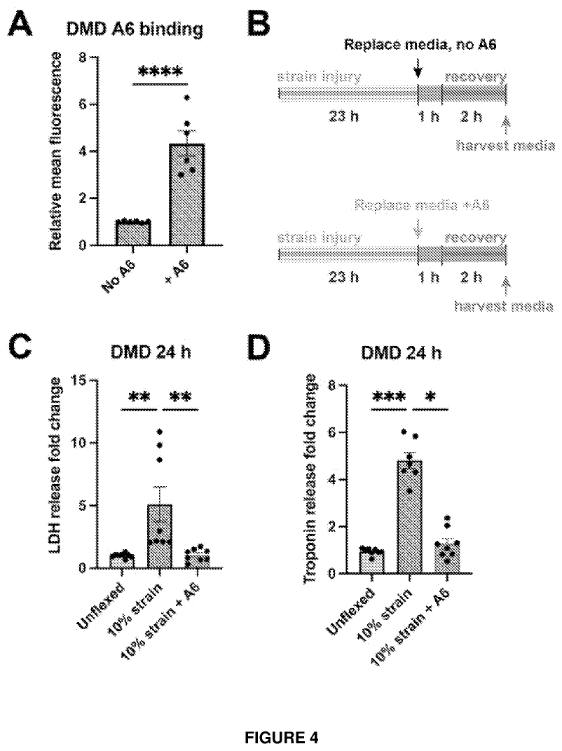

Annexin A6 promotes dystrophic iPSC-CM membrane repair. Knowing that dystrophic cells are highly prone to membrane injury, it was determined whether recombinant annexin A6 could enhance repair of severely injured DMD iPSC-CMs. Fluorescently labelled recombinant annexin A6 binding to DMD iPSC-CMs after a 24 h strain protocol was first assessed. As shown in FIG. 4A, relative mean fluorescent intensity increased by 3.4 (95% Cl: 2.2 to 4.5, ****p<0.0001) in treated strained DMD iPSC-CMs, demonstrating recombinant annexin A6 binding. DMD iPSC-CMs were subjected to the same 24 h, 10% strain injury protocol that is capable of injuring control iPSC-CMs (FIG. 4B). When recombinant annexin A6 was absent during the post-injury recovery period, LDH release fold change increased by 4.1 (95% Cl: 1.2 to 7.0, **p=0.005) compared to unflexed DMD iPSC-CMs. FIG. 4C shows that with the addition of recombinant annexin A6 during the post-injury recovery period, LDH levels were similar to unflexed iPSC-CM media (95% Cl: −2.8 to 2.9, p=0.9989) and significantly less than in media from flexed cells lacking annexin A6 (95% Cl: 1.2 to 6.9, **p=0.005). Troponin release fold change mirrored LDH levels, increasing 3.9 (95% Cl: 3.1 to 4.7, ****p<0.0001) post-injury in the absence of annexin A6 compared to unflexed controls (FIG. 4D). Treatment with recombinant annexin A6, reduced fold change troponin levels by 3.5 (95% Cl: 2.7 to 4.3, ****p<0.0001) with no significant difference compared to the unflexed condition (95% Cl: −0.4 to 1.1, p=0.52). Consistent with the effects seen on membrane repair in control iPSC-CMs, these results demonstrate efficacy of recombinant annexin A6 in promoting membrane in dystrophic iPSC-CMs.

DMD iPSC-CMs demonstrated an increased susceptibility to mechanical stress relative to healthy (non-dystrophic) controls seen as increased LDH release. Healthy control cells required equibiaxial strain for a longer duration to sustain a significant increase in LDH and troponin release. DMD iPSC-CM and healthy controls each demonstrated a decrease in LDH and troponin release after recombinant annexin A6 treatment.

### Discussion

In vivo, cardiomyocytes are under constant cyclic stress due to the cardiac cycle. Membrane damage and repair are part of normal physiology, however certain diseases are associated with excessive membrane damage (42,43). Previous work has demonstrated in both the in vitro and in vivo setting that physiologic stress of the rat myocardium with isoproterenol induces transient membrane damage (43). Consistent with the role of dystrophin in skeletal muscle, the mdx mouse, which harbors a premature stop codon in DMD and is the most commonly studied DMD animal model, has demonstrated an increased susceptibility to cardiomyocyte membrane injury by an increase in afterload or treatment with isoproterenol (38). Collectively, the literature supports membrane fragility as the primary deficit in DMD and consequent membrane damage as the initial insult with a host of downstream consequences (11-13, 19,20), and this is reflected by elevated serum biomarkers from both skeletal and cardiac origin in DMD patients (39,40).

Human iPSCs offer the advantage of harboring human specific mutations in an appropriate cellular environment (27,44). However, despite the ability to generate of iPSC-CMs, the conditions under which most cells are studied fail to simulate afterload and preload, and in the case of DMD cardiomyopathy, this is critical to creating micro-injury in the plasma membrane. Three dimensional engineered heart tissues can be used to improve the maturity of iPSC-CMs enabling measurements of contractility; however, at present, there is no readily available method for imparting dynamic mechanical stress (28). In a recent report, Sewanan et and colleagues simulated pressure volume loops in decellularized porcine myocardium engineered heart tissue seeded with iPSC-CMs (45). While precise time-dependent tailoring of the preload and afterload in an engineered heart tissue is a promising technique for studying the effects of cardiac therapeutics, at present, it requires a significant degree of technical engineering expertise and cannot easily be ported into a high-throughput method for studying drug effects. By employing flexible membranes capable of deformation by equibiaxial strain, iPSC-CMs were successfully stressed in a physiologically meaningful way for the study of DMD-associated cardiomyopathy with clinically relevant biomarker outputs.

This Example demonstrated an ability to enhance membrane repair in injury that occurs in human DMD iPSC-CMs. Furthermore, recombinant annexin A6 also promoted resealing of healthy control iPSC-CMs, highlighting a conserved mechanism in both normal and diseased cells.

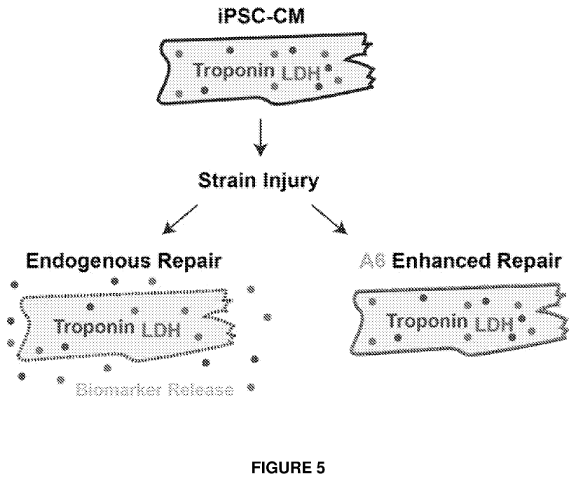

The schematic in FIG. 5 shows that recombinant annexin A6 enhances endogenous cardiomyocyte membrane repair processes. Given these findings, recombinant annexin A6 is useful in treating genetic forms of cardiomyopathy that lead to increased baseline membrane fragility, as well as pathologic injury such as myocardial infarction or acute pressure overload where activation of membrane repair processes are essential for recovery from an acute insult.

In this Example, an in vitro assay using physiologic mechanical stress by application of equibiaxial strain to iPSC-CMs was described. Using this assay, a dose dependent increase in susceptibility of biomarker release in DMD iPSC-CMs relative to controls, as well as response to a protein resealing therapeutic, was demonstrated. Specifically, it was demonstrated that recombinant annexin A6 is a protein-based membrane resealer that enhanced membrane repair after mechanical stress in Duchenne cardiomyocytes and non-dystrophic, control cardiomyocytes.

In summary, the data provided herein showed that recombinant annexin A6 promoted membrane resealing and reduced biomarker levels in DMD and healthy control iPSC-CMs, demonstrating its use as a treatment for DMD-related cardiomyopathy and general cardiac injury.

### REFERENCES CITED IN EXAMPLE 1

1. Koenig M, Hoffman E P, Bertelson C J, Monaco A P, Feener C, Kunkel L M. Complete cloning of the Duchenne muscular dystrophy (DMD) cDNA and preliminary genomic organization of the DMD gene in normal and affected individuals. Cell 1987; 50:509-17.
2. Bushby K, Finkel R, Birnkrant D J et al. Diagnosis and management of Duchenne muscular dystrophy, part 1: diagnosis, and pharmacological and psychosocial management. Lancet Neurol 2010; 9:77-93.
3. McNally E M, Kaltman J R, Benson D W et al. Contemporary cardiac issues in Duchenne muscular dystrophy. Working Group of the National Heart, Lung, and Blood Institute in collaboration with Parent Project Muscular Dystrophy. Circulation 2015; 131:1590-8.
4. Campbell K P, Kahl S D. Association of dystrophin and an integral membrane glycoprotein. Nature 1989; 338:259-62.
5. Ervasti J M, Kahl S D, Campbell K P. Purification of dystrophin from skeletal muscle. J Biol Chem 1991; 266:9161-5.
6. Ervasti J M, Campbell K P. Membrane organization of the dystrophin-glycoprotein complex. Cell 1991; 66:1121-31.
7. Briggs D C, Yoshida-Moriguchi T, Zheng T et al. Structural basis of laminin binding to the LARGE glycans on dystroglycan. Nat Chem Biol 2016; 12:810-4.
8. Ibraghimov-Beskrovnaya O, Ervasti J M, Leveille C J, Slaughter C A, Sernett S W, Campbell K P. Primary structure of dystrophin-associated glycoproteins linking dystrophin to the extracellular matrix. Nature 1992; 355:696-702.
9. Rybakova I N, Patel J R, Ervasti J M. The dystrophin complex forms a mechanically strong link between the sarcolemma and costameric actin. J Cell Biol 2000; 150:1209-14.
10. Klietsch R, Ervasti J M, Arnold W, Campbell K P, Jorgensen A O. Dystrophin-glycoprotein complex and laminin colocalize to the sarcolemma and transverse tubules of cardiac muscle. Circ Res 1993; 72:349-60.
11. Bloch R J, Gonzalez-Serratos H. Lateral force transmission across costameres in skeletal muscle. Exerc Sport Sci Rev 2003; 31:73-8.
12. Ervasti J M. Costameres: the Achilles' heel of Herculean muscle. J Biol Chem 2003; 278:13591-4.
13. Townsend D, Yasuda S, McNally E, Metzger J M. Distinct pathophysiological mechanisms of cardiomyopathy in hearts lacking dystrophin or the sarcoglycan complex. FASEB J 2011; 25:3106-14.
14. McDonald C M, Henricson E K, Abresch R T et al. Long-term effects of glucocorticoids on function, quality of life, and survival in patients with Duchenne muscular dystrophy: a prospective cohort study. Lancet 2018; 391:451-461.
15. Duboc D, Meune C, Pierre B et al. Perindopril preventive treatment on mortality in Duchenne muscular dystrophy: 10 years' follow-up. Am Heart J 2007; 154:596-602.
16. Duboc D, Meune C, Lerebours G, Devaux J Y, Vaksmann G, Becane H M. Effect of perindopril on the onset and progression of left ventricular dysfunction in Duchenne muscular dystrophy. J Am Coll Cardiol 2005; 45:855-7.
17. Silva M C, Magalhaes T A, Meira Z M et al. Myocardial Fibrosis Progression in Duchenne and Becker Muscular Dystrophy: A Randomized Clinical Trial. JAMA Cardiol 2017; 2:190-199.
18. Yancy C W, Jessup M, Bozkurt B et al. 2017 ACC/AHA/HFSA Focused Update of the 2013 ACCF/AHA Guideline for the Management of Heart Failure: A Report of the American College of Cardiology/American Heart Association Task Force on Clinical Practice Guidelines and the Heart Failure Society of America. Circulation 2017; 136:e137-e161.
19. Yasuda S, Townsend D, Michele D E, Favre E G, Day S M, Metzger J M. Dystrophic heart failure blocked by membrane sealant poloxamer. Nature 2005; 436:1025-9.
20. Houang E M, Sham Y Y, Bates F S, Metzger J M. Muscle membrane integrity in Duchenne muscular dystrophy: recent advances in copolymer-based muscle membrane stabilizers. Skelet Muscle 2018; 8:31.
21. Duan D. Systemic AAV Micro-dystrophin Gene Therapy for Duchenne Muscular Dystrophy. Mol Ther 2018; 26:2337-2356.
22. Kyrychenko V, Kyrychenko S, Tiburcy M et al. Functional correction of dystrophin actin binding domain mutations by genome editing. JCIInsight 2017; 2.
23. Sheikh O, Yokota T. Pharmacology and toxicology of eteplirsen and SRP-5051 for DMD exon 51 skipping: an update. Arch Toxicol 2022; 96:1-9.
24. Johnston J R, McNally E M. Genetic correction strategies for Duchenne Muscular Dystrophy and their impact on the heart. Prog Pediatr Cardiol 2021; 63.
25. Sayed N, Liu C, Wu J C. Translation of Human-Induced Pluripotent Stem Cells: From Clinical Trial in a Dish to Precision Medicine. J Am Coll Cardiol 2016; 67:2161-2176.
26. Karakikes I, Ameen M, Termglinchan V, Wu J C. Human induced pluripotent stem cell-derived cardiomyocytes: insights into molecular, cellular, and functional phenotypes. Circ Res 2015; 117:80-8.
27. Tu C, Chao B S, Wu J C. Strategies for Improving the Maturity of Human Induced Pluripotent Stem Cell-Derived Cardiomyocytes. Circ Res 2018; 123:512-514.
28. Breckwoldt K, Letuffe-Breniere D, Mannhardt I et al. Differentiation of cardiomyocytes and generation of human engineered heart tissue. Nat Protoc 2017; 12:1177-1197.
29. Stein J M, Mummery C L, Bellin M. Engineered models of the human heart: Directions and challenges. Stem Cell Reports 2021; 16:2049-2057.
30. Yamamoto K, Dang Q N, Maeda Y, Huang H, Kelly R A, Lee R T. Regulation of cardiomyocyte mechanotransduction by the cardiac cycle. Circulation 2001; 103:1459-64.
31. Lammerding J, Schulze P C, Takahashi T et al. Lamin A/C deficiency causes defective nuclear mechanics and mechanotransduction. J Clin Invest 2004; 113:370-8.
32. Swaggart K A, Demonbreun A R, Vo A H et al. Annexin A6 modifies muscular dystrophy by mediating sarcolemmal repair. Proc Natl Acad Sci USA 2014; 111:6004-9.
33. Demonbreun A R, Fallon K S, Oosterbaan C C et al. Recombinant annexin A6 promotes membrane repair and protects against muscle injury. J Clin Invest 2019; 129:4657-4670.
34. Kim E Y, Barefield D Y, Vo A H et al. Distinct pathological signatures in human cellular models of myotonic dystrophy subtypes. JCIInsight 2019; 4.
35. Gacita A M, Fullenkamp D E, Ohiri J et al. Genetic Variation in Enhancers Modifies Cardiomyopathy Gene Expression and Progression. Circulation 2021; 143:1302-1316.
36. Burridge P W, Holmstrom A, Wu J C. Chemically Defined Culture and Cardiomyocyte Differentiation of Human Pluripotent Stem Cells. Curr Protoc Hum Genet 2015; 87:21 3 1-21 315.
37. Buikema J W, Lee S, Goodyer W R et al. Wnt Activation and Reduced Cell-Cell Contact Synergistically Induce Massive Expansion of Functional Human iPSC-Derived Cardiomyocytes. Cell Stem Cell 2020; 27:50-63 e5.
38. Danialou G, Comtois A S, Dudley R et al. Dystrophin-deficient cardiomyocytes are abnormally vulnerable to mechanical stress-induced contractile failure and injury. FASEB J 2001; 15:1655-7.
39. Hathout Y, Brody E, Clemens P R et al. Large-scale serum protein biomarker discovery in Duchenne muscular dystrophy. Proc Natl Acad Sci USA 2015; 112:7153-8.
40. Spurney C F, Ascheim D, Charnas L et al. Current state of cardiac troponin testing in Duchenne muscular dystrophy cardiomyopathy: review and recommendations from the Parent Project Muscular Dystrophy expert panel. Open Heart 2021; 8.
41. Jaffe A S, Landt Y, Parvin C A, Abendschein D R, Geltman E M, Ladenson J H. Comparative sensitivity of cardiac troponin I and lactate dehydrogenase isoenzymes for diagnosing acute myocardial infarction. Clin Chem 1996; 42:1770-6.
42. McNeil P L, Steinhardt R A. Loss, restoration, and maintenance of plasma membrane integrity. J Cell Biol 1997; 137:1-4.
43. Clarke M S, Khakee R, McNeil P L. Loss of cytoplasmic basic fibroblast growth factor from physiologically wounded myofibers of normal and dystrophic muscle. J Cell Sci 1993; 106 (Pt 1):121-33.
44. Blinova K, Schocken D, Patel D et al. Clinical Trial in a Dish: Personalized Stem Cell-Derived Cardiomyocyte Assay Compared With Clinical Trial Results for Two QT-Prolonging Drugs. Clin Transl Sci 2019; 12:687-697.
45. Sewanan L R, Shen S, Campbell S G. Mavacamten preserves length-dependent contractility and improves diastolic function in human engineered heart tissue. Am J Physiol Heart Circ Physiol 2021; 320:H1112-H1123.
46. Cai C, Masumiya H, Weisleder N et al. MG53 nucleates assembly of cell membrane repair machinery. Nat Cell Biol 2009; 11:56-64.
47. Cao C M, Zhang Y, Weisleder N et al. MG53 constitutes a primary determinant of cardiac ischemic preconditioning. Circulation 2010; 121:2565-74.
48. Weisleder N, Takizawa N, Lin P et al. Recombinant MG53 protein modulates therapeutic cell membrane repair in treatment of muscular dystrophy. Sci Transl Med 2012; 4:139ra85.
49. Demonbreun A R, Quattrocelli M, Barefield D Y, Allen M V, Swanson K E, McNally E M. An actin-dependent annexin complex mediates plasma membrane repair in muscle. J Cell Biol 2016; 213:705-18.

### Example 2

Membrane instability and disruption underlie a myriad of acute and chronic disorders. Anxa6 encodes the membrane-associated protein annexin A6 and was identified as a genetic modifier of muscle repair and muscular dystrophy. To evaluate annexin A6's role in membrane repair in vivo, sequences encoding green fluorescent protein (GFP) were inserted into the last coding exon of Anxa6. Heterozygous Anxa6gfp mice expressed a normal pattern of annexin A6 with reduced Anxa6-GFP mRNA and protein. High-resolution imaging of wounded muscle fibers showed annexin A6GFP rapidly formed a repair cap at the site of injury. Injured cardiomyocytes and neurons also displayed repair caps after wounding, highlighting annexin A6-mediated repair as a feature in multiple cell types. By surface plasmon resonance, recombinant annexin A6 bound phosphatidylserine-containing lipids in a Ca²⁺ and dose-dependent fashion with appreciable binding at approximately 50 μM Ca²⁺ and sub-nM (0.34) affinity. Exogenously added recombinant annexin A6 localized to repair caps and improved muscle membrane repair capacity in a dose-dependent fashion without disrupting endogenous annexin A6 localization, indicating annexin A6 promotes repair from both intracellular and extracellular compartments. Annexin A6 was expressed in cardiomyocytes and neurons and formed a repair cap at the site of damage. Recombinant annexin A6 similarly localized to the site of membrane injury in cardiomyocytes and neurons, enhancing repair capacity in control and diseased cells. Thus, annexin A6 orchestrates repair in multiple cell types, and recombinant annexin A6 is useful in additional chronic disorders beyond skeletal muscle myopathies, including disorders of the heart, brain, and nerve.

In this Example, the in vivo role of annexin A6 in plasma membrane repair was evaluated by using CRISPR-Cas9 to engineer a green fluorescent protein (GFP) tag at the carboxyl-terminus of annexin A6 (A6). In this model, annexin A6 expression is driven from the Anxa6 gene locus and it is not overexpressed. When subjected to plasma membrane injury, genomically-encoded annexin A6GFP formed time-dependent repair caps in skeletal muscle, cardiomyocytes and neurons. Exogenously added recombinant annexin A6, labelled with an alternative fluorescent tag, targeted the endogenous repair cap at the site of membrane injury in muscle and neurons. Together, these data support a general model of cellular repair for which recombinant annexin A6 is a useful resealing agent.

### Methods

Animals. Wildtype mice from the 129T2/SvEmsJ background were bred and housed in a specific pathogen free facility on a 12-hour light/dark cycle and fed ad libitum in accordance with the Northwestern University's Institutional Animal Care and Use Committee regulations. 129T2/SvEmsJ (129T2) mice were originally purchased from the Jackson Laboratory (Bar Harbor, ME; Stock #002065). Two to three-month-old male and female mice were used for all experiments. mdxC57BL10 mice were obtained from Jackson Laboratory (Bar Harbor, ME; Stock #001801).

Generation of gene edited mice. The Anxa6-TurboGFP mouse line was generated by the Northwestern University Transgenic and Targeted Mutagenesis Facility. CRISPR/Cas9 technology was used to insert the TurboGFP coding sequence before the Anxa6 stop codon creating a fusion construct. Briefly, two guide RNA (gRNA) were identified using Broad Institute online software (https://portals.broadinstitute.org/gpp/public/analysis-tools/sgrna-design). The gRNA sequences are: gRNA6, 5′-GCTTGCTCTGTGTGGCGGAG-3′ (SEQ ID NO: 39), and gRNA10, 5′-AGAGGGGGCCCTCTGAGGTC-3′ (SEQ ID NO: 40). Each gRNA was cloned into the PX459 V 2.0 Cas9 vector (Addgene #62988) as previously described (PMID 24157548). A repair vector was synthesized then cloned into the pUC57 backbone by GeneWiz (South Plainfield, NJ). This vector encodes TurboGFP flanked by 700 base pair (bp) homology arms. Three silent mutations were introduced in Anxa6 to destroy the gRNA6 recognition site.

Both gRNA/Cas9 plasmids (0.5 μg/each) and the repair template (2 μg) were introduced into 129S6 embryonic stem (ES) cells via nucleofection (Nucleofector 2b, Lonza, Basel, Switzerland). After 24 h, ES cells were subjected to puromycin selection for 48 h. ES cell clones were isolated and genotyped for insertion of the repair template into the Anxa6 locus. Targeted clones were microinjected into blastocyst stage C57BL/6J (https://www.jax.org/strain/000664) embryos which were then surgically transferred into the reproductive tract of recipient females. Chimeric mice were genotyped for the Anxa6 TurboGFP allele.

PCR and Genomic DNA analysis. Genomic DNA was isolated from mouse tail tissues. Gene-edited mice were genotyped based on the presence of either the wildtype annexin A6 and/or turbo GFP. PCR was performed using the following primer sequences: (1) Forward primer: CTAGGCCGATGGCTGCTA (SEQ ID NO: 41), (2) Reverse primer for wild-type annexin A6: CAATGGCTTGGTCAGGTCAC (SEQ ID NO: 42), (3) Reverse primer for tGFP: ACTTCTCGATGCGGGTGTTGGTG (SEQ ID NO: 43). Products were amplified by PCR using Phusion High-Fidelity DNA Polymerase (NEB) with the following cycle conditions: initial denaturation 98° C., 45 seconds followed by 98° C., 10 seconds; 64° C., 30 seconds; 72° C. 30 seconds for 35 cycles, and a final extension 72° C. for 5 minutes. Products were run on 2% agarose gel with ethidium bromide. Additionally, Sanger sequencing was performed on the amplified product to verify in-frame GFP insertion. 129/S6 (SvEvTac) ES cell genomic DNA was isolated and sequenced to confirm the absence of the annexin A6 truncated polymorphism.

Plasmids. A plasmid encoding annexin A6 with a carboxyl-terminal turboGFP tag was obtained from Origene (Rockville, MD). Subcloning of annexin A6 to replace the GFP tag with tdTomato (Addgene) was performed by Mutagenix (Suwanee, GA). Constructs were sequenced to verify mutagenesis. Plasmid DNA was isolated using the Qiagen endo-free Maxi prep kit (Qiagen #12362).

Sequence comparison and schematics. Snapgene and Lasergene were used to view and align chromatograms.

Protein isolation. Muscles were dissected and flash frozen. Tissues were lysed in whole tissue lysis buffer (50 mM HEPES pH 7.5, 150 mM NaCl, 2 mM EDTA, 10 mM NaF, 10 mM Na-pyrophosphate, 10% glycerol, 1% Triton X-100, 1 mM phenyl-methylsulfonyl fluoride (PMSF), 1× Roche cOmplete™ Protease Inhibitor Cocktail (Cat #11697498001 CO-RO; Roche, Basel, Switzerland;) and homogenized using a bead beater tissue homogenizer (BioSpec).

Immunoblotting. The protein concentration of the muscle or cell lysate was determined using the Quick Start™ Bradford Protein Assay (Cat #5000205 Bio-Rad Laboratories, Hercules, CA). Proteins were heated to 70° C. in 2× Laemmli buffer and were separated on 4-15% Mini-PROTEAN® TGX™ Precast Protein Gels, 15-well, 15 μl (Cat #4561086; Bio-Rad Laboratories, Hercules, CA) and transferred to Immun-Blot PVDF Membranes for Protein Blotting (Cat #1620177; Bio-Rad Laboratories, Hercules, CA). Blocking and antibody incubations were done using StartingBlock T20 (TBS) Blocking Buffer (Cat #37543; Thermo Fisher Scientific, Waltham, MA). Primary antibodies used were: annexin A6 (Cat #31026; Abcam) and turbo GFP (Evrogen, cat #AB513) used at 1:1000 diluted in starting block. Secondary antibodies conjugated to horseradish peroxidase were used at 1:5000 (Jackson ImmunoResearch Laboratories, West Grove, PA). SuperSignal™ West Pico Chemiluminescent Substrate and SuperSignal™ West Femto Maximum Sensitivity Substrate (Cat #34080 and #34096; Thermo Fisher Scientific, Waltham, MA) were applied to membranes and membranes were visualized using an Invitrogen™ iBright™ CL1000 Imaging System (Cat #A32749; Thermo Fisher Scientific, Waltham, MA). Pierce™ Reversible Protein Stain Kit for PVDF Membranes (Cat #24585; Thermo Fisher Scientific, Waltham, MA) was used to stain the entire blot to ensure complete transfer and equal loading. Immunoblot bands were quantified using FIJI gel analysis tools.

Membrane Lipid Assay. Lipid strip assays were performed per manufacturer's instructions (Echelon P-6003-2). Briefly, membrane was blocked with 5 ml TBS-t+3% BSA for 1 hr at room temperature. Protein (1 ug/ml rANXA6 in TBS-t+3% BSA+1 mM calcium) was incubated on the membrane for 1 hr at room temperature. Membranes were rinsed 3×5 mins in TBS-t and then incubated with anti-HIS-HRP (MA1-21315-HRP, ThermoFischer) for 1 hr at room temperature diluted 1:500 in TBS-t+3% BSA. Membranes were rinsed 3×5 mins in TBS-t and developed with 2 mL K-TMBP (Echelon) for 2-20 minutes.

Electroporation and myofiber isolation. Flexor digitorum brevis (FDB) fibers were transfected with endo-free plasmid DNA by in vivo electroporation. Methods were described previously in (Demonbreun A R, Fallon K S, Oosterbaan C C, Bogdanovic E, Warner J L, Sell J J, et al. Recombinant annexin A6 promotes membrane repair and protects against muscle injury. The Journal of Clinical Investigation. 2019; 129(11):4657-70; Demonbreun A R, Quattrocelli M, Barefield D Y, Allen M V, Swanson K E, and McNally E M. An actin-dependent annexin complex mediates plasma membrane repair in muscle. The Journal of cell biology. 2016; 213(6):705-18; Demonbreun A R, and McNally E M. DNA Electroporation, Isolation and Imaging of Myofibers. Journal of visualized experiments: JoVE. 2015; 106(106):e53551; DiFranco M, Quinonez M, Capote J, and Vergara J. DNA transfection of mammalian skeletal muscles using in vivo electroporation. Journal of visualized experiments: JoVE. 2009; 32(32)). Briefly, fibers were dissociated in 0.2% BSA plus collagenase type II (Cat #17101, Thermo Fisher Scientific, Waltham, MA) for 90-120 minutes at 37 degrees in 10% CO₂. Fibers were then moved to Ringers solution and placed on MatTek confocal microscopy dishes (Cat #P35G-1.5-14-C, MatTek, Ashland MA).

Cardiomyocyte isolation. Mice were treated with 50 U heparin intraperitoneally 20 min before sacrifice. Mice were anesthetized under 5% vaporized isoflurane mixed with 100% oxygen. A thoracotomy was performed and the heart and lungs rapidly excised and submerged into ice-cold Tyrode solution without calcium (143-mM NaCl, 2.5-mM KCl, 16-mM MgCl₂, 11-mM glucose, 25-mM NaHCO₃, pH adjusted to 7.4). The ascending aorta was dissected out of the surrounding tissue and cannulated with an animal feeding needle (7900, Cadence Science, Staunton, Virginia) and secured with a 6-0 silk suture. The heart was initially perfused with 1 ml of ice-cold calcium-free Tyrode solution before being transferred to a Langendorff apparatus (Radnoti, Covina, California). Hearts were perfused with 37° C. calcium-free Tyrode solution using a constant pressure (65-cm vertical distance between the buffer reservoir and cannula tip) for 1 to 2 min before perfusion for 5.5 min with digestion solution (0.15% collagenase type 2 [Worthington Biochemical, Lakewood, New Jersey], 0.1% 2,3-butanedione monoxime, 0.1% glucose, 100-U/ml penicillin/streptomycin, 112-mM NaCl, 4.7-mM KCl, 0.6-mM KH₂PO₄, 40-μM CaCl₂, 0.6-mM Na₂HPO₄, 1.2-mM MgSO₄, 30-pM phenol red, 21.4-mM NaHCO₃, 10-mM HEPES, and 30-mM taurine; pH adjusted to 7.4). The heart was removed from the cannula, triturated with a transfer pipette, and filtered through a 100-μm cell strainer. Cardiomyocytes were allowed to pellet by gravity for 7 min, followed by aspiration of digestion media and washing with stop buffer (formulated identically to digestion solution except with no collagenase and with 1% bovine serum albumin). Cells were again allowed to gravity pellet followed by a wash in stop buffer without bovine serum albumin. Cardiomyocytes were tolerated to calcium by adding Tyrode buffer with 0.3-mM CaCl₂ dropwise. Cell culture dishes were coated with 20 μg/ml laminin (Cat #23017-015; Gibco, Thermo Fisher Scientific, Waltham, Massachusetts) for 1 h at room temperature. Laminin solution was aspirated followed by plating of cardiomyocytes for 1 h to allow cell adhesion before experimentation.

Multiphoton laser injury and imaging. Isolated fibers were subjected to laser-induced damage at room temperature using the Nikon A1R-MP multiphoton microscope as described previously (Demonbreun A R, Fallon K S, Oosterbaan C C, Bogdanovic E, Warner J L, Sell J J, et al. Recombinant annexin A6 promotes membrane repair and protects against muscle injury. The Journal of Clinical Investigation. 2019; 129(11):4657-70). Imaging was performed using a 25×1.1 NA objective directed by the NIS-Elements AR imaging software. Green fluorescence protein (GFP) and FM 4-64 were excited using a 920 nm wavelength laser and emission wavelengths of 575 nm and 629 nm were collected respectively. To induce laser damage on isolated myofibers, a diffraction limited spot (diameter approximately 410 nm) was created on the lateral membrane of the myofiber using a 920 nm wavelength laser at 10-15% laser power for 1 s. Time lapse images were collected as follows: one image was collected prior to damage, one image upon damage, then every 8 s for 80 s (10 images) followed by every 30 s for 5 min (10 images). At the end of the time lapsed image series, z-stack images were collected at 250 nm intervals through the damaged site on the myofiber directed by the NIS-Elements AR imaging software. Fluorescence intensity and cap area were measured using Fiji (NIH). To damage cardiomyocytes, the cells were isolated and plated on laminin coated MatTek confocal microscopy dishes as described above. The cells were incubated for 1 h at 37° C. to allow cell attachment. Prior to laser damage, the cells were incubated in Tyrode buffer containing 0.5 mM CaCl₂ and damaged as described. To damage neurons, the cells were grown in 35 mm culture dishes in growth media. Prior to damage, the cells were washed twice in PBS and incubated in Ringer's buffer containing 1 mM CaCl₂ and damaged as described above.

For recombinant protein studies, myofibers were isolated from mice as described above. Myofibers were incubated in specified concentrations of recombinant annexin A6 and 1 mM Ca²⁺ Ringers or BSA control. Cap size was assessed from acquired images in FIJI. FM 4-64 (Cat #T13320; ThermoFisher) (2.5 μm) was added to the myofibers just prior to imaging. Images were acquired and quantitated as described above. FM 4-64 fluorescence at endpoint was measured using FIJI. Isolated myofibers were treated with 20 μM wortmannin (Cat #12-338; Sigma).

Neuron Isolation and Immunoblot. Mixed cortical and hippocampal neurons were isolated from day 15.5-16.5 A6-GFP or C57B6 mouse embryos via dissociation at 37° C. in 0.25% trypsin. Neurons were plated in poly-l-lysine coated 12-well plates (750,000 cells per well) or Mat-Tek glass-bottomed 3 cm dishes (450,000 cells per dish) containing neurobasal media supplemented with 2% B-27, 500 pM glutamine, 10% horse serum and 2.5 pM glutamate. After 2 hours, the media was replaced with neurobasal media with 2% B-27, 500 pM glutamine. All cell culture reagents were from Thermo Scientific. For immunoblotting, cells in 12 well plates were lysed in in RIPA buffer (150 mM NaCl, 1% IGEPAL CA-630, 0.5% sodium deoxycholate, 0.1% SDS, 50 mM Tris pH 8, 1 mM PMSF) with Protease Inhibitor Cocktail Ill (Calbiochem) and Halt Phosphatase Inhibitor Cocktail (Thermo Scientific). Lysates were centrifuged at 10,000 rpm, 4° C., 10 min and the supernatant protein was quantified by BCA (Thermo Scientific). 10 ug protein were separated on Invitrogen NuPAGE Bolt 4-12% Bis-Tris gels and transferred overnight to PVDF (Millipore). Membranes were blocked in Pierce Superblock and then probed for 1 h at room temperature with either anti-Turbo GFP (Wako/FujiFilm Evrogen, 1:1000) in 10% SuperBlock in TBS 0.1% Triton, or anti-GAPDH (Cell Signaling #14C10) in 5% milk in TBS-0.1% Triton followed by horseradish peroxidase-conjugated anti-rabbit antibody (Vector Laboratories PI-1000, 1:5000). Blots were visualized using the Pierce reagents West Femto (Turbo-GFP) or SuperSignal West Pico (GAPDH) and signals were imaged using a FluorChemR imager (ProteinSimple) and then quantified with Alphaview software (ProteinSimple). Turbo-GFP signal was normalized to GAPDH, and Student's two-tailed t test was done using InStat software (GraphPad Software, Inc., San Diego, CA.)

Brain Sectioning and Imaging. Two to four-month old heterozygous A6-GFP or wild type mice were euthanized and transcardially perfused with ice-cold PBS containing protease and phosphatase inhibitors. After perfusion, the brain was bisected and one hemibrain was drop fixed in 4% paraformaldehyde/PBS and cryopreserved in 30% w/v sucrose/PBS for sectioning. The other hemibrain was flash frozen in LN2 for biochemical analysis. 30 μm coronal floating brain sections were cut and stained as follows. Sections were washed 3 times in Tris Buffered Saline, incubated in 16 mM glycine in Tris Buffered Saline with 0.25% Triton-X 100 (TBS-T), blocked first in 5% donkey serum in TBS-T, then with 1% BSA in TBS-T. Sections were incubated overnight at 4° C. with anti-Turbo GFP (Wako/FujiFilm Evrogen, 1:500) and mouse anti-NeuN (Millipore Sigma, MAB377, 1:1000) in 1% BSA TBS-T. The following day, they were incubated with 1:750 donkey anti-rabbit Alexa 488 and donkey anti-mouse 594 (ThermoFisher). All staining was performed at the same time. Sections were mounted with ProLong Gold Antifade (Cat #P36930, ThermoFisher) and images acquired on a Nikon A1R or W1 confocal microscope with a 20× or 40× objective, using NIS Elements software. All image acquisition settings were maintained the same between genotypes.

Recombinant Protein Production. Recombinant annexin A6 protein and annexin A6-tdTomato protein was generated by Evotec using E. coli and Expi293 cells and standard methods (Princeton, New Jersey). Media was purified using IMAC chromatography. The final recovery of purified recombinant annexin A6 protein was diluted in TBS with an endotoxin level at approximately 1.5 EU/mg, with a purity >80%. Recombinant annexin A6 was labelled with Alexa-488 using standard methods (Cat #A10235, ThermoFisher).

In vitro injury and binding. A stock of 50 ng/mL Listeriolysin-O (LLO) (Cat #ab83345; Abcam) in PBS without calcium and without magnesium (PBS−/−) prepared on ice. L6 rat myoblasts were trypsinized and resuspended in PBS−/− to achieve a concentration of 10,000 cells/uL. 1,000,000 cells were added to each tube of the prepared LLO and incubated on ice for 5 minutes. After 5 minutes, cells were pelleted, rinsed twice and resuspended in PBS with 0.45 nm Ca²⁺ with varying concentrations of Annexin A6-488( 0 to 100 μg/ml). Cells were incubated at 25° C. for 5 minutes. Two μL of Sytox Red dye (Cat #S34859; ThermoFisher) was added to each tube of cells and incubated at 25° C. for an additional 10 minutes. Cells were rinsed and then resuspended in 300 μL of PBS−/−. Flow cytometry was performed on the BD-Accuri C6 Flow Cytometer. A cell count of 30,000 was achieved for each tube. Analysis was performed using FlowJo software (Becton, Dickinson & Company).

Liposome preparation. The preparation of uniform approximately 100 nm diameter liposomes was carried out as described previously (Hamman et al., 2002). PS, Phosphatidylserine, SM, sphingomyelin, CH, cholesterol, PE, phosphatidylethanolamine, and PC, phosphatidylcholine were commercially purchased (Sigma-Aldrich). PS was re-suspended in chloroform:methanol solution to make a 10.7 mg/mL (27.77 mM) stock solution; PC in chloroform to make a 25 mg/mL stock (31.80 mM); PE in chloroform to make a 25 mg/mL stock (33.60 mM), CH in chloroform to make a 100 mg/mL stock (258.63 mM), and SM in methanol to make a 25 mg/mL stock (34.20 mM). Liposomes were prepared with PS (+PS) and without PS (−PS). The composition ratios for liposome preparations are +PS (3.0 PC: 1.5 PE: 3.0 CH: 1.5 SM: 1.0 PS) and −PS (3.0 PC: 1.5 PE: 3.0 CH: 1.5 SM). All lipids were at room temperature before preparing the +PS and −PS mixture. Each lipid mixture was dried for 15-20 minutes under a steady and gentle stream of nitrogen. Each dried lipid mixture was re-suspended in 1 mL of buffer (50 mM HEPES pH 7.3, 50 mM NaCl) to make a 10 mM of +PS and approximately 9 mM stock of −PS in glass vials. The glass vials were sealed with parafilm and sonicated for approximately 10 minutes. The liposome preparation was carried out using an Avanti mini extruder (https://avantilipids.com/divisions/equipment-products/mini-extruder-extrusion-technique). The lipid mixture was cycled through the extruder for 20-25 cycles. The liposome mixture was transferred to glass scintillation vials and stored at 2-8° C. until use.

Surface Plasmon Resonance (SPR) Binding Studies. All SPR studies were performed on a Biacore 8K+ instrument (Cytiva) at 25° C. A series S L1 chip (lipophilic groups are covalently attached to carboxymethylated dextran, making the surface suitable for direct attachment of lipid membrane vesicles) was used for the AnxA6/lipid interaction studies. Briefly, the L1 sensor chip (Cytiva) was equilibrated in running buffer (10 mM HEPES, pH 7.4, 150 mM NaCl), and conditioned with two 30 s injections of 40 mM octyl glucoside at 10 μl/min before liposome immobilization (Hodnik et al., 2010). 0.5 mM+PS and −PS liposomes (flow rate 2 μl/min) were captured onto the active and reference flow cell surfaces to approximately 10000 RU respectively to form the lipid bilayer. This was followed by two 60 s injections of 10 mM NaOH at 10 μl/min to remove any unbound liposomes on the L1 chip. Initially, the Ca²⁺ dependence of AnxA6 binding to +PS and −PS lipid was tested. The CaCl₂ concentration in the running buffer was varied between 0-2.5 mM (0, 26, 52, 104, 208, 417, 833, or 2500 pM) and the binding signal RU and kinetics of the AnxA6/lipid interactions were analyzed. This was followed by dose response kinetics studies on the AnxA6/+PS lipid interactions at 100 μM CaCl₂. Since −PS showed very minimal binding at 100 μM CaCl₂, it was used as a negative control lipid, and captured on the reference flow cell surface. +PS was captured on the active flow cell surface. Parallel dose response kinetics was run with 8 concentrations (0.78 nM-100 nM) at 2-fold dilutions on 8 different channels of the sensor chip. AnxA6 was injected at 50 μl/min with association time of 240 s and dissociation time of 600 s. All data we analyzed using the Biacore Insight Evaluation software (ver. 3.0.12; Cytiva). Raw sensograms were reference subtracted, blank-buffer subtracted before kinetic and affinity analysis to account for nonspecific binding and injection artifacts. Association (kₐ M⁻¹ s⁻¹) and dissociation (k_{d} s⁻¹) rate constants, and binding affinity (K_{D}) values were determined using a 1:1 kinetics binding model. The closeness of fit between the experimental data and fitted curves was assessed using χ2 (average squared residual).

Statistical analysis. Statistical analyses were performed with Prism (GraphPad, La Jolla, CA). Comparisons relied on ANOVA (1 way ANOVA for 1 variable). Otherwise, unpaired two-tailed t-tests were performed. P value less than or equal to 0.05 was considered significant. Data were presented as single values were appropriate. Error bars represent +/− standard error of the mean (SEM).

### Results

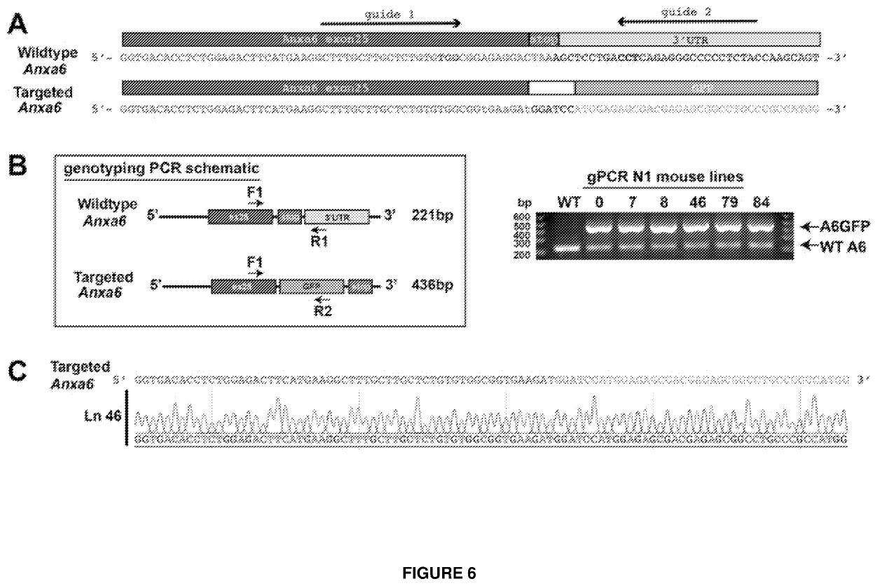

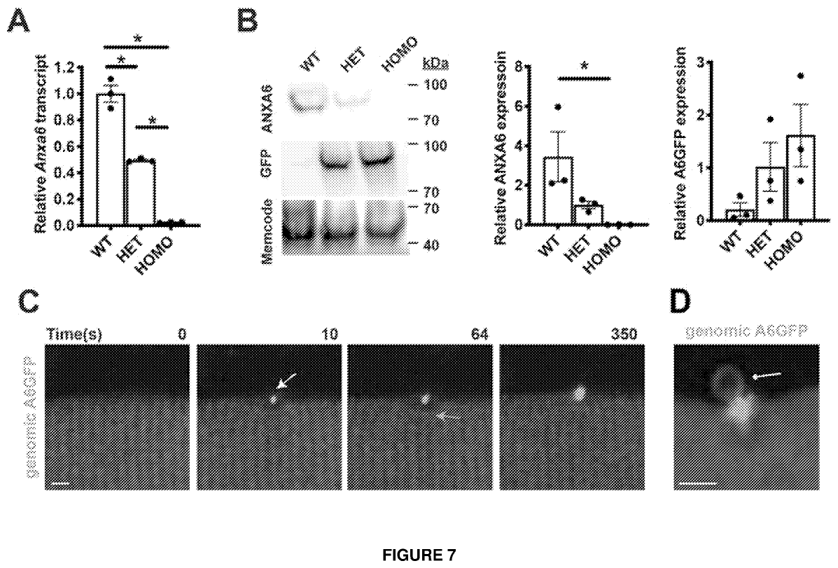

Generation of Anxa6gfp mice using gene editing. CRISPr/Cas9 was used to replace the stop codon in the last exon of the Anxa6 locus with sequences encoding GFP (FIGS. 6A, 6B, and 6C). A dual guide strategy was used to insert the GFP-encoding sequences into embryonic stem cells, which were subsequently injected into blastocysts to create founder mice (FIGS. 6A, 6B, and 6C). This strategy mirrors the carboxy-terminal GFP tags used in annexin A6 plasmid-mediated overexpression studies. Both heterozygous and homozygous Anxa6gfp mice were characterized. Quantitative PCR analysis documented a reduction in Anxa6 transcript in heterozygous mice compared to WT and further reduction in homozygous mice (FIG. 7A). Antibodies specific to annexin A6 and to GFP were used to evaluate protein expression in quadriceps muscles (FIG. 7B). A reduction in the expected amount of A6GFP protein compared to endogenous annexin A6 was observed, such that heterozygous Anxa6gfp mice expressed 3-8% of the total annexin A6 protein levels expressed in quadriceps (FIG. 7B). To confirm that A6GFP, which was expressed at lower than wildtype annexin A6 levels, localized to the site of membrane injury, flexor digitorum brevis myofibers were isolated from Anxa6gfp mice and imaged. Within seconds of laser-mediated injury, genomically-encoded A6GFP localized to the membrane lesion organizing into a repair cap (FIG. 7C). High magnification imaging revealed the presence of A6GFP containing membranous blebs emanating from the repair cap (FIG. 7D). These data combined showed that Anxa6gfp mice express genomically-encoded A6GFP protein in the expected pattern in muscle but with reduced A6GFP expression compared to endogenous annexin A6.

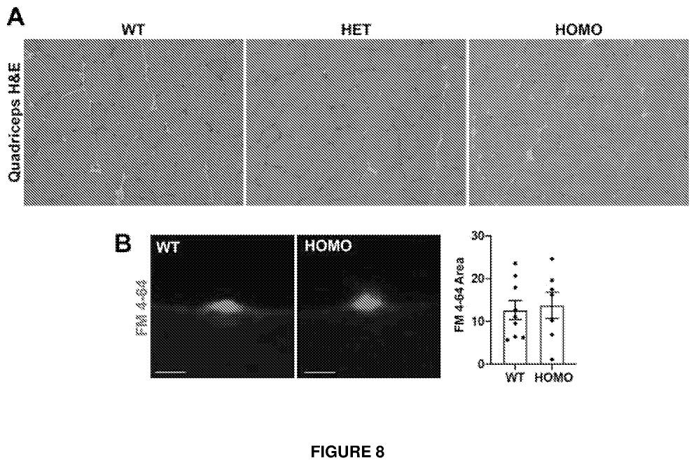

Normal muscle in Anxa6gfp mice with normal muscle repair. Prior studies have shown minimal phenotype in Anxa6-null mice with no discernable impact on any of the major organs including skeletal muscle, heart, and brain (Hawkins T E, Roes J, Rees D, Monkhouse J, and Moss S E. Immunological development and cardiovascular function are normal in annexin VI null mutant mice. Mol Cell Biol. 1999; 19(12):8028-32). Similarly, annexin A6GFP protein expression in heterozygous and homozygous mice resulted in no overt muscle, heart, or brain defects in the background of otherwise healthy mice. Specifically, no immune infiltrate, fibrosis, or internal nuclei was detected, and histologically, normal muscle was indistinguishable from annexin A6GFP-expressing muscle (FIG. 8A). Myofibers were incubated in FM 4-64, a fluorescent indicator dye that increases fluorescence intensity upon binding to exposed phosphatidylserine and used as a marker of membrane injury. Homozygous Anxa6gfp myofibers had similar levels of FM dye uptake compared to wildtype myofibers, confirming intact muscle repair capacity (FIG. 8B). Thus, the GFP tag on A6 did not alter the properties of A6 in muscle, consistent with prior studies in which A6GFP was expressed from a plasmid.

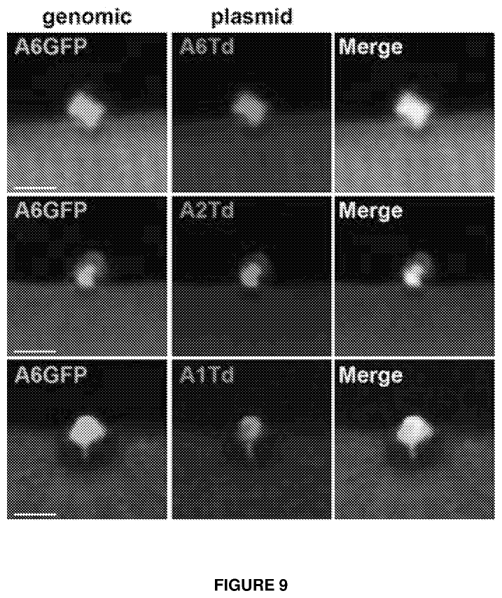

Genomically-encoded annexin A6GFP forms a repair cap with annexins A1 and A2 at the site of muscle membrane injury. Electroporation of annexin-encoding plasmids was previously used to demonstrate annexin A6 forming a repair cap with annexins A1 and A2, and these studies relied on annexin overexpression. To determine if genomically-encoded A6GFP expressed at lower than wildtype levels could still nucleate the annexin repair complex at the site of injury, Anxa6gfp myofibers were electroporated with annexin A6-tdTomato, A2-tdTomato or annexin A1-tdTomato plasmid and subjected to laser-induced injury. Genomically-encoded annexin A6GFP localized to the site of injury colocalizing with A6-tdTomato, A2-tdTomato and A1-tdTomato (FIG. 9). These data demonstrated that annexin A6GFP facilitated membrane repair across a broad range of concentrations including levels of expression lower than endogenous annexin A6.

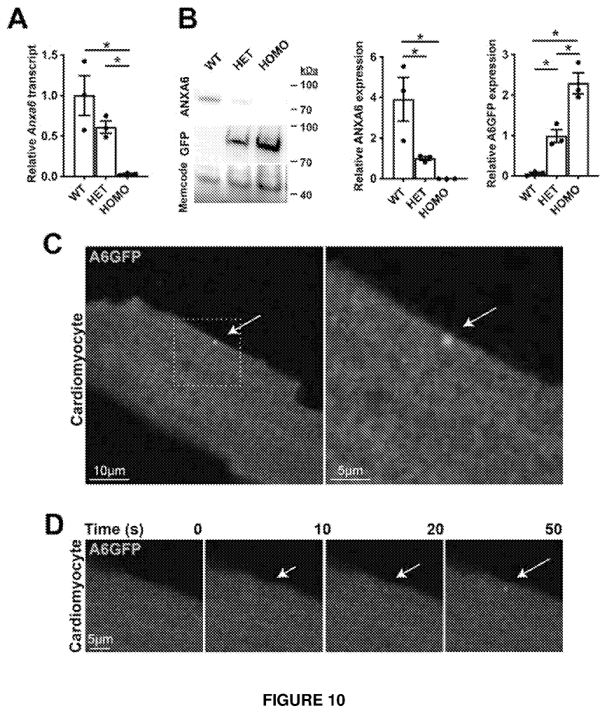

Annexin A6GFP is expressed in the heart and forms repair caps in injured cardiomyocytes. The degree to which annexin A6-containing membrane repair complexes are found outside of myofiber repair is not known. Similar to skeletal muscle, a reduction in cardiac Anxa6 transcript level in heterozygous and homozygous mice was documented utilizing quantitative PCR analysis (FIG. 10A). Immunoblot analysis with anti-annexin A6 antibody confirmed a reduction in ANXA6 protein in Anxa6gfp heterozygous and homozygous cardiac lysates, while anti-GFP antibody detected increasing amounts of annexin A6-GFP protein in Anxa6gfp heterozygous and homozygous cardiac lysates (FIG. 10B). Since skeletal myofibers and cardiomyocytes share many structural and functional features, we isolated and injured cardiomyocytes from Anxa6gfp mice to evaluate cardiomyocyte membrane repair (FIG. 10C). Anxa6gfp ventricular cardiomyocytes were isolated, and the laser injury protocol was modified for use on cardiomyocytes. Compared to skeletal myofibers, cardiomyocytes were exquisitely sensitive to laser injury. Accordingly, the laser power was reduced by approximately 50% and external calcium levels were reduced 50% (500 μM) to accommodate this increased sensitivity to injury. In uninjured cardiomyocytes, A6GFP localized in a sarcomeric pattern in live cells, in a pattern consistent with the known localization of cardiac annexin A6 (FIG. 10C) (Mishra S, Chander V, Banerjee P, Oh J G, Lifirsu E, Park W J, et al. Interaction of annexin A6 with alpha actinin in cardiomyocytes. BMC Cell Biol. 2011; 12:7). Within 10 seconds of laser-wounding, annexin A6GFP localized to the membrane lesion organizing into a repair cap in the cardiomyocyte (FIG. 10C). A magnified image of the white dotted box depicting a bright annexin A6GFP repair cap is shown in the image on the right (FIG. 10C). Timelapse images illustrating the progression of annexin A6GFP localization into the repair cap (arrow) in an isolated cardiomyocyte is shown through 50 seconds post injury (FIG. 10D). Thus, genomically-encoded annexin A6GFP localized to the site of membrane injury forming a repair cap at the membrane lesion in live, adult ventricular cardiomyocytes, consistent with a conserved role for annexin A6 in mediating membrane repair between cardiomyocytes and skeletal myofibers.

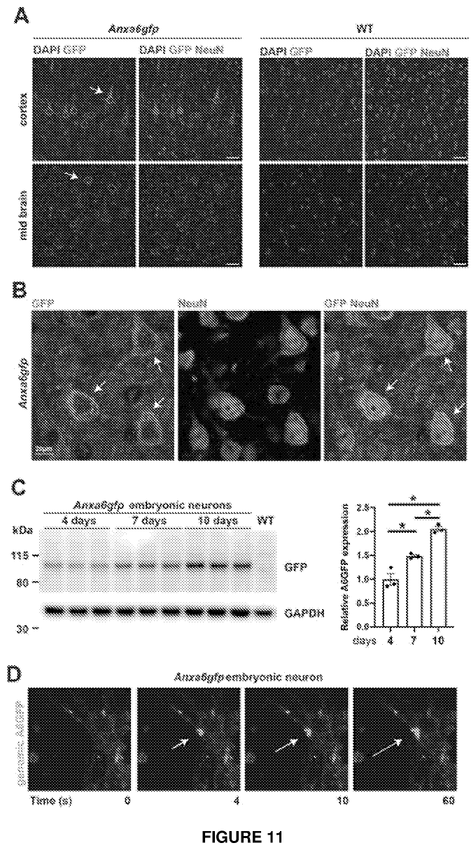

Annexin A6 localized to neuronal membrane lesions. Using the Anxa6gfp mouse model, whether endogenous annexin A6 translocation was a component of primary neuronal cell injury repair was evaluated. Brain imaging of Anxa6gfp mice using anti-GFP antibodies detected A6GFP protein, largely restricted to the plasma membrane, and this was well seen in cortical neurons, marked by NeuN positivity (FIGS. 11A and 11B). Neurons were isolated at embryonic day 15-16 and cultured under maturation conditions. As neuron maturation progressed from day 4 to day 10 in culture, annexin A6GFP levels significantly increased (FIG. 11C). Day 7 neurons were subjected to laser-induced membrane injury. Genomically-encoded annexin A6GFP localized to the site of neuron injury forming a repair cap visible within 1-2 seconds of injury, which persisted through the 60 seconds of imaging (FIG. 11D). Together with observations in skeletal myofibers and cardiomyocytes, these findings demonstrated a role for annexin A6 in cellular repair, underscoring the broad nature of this repair mechanism.

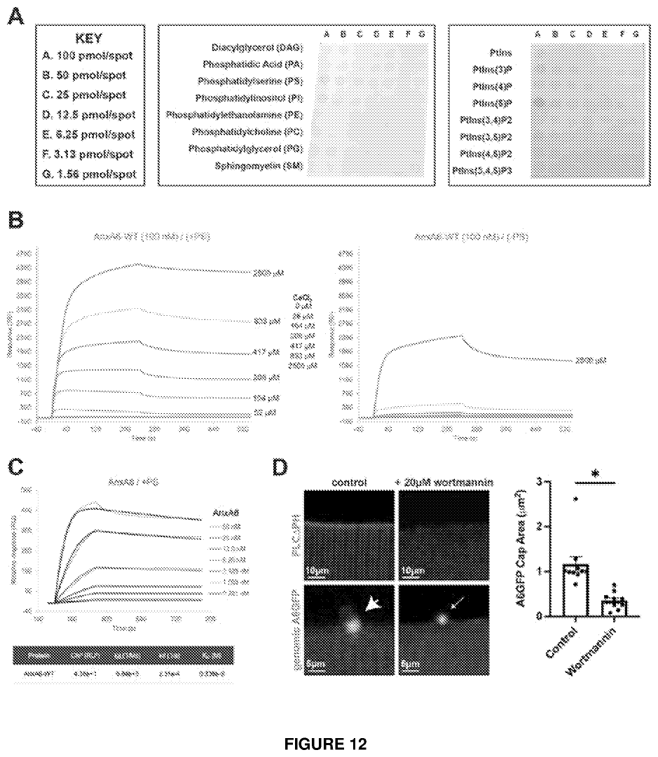

Recombinant annexin A6 binds phosphatidylserine in a calcium-dependent manner. Annexins are known calcium-dependent phospholipid binding proteins. Phosphatidylserine (PS) and phosphatidylethalomine (PE) are membrane lipids that normally are found in the inner plasma membrane leaflet and upon membrane injury flip to the outer leaflet. To evaluate phospholipid binding preference in vitro, recombinant annexin A6 was incubated on lipid arrays containing the common membrane lipids diacylglycerol (DAG), phosphatidic acid (PA), phosphatidylserine (PS), phosphatidylinositol (PI), phosphatidylethanolamine (PE), phosphatidylcholine (PC), phosphatidylglycerol (PG) and sphingomyelin (SM). Recombinant annexin A6 preferentially bound PS and PI (FIG. 12A). To further evaluate the Ca²⁺ dependency and kinetics of recombinant annexin A6 binding to PS, surface plasmon resonance (SPR) was performed +PS or −PS over a range of Ca²⁺ concentrations (0, 26, 52, 104, 208, 417, 833, and 2500 μM). Recombinant annexin A6 binding to PS increased with increasing concentrations of Ca²⁺, with appreciable binding at 50 μM and higher (FIG. 12B, left). Minimal recombinant annexin A6 binding occurred in the absence of PS with virtually no binding present below 200 μM Ca²⁺ (FIG. 12B, right). Given that minimal to no binding occurred at 100μM Ca²⁺ without PS, this was used as a negative control on the reference flow cell surface and for the interaction kinetics of annexin A6 with PS. Dose response kinetics had a very low dissociation rate (2.31×10-4) for the interaction and sub-nM binding affinity (0.34 nM) (FIG. 12C). It was concluded that PS is a substrate for annexin A6's membrane binding interactions during cell membrane repair.

Annexin A6 sensed phosphoinositides during cap formation. Biological membranes are composed of lipid microdomains that regulate cell signaling events and membrane trafficking (Simons K, and Toomre D. Lipid rafts and signal transduction. Nature reviews Molecular cell biology. 2000; 1(1):31-9; Parton R G, and del Pozo M A. Caveolae as plasma membrane sensors, protectors and organizers. Nature reviews Molecular cell biology. 2013; 14(2):98-112). Annexins bind phospholipids, including PS and phosphatidylinositol 4,5-bisphosphate (PIP2), in response to changes in Ca²⁺ levels (Illien F, Piao H-R, CouéM, Di Marco C, and Ayala-Sanmartin J. Lipid organization regulates annexin A2 Ca 2+-sensitivity for membrane bridging and its modulator effects on membrane fluidity. Biochimica et Biophysica Acta (BBA)-Biomembranes. 2012; 1818(11):2892-900). Additionally, PS and PIP2 have been implicated in cell fusion and membrane repair located at the site of damage (Demonbreun A R, Quattrocelli M, Barefield D Y, Allen M V, Swanson K E, and McNally E M. An actin-dependent annexin complex mediates plasma membrane repair in muscle. The Journal of cell biology. 2016; 213(6):705-18; Vaughan E M, You J S, Elsie Yu H Y, Lasek A, Vitale N, Hornberger T A, et al. Lipid domain-dependent regulation of single-cell wound repair. Mol Biol Cell. 2014; 25(12):1867-76; Leikina E, Melikov K, Sanyal S, Verma S K, Eun B, Gebert C, et al. Extracellular annexins and dynamin are important for sequential steps in myoblast fusion. J Cell Biol. 2013; 200(1):109-23). To determine the contribution of phosphoinositides during membrane repair, myofibers were incubated in wortmannin, a known inhibitor of phosphatidylinositol 3-kinase (PI3-K). When used at higher concentrations (20 pM), wortmannin also inhibits phosphatidylinositol 4-kinase (P14-K) leading to PIP and PIP2 depletion (Downing G J, Kim S, Nakanishi S, Catt K J, and Balla T. Characterization of a soluble adrenal phosphatidylinositol 4-kinase reveals wortmannin sensitivity of type Ill phosphatidylinositol kinases. Biochemistry. 1996; 35(11):3587-94; Nilius B, Mahieu F, Prenen J, Janssens A, Owsianik G, Vennekens R, et al. The Ca²⁺-activated cation channel TRPM4 is regulated by phosphatidylinositol 4,5-biphosphate. EMBO J. 2006; 25(3):467-78; Nakanishi S, Catt K J, and Balla T. A wortmannin-sensitive phosphatidylinositol 4-kinase that regulates hormone-sensitive pools of inositolphospholipids. Proceedings of the National Academy of Sciences of the United States of America. 1995; 92(12):5317-21). Depletion of PIP2 after wortmannin treatment was confirmed through electroporation of the PIP2 fluorescent biosensor, PLC-PH-EGFP, in wildtype myofibers, which displayed a visible reduction in PLC-PH-EGFP signal with treatment (FIG. 12D, top panel) (Stauffer T P, Ahn S, and Meyer T. Receptor-induced transient reduction in plasma membrane Ptdlns(4,5)P2 concentration monitored in living cells. Curr Biol. 1998; 8(6):343-6). Myofibers from Anxa6gfp mice were subsequently treated with 20 μM wortmannin and subjected to laser-induced injury. Genomically encoded annexin A6GFP cap size was significantly reduced (3.3 fold; P<0.002) by wortmannin treatment (FIG. 12D, bottom panel and graph). Combined, these results illustrated the dependency of annexin A6 repair cap formation on membrane lipid composition.

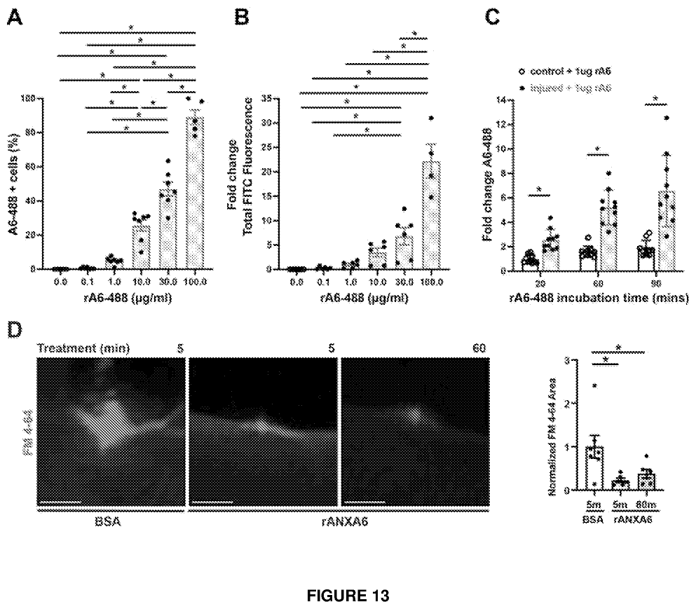

Recombinant annexin A6 binds injured membrane in a concentration-dependent and time-dependent manner. Skalman and colleagues overexpressed annexin A6 from a plasmid in HEK cells and found that it localized to the site of listeriolysin O (LLO)-induced membrane injury (Nygard Skalman L, Holst M R, Larsson E, and Lundmark R. Plasma membrane damage caused by listeriolysin O is not repaired through endocytosis of the membrane pore. Biol Open. 2018; 7(10)). To better understand the concentration and timing dynamics of recombinant annexin A6 binding of injured membrane, a quantitative, cell-based assay was developed that combined LLO-induced injury and flow cytometry using rat L6 myoblasts. At the amino acid level, rat annexin A6 protein is 94.6% similar to human annexin A6 and 98.3% similar to mouse annexin A6 (Demonbreun A R, Fallon K S, Oosterbaan C C, Bogdanovic E, Warner J L, Sell J J, et al. Recombinant annexin A6 promotes membrane repair and protects against muscle injury. The Journal of Clinical Investigation. 2019; 129(11):4657-70). L6 myoblasts were injured with LLO and then incubated with increasing concentrations of Alexa-488 labelled recombinant annexin A6 protein (referred to as rA6-488) in concentrations ranging from 0 to 100 μg/ml and fluorescence as a surrogate measure of binding quantified by flow cytometry. The percentage of rA6-488 positive cells increased with increasing concentrations of rA6-488, with nearly 100% of injured cells showing rA6-488 binding at 100 μg/ml (FIG. 13A). As another measure of binding, the total fluorescence intensity emitted by rA6-488 binding increased with increasing concentrations of protein with levels 22-fold higher at 100 μg/ml than at 1 μg/ml (FIG. 13B). Furthermore, when L6 injured myoblasts were incubated with the same concentration of rA6-488 but incubation time varied (20-90 mins), binding increased with increasing time (FIG. 13C). To determine if timing of recombinant annexin A6 treatment altered function, myofibers were pretreated with recombinant annexin A6 for 5 minutes or 60 minutes and then subjected to laser injury in the presence of FM 4-64 dye. FM 4-64 dye uptake was similarly reduced with both 5 minutes and 60 minutes of pretreatment compared to BSA controls (FIG. 13D). These data indicated that recombinant annexin A6 binds disrupted membrane and protects against membrane injury in a dose-dependent fashion.

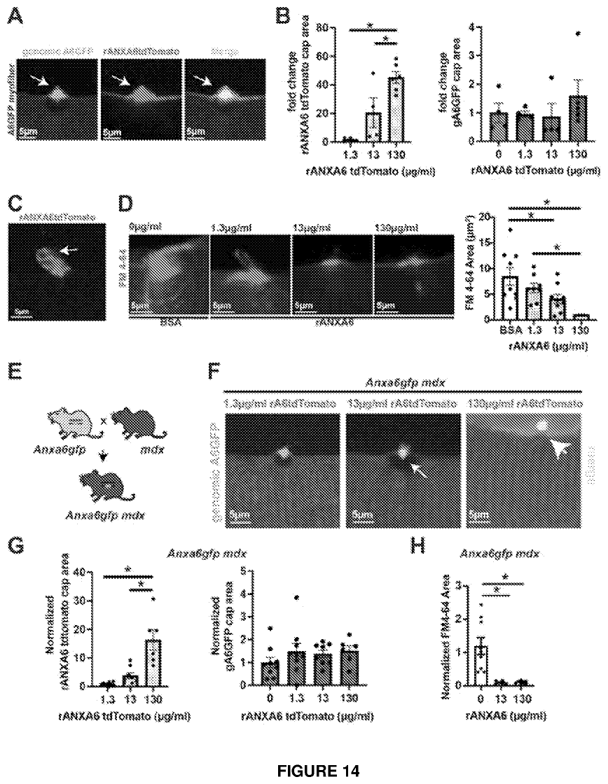

Recombinant annexin localized to the repair cap and enhanced repair. The above data demonstrated that annexin A6 expressed from the endogenous locus was recruited from its position within fibers to the plasma membrane to participate in resealing wounds. It was next evaluated whether exogenously added recombinant annexin A6 interacted with endogenously encoded annexin A6. To do this, Anxa6gfp myofibers were laser-injured in the presence of recombinant annexin A6 labelled with a carboxy terminal tdTomato fused tag (referred to as rA6-tdTomato). rA6-tdTomato localized to the site of membrane injury, colocalizing with genomically-encoded annexin A6GFP (FIG. 14A). rA6-tdTomato cap area increased with increasing concentrations (1.3-130 μg/ml) of available rA6-tdTomato protein (FIG. 14B, left). However, the presence of recombinant annexin rA6-tdTomato did not significantly increase genomically-encoded annexin A6GFP cap size (FIG. 14B, right). High-resolution imaging at the site of muscle membrane injury revealed the presence of rA6-tdTomato containing membranous blebs (FIG. 14C). To determine if the protective effect of rA6 was concentration-dependent, myofibers were incubated in a range of recombinant annexin A6 protein concentrations (0-130 pg/ml) or BSA control. Subsequently, myofibers were incubated in FM 4-64 dye and subjected to laser injury. Myofibers pretreated with recombinant annexin A6 had a dose-dependent reduction in FM dye uptake, compared to control myofibers (FIG. 14D), indicating higher levels of protection with increased concentrations of recombinant annexin A6 protein. These data demonstrated a role for annexin A6 in the immediate repair response required to seal membrane lesions independent of overexpression systems.

The role of annexin A6 in dystrophic muscle, which continually undergoes bouts of injury and repair, was assessed next. Anxa6gfp mice were crossed with the mdx model of Duchenne muscular dystrophy to generate Anxa6gfp mdx mice (FIG. 14E). The mdx mouse model lacks dystrophin expression resulting in a fragile sarcolemma that is prone to injury (Bulfield G, Siller W G, Wight P A, and Moore K J. X chromosome-linked muscular dystrophy (mdx) in the mouse. Proceedings of the National Academy of Sciences of the United States of America. 1984; 81(4):1189-92; Sicinski P, Geng Y, Ryder-Cook A S, Barnard E A, Darlison M G, and Barnard P J. The molecular basis of muscular dystrophy in the mdx mouse: a point mutation. Science. 1989; 244(4912):1578-80) Anxa6gfp mdx myofibers were isolated and laser-injured in the presence of rA6-tdTomato. Like healthy muscle, rA6-tdTomato localized to the site of dystrophic membrane injury, colocalizing with genomically-encoded annexin A6GFP at the lesion (FIGS. 14F and 14G). Genomically-encoded annexin A6GFP cap size in mdx myofibers was not significantly altered by the presence of rA6-tdTomato (FIGS. 14F and 14G). However, pretreatment with recombinant annexin A6 had a dose-dependent effect on myofiber FM dye uptake, reducing dye influx compared to control treated myofibers (FIG. 14H). Together these findings showed that recombinant annexin A6 protein increases exogenous cap size with increased concentrations, correlating with increased repair capacity in both healthy and dystrophic muscle.

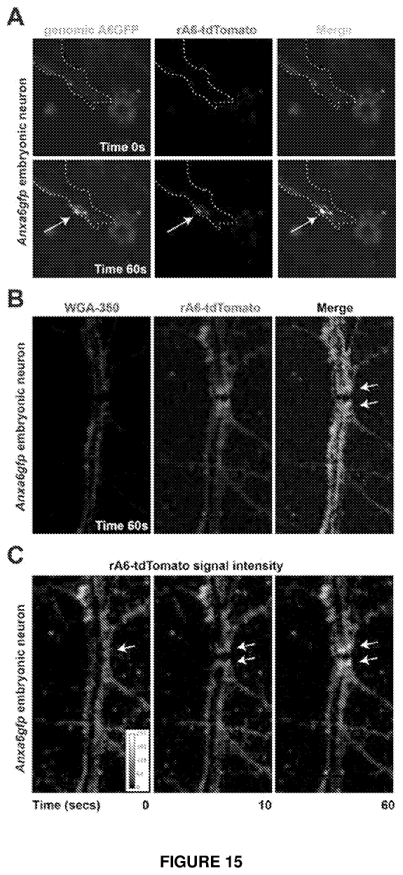

Recombinant annexin A6 localized to injured neurons. The process of neuron regeneration after axonal crush or severing requires resealing of the membrane prior to growth cone formation and regeneration (Geddis M S, and Rehder V. Initial stages of neural regeneration in Helisoma trivolvis are dependent upon PLA2 activity. J Neurobiol. 2003; 54(4):555-65). To evaluate the binding potential of recombinant annexin A6 to injured neurons, embryonic neurons were isolated and laser-injured in the presence of rA6-tdTomato. In the first injury assay, the neuronal membrane was nicked with the laser, similar to the protocol used for skeletal myofibers. With this type of injury, rA6-tdTomato localized to the site of neuron membrane injury, colocalizing with genomically-encoded annexin A6GFP at the repair cap (FIG. 15A). In the second injury assay, the laser was used to fully transect the neuronal process creating two stumps at the injury site. Wheat germ agglutinin (WGA) was used to label the neuron membrane, which showed clear disruption of the neuronal process after transection (FIG. 15B). Four seconds after transection, when the first image was acquired, rA6-tdTomato was detectable at the transected stumps (FIGS. 15B and 15C). rA6-tdTomato fluorescent intensity at the severed stumps continued to increase through the 60 seconds of imaging (FIG. 15C). Therefore, recombinant annexin A6 binds disrupted neuronal membranes after the smaller “nicking” injury and after full transection.

### DISCUSSION

Impact of annexin A6 expression level on membrane repair. Prior studies examining the role of annexin A6 in membrane repair relied on plasmid-mediated overexpression, which could alter the kinetics and efficiency of membrane repair. In this Example, a genomically-encoded annexin A6GFP, expressed at lower levels than normal annexin A6 protein, was utilized to document the role of annexin A6 in repair cap formation. This decrease in in vivo expression of total annexin A6 did not appear to have deleterious effects on muscle histology, and repair complex formation remained readily visible. The data provided herein showed that the small amount of functional annexin A6 is sufficient to promote repair, consistent with annexin A6 as a modifier gene and not a primary disease-causing gene (Swaggart K A, Demonbreun A R, Vo A H, Swanson K E, Kim E Y, Fahrenbach J P, et al. Annexin A6 modifies muscular dystrophy by mediating sarcolemmal repair. Proceedings of the National Academy of Sciences of the United States of America. 2014; 111(16):6004-9).

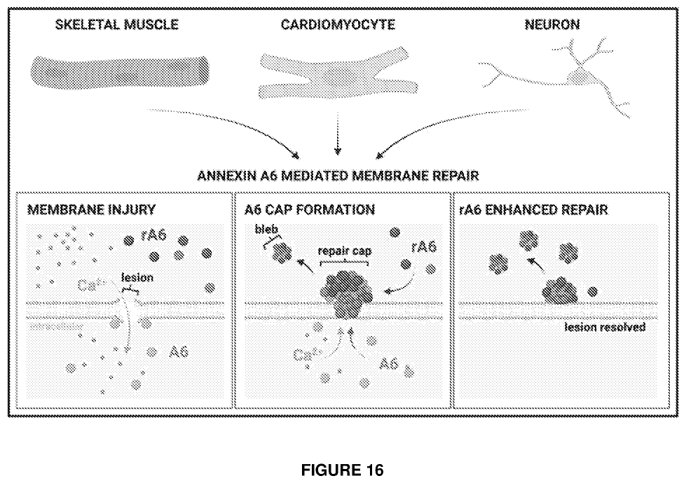

Recombinant annexins mediate closure of membrane lesions. Annexin A6 has been implicated in membrane folding and the constriction forces needed to pull wound edges together for eventual fusion (Boye T L, Jeppesen J C, Maeda K, Pezeshkian W, Solovyeva V, Nylandsted J, et al. Annexins induce curvature on free-edge membranes displaying distinct morphologies. Scientific reports. 2018; 8(1):10309; Boye T L, Maeda K, Pezeshkian W, Sonder S L, Haeger S C, Gerke V, et al. Annexin A4 and A6 induce membrane curvature and constriction during cell membrane repair. Nature communications. 2017; 8(1):1623). The studies by Boye and colleagues used artificial membranes and recombinant purified proteins. Data provided herein revealed A6GFP-positive membrane blebs emanating from the annexin repair cap (model in FIG. 16). Additionally, exogenously added recombinant annexin A6 protein also localized to blebs at the site of repair. Together these data indicate that annexin A6 protein can bind exposed membrane phospholipids at the site of injury from the cell interior or exterior. As shown herein, recombinant annexin A6 protein elicited a strong and significant dose-dependent improvement in repair. In some embodiments, and as described herein, an annexin cocktail containing multiple annexin proteins is contemplated.

In summary, this work demonstrated that annexin A6 forms repair caps which mediate resealing, and this process is conserved across multiple cell types. Importantly, it was shown that exogenously administered recombinant annexin A6 rapidly binds to the damaged membrane in skeletal muscle, cardiomyocytes and neurons suggesting a broad role for annexin A6 in repair.

### Example 3

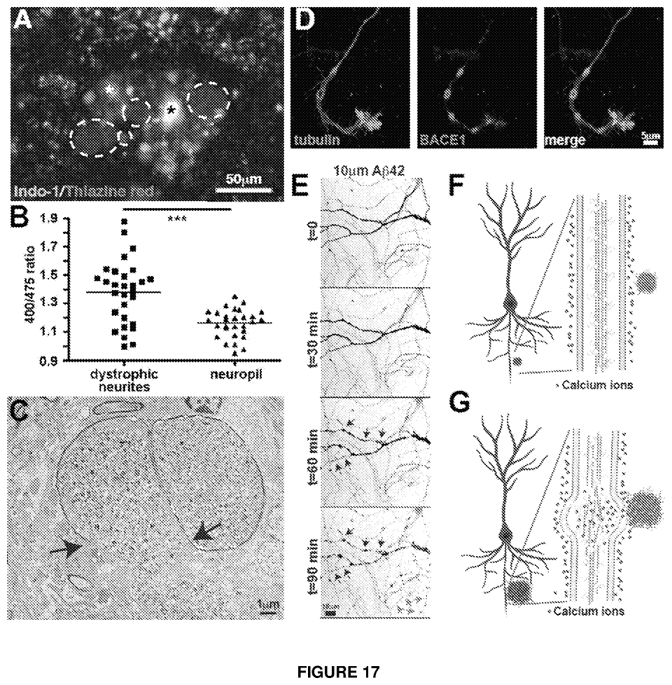

As at the border of the amyloid plaque causes calcium (Ca²⁺) influx in peri-plaque dystrophic neurites (DNs), resulting in microtubule disruption and axonal transport impairment (FIG. 17) [Kuchibhotla et al., Neuron 59, 214-225, doi:10.1016/j.neuron.2008.06.008 (2008). PMC2578820; Sadleir et al., Acta Neuropathol 132, 235-256, doi:10.1007/s00401-016-1558-9 (2016). PMC4947125]. Preliminary results also indicate that annexin A6, a membrane resealing protein involved in muscle myofiber repair, reduced the formation of plaque-associated DNs and the accumulation of tau phosphorylated at Thr181(p-tau181) and Thr231 (p-tau231) in DNs (FIGS. 11C, 18, 23, and 24). p-tau181 and p-tau231, together with p-tau217, have emerged as important early cerebrospinal fluid (CSF) and plasma biomarkers of AD that track with amyloid pathology. Importantly, results presented herein show that annexin A6 repairs Aβ3-damaged neuronal membranes and therefore represents a novel AD therapeutic. Additionally, DNs that surround amyloid plaques have been implicated in the seeding and spreading of pathologic AD tau, suggesting that the DN may act as the crucible in which Aβ3-induced pathogenic tau is formed. The connection between Aβ and tau is one of the great unsolved mysteries of AD. While it is becoming clear that amyloid deposition precedes tau phosphorylation and neurofibrillary tangle (NFT) formation, the link between these processes is poorly understood. As deposition in plaques occurs first, followed by the appearance of DNs containing neurotransmitter (suggesting pre-synaptic origin), then DNs with abnormal p-tau. Other studies support axonal origin of most DNs. It was hypothesized that DNs around plaques are sites of pathogenic tau phosphorylation and aggregation that play a role in AD tau seeding and spreading throughout the brain. CSF and plasma p-tau181, p-tau217, and p-tau231 effectively distinguished AD cases from controls and non-AD dementias and can predict which patients will develop disease over a decade before symptom onset. Increased p-tau181, p-tau217, and p-tau231 is one of the earliest pathologic tau changes associated with amyloid deposition. Results provided herein show that reducing Aβ3-induced neurite membrane damage decreased Ca²⁺ influx, microtubule disruption, axonal transport impairment, aberrant kinase activation, and the formation of pathologic p-tau, thus preserving neuronal function. Since abnormally phosphorylated and aggregated tau correlate with cognitive decline, it was critical to understand how amyloid deposition leads to AD tau seeding and spreading. Without wishing to be bound by any particular theory, disrupting the link between Aβ and tau pathologies may provide a therapeutic approach to AD by rendering amyloid plaques less able to form DNs and seed and spread pathologic tau.

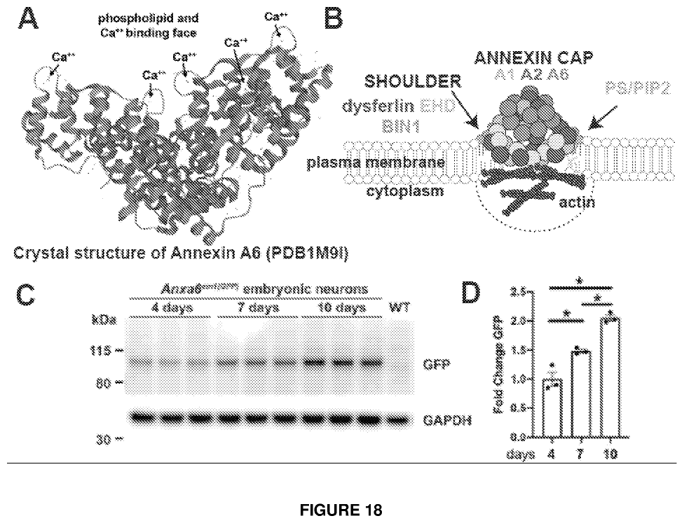

Experiments described in the present Example show that Aβ at the borders of plaques causes neurite membrane damage, Ca²⁺ leakage, microtubule disruption, axonal transport impairment, and formation of DNs (FIGS. 17F and 17G), which provide the environment for pathogenic tau phosphorylation, seeding, and spreading. The present Example also explored membrane repair in neurons. All cells have membrane repair capacity, but it is highly developed in large, terminally differentiated cells with extensive surface area that undergo stress and Ca²⁺ fluctuations, such as myofibers in muscle. In muscle, when the balance between membrane damage and repair is disrupted by genetic mutations, muscular dystrophies occur. Neurons are large post-mitotic cells with extensive plasma membrane area, and are likely more vulnerable to membrane damage due to multiple causes. Therefore, neurons must respond rapidly to repair membrane damage for preserving function and viability. Indeed, neurons express major membrane resealing proteins found in other cells, including annexin A6, Bin1, EHD proteins, and dysferlin, among others (FIG. 18). Cell membrane changes and rigidity associated with aging could overwhelm membrane repair mechanisms and render neurons more susceptible to Aβ-induced membrane damage, which could partly explain why age is the primary risk factor for AD. In muscle myofibers, annexin A6 is the first membrane resealing protein that is recruited by Ca²⁺ influx to the site of membrane damage (FIG. 18). Other annexins and resealing proteins then follow to form the membrane cap complex that seals the membrane breach and allows repair to occur. Although well studied in muscle myofibers, this mechanism is virtually unexplored in neurons. Microglia appear to have a role in the protection of neurites against Aβ-induced damage, as exemplified by the dramatic increase in DNs in 5XFAD;TREM2−/− mice in which microglia fail to surround and wall-off plaques.

Evidence suggests that the immediate vicinity of the amyloid plaque creates a locally toxic environment for neurons. Live imaging of 5XFAD brain slices loaded with ratiometric Ca²⁺ dye Indo-1 showed elevated Ca²⁺ in peri-plaque DNs (FIGS. 17A and 17B). EM of 5XFAD brain reveals axonal membrane breach in the vicinity of Aβ plaques (FIG. 17C). Primary neurons treated with Aβ42 in vitro display axonal swellings that accumulate BACE1 and show tubulin disorganization, reminiscent of DNs in vivo (FIGS. 17D and 17E). Therefore, it was hypothesized that contact of axons with Aβ causes membrane disruption and Ca²⁺ leak, which leads to aberrant kinase activation, microtubule disruption, impaired axonal transport, defective protein turnover, axonal swelling and DN formation, accumulation of amyloidogenic proteins like APP and BACE1, and elevated Aβ production in DNs (FIGS. 17F and 17G). Maintaining membrane integrity by enhancing repair mechanisms could reduce this toxic cascade in the vicinity of plaques, thus slowing disease progression.

The annexins exhibit broad tissue distribution and function in membrane and actin remodeling, immune cell modulation, and wound repair. As described herein, annexin A6 plays a key role in membrane resealing in skeletal muscle and heart. Like other annexins, A6 binds negatively charged phospholipids such as phosphatidylserine in the presence of Ca²⁺, but it is unique in having 8 annexin repeats making it particularly sensitive to Ca²⁺ (FIG. 18A). As has many deleterious effects on membranes, such as pore formation, detergent-like activity, deformation, thinning and increased rigidity, all of which can cause Ca²⁺ leakage into the cell. Because it was hypothesized that Aβ causes membrane damage leading to Ca²⁺ influx, dysregulated kinase pathways, microtubule instability and impaired vesicular transport, the present Example explored the role of annexin A6 in membrane resealing in vitro and in vivo.

Upon acute membrane injury, annexin A6 initiated the formation of a repair cap protein complex, recruiting annexins A1 and A2 to the site of damage (FIG. 18B). Annexin A6 and the other proteins in this complex, annexins A1 and A2, and the shoulder proteins BIN1, dysferlin, and the EHD family proteins, are all expressed in brain. Importantly, mutations in BIN1 are some of the strongest genetic risk factors associated with AD, suggesting that the membrane repair function of BIN1 may be compromised by these mutations in AD. Annexin A6 is key to initiating and forming the repair cap complex, since a truncation mutant consisting of only the N-terminal 32 amino acids of annexin A6, N32, and a missense mutation that is deficient in Ca²⁺ binding, E233A, both exert a strong dominant-negative effect on repair cap assembly and membrane repair. Previously, it was shown that recombinant annexin A6 or A5 reduced dye leakage from liposomes induced by Aβ and various membrane disruptors. Injection of exogenous recombinant annexin A2 was protective in a traumatic brain injury model by decreasing BBB permeability and promoting angiogenesis, and annexin A5 protected choroid plexus cells from damage by Aβ42. Injection of human recombinant annexin A1 acutely decreased blood brain barrier permeability in young 5XFAD and Tau P301 L mice as well, further suggesting beneficial effects of annexins in neurological disease. Although annexin A6 constitutive knockout (KO) mice were shown to have normal immune and cardiac function, more recently they were found to have increased sensitivity to painful mechanical stimuli due to loss of annexin A6 interaction with the Piezo2 channel. These in vivo results are intriguing, but do not directly address how annexin A6 plays a role in membrane resealing of neurons in the brain, nor the role of membrane repair in AD.

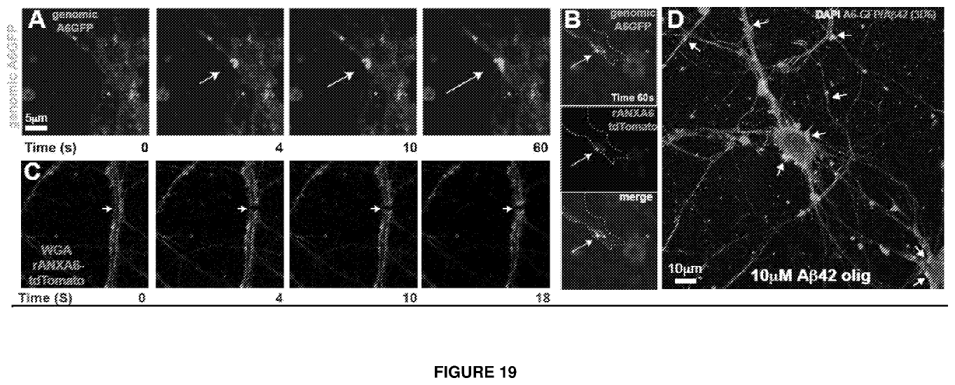

To investigate annexin A6 in membrane repair, a knockin mouse expressing GFP-tagged A6 (A6-GFP) from the endogenous mouse locus by CRISPR-Cas9 genome editing (Anxa6em1 (GFP) mice) was generated. In primary cortical neurons cultured from Anxa6em1(GFP) mouse embryos, A6-GFP increased as neurons matured in vitro (FIGS. 2C and 2D), suggesting an important neuronal function. Laser injury of Anxa6em1(GFP) primary neurons showed that A6-GFP accumulated within seconds at the site of membrane damage (FIG. 19A), very similar to the well-established muscle laser injury paradigm. In skeletal and heart muscle cells, exogenous recombinant annexin A6 localizes rapidly to the site of laser injury and enhances membrane repair. Similarly, exogenous recombinant tdTomato-tagged annexin A6 colocalized with genomically expressed A6-GFP at the laser injury site in both the neuronal cell body (FIG. 19B) and neurite (FIG. 19C) in Anxa6em1(GFP) primary neurons, suggesting that exogenous recombinant A6 may mediate neuronal membrane repair. To determine whether annexin A6 localizes to sites of membrane damage caused by Aβ, Anxa6em1 (GFP) primary neurons were treated with oligomeric Aβ42 followed by anti-GFP and anti-Aβ immunofluorescence microscopy (FIG. 19D). It was found that A6-GFP accumulated at sites where Aβ42 contacted the membrane, suggesting recruitment of A6-GFP to sites of Aβ-induced membrane injury.

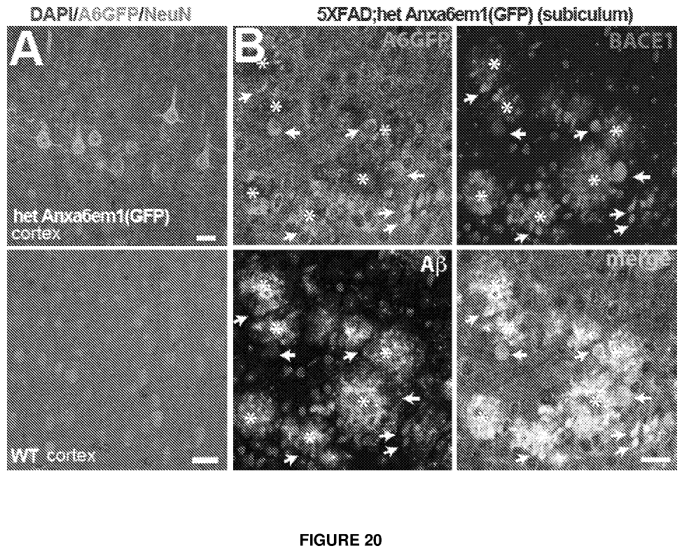

Next, Anxa6em1 (GFP) mouse brain sections were analyzed by immunofluorescence microscopy, which showed A6-GFP expression in neurons of cortex (FIG. 20A) and other brain regions, but not in microglia or astrocytes, indicating that neurons are major cell types that express annexin A6 in the brain. Anxa6em1 (GFP) mice were then crossed to 5XFAD mice74, which are a well-established early onset model of amyloid pathology. In addition to neuronal soma and dendrites, A6-GFP accumulated in DNs around amyloid plaques of Anxa6em1 (GFP); 5XFAD mice, as shown by colocalization with the DN marker BACE1 (FIG. 20B).

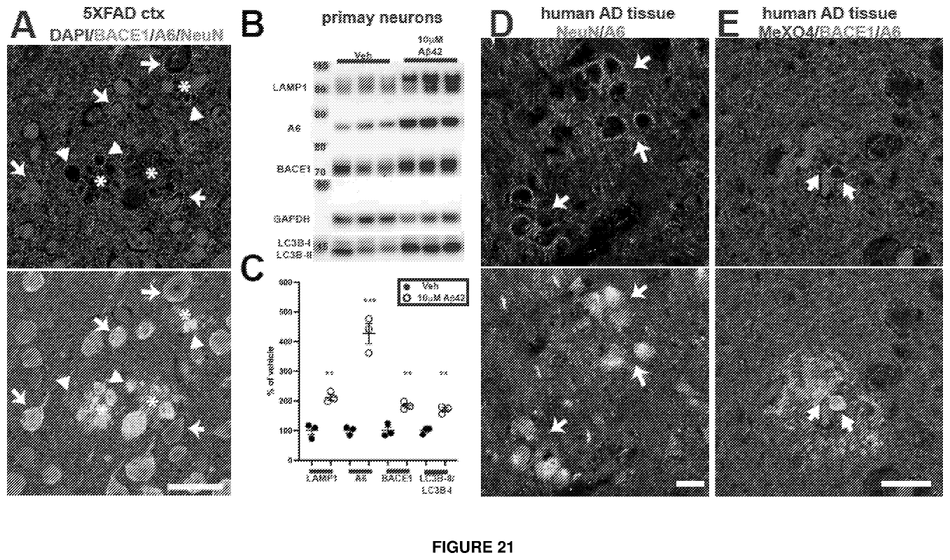

To confirm that A6-GFP localizes similarly to endogenous A6, 5XFAD brains were stained with an annexin A6 antibody. As with A6-GFP localization in Anxa6em1(GFP); 5XFAD brain, endogenous annexin A6 in 5XFAD brain was localized at the plasma membrane of neurons and DNs around plaques (FIG. 21A). Immunoblot analysis of wild-type primary mouse neurons treated for 72 hours with Aβ42 oligomers showed increased annexin A6 (4×) and DN markers BACE1, LAMP1, and LC3B-II (FIGS. 21B and 21C). In human AD hippocampus, annexin A6 also localized to the membrane of neurons and to BACE1-containing DNs near amyloid plaques (FIGS. 21D and 21E). Together, these results demonstrated that annexin A6 is localized to mouse and human neurons and DNs around amyloid plaques, and that Aβ increased annexin A6 and other proteins associated with DNs.

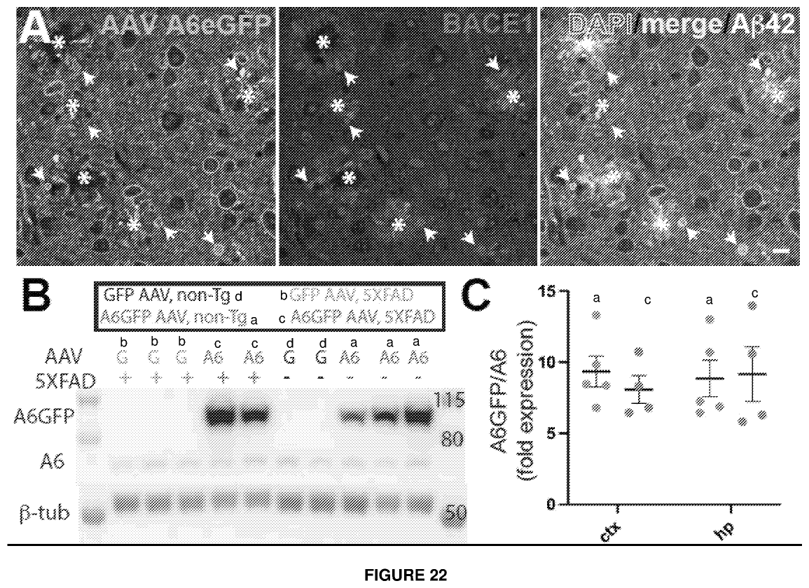

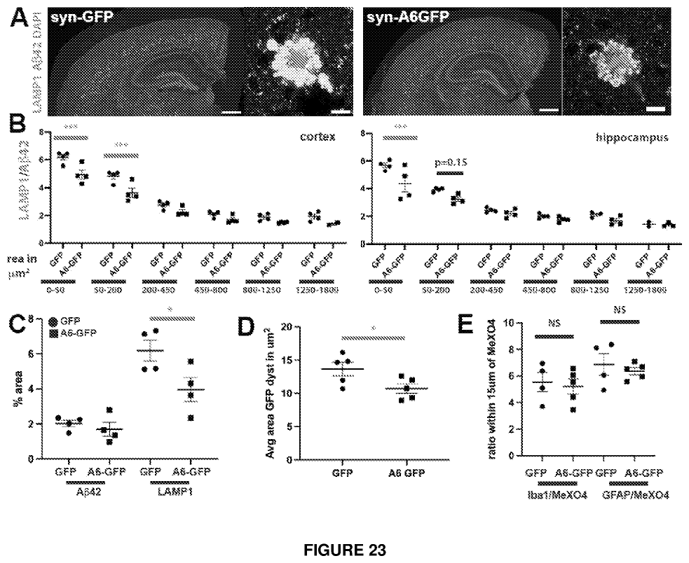

To test whether annexin A6 increases membrane repair and reduces DN formation, A6-GFP was overexpressed in 5XFAD mouse brain from an AAV8 vector driven by the neuron-specific human synapsin (syn) promoter77 (VectorBuilder). On P0, 5XFAD pups were ICV-injected with either syn-GFP AAV or syn-A6-GFP AAV, then aged to 4 months and brains harvested. GFP fluorescence revealed A6-GFP expression in neurons and a large number of DNs, similar to the endogenous A6 pattern (FIG. 22A). A6-GFP colocalized with BACE1 in DNs, indicating A6-GFP reached the correct location to repair membrane damage. Immunoblot analysis showed that A6-GFP was expressed approximately 8 fold higher than endogenous A6 (FIGS. 22B and 22C), which mice tolerated well with no obvious ill effects. To determine the effects of A6-GFP on amyloid pathology and DN formation, sections were stained with antibodies against Aβ42 and LAMP1, a well-established DN marker (FIG. 23A). Nikon NIS-Elements software was used to define each plaque and its associated DN halo and determine the ratio of the respective areas stained for LAMP1 and Aβ42 (FIG. 23B). Smaller amyloid plaques grow at a faster rate and have proportionally greater amounts of neuritic dystrophy, so plaques were stratified based on Aβ core size before averaging the ratios. As expected, smaller plaques had a higher LAMP1:Aβ42 ratio than larger plaques. Importantly, a significant decrease of the LAMP1:Aβ42 ratio of smaller plaques was observed in hippocampus and cortex of 5XFAD brains injected with A6-GFP AAV compared to those injected with GFP AAV (FIG. 23B). Smaller plaques are thought to be undergoing the most rapid growth and causing the greatest neurotoxicity, so reduction of dystrophic neurites around smaller plaques is likely to have beneficial effects. The total percent area positive for LAMP1 in the cortex was significantly decreased, while that of Aβ42 was unchanged (FIG. 23C), indicating that A6-GFP specifically reduced dystrophic neurites with minimal effects on Aβ deposits. Using higher magnification confocal images, it was observed that the area of individual GFP+ DNs was significantly reduced in A6-GFP AAV injected mice compared to those injected with GFP AAV (FIG. 23D). A6-GFP overexpression did not appear to affect microglial or astrocytic responses to amyloid plaques, as the ratio of microglial marker Iba1 to the amyloid stain MeXO4 and astrocyte marker GFAP to MeXO4 in the area within 15 μm of the plaque core was unchanged (FIG. 23E). Together, these results showed that annexin A6 overexpression reduced the formation of DNs but did not affect Aβ pathology.

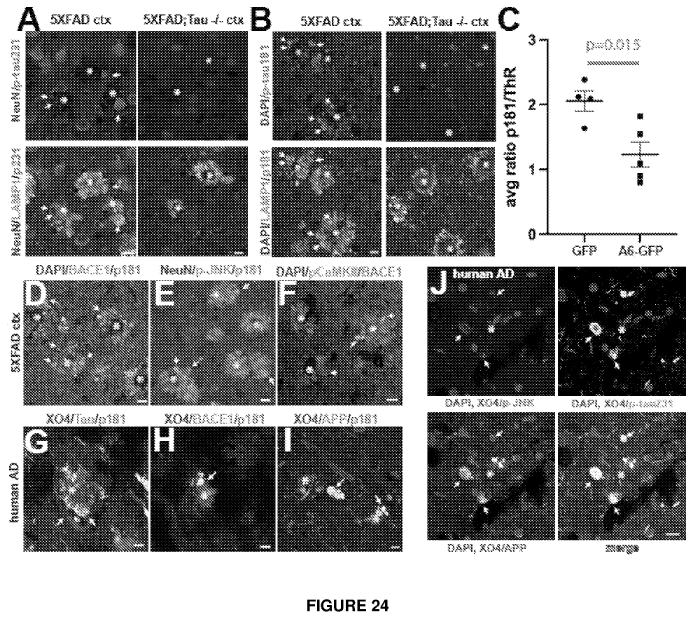

Previous work has suggested that plaques and associated neuritic dystrophy play a key role in pathologic tau seeding and spreading [He, Z. et al. Nat Med 24, 29-38; Leyns, C. E. G. et al. Nat Neurosci 22, 1217-1222; Parhizkar, S. et al. Nat Neurosci 22, 191-204]. Additionally, new phospho-tau biomarkers have been identified that track closely with A∇ pathology and predict AD years in advance [Barthelemy et al., J Exp Med 217, doi:10.1084/jem.20200861 (2020). PMC7596823; Barthelemy, N. R. et al. Nat Med 26, 398-407; Suarez-Calvet, M. et al. EMBO Mol Med 12, e12921, doi:10.15252/emmm.202012921 (2020); Mattsson-Carlgren, N. et al. Sci Adv 6, eaaz2387, doi:10.1126/sciadv.aaz2387 (2020)]. Using specific antibodies for p-tau231 (FIG. 24A) or p-tau181 (FIG. 24B), it was observed that p-tau231 and, even more noticeably, p-tau181 accumulated in DNs of 5XFAD brains, as shown by colocalization with LAMP1 and BACE1 (FIGS. 24A, 24B, and 24D). No signal for p-tau 231 or p-tau181 was detected in 5XFAD; Tau−/− brain (FIGS. 24A and 24B). Since A6-GFP overexpression reduced DNs in 5XFAD brain (FIG. 23), it was hypothesized that A6-GFP would also decrease p-tau associated with DNs. Indeed, a significant reduction in the ratio of p-tau181 to amyloid was observed in 5XFAD mice injected with syn-A6-GFP AAV compared to those injected with syn-GFP AAV (FIG. 24C). It was hypothesized that increased kinase activity in DNs could be responsible for p-tau accumulation. Activated, phosphorylated forms of two kinases, CaMKII and c-jun kinase (JNK), known to phosphorylate p-tau181, p-tau217, and p-tau23181,82, were colocalized with p-tau181 in 5XFAD DNs (FIG. 24D-24F). In human AD brain, p-tau181 colocalized with total tau, BACE1, and APP in DNs around plaques (FIG. 24G-24I) and p-tau 231 colocalized with p-JNK and the DN marker APP (FIG. 24J). These results showed that DNs are sites of early tau phosphorylation associated with amyloid pathology and that decreasing DN formation via annexin A6 overexpression will reduce p-tau181, a biomarker that predicts AD, and perhaps other pathologic p-tau isoforms.

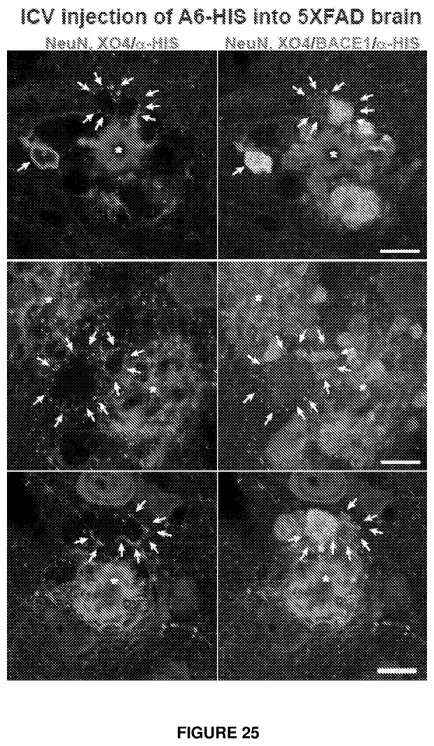

It has been shown in muscle that not only cellular overexpression of annexin A6, but exogenous recombinant A6 is able to enhance membrane repair [Demonbreun, A. R. et al. J Clin Invest 129, 4657-4670, doi:10.1172/JC1128840 (2019). PMC6819108](FIG. 25). Overexpressed and exogenous recombinant annexin A6 promote myofiber membrane resealing after laser injury and reduce dye influx to an equivalent amount [Demonbreun, A. R. et al. J Clin Invest 129, 4657-4670, doi:10.1172/JC1128840 (2019). PMC6819108]. This strongly suggested that recombinant annexin A6 could serve as a therapeutic agent, since it can home to sites of membrane damage from the extracellular space [Demonbreun, A. R. et al. J Clin Invest 129, 4657-4670, doi:10.1172/JC1128840 (2019). PMC6819108]. Recombinant A6 is also effective in acute (lytic damage from cardiotoxin injection) and chronic (muscular dystrophy due to sarcoglycan mutation) muscle injury models [Demonbreun, A. R. et al. J Clin Invest 129, 4657-4670, doi:10.1172/JC1128840 (2019). PMC6819108]. Importantly, following ICV injection into 5XFAD brain, HIS-tagged recombinant A6 (A6-HIS) localized to membranes of BACE1+ DNs (FIG. 25).

### Materials and Methods

AAV mediated A6-GFP expression. VectorBuilder cloned the mouse Annexin A6 cDNA (NM_013472.5) into an AAV expression vector with a C-terminal eGFP tag under control of the neuronal promoter synapsin (syn). They prepared serotype 8 AAV expressing either A6-GFP or GFP alone. Genomic titer was determined by quantitative PCR. 5XFAD transgene positive males were crossed to SJL/B6 hybrid females in timed matings to generate transgene negative and positive littermates. On P0, each pup in a litter was cryoanesthetized and 2 μl containing 2×10¹⁰ viral genomes of A6-GFP AA8 or GFP AAV8 was injected into the ventricle of each hemisphere using a 10 μl Hamilton syringe. Pups were placed in home cage on a heating pad to recover, then reunited with their mother. At 4.5 months of age, they were anesthetized with a lethal dose of ketamine/xylazine and transcardially perfused with PBS containing protease and phosphatase inhibitors. One hemibrain was dissected into hippocampus, cortex and midbrain which were snap-frozen separately. The other hemibrain was drop fixed in 10% buffered formalin overnight at 4° C., then transferred to 20% w/v sucrose in 1×PBS for 24 hours, then stored in 30% w/v sucrose in 1×PBS with azide. All animal work was done with the approval of the Northwestern University IACUC.

Immunofluorescence and microscopy. 10% formalin-fixed hemibrains were sectioned coronally at 30 μm on a freezing sliding microtome and collected in cryopreserve (30% w/v sucrose, 30% ethylene glycol in 1×PBS). Three sections were stained and imaged per mouse.

For quantification of the ratio between LAMP1 and Aβ42 the following staining protocol was used. 1:2000 rat monoclonal anti-LAMP1 (clone 1 D4B, DSHB) and 1:2000 rabbit monoclonal anti-A342 antibody (clone H31 L21, Invitrogen, #700254) in 1% BSA TBS 0.1% triton at 4° C. overnight. Following 3 TBS washes, sections were incubated for 2 hours at room temperature with secondary antibodies: 1:750 donkey anti-rat Alexa 568 (abcam), donkey anti-rabbit 647(ThermoFisher Scientific), 300 nM DAPI.

For quantification of the ratio between p-Tau 181 and Thiazine red plaque cores, the following staining protocol was used. 1:300 rabbit monoclonal anti-p-tau181 (clone D9F46, cell signaling #12885) in 1% BSA TBS 0.1% triton at 4° C. overnight. Following 3 TBS washes, sections were incubated for 2 hours at room temperature with secondary antibodies: donkey anti-rabbit 647(ThermoFisher Scientific), 300 nM DAPI and 1:15,000 dilution of 1 mg/ml Thiazine Red.

All sections were mounted with Prolong Gold (Molecular Probes) and images acquired on a Nikon Ti2 Eclipse widefield microscope with a 1 Ox objective, using NIS Elements software high content method to capture and tile whole sections. All image acquisition settings were maintained the same between treatment groups and genotypes. For image analysis, regions of interest (ROIs) were drawn in cortex and hippocampus. Using the General Analysis tool, thresholding was set to distinguish A342, and LAMP1 positive regions, or p-Tau181 and Thiazine red positive regions, then the percent area covered by each stain from the hippocampal or cortical ROI was calculated. Using the same sections, thresholding in the General Analysis tool was used to define sub-ROIs that correspond to individual plaques having both Aβ42 and LAMP1 or p-Tau181 and ThR positive pixels within cortical and hippocampal ROIs. The General Analysis tool was used to measure area covered by Aβ42 and by LAMP1 in a given sub-ROI (plaque) and the ratio between LAMP1:Aβ42 was calculated in Excel.

Statistics. Student's two-tailed t-test and ANOVA were performed using InStat software (GraphPad Software, Inc., San Diego, CA) to compare means of the various genotypes, genders, and treatment groups. *0.05>p>0.01**0.01>p>0.001***0.001>p>0.0001; Error bars=S.E.M.

A6-HIS Ventricle injection. A6-HIS was purified from E. coli using standard methods. A 5 month old 5XFAD male mouse was anesthetized with isoflurane in stereotaxic frame, a central incision made to expose skull. On the left side of brain, a small hole was drilled with 25 gauge needle at the site to target ventricle (B-L or A-P: −0.60 mm; M-L: +1.30 mm) then a 10 μl Hamilton syringe was used to inject 3 μl of 21 mg/ml A6-HIS at a depth of −2.00 mm, 3 times with 2-3 min break in between for a total of 9 μl, containing 189 μg A6-HIS, a dose of 6.3 mg/kg for a 30 gm mouse. After injection, the incision was stapled shut and the mouse recovered from anesthesia.

Three hours after surgery, the mouse was anesthetized with a lethal dose of ketamine/xylazine and transcardially perfused with PBS containing protease and phosphatase inhibitors. Both hemibrains were drop fixed in 10% buffered formalin overnight at 4° C., then transferred to 20% w/v sucrose in 1×PBS for 24 hours, then stored in 30% w/v sucrose in 1×PBS with azide.

10% formalin-fixed hemibrains were sectioned sagittally at 30 μm on a freezing sliding microtome and collected in cryopreserve (30% w/v sucrose, 30% ethylene glycol in 1×PBS). Three sections were stained and imaged per mouse.

To image A6-HIS localization, sections were antigen retrieved for 40 min at 80° C. in 1 mM sodium citrate at pH 9, then stained as follows: 1:500 chicken anti-NeuN (Millipore), 1:300 mouse monoclonal anti-BACE1 (clone 3D5) and 1:300 rabbit monoclonal anti-HIS (clone D3110, Cell signaling, #12698) in 1% BSA TBS 0.1% triton at 4° C. overnight. Following 3 TBS washes, sections were incubated for 2 hours at room temperature with secondary antibodies: 1:750 donkey anti-chicken Alexa 405 (Jackson Immunologicals), donkey anti-mouse 488 (ThermoFisher Scientific) donkey anti-rabbit 568 (ThermoFisher Scientific), Methoxy XO4 20 μM.

All sections were mounted with Prolong Gold (Molecular Probes) and images acquired on a Nikon A1R confocal microscope with a 60× objective, using NIS Elements software.

### Example 4

In this Example, the role of annexin A6 in DN formation is investigated. It is hypothesized that neuronal annexin A6 maintains membrane integrity during amyloid plaque growth, reducing Ca²⁺ influx, stabilizing microtubules and reducing DN formation. It is further hypothesized that overexpressed A6 will reduce Aβ-induced DN formation, while A6 deficiency or dominant negative A6 will increase DN pathology.

Whether overexpression of wild type or dominant negative (E233A) annexin A6 increases or decreases membrane repair, respectively, following laser injury in primary mouse neurons, as assessed by membrane-impermeant dye penetration and Ca²⁺ sensor imaging, is determined. The role of other repair cap components in neuronal membrane repair is also determined.

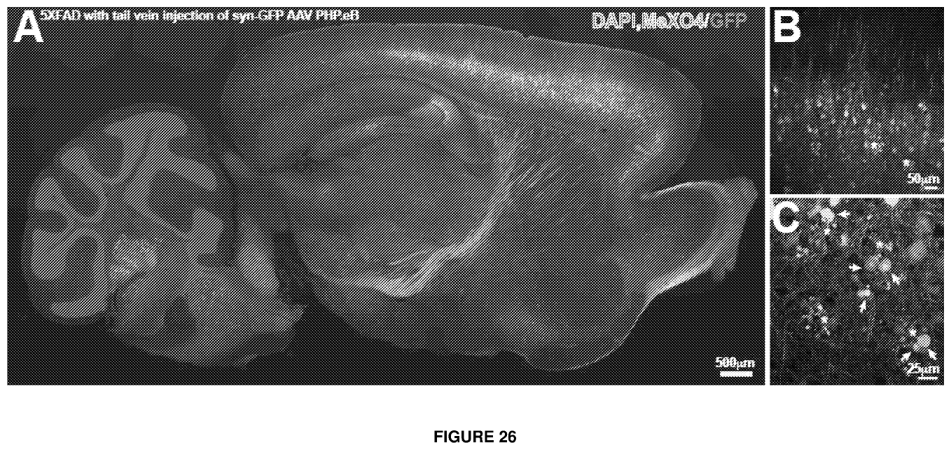

C57BL/6 E15.5 mouse primary neurons are isolated and plated in coverslip bottom dishes for imaging (MatTek) and 12-well plates for biochemical analysis. An AAV PHP.eB (blood-brain barrier penetrant AAV55; FIG. 26) vector is engineered to express syn-GFP, syn-A6-GFP, or syn-A6(E233A)-GFP (VectorBuilder). Dominant negative A6(E233A) has been studied and found to strongly inhibit membrane repair [Demonbreun, A. R. et al. J Clin Invest 129, 4657-4670, doi:10.1172/JC1128840 (2019)]. AAV vectors are added to neurons (1×10⁸ VG/μl; MOI=100-1000) and cultured for 7-14d. Parallel cultures are infected with AAV vectors that express shRNA constructs to knockdown repair cap shoulder proteins annexins A1 and A2, Bin1, and dysferlin. In addition, for annexins A1 and A2, primary neurons are infected with AAV vectors expressing dominant negative A1 and A2 (A1(D171A), A2(D161A)). At 7-14 days in vitro (DIV), neurons will undergo laser injury as described herein on a Nikon A1R-multiphoton confocal microscope with a Chameleon Vision titanium sapphire laser (690-1040 nm) and a 25× (1.1 NA) water immersion lens. To measure Ca²⁺ influx, neurons are co-infected with AAV expressing Ca²⁺ sensors jRCaMP1 or jRGECO185 (AddGene). To measure dye influx, the membrane-impermeant dye FM 4-64 (ThermoFisher) is added to the bath immediately before laser injury. Ca²⁺ influx and dye penetration are imaged on the same Nikon A1R-multiphoton confocal microscope used to cause the laser injury and quantified in time-series images using NIS-Elements software to assess the effect of A6 constructs on membrane resealing efficiency.

It is also determined whether overexpression of wild type or dominant negative annexin A6 increases or decreases membrane repair, respectively, in primary mouse neurons treated with Aβ42 oligomers and fibrils, as assessed by dye penetration, Ca²⁺ influx, neuritic beading, axonal transport, caspase 3 activation, and LDH release.

Primary neurons are prepared as described herein and plated on coverslip bottom dishes for imaging (MatTek) and 12-well plates for biochemical analysis. AAV PHP.eB expressing syn-GFP, syn-A6-GFP or syn-A6(E233A)-GFP is added to neurons and cultured for 7-14d, then treated with oligomeric or fibrillar Aβ42 (rPeptide) preparations, as per previous methods [Sadleir et al. Acta Neuropathol 132, 235-256, doi:10.1007/s00401-016-1558-9 (2016); Sadleir et al., J Biol Chem 287, 7224-7235, doi:10.1074/jbc.M111.333914 (2012)]. A high [Aβ42](10 μM) is used to simulate the high local [Aβ] experienced by peri-plaque DNs. For Ca²⁺ imaging, neurons is co-infected with AAV expressing jRCaMP1 or jRGECO185, then subjected to live imaging 0-6 hrs after Aβ42 addition. To measure neuritic beading, neurons are transfected with BacMam RFP-tubulin (ThermoFisher) two days before Aβ42 addition, then imaged 0-16 hours following Aβ42 treatment. To measure axonal transport, LysoTracker DeepRed (ThermoFisher) and MitoTracker (Cell Signaling) are added prior to Aβ42 addition, then imaged 0-16 hours following Aβ42 treatment. Imaging is performed on a Nikon W1 Dual CAM Spinning Disk confocal using a 40× objective. Cell viability is measured by cleaved caspase 3 immunoblot analysis, imaging propidium iodide uptake, and lactate dehydrogenase activity (Promega) in media.

It is determined whether overexpression of wild type or dominant negative (E233A) A6 in 5XFAD mice decreases or increases DN formation and glial activation around plaques, respectively. Dye penetration, Ca²⁺ influx, caspase 3 activation, behavior, and EM for microtubule integrity is assessed.

The AAV PHP.eB vector was able to cross the blood brain barrier and achieve widespread long-term expression of proteins in the brain (FIG. 26)). 5XFAD mice receive single tail vein injections of 1×10¹² VG of AAV PHP.eB syn-A6-GFP, AAV PHP.eB syn-A6(E233A)-GFP, and control AAV PHP.eB syn-GFP to express wild type and dominant negative A6-GFP fusion proteins in the brain. 5XFAD mice are well-established models of early Aβ pathology, with plaque deposition starting at 2 months of age, gene expression changes that are similar to those seen in human AD brain [Neuner et al., Neuron 101, 399-411 e395, doi:10.1016/j.neuron.2018.11.040 (2019). PMC6886697; Heuer et al., Learn Mem 27, 355-371, doi:10.1101/lm.051839.120 (2020)], and neuron loss with age [Oakley et al., J Neurosci 26, 10129-10140 (2006); Eimer et al., Molecular neurodegeneration 8, 2, doi:10.1186/1750-1326-8-2 (2013)].

In an AD prevention paradigm, 5XFAD mice (n=20/group; 10 males, 10 females) are tail vein-injected with AAV vectors at 1.5 months of age and harvested 3 or 8 months later to assess the effects of annexin A6-GFP fusion proteins on the formation of DNs and Aβ plaques. In an AD treatment paradigm, 5XFAD mice (n=20/group; 10 males, 10 females) are tail vein-injected at 4 months of age and harvested 5 months after injection. Before brain harvest, behavioral analyses is performed in the Northwestern Behavioral Phenotyping Core Facility. Behavioral assays include: 1) Novel object recognition, which assesses declarative memory by measuring the ratio of time spent exploring a novel object compared to a familiar one90, 2) Spontaneous alternation in the Y-maze, which tests working memory by measuring the amount of spontaneous alternation between the three arms of the maze for 5 min [Oakley et al., J Neurosci 26, 10129-10140 (2006)], 3) contextual and cued fear conditioning to measure the animal's ability to associate a space or tone with a foot shock, a test of hippocampal dependent memory, 4) Morris water maze, a sensitive test of spatial memory in which the animal learns to remember the location of a hidden platform in a tank of opaque water [Gobeske et al., PLoS One 4, e7506, doi:10.1371/journal.pone.0007506 (2009)].

At brain harvest, mice are transcardially perfused with PBS, the left hemibrain fixed in 10% PFA and cryopreserved in 30% sucrose/PBS for immunofluorescence microscopy, and the right hemibrain sub-dissected into cortex, hippocampus, and cerebellum (negative control) and flash frozen in LN2 for biochemical analysis. Floating coronal sections (30 μm) from fixed hemibrains are stained for amyloid pathology using anti-A342 antibody (Thermofisher), anti-Aβ3D6 antibody (Elan), amyloid dyes Thiazine red (ThR; Sigma) or MethoxyXO4 (MeXO4; HelloBio). DNs are assessed using antibodies recognizing the DN markers LAMP1 (1 D4B clone), BACE1 (Abcam), APP (Abcam), and reticulon 3 (RT3; Millipore). Microglia and astrocytes are assessed using antibodies recognizing Iba1 (all microglia), CD68 (activated microglia), GFAP (all astrocytes) and C3 (activated astrocytes). Using NIS-Elements software, the area ratio of DNs to Aβ plaques in cortex and hippocampus is determined as shown in FIG. 23 [Sadleir et al., PLoS One 17, e0263332, doi:10.1371/journal.pone.0263332 (2022); Sadleir et al., Curr Alzheimer Res 18, 283-297, doi:10.2174/1567205018666210713125333 (2021)]. Areas immunostained for Iba1, CD68, GFAP, and C3 are quantified in the peri-plaque region within 15 μm of the plaque core and in the cortex and hippocampus overall. Aβ+ percent area, plaque number, and plaque size is also quantified to determine the effects of A6-GFP and A6(E233A)-GFP overexpression on Aβ pathology. For mice aged to 9.5 months (late prevention paradigm timepoint), NeuN staining is quantified in the subiculum and layer 5 of the cortex to determine if A6-GFP and A6(E233A)-GFP decreases and increases neuron loss, respectively, in 5XFAD mice. Immunostaining with an antibody to activated cleaved caspase 3 (Cell Signaling Technologies), which increases with age in 5XFAD mice89, is performed to determine if A6-GFP and A6(E233A)-GFP decreases and increases apoptosis, respectively.

To address the mechanism by which membrane repair mediated by A6 decreases DNs, Ca²⁺ imaging is performed to determine if overexpression of A6 decreases resting Ca²⁺ levels in DNs. 5XFAD mice (n=20/group; 10 males, 10 females) receive single tail vein injections of 1×10¹² VG of AAV PHP.eB syn-A6-TdTomato, AAV PHP.eB syn-A6(E233A)-TdTomato, and control AAV PHP.eB syn-TdTomato at 1.5 months of age. At 4 months, brains are cut into 350 μm coronal slices on a vibratome and maintained at 37° C. in aerated artificial CSF (aCSF) for live multiphoton imaging. Brain slices are loaded with ratiometric Ca²⁺-sensitive dye Indo-1 AM (Invitrogen) and the far-red amyloid dye NIAD4 (Nomadics, Inc.) to mark plaques and Ca²⁺ imaging is performed on a Nikon A1R-multiphoton confocal microscope with Chameleon Vision titanium sapphire laser (690-1040 nm) and 25× (1.1 NA) water immersion lens (FIG. 17A). 400 nm (blue, Ca²⁺ bound):475 nm (green, Ca²⁺ free) ratio will be converted to [Ca²⁺]i as described [Oh et al., J Neurosci 33, 7905-7911, doi:10.1523/JNEUROSCI.5457-12.2013 (2013)] for individual DNs near plaques. Since overexpressed A6 in DNs are marked with red TdTomato fluorescence, Ca²⁺ bound:Ca²⁺ free ratios are compared between TdTomato-positive and TdTomato-negative DNs within individual animals. Additional 5XFAD mice are transduced with AAV vectors as described and brains prepared for EM to assess microtubule ultrastructure and density in peri-plaque DNs in cortex and in white matter tracts, following previously published procedures [Kandalepas et al., Acta Neuropathol 126, 329-352, doi:10.1007/s00401-013-1152-3 (2013)].

Based on results described herein (see FIGS. 23 and 24) showing that A6-GFP overexpression reduced DNs and p-tau181 in 5XFAD mice, and that endogenous or exogenous A6 was rapidly recruited to the site of laser injury in primary neurons, it is expected that overexpression of wild-type A6-GFP and dominant negative A6(E233A)-GFP in neurons will increase and decrease membrane repair, respectively, after laser injury.

Based on annexin A6 being the initiating protein in muscle membrane repair cap formation, it is expected that A6(E233A)-GFP will have the strongest inhibitory effect on membrane resealing, while knockdown or dominant negative constructs of annexins A1 and A2, Bin1, and dysferlin may have smaller but measurable effects on membrane repair. It is also expected that wild-type A6-GFP will reduce Ca²⁺ influx, microtubule depolymerization, and neuritic beading in Aβ42-treated primary neurons to improve axonal transport and neuronal survival. Data provided herein show that A6-GFP overexpression from birth reduces DN formation (FIG. 23). Therefore, it is expected that a decrease and increase in DNs will be seen with tail vein injection of AAV PHP.eB syn-A6-GFP and AAV PHP.eB syn-A6(E233A)-GFP, respectively, at 1.5 months, the time at which plaques are rapidly growing. It is further expected that overexpression of A6-GFP starting at 4 months, when numerous plaques have already formed, will still reduce the area ratio of DN to Aβ plaque, but not as strongly since plaques formed before 4 months of age may have irreversible membrane damage. As at 1.5 months, overexpression of A6(E233A)-GFP starting at 4 months is expected to exacerbate DN formation in 5XFAD mice. It is also expected that mice harvested at 8 months after AAV PHP.eB syn-A6-GFP injection at 1.5 months may have reduced Aβ burden, since BACE1 reduction in DNs around plaques should lead to slower rates of Aβ production and less plaque seeding [Peters et al., Acta Neuropathol 135, 695-710, doi:10.1007/s00401-017-1804-9 (2018). PMC5904228].

### Example 5

In this Example, the roles of annexin A6 and dystrophic neurites in the accumulation of pathologic phosphorylated tau proteoforms and the seeding and spreading of pathologic AD tau is determined.

It is determined whether membrane repair via annexin A6 protects against DN formation and accumulation of p-tau181 and other pathologic tau proteoforms in DNs resulting in decreased AD tau seeding and spreading. Whether dominant negative A6 increases DN formation leading to increased p-tau and elevated tau seeding and spreading is also determined. It is further determined if overexpression of wild type or dominant negative A6 decreases or increases p-tau accumulation and AD tau seeding and spreading, respectively, in DNs around plaques in 5XFAD mice.

AD tau seeds are isolated from severe human AD brain tissue obtained from the Northwestern Alzheimer's Disease Research Center. Briefly, frontal cortex gray matter is homogenized with a dounce homogenizer in 9 volumes (v/w) of high salt buffer with 0.1% sarkosyl and 10% sucrose, then centrifuged at 10,000 g. Pellets are re-extracted twice, supernatants combined, sarkosyl concentration increased to 1% and supernatant centrifuged at 45,000 g. Following PBS washes, the pellet is centrifuged at 250,000 g, resuspended in PBS via sonication, and centrifuged at 100,000 g. This pellet, containing 60-70% of tau, is resuspended in PBS via sonication, centrifuged at 10,000 g, and the final supernatant collected as AD Tau. Total protein concentration is assessed with BCA assay and tau concentration measured by ELISA. 15-30 μg tau per gram of tissue at 10-28% purity can be obtained with this method. When injected into brains of an amyloid mouse model such as 5XFAD, AD tau leads to the appearance of AT8+ DNs on the ipsilateral and contralateral sides, with greater plaque burden leading to increased AT8+ DNs and tau spreading [He et al., Nat Med 24, 29-38, doi:10.1038/nm.4443 (2018)]. While AT8 staining is a hallmark of human AD, this marker is very low in amyloid mouse models in the absence of injected AD tau. To verify that seeding-competent AD tau is successfully isolated, AD tau is injected into 5XFAD mice at 3 months, and tau seeding and spreading is assessed by AT8 immunostaining (ThermoFisher antibody MN1020) at 6 months.

To perform tau seeding experiments, cohorts of 5XFAD mice (n=20/group; 10 males, 10 females) are tail-vein injected with AAV PHP.eB syn-GFP, syn-A6-GFP or syn-A6(E233A)-GFP at 1.5 months, followed by unilateral stereotaxic injection at 3 months of 1 □g AD tau into the dentate gyrus (bregma, −2.5 mm; lateral, −2.0 mm; depth, −2.2 mm) and 1 μg AD tau into the overlying cortex (bregma, −2.5 mm; lateral, −2.0 mm; depth, −1.0 mm) using a syringe (Hamilton; syringe 80265-1702RNR and needle 7803-07)35. As a negative control, tau purified from a cognitively unimpaired, healthy brain without tau pathology is injected. Mouse brains are harvested at 6 months or 9 months of age (3 or 6 months after AD tau injection) after transcardial perfusion of PBS with protease and phosphatase inhibitors (Calbiochem). Whole brains are fixed 24-48 hrs in 4% PFA, then cryopreserved with 30% sucrose/PBS and sectioned coronally at 30 μm. The left cortex is nicked on the surface of the piriform cortex so that ipsilateral and contralateral sides can be identified during staining and imagining. Tau spreading is detected via AT8 immunostaining. DNs are quantified via immunostaining with antibodies recognizing BACE1, LAMP1, p-tau181, and RT3, and amyloid plaques via staining for Aβ42, 3D6, ThR or MeXO4 as described herein. Other p-tau proteoforms associated with the early Aβ-phase of AD (e.g., p-tau217, p-tau231) will also be assessed, as will known Tau kinases, such as p-JNK, p-CaMKII, p-ERK, and CDK5. It is expected that decreased DNs in A6-GFP overexpressing mice will result in reduced p-tau181, p-tau 231, phosphorylated tau kinases, and AT8 staining in DNs and decreased spread of AT8 immunoreactivity to the contralateral side. Iba1, GFAP, and NeuN immunostaining is also analyzed to assess whether expression of wild type or dominant negative A6-GFP affects microglial and astrocytic responses to plaques and neurodegeneration (at 9 months) in the context of seeding with human AD tau.

As indicated above, it is determined whether overexpression of wild type annexin A6 decreases DNs, p-tau, and the seeding and spreading of AD tau in 5XFAD;TREM2^{-/-} mice in the absence of TREM2 signaling in microglia.

Using approaches described herein, the effect of A6-GFP overexpression on the seeding and spreading of AD tau is investigated in 5XFAD;TREM2^{+/+} mice compared to 5XFAD;TREM2^{+/+}*mice. TREM2^{+/+} mice (JAX, strain 027197) are crossed with 5XFAD mice to generate 5XFAD;TREM2^{+/+} and 5XFAD;TREM2^{+/+} littermate mice (n=20/group; 10 males, 10 females), which are then tail vein-injected with AAV PHP.eB syn-A6-GFP or control AAV PHP.eB syn-GFP at 1.5 months, followed by unilateral stereotaxic injection at 3 months with 1 μg AD tau each into dentate gyrus and overlying cortex. Mice are harvested at 6 months or 9 months of age (3 or 6 months after AD tau injection) and whole brains processed for immunofluorescence microscopy. All analyses are performed as described herein. It is expected that even in TREM2−/− mice, A6-GFP overexpression is able to decrease DNs, p-tau181, AT8, and other p-tau proteoforms in DNs and reduce the spread of AD tau in the ipsilateral and contralateral sides of the brain, demonstrating a role for DNs in pathologic tau formation and spread.

Annexin A6 is also correlated with DNs, p-tau proteoforms, tau kinases, microtubule density, activated microglia and astrocytes, and amyloid and tau pathologies in human AD compared to 5XFAD brains. Fixed and frozen frontal cortex, hippocampus and cerebellum (as a control) from non-cognitively impaired amyloid negative (NCI−), NCI amyloid positive (NCI+), mild cognitively impaired amyloid positive (MCI+), and mild, moderate, and severe AD cases (n=10 cases/stage) are obtained from the NU ADRC. Frozen tissue is processed for immunoblot analysis for annexin A6, p-tau181, p-tau217, p-tau231, total tau (Tau5), AT8, p-JNK, p-CaMKII, p-ERK, CDK5, APP/Aβ (6E10 antibody), LAMP1, BACE1, reticulon 3, Iba1, CD68, GFAP, and C3. Immunoblots are quantified on a ProteinSimple blot imager and levels of the above proteins correlated with annexin A6 using Pearson's coefficient. Fixed tissue is cut into 30 μm floating sections and immunostained with antibodies recognizing the proteins above and stained with ThR or MeXO4 to label Aβ plaques and tau tangles and imaged on Nikon T1 widefield and A1 confocal microscopes. Images are quantified using NIS Elements software, immunostained areas calculated as described herein above, and correlated with annexin A6 levels using Pearson's coefficient.

Based on data provided herein showing a clear reduction in DN:amyloid ratio, DN area, and p-tau181:amyloid in DNs (FIGS. 23 and 24), it is expected that in the experiments described above, A6-GFP overexpression in neurons will reduce DNs, p-tau accumulation, and the spread of pathologic tau, whereas overexpression of dominant negative A6(E233A)-GFP will increase these phenotypes. While TREM2 KO increases tau spreading, TREM2 deficiency is not expected to prevent A6-GFP overexpression from decreasing DNs, p-tau, and AD tau seeding and spreading. Because increased p-tau181 in CSF and plasma is one of the earliest tau changes in AD, it is also expected that p-tau181+ DNs appear early in NCI+ brains, while AT8+ DNs appear late in MCI+ and AD brains, which have tau pathology and cognitive decline. It is further expected that A6 will have increased localization to DN membranes, especially in NCI+ brains with early-stage Aβ plaques.

### Example 6

In this Example, it is determined whether exogenous recombinant annexin A6 targeted to the brain is able to restore Ca²⁺ homeostasis and decrease pathologic p-tau, AD tau seeding and spreading, and dystrophic neurite formation around amyloid plaques in 5XFAD mice. Exogenous recombinant annexin A6 targeted to the brain is expected to promote neuronal membrane repair and decrease DN formation, p-tau accumulation, and pathologic tau seeding and spreading, rendering amyloid plaques less toxic to surrounding neurites, thus slowing disease progression.

It is also determined whether recombinant A6-HIS localizes to sites of Aβ42 oligomer or fibril contact with mouse primary neurons and reduces neuronal membrane damage, Ca²⁺ and dye influx, and neuritic beading. Site directed mutatgenesis is used to generate A6(E233A) with 6-HIS (pCMV6-AC-His backbone, PS100002, Origene) and TdTomato tags (PS10010, Origene) as described herein and then A6-HIS, A6(E233A)-HIS, A6-TdTomato, and A6(E233A)-TdTomato is expressed and purified in ExpiCHO or Expi293 cells at the Northwestern Recombinant Protein Production Core as described [Demonbreun et al., J Clin Invest 129, 4657-4670, doi:10.1172/JC1128840 (2019) and herein]. Primary neurons are isolated from E15.5 mouse cortex, cultured for 7 and 14 DIV, and then treated with oligomeric or fibrillar Aβ42 (10 μM) in the presence of A6-HIS or A6(E233A)-HIS (1, 10, 33, 100 μM). A similar experiment using A6-TdTomato and A6(E233A)-TdTomato is conducted for live-imaging time course studies of neuritic beading and axonal trafficking. After 1 hr of Aβ42 exposure, neurons are treated with fluorescently-conjugated wheat-germ agglutinin to label membranes, then fixed and stained with antibodies to Aβ42 and the 6-HIS tag (Cell Signaling, 12698). It is expected that A6-HIS will colocalize with Aβ42 at puncta on the cell membrane (FIG. 19D) in a A6-HIS concentration-dependent manner, similar to binding to injured muscle cells. A6(E233A)-HIS is expected to show greatly reduced localization to the membrane at sites of Aβ42 contact due to its inability to bind phospholipids in the presence of Ca²⁺[Demonbreun et al., J Clin Invest 129, 4657-4670, doi:10.1172/JC1128840 (2019)]. Correlation of A6-HIS and A6(E233A)-HIS with Aβ42 is determined by Pearson's Correlation coefficient in NIS-Elements. The effects of A6-HIS and A6(E233A)-HIS on Aβ42-induced Ca²⁺ and dye influx, microtubule disorganization, lysosome trafficking, accumulation of DN markers LAMP1 and BACE1, and viability is also determined, as described herein for neurons overexpressing A6-GFP or A6(E233A) from AAV. It is expected that adding exogenous A6-HIS will reduce Ca²⁺ and dye influx, neuritic beading, and accumulation of markers of DNs and apoptosis, while A6(E233A)-HIS will not be protective and may even have a detrimental effect. If the effects of exogenous A6-HIS are similar to those of overexpressed A6-GFP, however, then the protective effects of A6 likely derive from its membrane resealing properties, not from some other cellular function.

It is further determined whether recombinant annexin A6-HIS brain infusion via ICV injection or mini-pump implantation results in membrane localization on DNs around plaques in 5XFAD mice. It has been shown that A6 does not need to be expressed in the cell undergoing damage to improve membrane repair in mouse models of acute (cardiotoxin injection) or chronic (muscular dystrophy) muscle injury [Demonbreun et al., J Clin Invest 129, 4657-4670, doi:10.1172/JC1128840 (2019)]. Indeed, exogenous recombinant A6 binds to sites of membrane damage and facilitates membrane repair in muscle [Demonbreun et al., J Clin Invest 129, 4657-4670, doi:10.1172/JC1128840 (2019)]. Additionally, it is shown herein that exogenous recombinant A6 colocalizes with genomically expressed A6 at sites of laser damage in primary neurons (FIGS. 19B and 19C). To determine the minimal dose required for widespread A6-HIS membrane localization, single ICV injections in 5XFAD mice (5/group) of lower doses of A6-HIS and A6(E233A)-HIS (0.1 mg/kg, 0.33 mg/kg, 0.66 mg/kg, 1 mg/kg) are performed, harvested at longer timepoints (3, 6 and 18 hours), and then anti-HIS immunohistochemistry is conducted. A6-HIS staining intensities are compared at all doses and timepoints to find the membrane saturating dose. A short-term Alzet pump implantation (1-2 weeks; 5 mice/group) is then performed to administer a daily dose corresponding to the saturating dose, and 2-fold higher and lower, and the mice are harvested for immunohistochemistry.

For ICV injections, 5XFAD mice are anesthetized by isofluorane inhalation (ISOTHESIA, ndc 11695-6776-2, Henry Schein) delivered with a Basic Small Animal Anesthesia Device model R500IE (RWD Life Science Co. Ltd). The right lateral ventricle is located using the following stereotaxic coordinates: Aβ: −0.6; ML: +1.2; DV: −2.0. A small hole is drilled in the skull and the needle of a Hamilton syringe (80265-1702RNR, needle 7803-07) filled with A6-HIS or A6(E233A)-HIS lowered into the right lateral ventricle. For placement of the Alzet pump, mice are prepared the same, but instead a cannula from Brain infusion kit 3 (cat #0008851, Alzet) is inserted and the brain infusion kit cemented on the skull. A small subcutaneous pouch on the back of the mouse is made to fit Alzet mini-osmotic pump 2006 connected to the brain infusion kit.

As a functional test of the A6 saturating dose, 5XFAD mice (5/group) are implanted with mini-osmotic pumps to infuse A6-TdTomato or A6(E233A)-TdTomato at the saturating dose for 1-2 weeks. Mice are harvested for live-slice imaging with the Indo-1 Ca²⁺ sensor, as described herein. The Indo-1 400 nm/475 nm ratio is quantified in TdTomato positive and negative DNs, and it is expected that those with A6-TdTomato will have reduced Ca²⁺, while those with A6(E233A)-TdTomato will have increased Ca²⁺, compared to TdTomato negative DNs.

Whether osmotic minipump administration of recombinant A6-HIS decreases elevated Ca²⁺, p-tau accumulation, AD tau seeding and spreading, and DN formation in 5XFAD mice is also determined. Osmotic minipumps are used to chronically deliver recombinant A6-HIS into the lateral ventricle of 5XFAD mice. Osmotic minipumps have been successfully used for long-term administration a variety of proteins into the brain, such as antibodies [Dang et al., Cell Rep 27, 1073-1089 e1075, doi:10.1016/j.celrep.2019.03.084 (2019); Furuyama et al., Nature 567, 43-48, doi:10.1038/s41586-019-0942-8 (2019); Mastrella et al., Cancer Res 79, 2298-2313, doi:10.1158/0008-5472.CAN-18-0881 (2019); Roy et al., Immunity, doi:10.1016/j.immuni.2022.03.018 (2022)] and NGF [Benitez et al., Front Endocrinol (Lausanne) 12, 636600, doi:10.3389/fendo.2021.636600 (2021); Kawasaki et al., J Pharmacol Sci 140, 1-7, doi:10.1016/j.jphs.2019.02.011 (2019)], among others. The effect of A6-HIS is tested from 3 to 4.5 months of age, a time of very active Aβ plaque seeding and growth. At 3 months of age, groups of 20 5XFAD mice (10 males, 10 females) are implanted with osmotic minipumps (model 2006, Alzet) containing A6-HIS, A6(E233A)-HIS, or vehicle (artificial CSF). During the last week of infusion, mice undergo behavioral testing (Y-maze, fear conditioning, novel object recognition), and following 42 days of continuous infusion, mice are harvested for brain analysis. As described herein for analysis of mice overexpressing A6-GFP, brains are stained for amyloid plaques and DN markers and ratios (e.g., LAMP1:Aβ42) determined using NIS-Elements software. If extracellular recombinant A6-HIS functions similarly to intracellular AAV-expressed A6-GFP, a reduction in the DN:amyloid ratio is exptected. Reduction of p-tau proteoforms is confirmed by immunohistochemistry as described herein and Ca²⁺ imaging is performed with Indo-1 in brain slices as described herein. It is also determined if osmotic minipump administration of A6-HIS reduces AD tau seeding and spreading. 5XFAD mice will undergo osmotic minipump infusion of A6-HIS, A6(E233A)-HIS, or vehicle into the right lateral ventricle for 42 days, after which minipumps are replaced with new pumps and fresh A6 proteins. Mice are then injected with AD tau into the left dentate gyrus and overlying cortex as described herein. Following another 42 days of annexin A6 infusion, mice are harvested for brain analysis to determine the extent of AD tau seeding and spreading as described herein.

It is expected that recombinant A6-HIS will decrease Ca²⁺ influx, neuritic beading, and cell death in primary neuron cultures and reduce DNs, p-tau, and AD tau seeding and spreading in 5XFAD brain. If osmotic mini-pump administration of A6-HIS for 42 days is not long enough to have an effect on DNs, p-tau, and tau spreading, new mini-pumps are implanted with fresh A6-HIS for a total of 84 days of treatment, a length of time that has been effective for reducing DNs and p-tau181 when annexin A6 is expressed from AAV (FIGS. 23 and 24).

### ADDITIONAL REFERENCES

Akerboom, J., T. W. Chen, T. J. Wardill, L. Tian, J. S. Marvin, S. Mutlu, N. C. Calderon, F. Esposti, B. G. Borghuis, X. R. Sun, A. Gordus, M. B. Orger, R. Portugues, F. Engert, J. J. Macklin, A. Filosa, A. Aggarwal, R. A. Kerr, R. Takagi, S. Kracun, E. Shigetomi, B. S. Khakh, H. Baier, L. Lagnado, S. S. Wang, C. I. Bargmann, B. E. Kimmel, V. Jayaraman, K. Svoboda, D. S. Kim, E. R. Schreiter, and L. L. Looger. 2012. Optimization of a GCaMP calcium indicator for neural activity imaging. J Neurosci. 32:13819-13840.
Avila-Sakar, A. J., C. E. Creutz, and R. H. Kretsinger. 1998. Crystal structure of bovine annexin VI in a calcium-bound state. Biochimica et biophysica acta. 1387:103-116.
Babbin, B. A., M. G. Laukoetter, P. Nava, S. Koch, W. Y. Lee, C. T. Capaldo, E. Peatman, E. A. Severson, R. J. Flower, M. Perretti, C. A. Parkos, and A. Nusrat. 2008. Annexin A1 regulates intestinal mucosal injury, inflammation, and repair. J Immunol. 181:5035-5044.
Bansal, D., K. Miyake, S. S. Vogel, S. Groh, C. C. Chen, R. Williamson, P. L. McNeil, and K. P. Campbell. 2003. Defective membrane repair in dysferlin-deficient muscular dystrophy. Nature. 423:168-172.
Bashir, R., S. Britton, T. Strachan, S. Keers, E. Vafiadaki, M. Lako, I. Richard, S. Marchand, N. Bourg, Z. Argov, M. Sadeh, I. Mahjneh, G. Marconi, M. R. Passos-Bueno, S. Moreira Ede, M. Zatz, J. S. Beckmann, and K. Bushby. 1998. A gene related to Caenorhabditis elegans spermatogenesis factor fer-1 is mutated in limb-girdle muscular dystrophy type 2B. Nat Genet. 20:37-42.
Bement, W. M., C. A. Mandato, and M. N. Kirsch. 1999. Wound-induced assembly and closure of an actomyosin purse string in Xenopus oocytes. Curr Biol. 9:579-587.
Benz, J., A. Bergner, A. Hofmann, P. Demange, P. Gottig, S. Liemann, R. Huber, and D. Voges. 1996. The structure of recombinant human annexin VI in crystals and membrane-bound. J Mol Biol. 260:638-643.
Bi, G. Q., J. M. Alderton, and R. A. Steinhardt. 1995. Calcium-regulated exocytosis is required for cell membrane resealing. The Journal of cell biology. 131:1747-1758.
Bizzarro, V., A. Petrella, and L. Parente. 2012. Annexin A1: novel roles in skeletal muscle biology. J Cell Physiol. 227:3007-3015.
Blackwood, R. A., and J. D. Ernst. 1990. Characterization of Ca2(+)-dependent phospholipid binding, vesicle aggregation and membrane fusion by annexins. The Biochemical journal. 266:195-200.
Boye, T. L., J. C. Jeppesen, K. Maeda, W. Pezeshkian, V. Solovyeva, J. Nylandsted, and A. C. Simonsen. 2018. Annexins induce curvature on free-edge membranes displaying distinct morphologies. Sci Rep. 8:10309.
Boye, T. L., K. Maeda, W. Pezeshkian, S. L. Sonder, S. C. Haeger, V. Gerke, A. C. Simonsen, and J. Nylandsted. 2017. Annexin A4 and A6 induce membrane curvature and constriction during cell membrane repair. Nat Commun. 8:1623.
Buzhynskyy, N., M. Golczak, J. Lai-Kee-Him, O. Lambert, B. Tessier, C. Gounou, R. Berat, A. Simon, T. Granier, J. M. Chevalier, S. Mazeres, J. Bandorowicz-Pikula, S. Pikula, and A. R. Brisson. 2009. Annexin-A6 presents two modes of association with phospholipid membranes. A combined QCM-D, AFM and cryo-TEM study. Journal of structural biology. 168:107-116.
Cai, C., H. Masumiya, N. Weisleder, N. Matsuda, M. Nishi, M. Hwang, J. K. Ko, P. Lin, A. Thornton, X. Zhao, Z. Pan, S. Komazaki, M. Brotto, H. Takeshima, and J. Ma. 2009. MG53 nucleates assembly of cell membrane repair machinery. Nat Cell Biol. 11:56-64.
Carmeille, R., S. A. Degrelle, L. Plawinski, F. Bouvet, C. Gounou, D. Evain-Brion, A. R. Brisson, and A. Bouter. 2015. Annexin-A5 promotes membrane resealing in human trophoblasts. Biochimica et biophysica acta. 1853:2033-2044.
Cagliani, R., F. Magri, A. Toscano, L. Merlini, F. Fortunato, C. Lamperti, C. Rodolico, A. Prelle, M. Sironi, M. Aguennouz, P. Ciscato, A. Uncini, M. Moggio, N. Bresolin, and G. P. Comi. 2005. Mutation finding in patients with dysferlin deficiency and role of the dysferlin interacting proteins annexin A1 and A2 in muscular dystrophies. Human mutation. 26:283.
Ceco, E., S. Bogdanovich, B. Gardner, T. Miller, A. DeJesus, J. U. Earley, M. Hadhazy, L. R. Smith, E. R. Barton, J. D. Molkentin, and E. M. McNally. 2014. Targeting latent TGFbeta release in muscular dystrophy. Science translational medicine. 6:259ra144.
Christmas, P., J. Callaway, J. Fallon, J. Jones, and H. T. Haigler. 1991. Selective secretion of annexin 1, a protein without a signal sequence, by the human prostate gland. The Journal of biological chemistry. 266:2499-2507.
Davenport, N. R., K. J. Sonnemann, K. W. Eliceiri, and W. M. Bement. 2016. Membrane dynamics during cellular wound repair. Mol Biol Cell. 27:2272-2285.
Defour, A., S. Medikayala, J. H. Van der Meulen, M. W. Hogarth, N. Holdreith, A. Malatras, W. Duddy, J. Boehler, K. Nagaraju, and J. K. Jaiswal. 2017. Annexin A2 links poor myofiber repair with inflammation and adipogenic replacement of the injured muscle. Human molecular genetics. 26:1979-1991.
de Laat, B., R. H. Derksen, I. J. Mackie, M. Roest, S. Schoormans, B. J. Woodhams, P. G. de Groot, and W. L. van Heerde. 2006. Annexin A5 polymorphism (−1C->T) and the presence of anti-annexin A5 antibodies in the antiphospholipid syndrome. Annals of the rheumatic diseases. 65:1468-1472.
Demonbreun, A. R., M. V. Allen, J. L. Warner, D. Y. Barefield, S. Krishnan, K. E. Swanson, J. U. Earley, and E. M. McNally. 2016a. Enhanced Muscular Dystrophy from Loss of Dysferlin Is Accompanied by Impaired Annexin A6 Translocation after Sarcolemmal Disruption. Am J Pathol. 186:1610-1622.
Demonbreun, A. R., and E. M. McNally. 2015. DNA Electroporation, Isolation and Imaging of Myofibers. Journal of visualized experiments: JoVE. 106:e53551.
Demonbreun, A. R., and E. M. McNally. 2016. Plasma Membrane Repair in Health and Disease. Curr Top Membr. 77:67-96.
Demonbreun, A. R., M. Quattrocelli, D. Y. Barefield, M. V. Allen, K. E. Swanson, and E. M. McNally. 2016b. An actin-dependent annexin complex mediates plasma membrane repair in muscle. The Journal of cell biology. 213:705-718.
Demonbreun, A. R., A. E. Rossi, M. G. Alvarez, K. E. Swanson, H. K. Deveaux, J. U. Earley, M. Hadhazy, R. Vohra, G. A. Walter, P. Pytel, and E. M. McNally. 2014. Dysferlin and myoferlin regulate transverse tubule formation and glycerol sensitivity. Am J Pathol. 184:248-259.
Demonbreun, A. R., K. E. Swanson, A. E. Rossi, H. K. Deveaux, J. U. Earley, M. V. Allen, P. Arya, S. Bhattacharyya, H. Band, P. Pytel, and E. M. McNally. 2015. Eps 15 Homology Domain (EHD)-1 Remodels Transverse Tubules in Skeletal Muscle. PLoS One. 10:e0136679.
Deora, A. B., G. Kreitzer, A. T. Jacovina, and K. A. Hajjar. 2004. An annexin 2 phosphorylation switch mediates p11-dependent translocation of annexin 2 to the cell surface. The Journal of biological chemistry. 279:43411-43418.
DiFranco, M., M. Quinonez, J. Capote, and J. Vergara. 2009. DNA transfection of mammalian skeletal muscles using in vivo electroporation. Journal of visualized experiments: JoVE. 32.
Duann, P., H. Li, P. Lin, T. Tan, Z. Wang, K. Chen, X. Zhou, K. Gumpper, H. Zhu, T. Ludwig, P. J. Mohler, B. Rovin, W. T. Abraham, C. Zeng, and J. Ma. 2015. MG53-mediated cell membrane repair protects against acute kidney injury. Science translational medicine. 7:279ra236.
Echigoya Y, Lim K R Q, Nakamura A, and Yokota T. Multiple Exon Skipping in the Duchenne Muscular Dystrophy Hot Spots: Prospects and Challenges. J Pers Med 8, 2018.
Fiehn, W., J. B. Peter, J. F. Mead, and M. Gan-Elepano. 1971. Lipids and fatty acids of sarcolemma, sarcoplasmic reticulum, and mitochondria from rat skeletal muscle. The Journal of biological chemistry. 246:5617-5620.
Flanigan, K. M., E. Ceco, K. M. Lamar, Y. Kaminoh, D. M. Dunn, J. R. Mendell, W. M. King, A. Pestronk, J. M. Florence, K. D. Mathews, R. S. Finkel, K. J. Swoboda, E. Gappmaier, M. T. Howard, J. W. Day, C. McDonald, E. M. McNally, and R. B. Weiss. 2013. LTBP4 genotype predicts age of ambulatory loss in duchenne muscular dystrophy. Ann Neurol. 73:481-488.
Gerke, V., C. E. Creutz, and S. E. Moss. 2005. Annexins: linking Ca²⁺ signalling to membrane dynamics. Nat Rev Mol Cell Biol. 6:449-461.
Gerke, V., and S. E. Moss. 2002. Annexins: from structure to function. Physiol Rev. 82:331-371.
Grewal, T., M. Hoque, J. R. W. Conway, M. Reverter, M. Wahba, S. S. Beevi, P. Timpson, C. Enrich, and C. Rentero. 2017. Annexin A6-A multifunctional scaffold in cell motility. Cell Adh Migr. 11:288-304.
Goulet, F., K. G. Moore, and A. C. Sartorelli. 1992. Glycosylation of annexin I and annexin II. Biochemical and biophysical research communications. 188:554-558.
Hack A A, et al. Gamma-sarcoglycan deficiency leads to muscle membrane defects and apoptosis independent of dystrophin. J Cell Biol. 1998; 142(5):1279-1287.
Hannon, R., J. D. Croxtall, S. J. Getting, F. Roviezzo, S. Yona, M. J. Paul-Clark, F. N. Gavins, M. Perretti, J. F. Morris, J. C. Buckingham, and R. J. Flower. 2003. Aberrant inflammation and resistance to glucocorticoids in annexin 1−/− mouse. FASEB J. 17:253-255.
He, B., R. H. Tang, N. Weisleder, B. Xiao, Z. Yuan, C. Cai, H. Zhu, P. Lin, C. Qiao, J. Li, C. Mayer, J. Li, J. Ma, and X. Xiao. 2012. Enhancing muscle membrane repair by gene delivery of MG53 ameliorates muscular dystrophy and heart failure in delta-Sarcoglycan-deficient hamsters. Molecular therapy: the journal of the American Society of Gene Therapy. 20:727-735.
Hoffman, E. P., R. H. Brown, Jr., and L. M. Kunkel. 1987. Dystrophin: the protein product of the Duchenne muscular dystrophy locus. Cell. 51:919-928.
Jennische, E., and H. A. Hansson. 1986. Postischemic skeletal muscle injury: patterns of injury in relation to adequacy of reperfusion. Exp Mol Pathol. 44:272-280.
Jia, Y., K. Chen, P. Lin, G. Lieber, M. Nishi, R. Yan, Z. Wang, Y. Yao, Y. Li, B. A. Whitson, P. Duann, H. Li, X. Zhou, H. Zhu, H. Takeshima, J. C. Hunter, R. L. McLeod, N. Weisleder, C. Zeng, and J. Ma. 2014. Treatment of acute lung injury by targeting MG53-mediated cell membrane repair. Nat Commun. 5:4387.
Jimenez, A. J., and F. Perez. 2017. Plasma membrane repair: the adaptable cell life-insurance. Curr Opin Cell Biol. 47:99-107.
Jost, M., C. Thiel, K. Weber, and V. Gerke. 1992. Mapping of three unique Ca(2+)-binding sites in human annexin II. Eur J Biochem. 207:923-930.
Kaetzel, M. A., Y. D. Mo, T. R. Mealy, B. Campos, W. Bergsma-Schutter, A. Brisson, J. R. Dedman, and B. A. Seaton. 2001. Phosphorylation mutants elucidate the mechanism of annexin IV-mediated membrane aggregation. Biochemistry. 40:4192-4199.
Lauritzen, S. P., T. L. Boye, and J. Nylandsted. 2015. Annexins are instrumental for efficient plasma membrane repair in cancer cells. Semin Cell Dev Biol. 45:32-38.
Lennon, N. J., A. Kho, B. J. Bacskai, S. L. Perlmutter, B. T. Hyman, and R. H. Brown, Jr. 2003. Dysferlin interacts with annexins A1 and A2 and mediates sarcolemmal wound-healing. The Journal of biological chemistry. 278:50466-50473.
Li D, Mastaglia F L, Fletcher S, and Wilton S D. Precision Medicine through Antisense Oligonucleotide-Mediated Exon Skipping. Trends Pharmacol Sci 39: 982-994, 2018.
Ling, Q., A. T. Jacovina, A. Deora, M. Febbraio, R. Simantov, R. L. Silverstein, B. Hempstead, W. H. Mark, and K. A. Hajjar. 2004. Annexin II regulates fibrin homeostasis and neoangiogenesis in vivo. The Journal of clinical investigation. 113:38-48.
Liu, J., H. Zhu, Y. Zheng, Z. Xu, L. Li, T. Tan, K. H. Park, J. Hou, C. Zhang, D. Li, R. Li, Z. Liu, N. Weisleder, D. Zhu, P. Lin, and J. Ma. 2015. Cardioprotection of recombinant human MG53 protein in a porcine model of ischemia and reperfusion injury. Journal of molecular and cellular cardiology. 80:10-19.
McDade, J. R., A. Archambeau, and D. E. Michele. 2014. Rapid actin-cytoskeleton-dependent recruitment of plasma membrane-derived dysferlin at wounds is critical for muscle membrane repair. FASEB J. 28:3660-3670.
McNeil, A. K., U. Rescher, V. Gerke, and P. L. McNeil. 2006. Requirement for annexin A1 in plasma membrane repair. The Journal of biological chemistry. 281:35202-35207.
McNeil, P. L., and R. Khakee. 1992. Disruptions of muscle fiber plasma membranes. Role in exercise-induced damage. Am J Pathol. 140:1097-1109.
McNeil, P. L., and T. Kirchhausen. 2005. An emergency response team for membrane repair. Nat Rev Mol Cell Biol. 6:499-505.
Min Y L, Bassel-Duby R, and Olson E N. CRISPR Correction of Duchenne Muscular Dystrophy. Annu Rev Med, 2018.
Murphy, S., M. Zweyer, M. Henry, P. Meleady, R. R. Mundegar, D. Swandulla, and K. Ohlendieck. 2018. Proteomic analysis of the sarcolemma-enriched fraction from dystrophic mdx-4cv skeletal muscle. J Proteomics.
Petrof, B. J., J. B. Shrager, H. H. Stedman, A. M. Kelly, and H. L. Sweeney.
1993. Dystrophin protects the sarcolemma from stresses developed during muscle contraction. Proceedings of the National Academy of Sciences of the United States of America. 90:3710-3714.
Quattrocelli, M., D. Y. Barefield, J. L. Warner, A. H. Vo, M. Hadhazy, J. U. Earley, A. R. Demonbreun, and E. M. McNally. 2017a. Intermittent glucocorticoid steroid dosing enhances muscle repair without eliciting muscle atrophy. J Clin Invest. 127:2418-2432.
Quattrocelli, M., J. Capote, J. C. Ohiri, J. L. Warner, A. H. Vo, J. U. Earley, M. Hadhazy, A. R. Demonbreun, M. J. Spencer, and E. M. McNally. 2017b. Genetic modifiers of muscular dystrophy act on sarcolemmal resealing and recovery from injury. PLoS Genet. 13:e1007070.
Quattrocelli, M., I. M. Salamone, P. G. Page, J. L. Warner, A. R. Demonbreun, and E. M. McNally. 2017c. Intermittent Glucocorticoid Dosing Improves Muscle Repair and Function in Mice with Limb-Girdle Muscular Dystrophy. Am J Pathol. 187:2520-2535.
Reddy, A., E. V. Caler, and N. W. Andrews. 2001. Plasma membrane repair is mediated by Ca(2+)-regulated exocytosis of lysosomes. Cell. 106:157-169.
Rodriguez, A., P. Webster, J. Ortego, and N. W. Andrews. 1997. Lysosomes behave as Ca²⁺-regulated exocytic vesicles in fibroblasts and epithelial cells. J Cell Biol. 137:93-104.
Roostalu, U., and U. Strahle. 2012. In vivo imaging of molecular interactions at damaged sarcolemma. Dev Cell. 22:515-529.
Swaggart, K. A., A. R. Demonbreun, A. H. Vo, K. E. Swanson, E. Y. Kim, J. P. Fahrenbach, J. Holley-Cuthrell, A. Eskin, Z. Chen, K. Squire, A. Heydemann, A. A. Palmer, S. F. Nelson, and E. M. McNally. 2014. Annexin A6 modifies muscular dystrophy by mediating sarcolemmal repair. Proceedings of the National Academy of Sciences of the United States of America. 111:6004-6009.
Waddell, L. B., F. A. Lemckert, X. F. Zheng, J. Tran, F. J. Evesson, J. M. Hawkes, A. Lek, N. E. Street, P. Lin, N. F. Clarke, A. P. Landstrom, M. J. Ackerman, N. Weisleder, J. Ma, K. N. North, and S. T. Cooper. 2011. Dysferlin, annexin A1, and mitsugumin 53 are upregulated in muscular dystrophy and localize to longitudinal tubules of the T-system with stretch. J Neuropathol Exp Neurol. 70:302-313.
Wallner, B. P., R. J. Mattaliano, C. Hession, R. L. Cate, R. Tizard, L. K. Sinclair, C. Foeller, E. P. Chow, J. L. Browing, K. L. Ramachandran, and et al. 1986. Cloning and expression of human lipocortin, a phospholipase A2 inhibitor with potential anti-inflammatory activity. Nature. 320:77-81.
Weisleder, N., N. Takizawa, P. Lin, X. Wang, C. Cao, Y. Zhang, T. Tan, C. Ferrante, H. Zhu, P. J. Chen, R. Yan, M. Sterling, X. Zhao, M. Hwang, M. Takeshima, C. Cai, H. Cheng, H. Takeshima, R. P. Xiao, and J. Ma. 2012. Recombinant MG53 protein modulates therapeutic cell membrane repair in treatment of muscular dystrophy. Science translational medicine. 4:139ra185.
Yeung, T., B. Heit, J. F. Dubuisson, G. D. Fairn, B. Chiu, R. Inman, A. Kapus, M. Swanson, and S. Grinstein. 2009. Contribution of phosphatidylserine to membrane surface charge and protein targeting during phagosome maturation. The Journal of cell biology. 185:917-928.
Zaks, W. J., and C. E. Creutz. 1991. Ca(2+)-dependent annexin self-association on membrane surfaces. Biochemistry. 30:9607-9615.
Zhang Y, Long C, Bassel-Duby R, and Olson E N. Myoediting: Toward Prevention of Muscular Dystrophy by Therapeutic Genome Editing. Physiol Rev 98: 1205-1240, 2018.
Zweifach, A. 2000. FM1-43 reports plasma membrane phospholipid scrambling in T-lymphocytes. The Biochemical journal. 349:255-260.
Dias C, and Nylandsted J. Plasma membrane integrity in health and disease: significance and therapeutic potential. Cell Discov. 2021; 7(1):4.
Ammendolia D A, Bement W M, and Brumell J H. Plasma membrane integrity: implications for health and disease. BMC Biol. 2021; 19(1):71.
Andrews N W, and Corrotte M. Plasma membrane repair. Curr Biol. 2018; 28(8):R392-R7.
Cooper S T, and McNeil P L. Membrane Repair: Mechanisms and Pathophysiology. Physiol Rev. 2015; 95(4):1205-40.
Koerdt S N, Ashraf APK, and Gerke V. Annexins and plasma membrane repair. Curr Top Membr. 2019; 84:43-65.
McDade J R, and Michele D E. Membrane damage-induced vesicle-vesicle fusion of dysferlin-containing vesicles in muscle cells requires microtubules and kinesin. Human molecular genetics. 2014; 23(7):1677-86.
Lek A, Evesson F J, Lemckert F A, Redpath G M, Lueders A K, Turnbull L, et al. Calpains, cleaved mini-dysferlinC72, and L-type channels underpin calcium-dependent muscle membrane repair. J Neurosci. 2013; 33(12):5085-94.
Davenport N R, Sonnemann K J, Eliceiri K W, and Bement W M. Membrane dynamics during cellular wound repair. Mol Biol Cell. 2016; 27(14):2272-85.
Jimenez A J, Maiuri P, Lafaurie-Janvore J, Divoux S, Piel M, and Perez F. ESCRT machinery is required for plasma membrane repair. Science. 2014; 343(6174):1247136.
Bement W M, Mandato C A, and Kirsch M N. Wound-induced assembly and closure of an actomyosin purse string in Xenopus oocytes. Curr Biol. 1999; 9(11):579-87.
Babiychuk E B, Monastyrskaya K, Potez S, and Draeger A. Intracellular Ca(2+) operates a switch between repair and lysis of streptolysin 0-perforated cells. Cell Death Differ. 2009; 16(8):1126-34.
Babiychuk E B, Monastyrskaya K, Potez S, and Draeger A. Blebbing confers resistance against cell lysis. Cell Death Differ. 2011; 18(1):80-9.
Demonbreun A R, and McNally E M. Plasma Membrane Repair in Health and Disease. Curr Top Membr. 2016; 77:67-96.
Idone V, Tam C, Goss J W, Toomre D, Pypaert M, and Andrews N W. Repair of injured plasma membrane by rapid Ca²⁺-dependent endocytosis. The Journal of cell biology. 2008; 180(5):905-14.
McDade J R, Archambeau A, and Michele D E. Rapid actin-cytoskeleton-dependent recruitment of plasma membrane-derived dysferlin at wounds is critical for muscle membrane repair. FASEB J. 2014; 28(8):3660-70.
Corrotte M, Almeida P E, Tam C, Castro-Gomes T, Fernandes M C, Millis B A, et al. Caveolae internalization repairs wounded cells and muscle fibers. Elife. 2013; 2:e00926.
Grewal T, Hoque M, Conway J R W, Reverter M, Wahba M, Beevi S S, et al. Annexin A6-A multifunctional scaffold in cell motility. Cell Adh Migr. 2017; 11(3):288-304.
Gerke V, Creutz C E, and Moss S E. Annexins: linking Ca²⁺ signalling to membrane dynamics. Nat Rev Mol Cell Biol. 2005; 6(6):449-61.
Gerke V, and Moss S E. Annexins and membrane dynamics. Biochim Biophys Acta. 1997; 1357(2):129-54.
Gerke V, and Moss S E. Annexins: from structure to function. Physiol Rev. 2002; 82(2):331-71.
Perretti M, and Flower R J. Modulation of IL-1-induced neutrophil migration by dexamethasone and lipocortin 1. J Immunol. 1993; 150(3):992-9.
Garcia-Melero A, Reverter M, Hoque M, Meneses-Salas E, Koese M, Conway J R, et al. Annexin A6 and Late Endosomal Cholesterol Modulate Integrin Recycling and Cell Migration. J Biol Chem. 2016; 291(3):1320-35.
Demonbreun A R, Fallon K S, Oosterbaan C C, Bogdanovic E, Warner J L, Sell J J, et al. Recombinant annexin A6 promotes membrane repair and protects against muscle injury. The Journal of Clinical Investigation. 2019; 129(11):4657-70.
Demonbreun A R, Quattrocelli M, Barefield D Y, Allen M V, Swanson K E, and McNally E M. An actin-dependent annexin complex mediates plasma membrane repair in muscle. The Journal of cell biology. 2016; 213(6):705-18.
Roostalu U, and Strahle U. In vivo imaging of molecular interactions at damaged sarcolemma. Dev Cell. 2012; 22(3):515-29.
Foltz S J, Cui Y Y, Choo H J, and Hartzell H C. ANO5 ensures trafficking of annexins in wounded myofibers. The Journal of cell biology. 2021; 220(3).
Potez S, Luginbuhl M, Monastyrskaya K, Hostettler A, Draeger A, and Babiychuk E B. Tailored protection against plasmalemmal injury by annexins with different Ca²⁺ sensitivities. J Biol Chem. 2011; 286(20):17982-91.
Defour A, Medikayala S, Van der Meulen J H, Hogarth M W, Holdreith N, Malatras A, et al. Annexin A2 links poor myofiber repair with inflammation and adipogenic replacement of the injured muscle. Human molecular genetics. 2017; 26(11):1979-91.
Leikina E, Defour A, Melikov K, Van der Meulen J H, Nagaraju K, Bhuvanendran S, et al. Annexin A1 Deficiency does not Affect Myofiber Repair but Delays Regeneration of Injured Muscles. Sci Rep. 2015; 5:18246.
Swaggart K A, Demonbreun A R, Vo A H, Swanson K E, Kim E Y, Fahrenbach J P, et al. Annexin A6 modifies muscular dystrophy by mediating sarcolemmal repair. Proceedings of the National Academy of Sciences of the United States of America. 2014; 111(16):6004-9.
Doubell A F, Lazure C, Charbonneau C, and Thibault G. Identification and immunolocalisation of annexins V and VI, the major cardiac annexins, in rat heart. Cardiovasc Res. 1993; 27(7):1359-67.
de Jong R C M, Pluijmert N J, de Vries M R, Pettersson K, Atsma D E, Jukema J W, et al. Annexin A5 reduces infarct size and improves cardiac function after myocardial ischemia-reperfusion injury by suppression of the cardiac inflammatory response. Sci Rep. 2018; 8(1):6753.
Gunteski-Hamblin A M, Song G, Walsh R A, Frenzke M, Boivin G P, Dorn G W, 2nd, et al. Annexin VI overexpression targeted to heart alters cardiomyocyte function in transgenic mice. Am J Physiol. 1996; 270(3 Pt 2):H1091-100.
Song G, Harding S E, Duchen M R, Tunwell R, O'Gara P, Hawkins T E, et al. Altered mechanical properties and intracellular calcium signaling in cardiomyocytes from annexin 6 null-mutant mice. FASEB J. 2002; 16(6):622-4.
Kilinc D, Gallo G, and Barbee K A. Mechanically-induced membrane poration causes axonal beading and localized cytoskeletal damage. Exp Neurol. 2008; 212(2):422-30.
Prado G R, and LaPlaca M C. Neuronal Plasma Membrane Integrity is Transiently Disturbed by Traumatic Loading. Neurosci Insights. 2020; 15:2633105520946090.
LaPlaca M C, Prado G R, Cullen D K, and Irons H R. High rate shear insult delivered to cortical neurons produces heterogeneous membrane permeability alterations. Conf Proc IEEE Eng Med Biol Soc. 2006; 2006:2384-7.
LaPlaca M C, Prado G R, Cullen D, and Simon C M. Plasma membrane damage as a marker of neuronal injury. Annu Int Conf IEEE Eng Med Biol Soc. 2009; 2009:1113-6.
Liu N, Han S, Lu P H, and Xu X M. Upregulation of annexins I, II, and V after traumatic spinal cord injury in adult rats. J Neurosci Res. 2004; 77(3):391-401.
Cristante E, McArthur S, Mauro C, Maggioli E, Romero I A, Wylezinska-Arridge M, et al. Identification of an essential endogenous regulator of blood-brain barrier integrity, and its pathological and therapeutic implications. Proceedings of the National Academy of Sciences of the United States of America. 2013; 110(3):832-41.
Gussenhoven R, Klein L, Ophelders D, Habets D H J, Giebel B, Kramer B W, et al. Annexin A1 as Neuroprotective Determinant for Blood-Brain Barrier Integrity in Neonatal Hypoxic-Ischemic Encephalopathy. J Clin Med. 2019; 8(2).
Bartolome F, Krzyzanowska A, de la Cueva M, Pascual C, Antequera D, Spuch C, et al. Annexin A5 prevents amyloid-beta-induced toxicity in choroid plexus: implication for Alzheimer's disease. Sci Rep. 2020; 10(1):9391.
Skrahina T, Piljic A, and Schultz C. Heterogeneity and timing of translocation and membrane-mediated assembly of different annexins. Exp Cell Res. 2008; 314(5):1039-47.
Demonbreun A R, and McNally E M. DNA Electroporation, Isolation and Imaging of Myofibers. Journal of visualized experiments: JoVE. 2015; 106(106):e53551.
DiFranco M, Quinonez M, Capote J, and Vergara J. DNA transfection of mammalian skeletal muscles using in vivo electroporation. Journal of visualized experiments JoVE. 2009; 32(32).
Hawkins T E, Roes J, Rees D, Monkhouse J, and Moss S E. Immunological development and cardiovascular function are normal in annexin VI null mutant mice. Mol Cell Biol. 1999; 19(12):8028-32.
Mishra S, Chander V, Banerjee P, Oh J G, Lifirsu E, Park W J, et al. Interaction of annexin A6 with alpha actinin in cardiomyocytes. BMC Cell Biol. 2011; 12:7.
Simons K, and Toomre D. Lipid rafts and signal transduction. Nature reviews Molecular cell biology. 2000; 1(1):31-9.
Parton R G, and del Pozo M A. Caveolae as plasma membrane sensors, protectors and organizers. Nature reviews Molecular cell biology. 2013; 14(2):98-112.
Illien F, Piao H-R, CouéM, Di Marco C, and Ayala-Sanmartin J. Lipid organization regulates annexin A2 Ca 2+-sensitivity for membrane bridging and its modulator effects on membrane fluidity. Biochimica et Biophysica Acta (BBA)-Biomembranes. 2012; 1818(11):2892-900.
Vaughan E M, You J S, Elsie Yu H Y, Lasek A, Vitale N, Hornberger T A, et al. Lipid domain-dependent regulation of single-cell wound repair. Mol Biol Cell. 2014; 25(12):1867-76.
Leikina E, Melikov K, Sanyal S, Verma S K, Eun B, Gebert C, et al. Extracellular annexins and dynamin are important for sequential steps in myoblast fusion. J Cell Biol. 2013; 200(1):109-23.
Downing G J, Kim S, Nakanishi S, Catt K J, and Balla T. Characterization of a soluble adrenal phosphatidylinositol 4-kinase reveals wortmannin sensitivity of type Ill phosphatidylinositol kinases. Biochemistry. 1996; 35(11):3587-94.
Nilius B, Mahieu F, Prenen J, Janssens A, Owsianik G, Vennekens R, et al. The Ca²⁺-activated cation channel TRPM4 is regulated by phosphatidylinositol 4,5-biphosphate. EMBO J. 2006; 25(3):467-78.
Nakanishi S, Catt K J, and Balla T. A wortmannin-sensitive phosphatidylinositol 4-kinase that regulates hormone-sensitive pools of inositolphospholipids. Proceedings of the National Academy of Sciences of the United States of America. 1995; 92(12):5317-21.
Stauffer T P, Ahn S, and Meyer T. Receptor-induced transient reduction in plasma membrane Ptdlns(4,5)P2 concentration monitored in living cells. Curr Biol. 1998; 8(6):343-6.
Nygard Skalman L, Holst M R, Larsson E, and Lundmark R. Plasma membrane damage caused by listeriolysin O is not repaired through endocytosis of the membrane pore. Biol Open. 2018; 7(10).
Bulfield G, Siller W G, Wight P A, and Moore K J. X chromosome-linked muscular dystrophy (mdx) in the mouse. Proceedings of the National Academy of Sciences of the United States of America. 1984; 81(4):1189-92.
Sicinski P, Geng Y, Ryder-Cook A S, Barnard E A, Darlison M G, and Barnard P J. The molecular basis of muscular dystrophy in the mdx mouse: a point mutation. Science. 1989; 244(4912):1578-80.
Geddis M S, and Rehder V. Initial stages of neural regeneration in Helisoma trivolvis are dependent upon PLA2 activity. J Neurobiol. 2003; 54(4):555-65.
Croissant C, Gounou C, Bouvet F, Tan S, and Bouter A. Annexin-A6 in Membrane Repair of Human Skeletal Muscle Cell: A Role in the Cap Subdomain. Cells. 2020; 9(7):1742.
Boye T L, Jeppesen J C, Maeda K, Pezeshkian W, Solovyeva V, Nylandsted J, et al. Annexins induce curvature on free-edge membranes displaying distinct morphologies. Scientific reports. 2018; 8(1):10309-.
Boye T L, Maeda K, Pezeshkian W, Sonder S L, Haeger S C, Gerke V, et al. Annexin A4 and A6 induce membrane curvature and constriction during cell membrane repair. Nature communications. 2017; 8(1):1623-.
Kitmitto A, Baudoin F, and Cartwright E J. Cardiomyocyte damage control in heart failure and the role of the sarcolemma. J Muscle Res Cell Motil. 2019; 40(3-4):319-33.
Benevolensky D, Belikova Y, Mohammadzadeh R, TrouvéP, Marotte F, Russo-Marie F, et al. Expression and Localization of the Annexins II, V, and VI in Myocardium from Patients with End-Stage Heart Failure. Laboratory Investigation. 2000; 80(2):123-33.
Song G, Campos B, Wagoner L E, Dedman J R, and Walsh R A. Altered cardiac annexin mRNA and protein levels in the left ventricle of patients with end-stage heart failure. J Mol Cell Cardiol. 1998; 30(3):443-51.
TrouvéP, Legot S, Bélikova I, Marotte F, Bénévolensky D, Russo-Marie F, et al. Localization and quantitation of cardiac annexins II, V, and VI in hypertensive guinea pigs. Am J Physiol. 1999; 276(4):H1159-66.
Hendricks B K, and Shi R. Mechanisms of neuronal membrane sealing following mechanical trauma. Neurosci Bull. 2014; 30(4):627-44.
Alexander, G. C., Emerson, S. & Kesselheim, A. S. Evaluation of Aducanumab for Alzheimer Disease: Scientific Evidence and Regulatory Review Involving Efficacy, Safety, and Futility. JAMA 325, 1717-1718, doi:10.1001/jama.2021.3854 (2021).
Alexander, G. C. et al. Revisiting FDA Approval of Aducanumab. N Engl J Med 385, 769-771, doi:10.1056/NEJMp2110468 (2021).
Karran, E. & De Strooper, B. The amyloid hypothesis in Alzheimer disease: new insights from new therapeutics. Nat Rev Drug Discov 21, 306-318, doi:10.1038/s41573-022-00391-w (2022).
Hardy, J. Membrane damage is at the core of Alzheimer's disease. Lancet Neurol 16, 342, doi:10.1016/S1474-4422(17)30091-1 (2017).
Fernandez-Perez, E. J., Peters, C. & Aguayo, L. G. Membrane Damage Induced by Amyloid Beta and a Potential Link with Neuroinflammation. Curr Pharm Des 22, 1295-1304, doi:10.2174/138161282210160304111702 (2016).
Drolle, E., Negoda, A., Hammond, K., Pavlov, E. & Leonenko, Z. Changes in lipid membranes may trigger amyloid toxicity in Alzheimer's disease. PLoS One 12, e0182194, doi:10.1371/journal.pone.0182194 (2017). PMC5540602,
Kuchibhotla, K. V. et al. Abeta plaques lead to aberrant regulation of calcium homeostasis in vivo resulting in structural and functional disruption of neuronal networks. Neuron 59, 214-225, doi:10.1016/j.neuron.2008.06.008 (2008). PMC2578820,
Sadleir, K. R. et al. Presynaptic dystrophic neurites surrounding amyloid plaques are sites of microtubule disruption, BACE1 elevation, and increased Abeta generation in Alzheimer's disease. Acta Neuropathol 132, 235-256, doi:10.1007/s00401-016-1558-9 (2016). PMC4947125,
Barthelemy, N. R., Horie, K., Sato, C. & Bateman, R. J. Blood plasma phosphorylated-tau isoforms track CNS change in Alzheimer's disease. J Exp Med 217, doi:10.1084/jem.20200861 (2020). PMC7596823,
Barthelemy, N. R. et al. A soluble phosphorylated tau signature links tau, amyloid and the evolution of stages of dominantly inherited Alzheimer's disease. Nat Med 26, 398-407, doi:10.1038/s41591-020-0781-z (2020). PMC7309367,
Clark, C. et al. Plasma neurofilament light and phosphorylated tau 181 as biomarkers of Alzheimer's disease pathology and clinical disease progression. Alzheimers Res Ther 13, 65, doi:10.1186/s13195-021-00805-8 (2021). PMC7995778,
Frank, B. et al. Plasma p-tau181 shows stronger network association to Alzheimer's disease dementia than neurofilament light and total tau. Alzheimers Dement, doi:10.1002/alz.12508 (2021).
Karikari, T. K. et al. Head-to-head comparison of clinical performance of CSF phospho-tau T181 and T217 biomarkers for Alzheimer's disease diagnosis. Alzheimers Dement 17, 755-767, doi:10.1002/alz.12236 (2021). PMC8246793,
Mielke, M. M. et al. Plasma phospho-tau181 increases with Alzheimer's disease clinical severity and is associated with tau- and amyloid-positron emission tomography. Alzheimers Dement 14, 989-997, doi:10.1016/j.jalz.2018.02.013 (2018). PMC6097897,
Suarez-Calvet, M. et al. Novel tau biomarkers phosphorylated at T181, T217 or T231 rise in the initial stages of the preclinical Alzheimer's continuum when only subtle changes in Abeta pathology are detected. EMBO Mol Med 12, e12921, doi:10.15252/emmm.202012921 (2020). PMC7721364,
He, Z. et al. Amyloid-beta plaques enhance Alzheimer's brain tau-seeded pathologies by facilitating neuritic plaque tau aggregation. Nat Med 24, 29-38, doi:10.1038/nm.4443 (2018). PMC5760353,
Jagust, W. Imaging the evolution and pathophysiology of Alzheimer disease. Nat Rev Neurosci 19, 687-700, doi:10.1038/s41583-018-0067-3 (2018). PMC7032048,
Mattsson-Carlgren, N. et al. The implications of different approaches to define A T(N) in Alzheimer disease. Neurology 94, e2233-e2244, doi:10.1212/WNL.0000000000009485 (2020). PMC7357296,
Aschenbrenner, A. J., Gordon, B. A., Benzinger, T. L. S., Morris, J. C. & Hassenstab, J. J. Influence of tau PET, amyloid PET, and hippocampal volume on cognition in Alzheimer disease. Neurology 91, e859-e866, doi:10.1212/WNL.0000000000006075 (2018). PMC6133625,
Cho, H. et al. Progressive Tau Accumulation in Alzheimer Disease: 2-Year Follow-up Study. J Nucl Med 60, 1611-1621, doi:10.2967/jnumed.118.221697 (2019). PMC6836867,
Hanseeuw, B. J. et al. Association of Amyloid and Tau With Cognition in Preclinical Alzheimer Disease: A Longitudinal Study. JAMA Neurol 76, 915-924, doi:10.1001/jamaneurol.2019.1424 (2019). PMC6547132,
Hansson, O. Biomarkers for neurodegenerative diseases. Nat Med 27, 954-963, doi:10.1038/s41591-021-01382-x (2021).
Jack, C. R., Jr. et al. Hypothetical model of dynamic biomarkers of the Alzheimer's pathological cascade. Lancet Neurol 9, 119-128, doi:10.1016/S1474-4422(09)70299-6 (2010). PMC2819840,
Jack, C. R., Jr. et al. Longitudinal tau PET in ageing and Alzheimer's disease. Brain 141, 1517-1528, doi:10.1093/brain/awy059 (2018). PMC5917767,
La Joie, R. et al. Prospective longitudinal atrophy in Alzheimer's disease correlates with the intensity and topography of baseline tau-PET. Sci Transl Med 12, doi:10.1126/scitranslmed.aau5732 (2020). PMC7035952,
Ossenkoppele, R. et al. Discriminative Accuracy of [18F] flortaucipir Positron Emission Tomography for Alzheimer Disease vs Other Neurodegenerative Disorders. JAMA 320, 1151-1162, doi:10.1001/jama.2018.12917 (2018). PMC6233630,
Palmqvist, S. et al. Cerebrospinal fluid and plasma biomarker trajectories with increasing amyloid deposition in Alzheimer's disease. EMBO Mol Med 11, e11170, doi:10.15252/emmm.201911170 (2019). PMC6895602,
Benzing, W. C., Brady, D. R., Mufson, E. J. & Armstrong, D. M. Evidence that transmitter-containing dystrophic neurites precede those containing paired helical filaments within senile plaques in the entorhinal cortex of nondemented elderly and Alzheimer's disease patients. Brain Res 619, 55-68, doi:10.1016/0006-8993(93)91595-j (1993).
Benzing, W. C., Ikonomovic, M. D., Brady, D. R., Mufson, E. J. & Armstrong, D. M. Evidence that transmitter-containing dystrophic neurites precede paired helical filament and Alz-50 formation within senile plaques in the amygdala of nondemented elderly and patients with Alzheimer's disease. J Comp Neurol 334, 176-191, doi:10.1002/cne.903340203 (1993).
Blazquez-Llorca, L. et al. High plasticity of axonal pathology in Alzheimer's disease mouse models. Acta Neuropathol Commun 5, 14, doi:10.1186/s40478-017-0415-y (2017). PMC5296955,
Brendza, R. P. et al. PDAPP; YFP double transgenic mice: a tool to study amyloid-beta associated changes in axonal, dendritic, and synaptic structures. J Comp Neurol 456, 375-383, doi:10.1002/cne.10536 (2003).
Kandalepas, P. C. et al. The Alzheimer's beta-secretase BACE1 localizes to normal presynaptic terminals and to dystrophic presynaptic terminals surrounding amyloid plaques. Acta Neuropathol 126, 329-352, doi:10.1007/s00401-013-1152-3 (2013). PMC3753469,
Woodhouse, A., Vickers, J. C., Adlard, P. A. & Dickson, T. C. Dystrophic neurites in TgCRND8 and Tg2576 mice mimic human pathological brain aging. Neurobiol Aging 30, 864-874, doi:10.1016/j.neurobiolaging.2007.09.003 (2009).
Su, J. H., Cummings, B. J. & Cotman, C. W. Identification and distribution of axonal dystrophic neurites in Alzheimer's disease. Brain Res 625, 228-237, doi:10.1016/0006-8993(93)91063-x (1993).
Leyns, C. E. G. et al. TREM2 function impedes tau seeding in neuritic plaques. Nat Neurosci 22, 1217-1222, doi:10.1038/s41593-019-0433-0 (2019). PMC6660358,
Ammendolia, D. A., Bement, W. M. & Brumell, J. H. Plasma membrane integrity: implications for health and disease. BMC Biol 19, 71, doi:10.1186/s12915-021-00972-y (2021). PMC8042475,
Horn, A. & Jaiswal, J. K. Cellular mechanisms and signals that coordinate plasma membrane repair. Cell Mol Life Sci 75, 3751-3770, doi:10.1007/s00018-018-2888-7 (2018). PMC6541445,
Cooper, S. T. & Head, S. I. Membrane Injury and Repair in the Muscular Dystrophies. Neuroscientist 21, 653-668, doi:10.1177/1073858414558336 (2015).
Croissant, C., Carmeille, R., Brevart, C. & Bouter, A. Annexins and Membrane Repair Dysfunctions in Muscular Dystrophies. Int J Mol Sci 22, doi:10.3390/ijms22105276 (2021). PMC8155887,
Quattrocelli, M. et al. Genetic modifiers of muscular dystrophy act on sarcolemmal resealing and recovery from injury. PLoS Genet 13, e1007070, doi:10.1371/journal.pgen.1007070 (2017). PMC5669489,
Eberhard, D. A., Brown, M. D. & VandenBerg, S. R. Alterations of annexin expression in pathological neuronal and glial reactions. Immunohistochemical localization of annexins I, II (p36 and p11 subunits), IV, and VI in the human hippocampus. Am J Pathol 145, 640-649 (1994). PMC1890341,
Sanchez-Ponce, D., DeFelipe, J., Garrido, J. J. & Munoz, A. In vitro maturation of the cisternal organelle in the hippocampal neuron's axon initial segment. Mol Cell Neurosci 48, 104-116, doi:10.1016/j.mcn.2011.06.010 (2011).
Yamatani, H., Kawasaki, T., Mita, S., Inagaki, N. & Hirata, T. Proteomics analysis of the temporal changes in axonal proteins during maturation. Dev Neurobiol 70, 523-537, doi:10.1002/dneu.20794 (2010).
Gao, P., Ye, L., Cheng, H. & Li, H. The Mechanistic Role of Bridging Integrator 1 (BIN1) in Alzheimer's Disease. Cell Mol Neurobiol 41, 1431-1440, doi:10.1007/s10571-020-00926-y (2021).
Tan, M. S., Yu, J. T. & Tan, L. Bridging integrator 1 (BIN1): form, function, and Alzheimer's disease. Trends Mol Med 19, 594-603, doi:10.1016/j.molmed.2013.06.004 (2013).
Braun, A. et al. EHD proteins associate with syndapin I and II and such interactions play a crucial role in endosomal recycling. Mol Biol Cell 16, 3642-3658, doi:10.1091/mbc.e05-01-0076 (2005). PMC1182304,
George, M. et al. Shared as well as distinct roles of EHD proteins revealed by biochemical and functional comparisons in mammalian cells and C. elegans. BMC Cell Biol 8, 3, doi:10.1186/1471-2121-8-3 (2007). PMC1793994,
Buggia-Prevot, V. et al. A function for EHD family proteins in unidirectional retrograde dendritic transport of BACE1 and Alzheimer's disease Abeta production. Cell Rep 5, 1552-1563, doi:10.1016/j.celrep.2013.12.006 (2013). PMC3932704,
Galvin, J. E., Palamand, D., Strider, J., Milone, M. & Pestronk, A. The muscle protein dysferlin accumulates in the Alzheimer brain. Acta Neuropathol 112, 665-671, doi:10.1007/s00401-006-0147-8 (2006). PMC1705477,
Dias, C. & Nylandsted, J. Plasma membrane integrity in health and disease: significance and therapeutic potential. Cell Discov 7, 4, doi:10.1038/s41421-020-00233-2 (2021). PMC7813858,
Demonbreun, A. R. et al. Enhanced Muscular Dystrophy from Loss of Dysferlin Is Accompanied by Impaired Annexin A6 Translocation after Sarcolemmal Disruption. Am J Pathol 186, 1610-1622, doi:10.1016/j.ajpath.2016.02.005 (2016). PMC4901136,
Demonbreun, A. R. et al. Recombinant annexin A6 promotes membrane repair and protects against muscle injury. J Clin Invest 129, 4657-4670, doi:10.1172/JC1128840 (2019). PMC6819108,
Demonbreun, A. R. et al. An actin-dependent annexin complex mediates plasma membrane repair in muscle. J Cell Biol 213, 705-718, doi:10.1083/jcb.201512022 (2016). PMC4915191,
Wang, Y. et al. TREM2 lipid sensing sustains the microglial response in an Alzheimer's disease model. Cell 160, 1061-1071, doi:10.1016/j.cell.2015.01.049 (2015). PMC4477963,
Chan, K. Y. et al. Engineered AAVs for efficient noninvasive gene delivery to the central and peripheral nervous systems. Nat Neurosci 20, 1172-1179, doi:10.1038/nn.4593 (2017). PMC5529245,
Condello, C., Yuan, P., Schain, A. & Grutzendler, J. Microglia constitute a barrier that prevents neurotoxic protofibrillar Abeta42 hotspots around plaques. Nat Commun 6, 6176, doi:10.1038/ncomms7176 (2015). PMC4311408,
Gowrishankar, S. et al. Massive accumulation of luminal protease-deficient axonal lysosomes at Alzheimer's disease amyloid plaques. Proc Natl Acad Sci USA 112, E3699-3708, doi:10.1073/pnas.1510329112 (2015). PMC4507205,
Grewal, T. et al. Annexin Animal Models-From Fundamental Principles to Translational Research. Int J Mol Sci 22, doi:10.3390/ijms22073439 (2021). PMC8037771,
Lizarbe, M. A., Barrasa, J. I., Olmo, N., Gavilanes, F. & Turnay, J. Annexin-phospholipid interactions. Functional implications. Int J Mol Sci 14, 2652-2683, doi:10.3390/ijms14022652 (2013). PMC3588008,
Park, J. C. et al. Annexin A1 restores Abeta1-42-induced blood-brain barrier disruption through the inhibition of RhoA-ROCK signaling pathway. Aging Cell 16, 149-161, doi:10.11111/acel.12530 (2017). PMC5242298,
Ho, M. et al. Disruption of muscle membrane and phenotype divergence in two novel mouse models of dysferlin deficiency. Hum Mol Genet 13, 1999-2010, doi:10.1093/hmg/ddh212 (2004).
Lambert, J. C. et al. Evidence of the association of BIN1 and PICALM with the A D risk in contrasting European populations. Neurobiol Aging 32, 756 e711-755, doi:10.1016/j.neurobiolaging.2010.11.022 (2011).
Seshadri, S. et al. Genome-wide analysis of genetic loci associated with Alzheimer disease. JAMA 303, 1832-1840, doi:10.1001/jama.2010.574 (2010). PMC2989531,
De Rossi, P. et al. Aberrant accrual of BIN1 near Alzheimer's disease amyloid deposits in transgenic models. Brain Pathol 29, 485-501, doi:10.1111/bpa.12687 (2019). PMC6542723,
De Rossi, P. et al. Predominant expression of Alzheimer's disease-associated BIN1 in mature oligodendrocytes and localization to white matter tracts. Molecular neurodegeneration 11, 59, doi:10.1186/s13024-016-0124-1 (2016). PMC4973113,
Swaggart, K. A. et al. Annexin A6 modifies muscular dystrophy by mediating sarcolemmal repair. Proc Natl Acad Sci USA 111, 6004-6009, doi:10.1073/pnas.1324242111 (2014). PMC4000833,
Creutz, C. E., Hira, J. K., Gee, V. E. & Eaton, J. M. Protection of the membrane permeability barrier by annexins. Biochemistry 51, 9966-9983, doi:10.1021/bi3013559 (2012).
Cheng, C. et al. Recombinant Annexin A2 Administration Improves Neurological Outcomes After Traumatic Brain Injury in Mice. Front Pharmacol 12, 708469, doi:10.3389/fphar.2021.708469 (2021). PMC8363504,
Bartolome, F. et al. Annexin A5 prevents amyloid-beta-induced toxicity in choroid plexus: implication for Alzheimer's disease. Sci Rep 10, 9391, doi:10.1038/s41598-020-66177-5 (2020). PMC7286910,
Ries, M. et al. Annexin A1 restores cerebrovascular integrity concomitant with reduced amyloid-beta and tau pathology. Brain 144, 1526-1541, doi:10.1093/brain/awab050 (2021). PMC8262982,
Hawkins, T. E., Roes, J., Rees, D., Monkhouse, J. & Moss, S. E. Immunological development and cardiovascular function are normal in annexin VI null mutant mice. Mol Cell Biol 19, 8028-8032, doi:10.1128/MCB.19.12.8028 (1999). PMC84887,
Raouf, R. et al. Inhibition of somatosensory mechanotransduction by annexin A6. Sci Signal 11, doi:10.1126/scisignal.aao2060 (2018). PMC6485395,
Demonbreun, A. R. et al. A conserved annexin A6-mediated membrane repair mechanism in muscle, heart, and nerve. JCIInsight 2022 Jul. 22; 7(14): el58107.
Oakley, H. et al. Intraneuronal beta-amyloid aggregates, neurodegeneration, and neuron loss in transgenic mice with five familial Alzheimer's disease mutations: potential factors in amyloid plaque formation. J Neurosci 26, 10129-10140 (2006).
Peters, F. et al. BACE1 inhibition more effectively suppresses initiation than progression of beta-amyloid pathology. Acta Neuropathol 135, 695-710, doi:10.1007/s00401-017-1804-9 (2018). PMC5904228,
Zhao, J. et al. Beta-site amyloid precursor protein cleaving enzyme 1 levels become elevated in neurons around amyloid plaques: implications for Alzheimer's disease pathogenesis. J Neurosci 27, 3639-3649 (2007).
Glover, C. P., Bienemann, A. S., Heywood, D. J., Cosgrave, A. S. & Uney, J. B. Adenoviral-mediated, high-level, cell-specific transgene expression: a SYN1-WPRE cassette mediates increased transgene expression with no loss of neuron specificity. Mol Ther 5, 509-516, doi:10.1006/mthe.2002.0588 (2002).
Condello, C., Schain, A. & Grutzendler, J. Multicolor time-stamp reveals the dynamics and toxicity of amyloid deposition. Sci Rep 1, 19, doi:10.1038/srep00019 (2011). PMC3216507,
Parhizkar, S. et al. Loss of TREM2 function increases amyloid seeding but reduces plaque-associated ApoE. Nat Neurosci 22, 191-204, doi:10.1038/s41593-018-0296-9 (2019). PMC6417433,
Mattsson-Carlgren, N. et al. Abeta deposition is associated with increases in soluble and phosphorylated tau that precede a positive Tau PET in Alzheimer's disease. Sci Adv 6, eaaz2387, doi:10.1126/sciadv.aaz2387 (2020). PMC7159908,
Martin, L. et al. Tau protein kinases: involvement in Alzheimer's disease. Ageing Res Rev 12, 289-309, doi:10.1016/j.arr.2012.06.003 (2013).
Yarza, R., Vela, S., Solas, M. & Ramirez, M. J. c-Jun N-terminal Kinase (JNK) Signaling as a Therapeutic Target for Alzheimer's Disease. Front Pharmacol 6, 321, doi:10.3389/fphar.2015.00321 (2015). PMC4709475,
Jost, M., Thiel, C., Weber, K. & Gerke, V. Mapping of three unique Ca(2+)-binding sites in human annexin II. Eur J Biochem 207, 923-930, doi:10.1111/j.1432-1033.1992.tb17125.x (1992).
McNeil, A. K., Rescher, U., Gerke, V. & McNeil, P. L. Requirement for annexin A1 in plasma membrane repair. J Biol Chem 281, 35202-35207, doi:10.1074/jbc.M606406200 (2006).
Dana, H. et al. Sensitive red protein calcium indicators for imaging neural activity. Elife 5, doi:10.7554/eLife.12727 (2016). PMC4846379,
Sadleir, K. R. & Vassar, R. Cdk5 protein inhibition and Abeta42 increase BACE1 protein level in primary neurons by a post-transcriptional mechanism: implications of CDK5 as a therapeutic target for Alzheimer disease. J Biol Chem 287, 7224-7235, doi:10.1074/jbc.M111.333914 (2012). PMC3293556,
Neuner, S. M., Heuer, S. E., Huentelman, M. J., O'Connell, K. M. S. & Kaczorowski, C. C. Harnessing Genetic Complexity to Enhance Translatability of Alzheimer's Disease Mouse Models: A Path toward Precision Medicine. Neuron 101, 399-411 e395, doi:10.1016/j.neuron.2018.11.040 (2019). PMC6886697,
Heuer, S. E. et al. Identifying the molecular systems that influence cognitive resilience to Alzheimer's disease in genetically diverse mice. Learn Mem 27, 355-371, doi:10.1101/lm.051839.120 (2020). PMC7433658,
Eimer, W. A. & Vassar, R. Neuron loss in the 5XFAD mouse model of Alzheimer's disease correlates with intraneuronal Abeta42 accumulation and Caspase-3 activation. Molecular neurodegeneration 8, 2, doi:10.1186/1750-1326-8-2 (2013). 3552866,
Gobeske, K. T. et al. BMP signaling mediates effects of exercise on hippocampal neurogenesis and cognition in mice. PLoS One 4, e7506, doi:10.1371/journal.pone.0007506 (2009). PMC2759555,
Sadleir, K. R., Popovic, J., Khatri, A. & Vassar, R. Oral nimodipine treatment has no effect on amyloid pathology or neuritic dystrophy in the 5XFAD mouse model of amyloidosis. PLoS One 17, e0263332, doi:10.1371/journal.pone.0263332 (2022). PMC8809624,
Sadleir, K. R. et al. Pregabalin Treatment does not Affect Amyloid Pathology in 5XFAD Mice. Curr Alzheimer Res 18, 283-297, doi:10.2174/1567205018666210713125333 (2021).
Oh, M. M., Oliveira, F. A., Waters, J. & Disterhoft, J. F. Altered calcium metabolism in aging CA1 hippocampal pyramidal neurons. J Neurosci 33, 7905-7911, doi:10.1523/JNEUROSCI.5457-12.2013 (2013). PMC3679661,
Sadleir, K. R., Eimer, W. A., Kaufman, R. J., Osten, P. & Vassar, R. Genetic inhibition of phosphorylation of the translation initiation factor elF2alpha does not block Abeta-dependent elevation of BACE1 and APP levels or reduce amyloid pathology in a mouse model of Alzheimer's disease. PLoS One 9, e101643, doi:10.1371/journal.pone.0101643 (2014). PMC4081565,
Levites, Y. et al. Intracranial adeno-associated virus-mediated delivery of anti-pan amyloid beta, amyloid beta40, and amyloid beta42 single-chain variable fragments attenuates plaque pathology in amyloid precursor protein mice. J Neurosci 26, 11923-11928, doi:10.1523/JNEUROSCI.2795-06.2006 (2006). PMC6674861,
Gratuze, M. et al. APOE Antibody Inhibits Abeta-Associated Tau Seeding and Spreading in a Mouse Model. Ann Neurol, doi:10.1002/ana.26351 (2022).
Gratuze, M. et al. Activated microglia mitigate Abeta-associated tau seeding and spreading. J Exp Med 218, doi:10.1084/jem.20210542 (2021). PMC8190588
Condello, C., Yuan, P. & Grutzendler, J. Microglia-Mediated Neuroprotection, TREM2, and Alzheimer's Disease: Evidence From Optical Imaging. Biol Psychiatry 83, 377-387, doi:10.1016/j.biopsych.2017.10.007 (2018). PMC5767550,
Clayton, K. et al. Plaque associated microglia hyper-secrete extracellular vesicles and accelerate tau propagation in a humanized APP mouse model. Molecular neurodegeneration 16, 18, doi:10.1186/s13024-021-00440-9 (2021). PMC7986521,
Dang, T. C. et al. Powerful Homeostatic Control of Oligodendroglial Lineage by PDGFRalpha in Adult Brain. Cell Rep 27, 1073-1089 e1075, doi:10.1016/j.celrep.2019.03.084 (2019).
Furuyama, K. et al. Diabetes relief in mice by glucose-sensing insulin-secreting human alpha-cells. Nature 567, 43-48, doi:10.1038/s41586-019-0942-8 (2019). PMC6624841,
Mastrella, G. et al. Targeting APLN/APLNR Improves Antiangiogenic Efficiency and Blunts Proinvasive Side Effects of VEGFA/VEGFR2 Blockade in Glioblastoma. Cancer Res 79, 2298-2313, doi:10.1158/0008-5472.CAN-18-0881 (2019).
Roy, E. R. et al. Concerted type I interferon signaling in microglia and neural cells promotes memory impairment associated with amyloid beta plaques. Immunity, doi:10.1016/j.immuni.2022.03.018 (2022).
Benitez, A., Riquelme, R., Del Campo, M., Araya, C. & Lara, H. E. Nerve Growth Factor: A Dual Activator of Noradrenergic and Cholinergic Systems of the Rat Ovary. Front Endocrinol (Lausanne) 12, 636600, doi:10.3389/fendo.2021.636600 (2021). PMC7947612,
Kawasaki, H. et al. Nerve growth factor (NGF) has an anti-tumor effects through perivascular innervation of neovessels in HT1080 fibrosarcoma and HepG2 hepatitis tumor in nude mice. J Pharmacol Sci 140, 1-7, doi:10.1016/j.jphs.2019.02.011 (2019).
Grathwohl, S. A. & Jucker, M. Replacement of osmotic minipumps to extend the intracerebral infusion time of compounds into the mouse brain. Biotechniques 55, 75-78, doi:10.2144/000114064 (2013).
Di Meco, A. et al. Poloxamer-188 Exacerbates Brain Amyloidosis, Presynaptic Dystrophies, and Pathogenic Microglial Activation in 5XFAD Mice. Curr Alzheimer Res, doi:10.2174/1567205019666220509143823 (2022).
Thorne, R. G., Pronk, G. J., Padmanabhan, V. & Frey, W. H., 2nd. Delivery of insulin-like growth factor-I to the rat brain and spinal cord along olfactory and trigeminal pathways following intranasal administration. Neuroscience 127, 481-496, doi:10.1016/j.neuroscience.2004.05.029 (2004).
De Rosa, R. et al. Intranasal administration of nerve growth factor (NGF) rescues recognition memory deficits in AD11 anti-NGF transgenic mice. Proc Natl Acad Sci USA 102, 3811-3816, doi:10.1073/pnas.0500195102 (2005). PMC553297,
Capsoni, S. et al. Intranasal “painless” human Nerve Growth Factor [corrected] slows amyloid neurodegeneration and prevents memory deficits in App X PS1 mice. PLoS One 7, e37555, doi:10.1371/journal.pone.0037555 (2012). PMC3364340,
Jiang, Y. et al. Intranasal brain-derived neurotrophic factor protects brain from ischemic insult via modulating local inflammation in rats. Neuroscience 172, 398-405, doi:10.1016/j.neuroscience.2010.10.054 (2011).
Vaka, S. R., Murthy, S. N., Balaji, A. & Repka, M. A. Delivery of brain-derived neurotrophic factor via nose-to-brain pathway. Pharm Res 29, 441-447, doi:10.1007/s11095-011-0572-9 (2012). PMC5650683,

What is claimed is:

1. A method of treating a patient suffering from a disorder comprising administering to the patient a therapeutically effective amount of a composition comprising an agent that increases the activity of an annexin protein, wherein the disorder is stroke, dementia, Alzheimer Dementia, Frontotemporal Dementia, Parkinsons Disease, spinal cord injury, small vessel disease, transient ischemic attack, cerebrovascular accident, dementia due to small vessel disease, Guillain Barré, Acute Inflammatory Demyelinating, Polyradiculopathy, Peripheral nerve disease, neuropathy, diabetic neuropathy, acute myocardial infarction, hypertrophic cardiomyopathy, dilated cardiomyopathy, myocarditis, arrhythmogenic cardiomyopathy, restrictive cardiomyopathy, ischemic cardiomyopathy, myocardiac injury acute, or myocardial injury.

2. A method of delaying onset, enhancing recovery from cellular membrane injury, or preventing a disorder comprising administering to a patient in need thereof a therapeutically effective amount of a composition comprising an agent that increases the activity of an annexin protein, wherein the disorder is stroke, dementia, Alzheimer Dementia, Frontotemporal Dementia, Parkinsons Disease, spinal cord injury, small vessel disease, transient ischemic attack, cerebrovascular accident, dementia due to small vessel disease, Guillain Barré, Acute Inflammatory Demyelinating, Polyradiculopathy, Peripheral nerve disease, neuropathy, diabetic neuropathy, acute myocardial infarction, hypertrophic cardiomyopathy, dilated cardiomyopathy, myocarditis, arrhythmogenic cardiomyopathy, restrictive cardiomyopathy, ischemic cardiomyopathy, myocardiac injury acute, or myocardial injury.

3. The method of claim 1 or claim 2, wherein the agent is a recombinant protein, a steroid, a polynucleotide capable of expressing an annexin protein, or a combination thereof.

4. The method of claim 3, wherein the steroid is a corticosteroid or a glucocorticoid.

5. The method of claim 3, wherein the recombinant protein is an annexin protein or a modified form thereof.

6. The method of claim 5, wherein the annexin protein is annexin A6 (SEQ ID NO: 7, SEQ ID NO: 8, SEQ ID NO: 37, or a combination thereof) or a modified form thereof.

7. The method of any one of claims 1-6, further comprising administering an effective amount of a second agent, wherein the second agent is mitsugumin 53 (MG53), a modulator of latent TGF-β binding protein 4 (LTBP4), a modulator of transforming growth factor β (TGF-β activity, a modulator of androgen response, a modulator of an inflammatory response, a promoter of muscle growth, a chemotherapeutic agent, a modulator of fibrosis, or a combination thereof.

8. The method of any one of claims 3-7, wherein the polynucleotide is associated with a nanoparticle.

9. The method of any one of claims 3-8, wherein the polynucleotide is contained in a vector.

10. The method of claim 9, wherein the vector is within a chloroplast.

11. The method of claim 9 wherein the vector is a viral vector.

12. The method of claim 11 wherein the viral vector is a herpes virus vector, an adeno-associated virus (AAV) vector, an adeno virus vector, or a lentiviral vector.

13. The method of claim 12 wherein the AAV vector is recombinant AAV5, AAV6, AAV8, AAV9, or AAV74.

14. The method of claim 13, wherein the AAV74 vector is AAVrh74.

15. The method of any one of claims 1-14, wherein the composition increases the activity of annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 37, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), annexin A13 (SEQ ID NO: 17 or SEQ ID NO: 18), or a combination thereof.

16. The method of claim 15, wherein the composition increases the activity of annexin A1 (SEQ ID NO: 1), annexin A2 (SEQ ID NO: 2 or SEQ ID NO: 3), and annexin A6 (SEQ ID NO: 7, SEQ ID NO: 8, SEQ ID NO: 37, or a combination thereof).

17. The method of claim 15, wherein the composition increases the activity of annexin A2 (SEQ ID NO: 2 or SEQ ID NO: 3) and annexin A6 (SEQ ID NO: 7, SEQ ID NO: 8, SEQ ID NO: 37, or a combination thereof).

18. The method of claim 15, wherein the composition increases the activity of annexin A1 (SEQ ID NO: 1) and annexin A6 (SEQ ID NO: 7, SEQ ID NO: 8, SEQ ID NO: 37, or a combination thereof).

19. The method of claim 15, wherein the composition increases the activity of annexin A6 (SEQ ID NO: 7, SEQ ID NO: 8, SEQ ID NO: 37, or a combination thereof).

20. The method of any one of claims 1-19, wherein the composition is the pharmaceutical composition of any one of claims 21-41.

21. A pharmaceutical composition comprising an annexin protein, or a modified form thereof, and a pharmaceutically acceptable carrier, buffer, and/or diluent.

22. The pharmaceutical composition of claim 21, wherein the annexin protein is annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 37, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), annexin A13 (SEQ ID NO: 17 or SEQ ID NO: 18), or a combination thereof.

23. The pharmaceutical composition of claim 21 or claim 22, wherein the annexin protein is annexin A6 (SEQ ID NO: 7, SEQ ID NO: 8, SEQ ID NO: 37, or a combination thereof).

24. The pharmaceutical composition of claim 21 or claim 22, wherein the pharmaceutical composition comprises annexin A1 (SEQ ID NO: 1), annexin A2 (SEQ ID NO: 2 or SEQ ID NO: 3), and annexin A6 (SEQ ID NO: 7, SEQ ID NO: 8, SEQ ID NO: 37, or a combination thereof).

25. The pharmaceutical composition of claim 21 or claim 22, wherein the pharmaceutical composition comprises annexin A2 (SEQ ID NO: 2 or SEQ ID NO: 3) and annexin A6 (SEQ ID NO: 7, SEQ ID NO: 8, SEQ ID NO: 37, or a combination thereof).

26. The pharmaceutical composition of claim 21 or claim 22, wherein the pharmaceutical composition comprises annexin A1 (SEQ ID NO: 1) and annexin A6 (SEQ ID NO: 7, SEQ ID NO: 8, SEQ ID NO: 37, or a combination thereof).

27. The pharmaceutical composition of any one of claims 21-26, further comprising a steroid.

28. The pharmaceutical composition of claim 27, wherein the steroid is a corticosteroid or a glucocorticoid.

29. The pharmaceutical composition of any one of claims 21-28, further comprising an effective amount of a second agent, wherein the second agent is mitsugumin 53 (MG53), a modulator of latent TGF-β binding protein 4 (LTBP4), a modulator of transforming growth factor β (TGF-β activity, a modulator of androgen response, a modulator of an inflammatory response, a promoter of muscle growth, a chemotherapeutic agent, a modulator of fibrosis, or a combination thereof.

30. The pharmaceutical composition of any one of claims 21-29, wherein purity of the annexin protein in the composition is about 90% or higher as measured by standard release assay.

31. The pharmaceutical composition of any one of claims 21-30, wherein the composition has an endotoxin level that is less than about 0.50000 endotoxin units per milligram (EU/mg).

32. A pharmaceutical composition comprising annexin A6 (SEQ ID NO: 7, SEQ ID NO: 8, SEQ ID NO: 37, or a combination thereof), or a modified form thereof, and a pharmaceutically acceptable carrier, buffer, and/or diluent.

33. The pharmaceutical composition of claim 32, further comprising annexin A1 (SEQ ID NO: 1), or a modified form thereof, and annexin A2 (SEQ ID NO: 2 or SEQ ID NO: 3), or a modified form thereof.

34. The pharmaceutical composition of claim 32, further comprising annexin A2 (SEQ ID NO: 2 or SEQ ID NO: 3), or a modified form thereof.

35. The pharmaceutical composition of claim 32, further comprising annexin A1 (SEQ ID NO: 1), or a modified form thereof.

36. The pharmaceutical composition of any one of claims 32-35, further comprising a steroid.

37. The pharmaceutical composition of claim 36, wherein the steroid is a corticosteroid or a glucocorticoid.

38. The pharmaceutical composition of any one of claims 32-37, further comprising an effective amount of a second agent, wherein the second agent is mitsugumin 53 (MG53), a modulator of latent TGF-β binding protein 4 (LTBP4), a modulator of transforming growth factor β (TGF-β activity, a modulator of androgen response, a modulator of an inflammatory response, a promoter of muscle growth, a chemotherapeutic agent, a modulator of fibrosis, or a combination thereof.

39. The pharmaceutical composition of any one of claims 32-38, wherein purity of the annexin protein in the composition is about 90% or higher as measured by standard release assay.

40. The pharmaceutical composition of any one of claims 32-39, wherein the composition has an endotoxin level that is less than about 0.50000 endotoxin units per milligram (EU/mg).

41. The pharmaceutical composition of any one of claims 32-40, wherein the annexin A6 (SEQ ID NO: 7, SEQ ID NO: 8, SEQ ID NO: 37, or a combination thereof), or a modified form thereof, is produced in a prokaryotic cell.

42. A method of treating a patient suffering from a nerve injury comprising administering to the patient a therapeutically effective amount of a composition comprising an agent that increases the activity of an annexin protein.

43. A method of delaying onset, enhancing recovery from a nerve injury, or preventing a nerve injury, comprising administering to a patient in need thereof a therapeutically effective amount of a composition comprising an agent that increases the activity of an annexin protein.

44. The method of claim 42 or claim 43, wherein the nerve injury is an acute nerve injury or a chronic nerve injury.

45. The method of any one of claims 42-44, wherein the nerve injury is a partially transected nerve, a wholly transected nerve, a nerve injury due to ischemia, a nerve injury due to infection, a nerve injury due to trauma, or a combination thereof.

46. The method of any one of claims 42-45, wherein the patient has a crush injury, a concussion, traumatic brain injury (TBI), or peripheral nerve disease.

47. A method comprising administering a therapeutically effective amount of a composition comprising an agent that increases the activity of an annexin protein to a patient, wherein the patient has an elevated serum or plasma level of lactate dehydrogenase (LDH), cardiac troponin T, cardiac troponin I, creatine kinase (CK), or a combination thereof, relative to a control level.

48. A method of reducing serum or plasma level of lactate dehydrogenase (LDH), cardiac troponin T, cardiac troponin I, creatine kinase (CK), or a combination thereof, in a patient in need thereof, comprising administering a therapeutically effective amount of a composition comprising an agent that increases the activity of an annexin protein to the patient, thereby reducing the serum or plasma level of lactate dehydrogenase (LDH), cardiac troponin T, cardiac troponin I, creatine kinase (CK), or a combination thereof in the patient.

49. The method of claim 47 or claim 48, wherein the serum or plasma level of LDH in the patient prior to administration of the agent is elevated about 1.25-fold or more over a normal control range.

50. The method of any one of claims 47-49, wherein the serum or plasma level of cardiac troponin T and/or cardiac troponin I in the patient prior to administration of the agent is elevated about 1.25-fold or more over a normal control range.

51. The method of any one of claims 47-50, wherein the serum or plasma level of CK in the patient prior to administration of the agent is elevated about 1.25-fold or more over a normal control range.

52. The method of any one of claims 47-51, wherein the serum or plasma level of LDH in the patient is reduced by about 25% or more 24-48 hours after administration of the agent.

53. The method of any one of claims 47-52, wherein the serum or plasma level of cardiac troponin T and/or cardiac troponin I in the patient is reduced by about 25% or more 24-48 hours after administration of the agent.

54. The method of any one of claims 47-53, wherein the serum or plasma level of CK in the patient is reduced by about 25% or more 24-48 hours after administration of the agent.

55. The method of any one of claims 42-54, wherein the agent is a recombinant protein, a steroid, a polynucleotide capable of expressing an annexin protein, or a combination thereof.

56. The method of claim 55, wherein the steroid is a corticosteroid or a glucocorticoid.

57. The method of claim 55, wherein the recombinant protein is an annexin protein.

58. The method of claim 57, wherein the annexin protein is annexin A6 (SEQ ID NO: 7, SEQ ID NO: 8, SEQ ID NO: 37, or a combination thereof) or a modified form thereof.

59. The method of any one of claims 47-58, wherein the patient suffers from an acute injury.

60. The method of claim 59, wherein the acute injury results from surgery, a burn, a toxin, a chemical, radiation-induced injury, acute myocardial injury, acute muscle injury, acute lung injury, acute epithelial injury, acute epidermal injury, acute kidney injury, acute liver injury, vascular injury, an excessive mechanical force, trauma, acute brain injury from stroke or trauma, myositis, or acute cardiac injury.

61. The method of any one of claims 47-58, wherein the patient suffers from a chronic disorder.

62. The method of claim 61, wherein the chronic disorder is Becker Muscular Dystrophy (BMD), Duchenne Muscular Dystrophy (DMD), Limb Girdle Muscular Dystrophy, Friedreich's Ataxia, congenital Muscular Dystrophy, Emery-Dreifuss Muscular Dystrophy (EDMD), Myotonic Dystrophy, Fascioscapulohumeral Dystrophy (FSHD), Oculopharyngeal Muscular Dystrophy, Distal Muscular Dystrophy, cystic fibrosis, pulmonary fibrosis, muscle atrophy, cerebral palsy, an epithelial disorder, an epidermal disorder, a kidney disorder, a liver disorder, sarcopenia, chronic cardiac injury, or cardiomyopathy (hypertrophic, dilated, congenital, arrhythmogenic, restrictive, ischemic, heart failure).

63. The method of claim 62, wherein the cardiomyopathy is hypertrophic, dilated, congenital, arrhythmogenic, restrictive, ischemic, Friedreich Ataxia, or heart failure.

64. The method of any one of claims 42-63, further comprising administering an effective amount of a second agent, wherein the second agent is mitsugumin 53 (MG53), a modulator of latent TGF-β binding protein 4 (LTBP4), a modulator of transforming growth factor β (TGF-β activity, a modulator of androgen response, a modulator of an inflammatory response, a promoter of muscle growth, a chemotherapeutic agent, a modulator of fibrosis, or a combination thereof.

65. The method of any one of claims 55-64, wherein the polynucleotide is associated with a nanoparticle.

66. The method of any one of claims 55-65, wherein the polynucleotide is contained in a vector.

67. The method of claim 66, wherein the vector is within a chloroplast.

68. The method of claim 66 wherein the vector is a viral vector.

69. The method of claim 68 wherein the viral vector is a herpes virus vector, an adeno-associated virus (AAV) vector, an adeno virus vector, or a lentiviral vector.

70. The method of claim 69 wherein the AAV vector is recombinant AAV5, AAV6, AAV8, AAV9, or AAV74.

71. The method of claim 70, wherein the AAV74 vector is AAVrh74.

72. The method of any one of claims 42-71, wherein the composition increases the activity of annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 37, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), annexin A13 (SEQ ID NO: 17 or SEQ ID NO: 18), or a combination thereof.

73. The method of claim 72, wherein the composition increases the activity of annexin A1 (SEQ ID NO: 1), annexin A2 (SEQ ID NO: 2 or SEQ ID NO: 3), and annexin A6 (SEQ ID NO: 7, SEQ ID NO: 8, SEQ ID NO: 37, or a combination thereof).

74. The method of claim 72, wherein the composition increases the activity of annexin A2 (SEQ ID NO: 2 or SEQ ID NO: 3) and annexin A6 (SEQ ID NO: 7, SEQ ID NO: 8, SEQ ID NO: 37, or a combination thereof).

75. The method of claim 72, wherein the composition increases the activity of annexin A1 (SEQ ID NO: 1) and annexin A6 (SEQ ID NO: 7, SEQ ID NO: 8, SEQ ID NO: 37, or a combination thereof).

76. The method of claim 72, wherein the composition increases the activity of annexin A6 (SEQ ID NO: 7, SEQ ID NO: 8, SEQ ID NO: 37, or a combination thereof).

77. The method of any one of claims 42-76, wherein the composition is the pharmaceutical composition of any one of claims 78-98.

78. A pharmaceutical composition comprising an annexin protein, or a modified form thereof, and a pharmaceutically acceptable carrier, buffer, and/or diluent.

79. The pharmaceutical composition of claim 78, wherein the annexin protein is annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 37, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), annexin A13 (SEQ ID NO: 17 or SEQ ID NO: 18), or a combination thereof.

80. The pharmaceutical composition of claim 78 or claim 79, wherein the annexin protein is annexin A6 (SEQ ID NO: 7, SEQ ID NO: 8, SEQ ID NO: 37, or a combination thereof).

81. The pharmaceutical composition of claim 78 or claim 79, wherein the pharmaceutical composition comprises annexin A1 (SEQ ID NO: 1), annexin A2 (SEQ ID NO: 2 or SEQ ID NO: 3), and annexin A6 (SEQ ID NO: 7, SEQ ID NO: 8, SEQ ID NO: 37, or a combination thereof).

82. The pharmaceutical composition of claim 78 or claim 79, wherein the pharmaceutical composition comprises annexin A2 (SEQ ID NO: 2 or SEQ ID NO: 3) and annexin A6 (SEQ ID NO: 7, SEQ ID NO: 8, SEQ ID NO: 37, or a combination thereof).

83. The pharmaceutical composition of claim 78 or claim 79, wherein the pharmaceutical composition comprises annexin A1 (SEQ ID NO: 1) and annexin A6 (SEQ ID NO: 7, SEQ ID NO: 8, SEQ ID NO: 37, or a combination thereof).

84. The pharmaceutical composition of any one of claims 78-83, further comprising a steroid.

85. The pharmaceutical composition of claim 84, wherein the steroid is a corticosteroid or a glucocorticoid.

86. The pharmaceutical composition of any one of claims 78-85, further comprising an effective amount of a second agent, wherein the second agent is mitsugumin 53 (MG53), a modulator of latent TGF-β binding protein 4 (LTBP4), a modulator of transforming growth factor β (TGF-β activity, a modulator of androgen response, a modulator of an inflammatory response, a promoter of muscle growth, a chemotherapeutic agent, a modulator of fibrosis, or a combination thereof.

87. The pharmaceutical composition of any one of claims 78-86, wherein purity of the annexin protein in the composition is about 90% or higher as measured by standard release assay.

88. The pharmaceutical composition of any one of claims 78-87, wherein the composition has an endotoxin level that is less than about 0.50000 endotoxin units per milligram (EU/mg).

89. A pharmaceutical composition comprising annexin A6 (SEQ ID NO: 7, SEQ ID NO: 8, SEQ ID NO: 37, or a combination thereof), or a modified form thereof, and a pharmaceutically acceptable carrier, buffer, and/or diluent.

90. The pharmaceutical composition of claim 89, further comprising annexin A1 (SEQ ID NO: 1), or a modified form thereof, and annexin A2 (SEQ ID NO: 2 or SEQ ID NO: 3), or a modified form thereof.

91. The pharmaceutical composition of claim 89, further comprising annexin A2 (SEQ ID NO: 2 or SEQ ID NO: 3), or a modified form thereof.

92. The pharmaceutical composition of claim 89, further comprising annexin A1 (SEQ ID NO: 1), or a modified form thereof.

93. The pharmaceutical composition of any one of claims 89-92, further comprising a steroid.

94. The pharmaceutical composition of claim 93, wherein the steroid is a corticosteroid or a glucocorticoid.

95. The pharmaceutical composition of any one of claims 89-94, further comprising an effective amount of a second agent, wherein the second agent is mitsugumin 53 (MG53), a modulator of latent TGF-β binding protein 4 (LTBP4), a modulator of transforming growth factor β (TGF-β activity, a modulator of androgen response, a modulator of an inflammatory response, a promoter of muscle growth, a chemotherapeutic agent, a modulator of fibrosis, or a combination thereof.

96. The pharmaceutical composition of any one of claims 89-95, wherein purity of the annexin protein in the composition is about 90% or higher as measured by standard release assay.

97. The pharmaceutical composition of any one of claims 89-96, wherein the composition has an endotoxin level that is less than about 0.50000 endotoxin units per milligram (EU/mg).

98. The pharmaceutical composition of any one of claims 89-97, wherein the annexin A6 (SEQ ID NO: 7, SEQ ID NO: 8, SEQ ID NO: 37, or a combination thereof), or a modified form thereof is produced in a prokaryotic cell.

99. A method of treating preclinical Alzheimer's disease or mild-to-moderate congnitive impairment comprising administering a therapeutically effective amount of a composition comprising an agent that increases the activity of an annexin protein to a patient in need thereof.

100. The method of claim 99, wherein the patient has a plasma level of Aβ₄₂ that is greater than zero and less than about 1000 picograms per milliliter (pg/ml).

101. The method of claim 99 or claim 100, wherein the patient has a cerebrospinal fluid (CSF) ratio of Aβ42/Aβ40 ratio that is less than about 0.07.

102. The method of any one of claims 99-101, wherein the patient has a serum or plasma level of phosphorylated tau protein that is about 24 picograms per milliliter (pg/ml) or greater.

103. The method of any one of claims 99-102, wherein the patient has an amount of phosphorylated tau protein in their cerebrospinal fluid (CSF) that is about 52 picograms per milliliter (pg/ml) or greater.

104. The method of any one of claims 99-103, wherein the patient has an amount of amyloid plaques in their brain that is about 10 to about 60 centiloids.

105. The method of any one of claims 99-104, wherein the patient was previously diagnosed with preclinical Alzheimer's disease or mild-to-moderate cognitive impairment via cognitive testing.

106. The method of any one of claims 99-104, wherein the method includes diagnosing the patient with preclinical Alzheimer's disease or mild-to-moderate cognitive impairment via cognitive testing.

107. The method of any one of claims 99-106, wherein the patient is amyloid positive, tau negative, and neurodegeneration negative.

108. The method of any one of claims 99-106, wherein the patient is amyloid positive, tau positive, and neurodegeneration negative.

109. The method of any one of claims 99-106, wherein the patient is amyloid positive, tau negative, and neurodegeneration positive.

110. The method of any one of claims 99-106, wherein the patient is amyloid positive, tau positive, and neurodegeneration positive.

111. A method comprising administering a therapeutically effective amount of a composition comprising an agent that increases the activity of an annexin protein to a patient having preclinical Alzheimer's disease or mild-to-moderate cognitive impairment.

112. The method of claim 111, wherein the patient has:
(i) a cerebrospinal fluid (CSF) ratio of Aβ42/Aβ40 ratio that is less than about 0.07;
(ii) a plasma level of Aβ42 that is greater than zero and less than about 1000 pg/ml;
(iii) a serum or plasma level of phosphorylated tau protein that is about 24 pg/ml or greater;
(iv) an amount of phosphorylated tau protein in their cerebrospinal fluid (CSF) that is about 52 pg/ml or greater; and/or
(v) an amount of amyloid plaques in their brain that is about 10 to about 60 centiloids.

113. The method of claim 111 or claim 112, wherein the patient was previously diagnosed with preclinical Alzheimer's disease or mild-to-moderate cognitive impairment via cognitive testing.

114. The method of claim 111 or claim 112, wherein the method includes diagnosing the patient with preclinical Alzheimer's disease or mild-to-moderate cognitive impairment via cognitive testing.

115. The method of any one of claims 99-114, wherein the phosphorylated tau protein is p-tau181, p-tau231, p-tau217, or a combination thereof.

116. The method of claim 115, wherein the phosphorylated tau protein is p-tau181.

117. The method of any one of claims 99-116, wherein the agent is a recombinant protein, a steroid, a polynucleotide capable of expressing an annexin protein, or a combination thereof.

118. The method of claim 117, wherein the steroid is a corticosteroid or a glucocorticoid.

119. The method of claim 117, wherein the recombinant protein is an annexin protein or a modified form thereof.

120. The method of claim 119, wherein the annexin protein is annexin A6 (SEQ ID NO: 7, SEQ ID NO: 8, SEQ ID NO: 37, or a combination thereof) or a modified form thereof.

121. The method of any one of claims 99-120, further comprising administering an effective amount of a second agent, wherein the second agent is an acetylcholinesterase inhibitor, a mild to moderate NMDA-receptor antagonist, an anti-amyloid antibody, a beta-secretase enzyme inhibitor, an anti-tau antibody, a modulator of microglial activity, mitsugumin 53 (MG53), a modulator of latent TGF-β binding protein 4 (LTBP4), a modulator of transforming growth factor β (TGF-β activity, a modulator of androgen response, a modulator of an inflammatory response, a chemotherapeutic agent, or a combination thereof.

122. The method of any one of claims 117-121, wherein the polynucleotide is associated with a nanoparticle.

123. The method of any one of claims 117-122, wherein the polynucleotide is contained in a vector.

124. The method of claim 123, wherein the vector is within a chloroplast.

125. The method of claim 123 wherein the vector is a viral vector.

126. The method of claim 125 wherein the viral vector is a herpes virus vector, an adeno-associated virus (AAV) vector, an adeno virus vector, or a lentiviral vector.

127. The method of claim 126 wherein the AAV vector is recombinant AAV1, AAV2, AAV4, AAV5, AAV7, AAV8, AAV9, AAVrh10, AAV PHP.B, or AAV PDP.eB.

128. The method of any one of claims 99-127, wherein the composition increases the activity of annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 37, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), annexin A13 (SEQ ID NO: 17 or SEQ ID NO: 18), or a combination thereof.

129. The method of claim 128, wherein the composition increases the activity of annexin A1 (SEQ ID NO: 1), annexin A2 (SEQ ID NO: 2 or SEQ ID NO: 3), and annexin A6 (SEQ ID NO: 7, SEQ ID NO: 8, SEQ ID NO: 37, or a combination thereof).

130. The method of claim 128, wherein the composition increases the activity of annexin A2 (SEQ ID NO: 2 or SEQ ID NO: 3) and annexin A6 (SEQ ID NO: 7, SEQ ID NO: 8, SEQ ID NO: 37, or a combination thereof).

131. The method of claim 128, wherein the composition increases the activity of annexin A1 (SEQ ID NO: 1) and annexin A6 (SEQ ID NO: 7, SEQ ID NO: 8, SEQ ID NO: 37, or a combination thereof).

132. The method of claim 128, wherein the composition increases the activity of annexin A6 (SEQ ID NO: 7, SEQ ID NO: 8, SEQ ID NO: 37, or a combination thereof).

133. The method of any one of claims 99-132, wherein the composition is the pharmaceutical composition of any one of claims 78-98.
